(19) United States
(12) Patent Application Publication
(10) Pub. No.: US 2023/0366885 A1
(43) Pub. Date: Nov. 16, 2023

(54) DNA CONSTRUCTS, RECOMBINANT CELLS COMPRISING THEREOF, BACTERIAL PROBES, METHODS FOR THEIR PREPARATION, AND METHOD OF USING THEREOF

(71) Applicant: YEDA RESEARCH AND DEVELOPMENT CO. LTD., Rehovot (IL)

(72) Inventors: David MARGULIES, Rehovot (IL); Leila Motiei, Rehovot (IL); Naama Lahav, Rehovot (IL); Pragati Prasad, Rehovot (IL)

(73) Assignee: YEDA RESEARCH AND DEVELOPMENT CO. LTD., Rehovot (IL)

(21) Appl. No.: 18/350,066

(22) Filed: Jul. 11, 2023

Related U.S. Application Data
(63) Continuation-in-part of application No. 17/614,563, filed on Nov. 29, 2021.
(60) Provisional application No. 63/388,251, filed on Jul. 12, 2022.

(51) Int. Cl. G01N 33/58; G01N 33/574

(52) U.S. Cl. CPC G01N 33/582; G01N 33/574

(57) ABSTRACT

The disclosure presented herein provides DNA constructs, recombinant cells comprising thereof, system comprising thereof, bacterial probes, and/or a recombinant cell decorated with various labels and/or synthetic agents, wherein said labels and/or synthetic agents can be reversibly modified or removed from the cells. Also disclosed herein are methods for decorating and/or modifying cells, preferably bacteria cells, and methods for using thereof.

### CROSS-REFERENCE TO RELATED APPLICATIONS

This application is a Continuation-In-Part of U.S. patent application Ser. No. 17/614,563, which is a National Phase application of PCT International Application No. PCT/IL2019/050639, International Filing Date Jun. 5, 2019, both are hereby incorporated by reference in their entirely.

This application claims priority from U.S. Provisional Patent Application No. 63/388,251, filed Jul. 12, 2022, which is hereby incorporated by reference, in its entirely.

### SEQUENCE LISTING STATEMENT

The instant application contains a Sequence Listing which has been submitted electronically in XML file format and is hereby incorporated by reference in its entirety. Said XML copy, created on Jun. 27, 2023, is named P-587086-US1_ST26.xml and is 42,242 bytes in size.

### FIELD OF THE INVENTION

The disclosure presented herein provides DNA constructs, recombinant cells comprising thereof, system comprising thereof, bacterial probes, and/or a recombinant cell decorated with various labels and/or synthetic agents, wherein said labels and/or synthetic agents can be reversibly modified or removed from the cells. Also disclosed herein are methods for decorating and/or modifying cells, preferably bacteria cells, and methods for using thereof.

### BACKGROUND

Fluorescence labeling is one of the most powerful analytical tools used to study protein expression and localization in intact cells. A common method to label the proteins of interest (POIs) in a cellular environment is by fusing them to fluorescent proteins (FPs) (FIG. 8A). However, because this approach requires genetic engineering, it is not suitable for various medical diagnostic applications, which involve the classification of cells (e.g., cancer cells) according to the expression of specific cell surface proteins (CSPs). To detect, image, and classify CSPs of native (non-engineered) cells, immunofluorescence (IF) is commonly used (FIG. 8B). IF, however, is generally costly and laborious because it involves the manufacturing of non-homogeneous populations of fluorescently labeled monoclonal antibodies (Abs), as well as target identification via sequential incubation steps that frequently involve the use of both primary and secondary Abs (FIG. 8B). Moreover, because fluorescent Abs do not generally interact with small-molecule binding sites, they cannot be used to track the interaction between CSPs and small-molecule agonists or antagonists. An alternative approach to fluorescently label CSPs of non-engineered cells is by using molecular probes consisting of a fluorophore conjugated to a small-molecule- or a peptide-based ligand (FIG. 8C). Although this approach enabled the creation of homogeneous populations of structurally identical probes, which can be used to label and detect a wide range of POIs, image CSPs in living cell, or perform live cell-based screening of CSP inhibitors, this labeling method has some limitations when compared to IF. One limitation is the difficulty to maintain the high binding affinity of a small-molecule ligand toward the POI following its conjugation to a fluorescent dye. This decrease in affinity often requires using a large excess of probes, which can lead to the generation of a strong background signal. Because removal of an excess of unbound probes by washing could lead to the dissociation of the probe-POI complex, efficient CSP labeling frequently requires developing unique sets of fluorescent molecular probes, for example, probes that covalently bind to their targets or that can generate a ‘turn-on’ emission signal upon binding. Therefore, unlike fluorescent Abs, which can be used to label various targets with a wide range of fluorescent dyes, obtaining synthetic probes that can label CSPs in living cells with high binding affinity, target versatility, and color variability remains challenging. The difficulty to obtain efficient CSP binding probes complicates using them in multiplexed protein detection, as well as complicates enhancing the intensity of their fluorescence signal by replacing the fluorescent reporter or by integrating several fluorophores into a single synthetic probe.

One way to enhance the binding affinity of synthetic agents to their biological targets is to utilize the multivalency effect. With this strategy, multiple synthetic ligands are attached to a single scaffold to afford multivalent CSP binders that exhibit binding cooperativity and consequently, an increased affinity. In this context, it has been recently shown that engineered bacteria decorated with a folate-bearing DNA duplex can bind to KB cancer cells overexpressing the folate receptor. One advantage of synthetic protein binders generated from modified oligodeoxynucleotides (ODNs), over Abs (FIG. 8B) or binders based on unimolecular synthetic ligands (FIG. 8C), is the structural modularity of synthetic ODNs and their ability to self-assemble into well-defined constructs. Therefore, it was hypothesized that by systematically modifying the structures of the modified DNA duplexes, it might be possible to diversify, optimize, and generalize the properties of chemically modified bacterial probes. Such steps may enable the development of a wide range of fluorescent bacterial probes, termed herein B-probes, which can complement the current CSP labeling tools that mainly rely on Abs or small-molecule-based ligands (FIG. 8).

In recent years, considerable attention has been devoted to developing oligodeoxynucleotide (ODN)-small molecule conjugates that, similar to CSPs, can respond to external stimuli and undergo dynamic structural changes that enables them to reversibly interact with proteins and mediate their functions. Similarly, cell surfaces were modified by attaching synthetic agents to them.

It is clear that there remains a critical need for systems capable of decorating or coating cell membranes with proteins of interest. Ideally, such a system would allow easy proteins attachment to the cell membrane, and would allow controlling the structure, composition, binding interactions, and concentrations of said proteins on the cell membrane. Further, an ideal system would allow controlling and reversing such parameters by easily administering extremal chemical signals. Such a system would provide a great level of control over cellular processes in vitro or in the living organism.

### SUMMARY OF THE INVENTION

In some embodiments, this invention is directed to a DNA construct comprising:
a. a first compound comprising a first oligonucleotide (ODN-1) covalently bound to a His-tag specific binder, either directly or through a first linker;
b. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and
c. a third compound comprising a DNA duplex (dsDNA) appended with a second hanging strand complementary to said first hanging strand, and further appended with at least two fluorescent dyes.

In some embodiments, the first compound is bound to the second compound through hybridization of ODN-1 and ODN-2. In some embodiments, the third compound is bound to the second compound through hybridization of the first hanging strand and the second hanging strand. In some embodiments, the first and/or the second hanging strand comprises between 5-50 oligonucleotides. In some embodiments, the first and/or the second hanging strand comprises between 10-20 oligonucleotides (e.g., 16). In some embodiments, the ODN-1 is 5-100 bases long. In some embodiments, ODN-1 is 5-25 bases long. In some embodiments, the second oligonucleotide (ODN-2) is longer than said first oligonucleotide (ODN-1).

In some embodiments, the His-tag specific binder is capable of binding to an affinity tag comprising a poly-histidine peptide. In some embodiments, the His-tag specific binder comprises a moiety represented by the structure of formula E, formula E(a) or formula E(b) as described hereinbelow; each represents a separate embodiment according to this invention.

In some embodiments, the first linker comprises at least one oligoethyleneglycol (OEG) moiety. In some embodiments, the first linker comprises at least one phosphate moiety. In some embodiments, the first linker comprises at least one thioalkyl moiety. In some embodiments, the first linker comprises any combination of oligoethyleneglycol (OEG) moieties, phosphate moieties, and/or thioalkyl moieties.

In some embodiments, the first linker comprises the following monomer: —[(CH₂O)ₖ—PO₃H]ₗ—. In some embodiments, the first linker is represented by the following formula: —[(CH₂O)ₖ—PO₃H]ₗ—(CH₂)_{w}—S, wherein k and l are as described hereinbelow.

In some embodiments, the synthetic agent of said second compound is bound to the 3′ end or to the 5′ end of said second oligonucleotide. In some embodiments, the synthetic agent of said second compound is a chemical or a biological moiety. In some embodiments, the synthetic agent of said second compound is naturally occurring or a synthetic compound. In some embodiments, the synthetic agent of said second compound comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent of said second compound comprises a CSP binder, a cancer cell binder, or a protein binder; each represents a separate embodiment according to this invention. In some embodiments, the synthetic agent of said second compound can interact with a specific CSP on a cancer cell.

In some embodiments, the CSP binder, a cancer cell binder, or a protein binder comprises a biotin, a folate, an anisamide, or a glutamate urea; each represents a separate embodiment according to this invention. In some embodiments, the CSP of said CSP binder is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof; each represents a separate embodiment according to this invention. In some embodiments, the cancer cell of said cancer cell binder, is KB cell (cervical cancer cell), MDA-MB-435 (melanoma cell), or LNCaP (prostate cancer cell); each represents a separate embodiment according to this invention.

In some embodiments, the DNA duplex (dsDNA) comprises at least 4 fluorescent dyes. In some embodiments, the fluorescent dyes of said DNA duplex (dsDNA) are located 4-6 bases apart from each other. In some embodiments, the fluorescent dyes of said DNA duplex (dsDNA) are located on the longer oligonucleotide strand that comprises the second hanging strand (ssDNA-long). In some embodiments, the ssDNA-long comprises between 10-50 oligonucleotides including the second hanging strand (e.g., 45). In some embodiments, the DNA duplex (dsDNA) comprises a longer oligonucleotide strand (ssDNA-long) represented by a sequence comprising at least 80% homology to SEQ ID NO. 22 or 23. In some embodiments, the DNA duplex (dsDNA) comprises a shorter oligonucleotide strand (ssDNA-short) represented by a sequence comprising at least 80% homology to SEQ ID NO.: 24.

In some embodiments, the fluorescent dyes are selected from: dansyl, fluorescein (6-FAM), FAM, cyanine dyes (e.g. Cy3, Cy5, Cy7, etc), sulfoindocyanine, nile red, Rhodamine dyes (e.g., Rhodamine 123, Rhodamine Red-X, etc.), perylene, fluorenyl, coumarin, 7-methoxycoumarin (Mca), dabcyl, NBD, Nile blue, TAMRA, BODIPY dyes, FITC (Fluorescein isothiocyanate), Thiazole orange, Quinoline blue, Thiazole red, phycoerythrin (PE), Acridine Orange, Alexa Fluor dyes (e.g., Alexa Fluor 488, Alexa Fluor 555, Alexa Fluor 647, etc.), Cascade Blue, DAPI (4′,6-diamidino-2-phenylindole), DiI (1,1′-Dioctadecyl-3,3,3′,3′-tetramethylindocarbocyanine perchlorate), Ethidium Bromide, GFP (Green Fluorescent Protein), Hoechst dyes (e.g., Hoechst 33342, Hoechst 33258, etc.), Indo-1, Lucifer Yellow, MitoTracker dyes (e.g., MitoTracker Green, MitoTracker Red, etc.), Oregon Green, Propidium Iodide, SYBR Green, Texas Red, YOYO-1, ZsGreen or derivative thereof; each represents a separate embodiment according to this invention.

In some embodiments, the first compound is represented by the structure of the nickel complexes of compound 103-106 as described hereinbelow; each represents a separate embodiment according to this invention.

In some embodiments, the second compound is represented by the structure of compound 205-214, 220, 230 and 240 as described hereinbelow; each represents a separate embodiment according to this invention.

In some embodiments, the construct comprises compound 105. In some embodiments, the construct comprises compound 208, as described hereinbelow. In some embodiments, the construct comprises ssDNA-long or ssDNA-long′, as described hereinbelow. In some embodiments, the construct comprises ssDNA-short, as described hereinbelow. In some embodiments, the construct comprises compound 105, compound 208, ssDNA-long or ssDNA-long′, and ssDNA-short, as described hereinbelow.

In some embodiments, this invention is directed to a system comprising:
a. a recombinant cell ectopically expressing a polypeptide, wherein said polypeptide comprises a membranal anchoring domain and an extracellular binding domain; and
b. a DNA construct according to this invention.

In some embodiments, the first compound of said DNA construct is bound to the second compound through hybridization of ODN-1 and ODN-2. In some embodiments, the third compound of said DNA construct is bound to the second compound through hybridization of the first hanging strand and the second hanging strand. In some embodiments, the His-tag specific binder of said DNA construct, comprises affinity to said extracellular binding domain of said polypeptide. In some embodiments, the DNA construct is bound to said recombinant cell in the presence of Ni²⁺ ions.

In some embodiments, the system does not perturb said cell's function. In some embodiments, the system can be reversibly modified. In some embodiments, the recombinant cell is a bacteria. In some embodiments, the bacteria is a His-OmpC expressing bacteria. In some embodiments, the polypeptide is a cell surface protein (CSP) comprising a histidine tag (e.g., His-OmpC). In some embodiments, the membranal anchoring domain of said polypeptide comprises a transmembranal protein or a part of it, an artificial polypeptide, or a combination thereof, each represents a separate embodiment according to this invention. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the extracellular binding domain of said polypeptide comprises a poly-histidine tag.

In some embodiments, the system further comprises a fourth compound comprising a third oligonucleotide (ODN-3) complementary to said ODN-2. In some embodiments, ODN-3 comprises higher affinity to said ODN-2 than the affinity of said ODN-2 to said ODN-1.

In some embodiments, this invention is directed to a recombinant cell bound to a DNA construct according to this invention, wherein the recombinant cell is ectopically expressing a polypeptide, which comprises a membranal anchoring domain and an extracellular binding domain, wherein the extracellular binding domain comprises a poly-histidine affinity tag, which is bound to said DNA construct, via the binding of the His-tag specific binder to the poly histidine affinity tag of the polypeptide, in the presence of Ni²⁺ ions.

In some embodiments, the cell is a bacteria. In some embodiments, the bacteria is a His-OmpC expressing bacteria.

In some embodiments, the polypeptide is a cell surface protein (CSP). In some embodiments, the polypeptide comprises an outer membrane protein C (OmpC); receptor tyrosine kinases (RTKs); Ion channel linked receptors; Enzyme-linked receptors; G protein-coupled receptors or any combination thereof. In some embodiments, the membranal anchoring domain of said polypeptide comprises a transmembranal protein or a part of it, an artificial polypeptide, or a combination thereof, each represents a separate embodiment according to this invention. In some embodiments, the cell surface protein (CSP) is a histidine tagged outer membrane protein C (His-OmpC).

In some embodiments, this invention is directed to a method for labeling a cancer cell, said method comprises incubating the recombinant cell bound to the DNA construct according to this invention, with a cancer cell, wherein said cancer cell comprises a CSP, and the synthetic agent of said DNA construct of said recombinant cell, is a CSP binder, which comprises binding affinity to said CSP.

In some embodiments, the labeling is carried out in a cellular environment. In some embodiments, the CSP binder targets a small-molecule binding site in the cancer cell CSP. In some embodiments, the CSP binder comprises a biotin, a folate, an anisamide, a glutamate urea, or derivative thereof, each represents a separate embodiment according to this invention. In some embodiments, the CSP is overexpressed or selectively expressed in said cancer cell. In some embodiments, the recombinant cell is a native cell, a living cell or an engineered cell; each represents a separate embodiment according to this invention. In some embodiments, the recombinant cell is a bacteria. In some embodiments, the interaction between the recombinant cell and the cancer cell is multivalent. In some embodiments, the cancer cell is KB cell (cervical cancer cell), MDA-MB-435 (melanoma cell), or LNCaP (prostate cancer cell); each represents a separate embodiment according to this invention.

In some embodiments, this invention is directed to a method for binding a first cell to a second cell, said method comprises incubating a recombinant cell according to this invention (a first cell) with a second cell, wherein the second cell comprises a CSP, and said synthetic agent of said DNA construct of said first cell, comprises a CSP binder, which comprises a binding affinity to said CSP.

In some embodiments, the first cell is a native cell, a living cell or an engineered cell; each represents a separate embodiment according to this invention. In some embodiments, the first cell is a bacteria. In some embodiments, the second cell is a cancer cell. In some embodiments, the cancer cell is KB cell (cervical cancer cell), MDA-MB-435 (melanoma cell), or LNCaP (prostate cancer cell); each represents a separate embodiment according to this invention. In some embodiments, the CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof, each represents a separate embodiment according to this invention. In some embodiments, the CSP is selectively expressed or overexpressed in said second cell. In some embodiments, the CSP binder comprises a biotin, a folate, an anisamide, a glutamate urea, an antibody or derivative thereof, each represents a separate embodiment according to this invention. In some embodiments, the method is taking place in a cellular environment. In some embodiments, the interaction between the first cell and the second cell is multivalent.

In some embodiments, this invention is directed to a method for binding a cell to a protein of interest (POI), said method comprises incubating a sample comprising a POI with the recombinant cell according to this invention, wherein said synthetic agent of said DNA construct is a protein binder, which comprises binding affinity to said POI.

In some embodiments, the method is taking place in a cellular environment. In some embodiments, the POI is a cell surface protein (CSP). In some embodiments, the CSP is a polypeptide or a protein, which is overexpressed or selectively expressed on the surface of a cell. In some embodiments, the cell is a cancer cell. In some embodiments, the protein binder is selective to said POI. In some embodiments, the protein binder is a cell surface protein (CSP) binder, a small molecule ligand, an antibody, a peptide, a polypeptide, a protein or a part thereof, each represents a separate embodiment according to this invention.

In some embodiments, this invention is directed to a method for detecting and/or labeling a protein of interest (POI) in a cellular environment, said method comprises:
a. imaging a sample comprising a protein of interest (POI) in cellular environment;
b. incubating the sample of (a) with a recombinant cell according to this invention as described hereinbelow, wherein said synthetic agent of said DNA construct of said cell is a protein binder, which comprises affinity to said POI;
c. washing the sample of (b) from excess of said cell; and
d. imaging the fluorescence of said sample;
wherein increase in the fluorescence signal is indicative of the presence of said POI in said cellular environment, thereby detecting and/or labeling said protein of interest (POI) in said cellular environment.

In some embodiments, the cellular environment comprises living cells. In some embodiments, the POI is a cell surface protein (CSP). In some embodiments, the CSP is a polypeptide or a protein, which is overexpressed or selectively expressed on the surface of a cell. In some embodiments, the cell is a cancer cell. In some embodiments, the CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof, each represents a separate embodiment according to this invention. In some embodiments, the protein binder is selective to said POI. In some embodiments, the fluorescence signal is measured by a fluorescence microscope or by recording the emission with a spectrophotometer at a particular wavelength.

In some embodiments, this invention is directed to a method for measuring the interaction between a protein of interest (POI) and a potential ligand for said POI, said method comprises
a. imaging a sample comprising a protein of interest (POI);
b. incubating the sample comprising a POI with a recombinant cell according tot his inventio, and with a potential ligand for said POI, wherein said synthetic agent of said DNA construct of said cell comprises affinity to said POI;
c. washing the sample from excess of said cell and ligand;
d. measuring the fluorescence imaging of said sample;
e. comparing the measured fluorescence of the sample of (d) with the fluorescence measured from incubating a control sample comprising a protein of interest (POI) with the cell according to this invention followed by washing excess of said cell (i.e. a control);
wherein reduction in the fluorescence signal with respect to the control is indicative of the interaction between said POI and said potential ligand.

In some embodiments, the POI is a cell surface protein (CSP). In some embodiments, the potential ligand is a protein binder, a peptide, small molecule, modulator, agonist, antagonist, or any combination thereof, each represents a separate embodiment according to this invention. In some embodiments, the synthetic agent is a protein binder. In some embodiments, the protein binder is selective to said POI. In some embodiments, the potential ligand is added after, before or concurrently with said cell in step (b); each represents a separate embodiment according to this invention. In some embodiments, the fluorescence signal is measured by a fluorescence microscope or by recording the emission with a spectrophotometer at a particular wavelength.

In some embodiments, this invention is directed to a method for cell-based screening for potential ligands for a protein of interest (POI), said method comprises:
a. imaging a sample comprising a protein of interest (POI);
b. incubating the sample comprising a POI with a recombinant cell according to this inventio as described hereinbelow, and with a potential ligand, wherein said synthetic agent of said DNA construct of said cell comprises affinity to said POI;
c. washing the sample from excess of said cell and ligand;
d. measuring the fluorescence imaging of said sample;
e. comparing the measured fluorescence of the sample of (d) with the fluorescence measured from incubating a sample comprising a protein of interest (POI) with a recombinant cell according to this invention as described hereinbelow, followed by washing excess of said cell (i.e. a control);
wherein reduction in the fluorescence signal with respect to the control is indicative of the interaction between said POI and said potential ligand thereby screening for potential ligands for said POI.

In some embodiments, the cell-based screening is performed in living cells. In some embodiments, the potential ligand is a protein binder, a peptide, small molecule, modulator, agonist or antagonist; each represents a separate embodiment according to this invention. In some embodiments, the synthetic agent is a protein binder, a drug, or a small molecule; each represents a separate embodiment according to this invention. In some embodiments, the protein binder, drug or small molecule is selective to said POI. In some embodiments, the potential ligand is added after, before or concurrently with said cell in step (b); each represents a separate embodiment according to this invention. In some embodiments, the fluorescence signal is measured by a fluorescence microscope or by recording the emission with a spectrophotometer at a particular wavelength.

### BRIEF DESCRIPTION OF THE DRAWINGS

The patent of application file contains at least one drawing executed in color. Copies of this patent or patent application publication with color drawings will be provided by the office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1B show the design of an artificial receptors system. FIG. 1A shows an embodiment to decorate E. coli with artificial receptors appended with a specific functionality (X). A first molecule X-ODN-1 binds a hexa-histidine tag (His-tag) fused to recombinant OmpC. Recombinant OmpC is inserted into the cell membrane. Reversibility of this process is achieved by subjecting the bacteria to EDTA. A further way to introduce an unnatural recognition motif (Y) to the bacterial surface is adding to the bacteria-bound ODN-1 an ODN-1 complementary strand modified with the desired functionality (Y-ODN-2). Y-ODN-2 can be selectively removed by adding a complementary strand, ODN-3. FIG. 1B shows the structure of X-ODN-1.

FIGS. 2A-2E shows reversible, non-covalent modification of bacterial membrane with a synthetic receptor. FIG. 2A shows fluorescence images of: (i) E. coli expressing His-OmpC incubated with 500 nM of Compound 100 and Ni (II), (ii) Native bacteria (that lack His-tag) incubated with 500 nM of Compound 100 and Ni (II), (iii) E. coli expressing His-OmpC incubated with 500 nM of Compound 100 in the absence of Ni (II), and (iv) E. coli expressing His-OmpC incubated with 500 nM of Cy5-ODN (that lacks the NTA group) and Ni (II). FIG. 2B shows flow cytometry analysis of His-tagged bacteria (right hand side peak) and native bacteria (left hand side peak) incubated with Compound 101. FIG. 2C shows fluorescence images of E. coli expressing His-OmpC decorated with Compound 100 in the presence of increasing concentrations of EDTA (0, 5, and 10 mM) (left), and following subsequent addition of Compound 100 in the absence of Ni (II) (right). FIG. 2D shows the growth curve of E. coli expressing His-OmpC (black) and of the same bacteria decorated with Compound 101 (grey). FIG. 2E shows bright field (top) and fluorescence images (bottom) of bacteria decorated Compound 101 monitored at 0, 12, and 24 hours.

FIGS. 3A-3B show the reversible modification of membrane-bound synthetic receptor using complementary strands. FIG. 3A shows a schematic illustration of the methods used in the experiment. His-tagged bacteria were sequentially modified by attaching them with ectopic molecules. First, cells were attached with a compound comprising TAMRA. Then, TAMRA was removed by incubating the cells with ODN-3. Then, cells were attached with a compound comprising Cy5. Then Cy5 was detached by incubating the cells with ODN-3. Then, cells were attached with a compound comprising FAM. Then, cells were attached with Cy5. Then Cy5 was detached by incubating the cells with ODN-3. FIG. 3B shows microscopic images of the emissions of TAMRA, Cy5, and FAM using 590 nm, 700/775 nm, and 510/550 nm emission filters, respectively.

FIGS. 4A-4D show experimental modifications of bacterial cell surface luminescence. FIG. 4A shows a schematic illustration of the experiment. (i) Different sub-populations of His-tagged cells were incubated with three types of ODN-1: Compound 102, Compound 103, and Compound 104. (ii) cells were incubated with three types of ODN-2: Compound 202, Compound 203, and Compound 204, complementary to Compound 102, Compound 103, and Compound 104, respectively. Compound 202, Compound 203, and Compound 204 were appended with FAM, TAMRA, and CY5, respectively.

FIG. 4B shows a fluorescence overlay image of the labeled mixed population. Bacteria were imaged using 488 nm, 561 nm, and 647 nm excitation lasers and 488/50, 610/60, and 685/50 emission filters. FIG. 4C shows percentages of each sub-population counted and averaged from six different frames. FIG. 4D shows a flow cytometry analysis of the mixed population.

FIGS. 5A-5G show bacteria decorated to interact with proteins expressed by cancer cells. FIG. 5A shows a schematic illustration of an experiment in which modified His-tagged bacteria were treated with Alexa 647-modified streptavidin (Alexa-SA). Left: Bacteria were modified with a duplex generated from ODN-1 and Compound 205. Right: Bacteria were modified with a duplex lacking biotin. FIG. 5B shows Alexa-SA fluorescence in the cells incubated with Alexa 647-modified streptavidin. FIG. 5C shows images recorded following the incubation of the bacteria bound to Alexa-SA with ODN-3. FIG. 5D shows a schematic illustration of an experiment in which decorated bacteria were incubated with KB-cells. Left: Bacteria decorated with a duplex consisting of ODN-1 and TAMRA-labeled Compound 206. Right: Bacteria decorated with a duplex that lacks the folate group. FIG. 5E shows TAMRA-labeling of KB cells incubated with bacteria decorated with folate. FIG. 5F shows fluorescent images obtained after treating the bacteria that are bound to KB cells with ODN-3. FIG. 5G shows that incubating a KB-cell with a duplex consisting of ODN-1 and TAMRA-folate-ODN-2 (Compound 206), in the absence of bacteria, did not lead to fluorescent KB-cell labeling.

FIGS. 6A-6B shows bacteria decorated to interact to a non-biological surface. FIG. 6A shows microscopic images of: (i) bear gold substrate after incubation with unmodified bacteria, (ii) passivated gold substrate after incubation with unmodified bacteria, and (iii) passivated gold substrate following incubation with bacteria modified with a thiol-modified duplex (ODN-1:Compound 207). FIG. 6B shows the average bacteria count on passivated gold surfaces, which corresponds to an image area of ˜0.0165 mm².

FIGS. 7A-7B show super-resolution images of His-tagged bacteria decorated with an ODN-1:Compound 201 duplex. FIG. 7A shows whole bacteria. FIG. 7B shows a transverse cut viewed from the plane of the cell axis.

FIGS. 8A-8C depict a schematic representation of fluorescent CSP labeling using (FIG. 8A) FP fusion, (FIG. 8B) IF, or (FIG. 8C) synthetic probes that target small-molecule binding sites of CSPs

FIGS. 9A-9C depict the system design principles. FIG. 9A depicts a schematic illustration of the way B-probes are created and utilized. Incubating His-bacteria with a DNA duplex appended with a fluorophore and binders for a His-tag and an CSP (duplex N) affords a B-probe (step 1) that can label a specific class of cancer cells (step 2). FIG. 9B depicts a schematic overview of the formation of B-probes 1-3 from modified DNA duplexes 1-3. B-probes 1-3 were designed to label cervical, melanoma, and prostate cancer cells, respectively. FIG. 9C depicts the structures of the modified ODNs used to generate duplexes 1-3.

FIGS. 10A-10D depict merged bright-field and fluorescence images of KB cells (left), MDA-MB-435 cells (middle), and LNCaP cells (right) following incubation with: (FIG. 10A) PE-anti-folR Ab (left), Alexafluor647-anti-sigmaR1 Ab (middle), and Alexafluor488-anti-PSMA Ab (right); (FIG. 10B) B-probe 1 (left), B-probe 2 (middle), and B-probe 3 (right); (FIG. 10C) Bacteria linked to duplexes lacking the CSP binders, namely, duplexes generated from tri-NTA-ODN₁ (Compound 105) and TAMRA-ODN₂ (Compound 240) (left), Cy5-ODN₂ (Compound 220) (middle), or FAM-ODN₂ (Compound 230) (right); and (FIG. 10D) 500 nM duplex 1 (left), duplex 2 (middle), and duplex 3 (right) in the absence of His-bacteria.

FIG. 11 depicts immunofluorescence studies as depicted in merged bright-field and fluorescence images of (top) LNCaP cells, (middle) MDA-MB-435 cells, and (bottom) KB cells after incubation with PE-anti-FolR, Alexafluor647-anti-sigmaR1, and Alexafluor488-anti-PSMA Abs.

FIG. 12 depicts a selective labeling of cancer cells with a B-probe mixture. Right: Schematic illustration of an experiment in which a mixture of B-probes 1-3 was used to identify distinct cancer cell types, namely, KB cells (top), MDA-MB-435 cells (middle), and LNCaP cells (bottom). Left: The corresponding bright-field and fluorescence images of the labeled cells and their overlay. The cells were imaged using excitation and emission filters suitable for detecting each B-probe type.

FIG. 13 depicts bright-field (I) and fluorescence images (II-VI) of FFPE tissue sections from the MDA-MB-435 xenograft mouse model, following incubation with (I) Hematoxylin and Eosin stain, (II) Alexafluor647-anti-SigmaR1 Ab, (III) B-probe 2, (IV) Alexafluor647-IgG2b isotype control, (V) a derivative of B-probe 2 that lacks the CSP targeting unit (i.e., An), and (VI) duplex 2.

FIGS. 14A-14F depict studies towards the development of 2^{nd} generation probes. FIG. 14A depicts a schematic illustration of the structure of the 1^{st} (left) and the 2^{nd} generation (right) B-probes, which were designed to produce stronger emission signals. FIG. 14B depicts the emission spectra generated by duplexes 4, 5, and 6. FIG. 14C depicts a pictorial representation of these duplexes. FIG. 14D depicts a model of the multi-dye-modified domain of duplex 6 that shows that the six fluorescein molecules are spatially separated. FIG. 14E depicts merged bright-field and fluorescence images of His-bacteria labeled with duplex 4 (I), duplex 5 (II), and duplex 6 (III). FIG. 14F depicts the corresponding average fluorescence intensity values generated from these labeled bacterial cells.

FIG. 15 depicts merged bright-field and fluorescence images of His-bacteria upon incubation with 100 nM of (left) duplex 4, (middle) duplex 5, and (right) duplex 6 in the absence of Ni⁺².

FIGS. 16A-16E depict the development of brighter, 2^{nd} generation B-probes, which intended to label cancer cells with higher efficiency. FIG. 16A depicts a schematic representation showing the assembly steps used to form B-probes 4 and 5. FIG. 16B depicts the emission spectra of duplex 3 (bottom line), construct 1 (top line), and construct 2 (middle line) measured under an excitation wavelength of 495 nm. FIG. 16C depicts the fluorescence images of His-bacteria following incubation with 100 nM of duplex 3 (B-probe 3, top) or construct 1 (B-probe 4, bottom) in the presence ofNi²⁺ (500 nM). FIG. 16D depicts the merged bright-field and fluorescence images of LNCaP cells post-labeling with B-probe 3 (left) or B-probe 4 (right). FIG. 16E depicts the corresponding average fluorescence intensity values.

FIG. 17 depicts the native agarose gel electrophoresis stained with ethidium bromide showing the formation of the (left) construct 1, and (right) construct 2.

FIG. 18A-18B. FIG. 18A depicts fluorescence images of (left) B-probe 3, (middle) B-probe 4, and (right) B-probe 5 formed upon incubation with duplex 3, construct 1 and construct 2, respectively. B-probes were prepared following incubation of bacteria with 100 nM of each duplex and 500 nM of NiCl₂. FIG. 18B depicts a bar graph, which represents the fluorescence intensities obtained from these images.

FIGS. 19A-19C depict B-probes' internalization into cancer cells. FIG. 19A depicts LNCaP cells labeled with B-probe 3 at time t=0 (left) and t=45 min (right). FIG. 19B depicts (I) Co-imaging of B-probe 4 (dots) and LNCaP cells expressing mCherry-labeled membrane protein (major shape). (II) zoom-in image, where the LNCaP cell membrane is visualized as transparent. (III) The same image in which the LNCaP cell membrane is viewed in an opaque mode. The images show that bacteria 3-7 are either entirely or partially engulfed in the cell. FIG. 19C depicts co-images obtained from a negative control experiment in which the same LNCaP cells were incubated with a derivative of B-probe 4 that lacks the GLA unit.

FIG. 20 depicts live LNCaP cells expressing mCherry-CaaX incubated with B-probe 3. Arrows indicate two bacteria, which are photo-bleached.

FIG. 21 depicts the displacement studies with small molecules. (Left) Bright-field, (middle) fluorescence, and (right) overlay images of KB cells after incubation with 1 μM folic acid followed by incubation with B-probe 1.

FIGS. 22A-22B depict two representative tumor set of images (20×magnification) showing the whole tumor section stained with (a) hematoxylin and eosin stain, (b) Alexafluor-647-anti-SigmaR1 Ab, (c) B-probe 2, (d) duplex 2, (e) Alexafluor647-IgG2b isotype control, and (f) bacteria modified with duplex generated from tri-NTA-ODN₁ (Compound 105) and Cy5-ODN₂ (Compound 220) (lacking anisamide).

FIG. 23 depicts Z-stacks of 0.93 μm increments of selected live LNCaP cells expressing mCherry-CaaX incubated with B-probe 4.

FIG. 24 depicts a specific example of the components that are comprised in the DNA construct according to this invention. Compound 105 (tri-NTA-ODN₁) and Compound 208 (ODN_{2b}-GLA) (first DNA duplex), and ssDNA-long or ssDNA-long′ (ODN_{4b} or ODN_{4b′}, respectively) and ssDNA-short (ODN₃) (second DNA duplex).

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### DNA Construct for Decorating Cell Membranes (1^{st} Generation)

In some embodiments, disclosed herein is a DNA construct comprising:
a. a first compound comprising a first oligonucleotide (ODN-1) covalently bound to a binder, either directly or through a first linker;
b. a second compound comprising a second oligonucleotide (ODN-2) covalently bound to a synthetic agent, either directly or through a second linker, wherein said second oligonucleotide is complementary to said first oligonucleotide.

In some embodiments, the first compound is bound to the second compound through hybridization of ODN-1 and ODN-2.

In some embodiments, the first compound of the DNA construct according to this invention is as described under the title “The first Compound (X-ODN-1)” hereinbelow.

In some embodiments, the first compound of the DNA construct according to this invention is represented by the structure of formula J, H, H(complex), H(a), H(a)(complex), H(b) and H(b)(complex) described hereinbelow; each represents a separate embodiment according to this invention. In some embodiments, the first compound is represented by the structure of any one of compounds 100-106; each represents a separate embodiment according to this invention. In some embodiments, the first compound is represented by the structure of compound 105.

In some embodiments, the first oligonucleotide (ODN-1) of the DNA construct according to this invention is as described under the title “ODN-1 (or ODN1)” hereinbelow.

In some embodiments, the first oligonucleotide (ODN-1) is 1-1000; 3-500; 4-250; 5-100; 10-80; 10-50; 15-35; 5-25; 15-25; 17-23; 15-20; 20-30; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first oligonucleotide comprises a sequence comprising at least 80% homology to any of SEQ ID NOs.: 1-5. In some embodiments, the first oligonucleotide sequence is represented by any one of SEQ ID NOs.: 1-5. In some embodiments, the first oligonucleotide comprises a sequence comprising at least 80% homology to any of SEQ ID NOs.: 3. In some embodiments, the first oligonucleotide sequence is represented by any one of SEQ ID NOs.: 3.

In some embodiments, the first compound is attached to the second compound via the hybridization of the first oligonucleotide (ODN-1) to the second oligonucleotide (ODN-2).

In some embodiments, the second compound of the DNA construct according to this invention is as described under the title “The second Compound (Y-ODN-2)” hereinbelow.

In some embodiments, the second compound of the DNA construct according to this invention is represented by the structure of formula K as described hereinbelow. In some embodiments, the second compound is represented by the structure of any one of compounds 200-214, 220, 230, 240 and 250; each represents a separate embodiment according to this invention.

In some embodiments, the second oligonucleotide (ODN-2) of the DNA construct according to this invention is as described under the title “ODN-2 (or ODN2)” hereinbelow.

In some embodiments, the second oligonucleotide (ODN-2) is 1-1000; 3-500; 4-250; 5-100; 10-80; 10-50; 15-35; 5-45; 15-55; 17-23; 15-20; 20-40; 30-40; 30-35; 10; 11; 12; 13; 14; 15; 16; 17; 18; 19; 20; 21; 22; 23; 24; 25; 26; 27; 28; 29; 30; 31; 32;33; 34; 35;36; 37; 38; 39; 40 bases long; each represents a separate embodiment according to this invention. In some embodiments, the second oligonucleotide comprises a sequence comprising at least 80% homology to any of SEQ ID NOs.: 6-9. In some embodiments, the second oligonucleotide sequence is represented by any one of SEQ ID NOs.: 6-9.

In some embodiments, the second oligonucleotide (ODN-2) is longer than the first oligonucleotide (ODN-1). In some embodiments, the second oligonucleotide (ODN-2) is shorter than the first oligonucleotide (ODN-1). In some embodiments, the second oligonucleotide (ODN-2) and the first oligonucleotide (ODN-1) are of the same length.

In some embodiments, the second oligonucleotide (ODN-2) comprises a hanging strand (a toehold region).

In some embodiments, the hanging strand of the DNA construct according to this invention is as described under the title “hanging strand” hereinbelow.

In some embodiments, the hanging strand of ODN-2 is 1-1000; 3-500; 4-250; 5-100; 1-100; 3-50; 4-25; 5-50; 10-80; 10-50; 5-20; 5-15; 7-18; 5-45; 7-35; 15-30; 15-35; 20-30; 2; 4; 5; 6; 7; 8; 9; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the hanging strand is at least 2, at least 4, at least 8, at least 12, at least 16, at least 20, or at least 30 nucleotides in length; each is a separate embodiment according to this invention. In some embodiments, the hanging strand is no more than about 50 nucleotides in length. In some embodiments, the hanging strand is 26 bases long. In some embodiments, the hanging strand is 16 bases long. In some embodiments, the hanging strand is 10 bases long.

In some embodiments, the binder of the DNA construct according to this invention is as described under the title “Binder (Y1)” hereinbelow.

In some embodiments, the binder comprised in the first compounds of the DNA construct according to this invention, is capable of binding an affinity tag comprises in a polypeptide. In some embodiments, the binder is a His-tag specific binder. In some embodiments, the His-tag specific binder, is capable of binding to an affinity tag comprising a poly-histidine peptide.

In some embodiments, the His-tag specific binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) as described hereinbelow; each represents a separate embodiment according to this invention.

In some embodiments, the synthetic agent of the DNA construct according to this invention is as described under the title “synthetic agent” hereinbelow.

In some embodiments, the synthetic agent of the second compound is bound to the 3′ end of the second oligonucleotide. In some embodiments, the synthetic agent of the second compound is bound to the 5′ end of the second oligonucleotide. In some embodiments, the synthetic agent of the second compound is bound to the 3′ end or to the 5′ end of the second oligonucleotide.

In some embodiments, the synthetic agent comprises a molecular marker, a labeling moiety, a fluorescent dye, an adhesion molecule, a cancer cell binder, a cell surface protein (CSP) binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent comprises a labeling moiety. In some embodiments, the synthetic agent comprises a fluorescent dye. In some embodiments, the synthetic agent comprises a CSP binder.

In some embodiments, the synthetic agent comprises a protein binder or derivative thereof. In some embodiments, the protein binder is selected from a list comprising of: Antibodies: (i.e., immunoglobulins), Enzymes, Hormones, (e.g., insulin, estrogen, and testosterone), Receptors, Lectins, Carrier proteins (e.g., hemoglobin and albumin), DNA-binding proteins (e.g., transcription factors and DNA polymerases), RNA-binding proteins (e.g., splicing factors and ribosomal proteins), Chaperones, Metal-binding proteins (e.g. metalloproteins), Drugs (e.g., antibiotics, antiviral drugs, anticancer agents), Inhibitors (e.g., statins, protease inhibitors), Agonists, antagonists, Natural products (e.g., resveratrol), Ligands, Metabolites (e.g., adenosine triphosphate (ATP)), Toxins (e.g., botulinum toxin, ricin), Dyes and fluorescent probes; each represent a separate embodiment according to this invention. In some embodiments, the protein binder comprises: a folate, anisamide (An), glutamate urea (GLA), biotin, an antibody, marimastat, ethacrynic acid, bisethacrynic acid, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA); each represents a separate embodiment according to this invention. In some embodiments, the protein binder comprises a biotin, a folate, an anisamide, a glutamate urea, an antibody; each represents a separate embodiment according to this invention. In some embodiments, the protein binder comprises a biotin derivative, a folate derivative, an anisamide derivative, a glutamate urea derivative, or an antibody derivative; each represents a separate embodiment according to this invention.

In some embodiments, the synthetic agent comprises a cancer cell binder or derivative thereof. In some embodiments, the cancer cell binder comprises a biotin, a folate, an anisamide, a glutamate urea, an antibody; each represents a separate embodiment according to this invention. In some embodiments, the cancer cell binder comprises a biotin derivative, a folate derivative, an anisamide derivative, a glutamate urea derivative, or an antibody derivative; each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention.

In some embodiments, the synthetic agent is a CSP binder. In some embodiments, the CSP binder can interact with a specific CSP on a cancer cell. In some embodiments, the CSP binder comprises a biotin, a folate, an anisamide, a glutamate urea, an antibody or derivative thereof, each represents a separate embodiment according to this invention. In some embodiments, the CSP binder comprises a biotin derivative, a folate derivative, an anisamide derivative, a glutamate urea derivative, or an antibody derivative; each represents a separate embodiment according to this invention.

In some embodiments, the DNA construct according to this invention comprises a first linker (L₁) and/or a second linker (L₂) as described hereinbelow under the title(s) “A First Linker (L₁)” and/or “The second linker (L₂)”.

In some embodiments, the first and/or the second linker comprises at least one oligoethyleneglycol (OEG) moiety, at least one phosphate moiety, at least one thioalkyl moiety or any combination thereof, each represents a separate embodiment according to this invention. In some embodiments, the first and/or the second linker comprise two; three; four; five; six; seven; eight; nine or ten sequentially arranged oligoethylene glycol (OEG) spacers; each represents a separate embodiment according to this invention. In some embodiments, the first and/or the second linker comprise two sequentially arranged oligoethylene glycol (OEG) spacers. In some embodiments, the first and/or the second linker comprise five sequentially arranged oligoethylene glycol (OEG) spacers. In some embodiments, the first and/or the second linker comprises the following monomer:

—[(CH₂O)ₖ—PO₃H]ₗ—
wherein
   k and l are each independently an integer number between 0 and 10; and
   w is an integer number between 1 and 10.

In some embodiments, the first and/or the second linker comprises one; two; three; four; five; six; seven; eight; nine; or ten monomeric units; each represents a separate embodiment according to this invention. In some embodiments, the first and/or the second linker comprises two monomeric units. In some embodiments, the first and/or the second linker comprises five monomeric units.

In some embodiments, the first and/or the second linker is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

In some embodiments, the first linker is absent. In some embodiments, the second linker is absent. In some embodiments, 1 is 2. In some embodiments, 1 is 5.

In some embodiments, the DNA construct further comprises a labeling moiety. In some embodiments, the synthetic agent is a labeling moiety.

In some embodiments, the labeling moiety of the DNA construct according to this invention is as described under the title “labeling moiety (F and F₂)” hereinbelow.

In some embodiments, the labeling moiety is attached to the first compound. In some embodiments, the labeling moiety is attached to the second compound. In some embodiments, the labeling moiety is a fluorescent dye. In some embodiments, the DNA construct further comprises a fluorescent dye. In some embodiments, the DNA construct further comprises at least one fluorescent dye. In some embodiments, the synthetic agent is a fluorescent dye. In some embodiments, the fluorescent dye comprised in the DNA construct, is as described herein below for “fluorescent dye”. In some embodiments, the fluorescent dye is selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof; each represents a separate embodiment according to this invention.

### DNA Construct for Decorating Cell Membranes (monoODN)

In some embodiments, disclosed herein is a DNA construct comprising:
a. a first compound comprising a first oligonucleotide (ODN-1) covalently bound to a His-tag specific binder, either directly or through a first linker;
b. a second compound comprising a second oligonucleotide (ODN-2) covalently bound to a synthetic agent, either directly or through a second linker, wherein said second oligonucleotide is complementary to said first oligonucleotide and wherein said ODN-2 comprises a hanging strand appended with at least two fluorescent dyes.

In some embodiments, the first compound is bound to the second compound through hybridization of ODN-1 and ODN-2.

In some embodiments, the DNA construct further comprises a third oligonucleotide (ODN-3) that is complementary to the hanging strand. In some embodiments, the third oligonucleotide is 1-1000; 3-500; 4-250; 5-100; 10-80; 10-50; 15-35; 5-25; 15-25; 17-30; 15-30; 20-35; 10; 15; 16; 17; 18; 19; 20; 21; 22; 23; 24; 25; 26; 27; 28; 29; 30; 31; 32; 33; 34; 35; 36; 37; 38; 39; 40 bases long; each represents a separate embodiment according to this invention. In some embodiments, the third oligonucleotide comprises a sequence comprising at least 80% homology to SEQ ID NO.: 24. In some embodiments, the third oligonucleotide sequence is represented by SEQ ID NO.: 24.

In some embodiments, the first compound of the DNA construct according to this invention is as described under the title “The first Compound (X-ODN-1)” hereinbelow.

In some embodiments, the first compound of the DNA construct according to this invention is represented by the structure of formula J, H, H(complex), H(a), H(a)(complex), H(b) and H(b)(complex) described hereinbelow; each represents a separate embodiment according to this invention. In some embodiments, the first compound of the DNA construct according to this invention is represented by the structure of formula J, H, H(complex), H(a), H(a)(complex), H(b) and H(b)(complex) described hereinbelow; wherein F and L₃ are absent. In some embodiments, the first compound is represented by the structure of any one of compounds 100-106; each represents a separate embodiment according to this invention. In some embodiments, the first compound is represented by the structure of compound 105.

In some embodiments, the first oligonucleotide (ODN-1) of the DNA construct according to this invention is as described under the title “ODN-1 (or ODN1)” hereinbelow.

In some embodiments, the first oligonucleotide (ODN-1) is 1-1000; 3-500; 4-250; 5-100; 10-80; 10-50; 15-35; 5-25; 15-25; 17-23; 15-20; 20-30; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first oligonucleotide comprises a sequence comprising at least 80% homology to any of SEQ ID NOs.: 1-5. In some embodiments, the first oligonucleotide sequence is represented by any one of SEQ ID NOs.: 1-5. In some embodiments, the first oligonucleotide comprises a sequence comprising at least 80% homology to any of SEQ ID NOs.: 3. In some embodiments, the first oligonucleotide sequence is represented by any one of SEQ ID NOs.: 3.

In some embodiments, the first compound is attached to the second compound via the hybridization of the first oligonucleotide (ODN-1) to the second oligonucleotide (ODN-2).

In some embodiments, the second compound of the DNA construct according to this invention is as described under the title “The second Compound (Y-ODN-2)” hereinbelow.

In some embodiments, the second compound of the DNA construct according to this invention is represented by the structure of formula K as described hereinbelow. In some embodiments, the second compound is represented by the structure of Compound 250.

In some embodiments, the second oligonucleotide (ODN-2) of the DNA construct according to this invention is as described under the title “ODN-2 (or ODN2)” hereinbelow.

In some embodiments, the second oligonucleotide (ODN-2) is 1-1000; 3-500; 4-250; 5-100; 10-80; 10-50; 15-35; 5-45; 15-55; 17-23; 15-20; 20-40; 30-40; 30-35; 10; 15; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50 bases long; each represents a separate embodiment according to this invention. In some embodiments, the second oligonucleotide comprises a sequence comprising at least 80% homology to any of SEQ ID NOs.: 6-9, 22, 23, and 25. In some embodiments, the second oligonucleotide sequence is represented by any one of SEQ ID NOs.: 6-9, 22, 23, and 25. In some embodiments, the second oligonucleotide comprises a sequence comprising at least 80% homology to SEQ ID NO.: 22 or 23. In some embodiments, the second oligonucleotide sequence is represented by any one of SEQ ID NOs.: 22 or 23.

In some embodiments, the second oligonucleotide (ODN-2) is longer than the first oligonucleotide (ODN-1). In some embodiments, the second oligonucleotide (ODN-2) is shorter than the first oligonucleotide (ODN-1). In some embodiments, the second oligonucleotide (ODN-2) and the first oligonucleotide (ODN-1) are of the same length.

In some embodiments, the second oligonucleotide (ODN-2) comprises a hanging strand (a toehold region).

In some embodiments, the hanging strand of the DNA construct according to this invention is as described under the title “hanging strand” hereinbelow.

In some embodiments, the hanging strand (toehold region) of the second oligonucleotide (ODN-2) is 1-1000; 3-500; 4-250; 5-100; 1-100; 3-50; 4-25; 5-50; 10-80; 10-50; 5-20; 5-15; 7-18; 5-45; 7-35; 15-30; 15-35; 20-30; 2; 4; 5; 6; 7; 8; 9; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first hanging strand is 26 bases long. In some embodiments, the first hanging strand is 16 bases long. In some embodiments, the first hanging strand is 10 bases long. In some embodiments, the hanging strand is at least 2, at least 4, at least 8, at least 12, at least 16, at least 20, or at least 30 nucleotides in length; each is a separate embodiment according to this invention. In some embodiments, the hanging strand is no more than about 50 nucleotides in length.

In some embodiments, the hanging strand is appended with at least 2; 3; 4; 5; 6; 7; 8; 9 or 10 fluorescent dyes; each represents a separate embodiment according to this invention. In some embodiments, the hanging strand comprises 2; 3; 4; 5; 6; 7; 8; 9 or 10 fluorescent dyes; each represents a separate embodiment according to this invention. In some embodiments, the hanging strand comprises no more than 2; 3; 4; 5; 6; 7; 8; 9 or 10 fluorescent dyes; each represents a separate embodiment according to this invention. In some embodiments, the hanging strand is appended with 6 fluorescent dyes.

In some embodiments, the fluorescent dyes attached to said hanging strand are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the fluorescent dyes attached to said hanging strand are located 4-6 bases apart from each other. In some embodiments, the fluorescent dyes attached to said hanging strand are located 5 bases apart from each other.

In some embodiments, the fluorescent dyes are covalently attached to the hanging strand. In some embodiments, the fluorescent dyes are covalently attached to a certain nucleotide of the hanging strand. In some embodiments, the fluorescent dyes are covalently attached to a Thymine nucleotide of the hanging strand.

In some embodiments, the His-tag specific binder comprised in the first compounds of the DNA construct according to this invention, is capable of binding to an affinity tag comprising a poly-histidine peptide.

In some embodiments, the His-tag specific binder of the DNA construct according to this invention is as described for His-tag binder under the title “Binder (Y1)” hereinbelow.

In some embodiments, the His-tag specific binder of the DNA construct according to this invention comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) as described hereinbelow; each represents a separate embodiment according to this invention.

In some embodiments, the synthetic agent of the DNA construct according to this invention is as described under the title “synthetic agent” hereinbelow.

In some embodiments, the synthetic agent of the second compound is bound to the 3′ end of the second oligonucleotide. In some embodiments, the synthetic agent of the second compound is bound to the 5′ end of the second oligonucleotide. In some embodiments, the synthetic agent of the second compound is bound to the 3′ end or to the 5′ end of the second oligonucleotide.

In some embodiments, the synthetic agent comprises a molecular marker, a labeling moiety, a fluorescent dye, an adhesion molecule, a cancer cell binder, a cell surface protein (CSP) binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent comprises a labeling moiety. In some embodiments, the synthetic agent comprises a fluorescent dye. In some embodiments, the synthetic agent comprises a CSP binder.

In some embodiments, the synthetic agent comprises a protein binder or derivative thereof. In some embodiments, the protein binder comprises a biotin, a folate, an anisamide, a glutamate urea, an antibody; each represents a separate embodiment according to this invention. In some embodiments, the protein binder comprises a biotin derivative, a folate derivative, an anisamide derivative, a glutamate urea derivative, an antibody derivative; each represents a separate embodiment according to this invention.

In some embodiments, the synthetic agent comprises a cancer cell binder or derivative thereof. In some embodiments, the cancer cell binder comprises a biotin, a folate, an anisamide, a glutamate urea, an antibody; each represents a separate embodiment according to this invention. In some embodiments, the cancer cell binder comprises a biotin derivative, a folate derivative, an anisamide derivative, a glutamate urea derivative, an antibody derivative; each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention.

In some embodiments, the synthetic agent is a CSP binder. In some embodiments, the CSP binder can interact with a specific CSP on a cancer cell. In some embodiments, the CSP binder comprises a biotin, a folate, an anisamide, a glutamate urea, an antibody or derivative thereof, each represents a separate embodiment according to this invention. In some embodiments, the CSP binder comprises a biotin derivative, a folate derivative, an anisamide derivative, a glutamate urea derivative, an antibody derivative; each represents a separate embodiment according to this invention.

In some embodiments, the DNA construct according to this invention comprises a first linker (L₁) and/or a second linker (L₂) as described hereinbelow under the title(s) “A First Linker (L₁)” or “The second linker (L₂)”.

In some embodiments, the first and/or the second linker comprises at least one oligoethyleneglycol (OEG) moiety, at least one phosphate moiety, at least one thioalkyl moiety or any combination thereof, each represents a separate embodiment according to this invention. In some embodiments, the first and/or the second linker comprise two; three; four; five; six; seven or eight sequentially arranged oligoethylene glycol (OEG) spacers; each represents a separate embodiment according to this invention. In some embodiments, the first and/or the second linker comprise two sequentially arranged oligoethylene glycol (OEG) spacers. In some embodiments, the first and/or the second linker comprise five sequentially arranged oligoethylene glycol (OEG) spacers. In some embodiments, the first and/or the second linker comprises the following monomer:

—[(CH₂O)ₖ—PO₃H]ₗ—
wherein
   k and l are each independently an integer number between 0 and 10; and
   w is an integer number between 1 and 10.

In some embodiments, the first and/or the second linker comprises one; two; three; four; five; six; seven; eight; nine; or ten monomeric units; each represents a separate embodiment according to this invention. In some embodiments, the first and/or the second linker comprises two monomeric units. In some embodiments, the first and/or the second linker comprises five monomeric units.

In some embodiments, the first and/or the second linker is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

In some embodiments, the first linker is absent. In some embodiments, the second linker is absent. In some embodiments, 1 is 2. In some embodiments, 1 is 5.

In some embodiments, the fluorescent dyes, which are comprised in the DNA construct, are as described herein below for “fluorescent dye”. In some embodiments, the fluorescent dyes are selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof, each represents a separate embodiment according to this invention.

### DNA Construct for Decorating Cell Membranes (2^{nd} Generation)

In some embodiments, disclosed herein is a DNA construct comprising:
a. a first compound comprising a first oligonucleotide (ODN-1) covalently bound to a His-tag specific binder, either directly or through a first linker;
b. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and c. a third compound comprising a DNA duplex (dsDNA) appended with a second hanging strand complementary to said first hanging strand, and further appended with at least two fluorescent dyes.

In some embodiments, the first compound is bound to the second compound through hybridization of ODN-1 and ODN-2. In some embodiments, the third compound is bound to the second compound through hybridization of the first hanging strand and the second hanging strand.

In some embodiments, the first compound of the DNA construct according to this invention is as described under the title “The first Compound (X-ODN-1)” hereinbelow.

In some embodiments, the first compound of the DNA construct according to this invention is represented by the structure of formula J, H, H(complex), H(a), H(a)(complex), H(b) and H(b)(complex) described hereinbelow; each represents a separate embodiment according to this invention. In some embodiments, the first compound of the DNA construct according to this invention is represented by the structure of formula J, H, H(complex), H(a), H(a)(complex), H(b) and H(b)(complex) described hereinbelow; wherein F and L₃ are absent. In some embodiments, the first compound is represented by the structure of any one of compounds 100-106; each represents a separate embodiment according to this invention. In some embodiments, the first compound is represented by the structure of compound 105.

In some embodiments, the first oligonucleotide (ODN-1) of the DNA construct according to this invention is as described under the title “ODN-1 (or ODN1)” hereinbelow.

In some embodiments, the first oligonucleotide (ODN-1) is 1-1000; 3-500; 4-250; 5-100; 10-80; 10-50; 15-35; 5-25; 15-25; 17-23; 15-20; 20-30; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first oligonucleotide comprises a sequence comprising at least 80% homology to any of SEQ ID NOs.: 1-5. In some embodiments, the first oligonucleotide sequence is represented by any one of SEQ ID NOs.: 1-5. In some embodiments, the first oligonucleotide comprises a sequence comprising at least 80% homology to any of SEQ ID NOs.: 3. In some embodiments, the first oligonucleotide sequence is represented by any one of SEQ ID NOs.: 3.

In some embodiments, the first compound is attached to the second compound via the hybridization of the first oligonucleotide (ODN-1) to the second oligonucleotide (ODN-2).

In some embodiments, the second compound of the DNA construct according to this invention is as described under the title “The second Compound (Y-ODN-2)” hereinbelow.

In some embodiments, the second compound of the DNA construct according to this invention is represented by the structure of formula K as described hereinbelow. In some embodiments, F₂ and L₄ are absent. In some embodiments, the second compound is represented by the structure of any one of compounds 200-214; each represents a separate embodiment according to this invention. In some embodiments, the second compound is represented by the structure of any one of compounds 204, 205 or 208; each represents a separate embodiment according to this invention. In some embodiments, the second compound is represented by the structure of compound 208.

In some embodiments, the second oligonucleotide (ODN-2) of the DNA construct according to this invention is as described under the title “ODN-2 (or ODN2)” hereinbelow.

In some embodiments, the second oligonucleotide (ODN-2) is 1-1000; 3-500; 4-250; 5-100; 10-80; 10-50; 15-35; 5-45; 15-55; 17-23; 15-20; 20-40; 30-40; 33-37; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40 bases long; each represents a separate embodiment according to this invention. In some embodiments, the second oligonucleotide comprises a sequence comprising at least 80% homology to any of SEQ ID NOs.: 6-9. In some embodiments, the second oligonucleotide sequence is represented by any one of SEQ ID NOs.: 6-9.

In some embodiments, the second oligonucleotide (ODN-2) is longer than the first oligonucleotide (ODN-1). In some embodiments, the second oligonucleotide (ODN-2) is shorter than the first oligonucleotide (ODN-1). In some embodiments, the second oligonucleotide (ODN-2) and the first oligonucleotide (ODN-1) are of the same length.

In some embodiments, the second oligonucleotide (ODN-2) comprises a first hanging strand (a first toehold region).

In some embodiments, the first hanging strand of the DNA construct according to this invention is as described under the title “hanging strand” hereinbelow.

In some embodiments, the first hanging strand (first toehold region) of the second oligonucleotide is 1-1000; 3-500; 4-250; 5-100; 1-100; 3-50; 4-25; 5-50; 10-80; 10-50; 5-20; 5-15; 7-18; 5-45; 7-35; 15-30; 15-35; 20-30; 2; 4; 5; 6; 7; 8; 9; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first hanging strand is 26 bases long. In some embodiments, the first hanging strand is 16 bases long. In some embodiments, the first hanging strand is 10 bases long. In some embodiments, the first hanging strand is at least 2, at least 4, at least 8, at least 12, at least 16, at least 20, or at least 30 nucleotides in length; each is a separate embodiment according to this invention. In some embodiments, the first hanging strand is no more than about 50 nucleotides in length.

In some embodiments, the DNA construct according to this invention comprises a third compound as described hereinbelow under the title “The Third Compound”.

In some embodiments, the third compound of the DNA construct according to this invention comprises a DNA duplex (dsDNA), a hanging strand, and at least one labeling moiety. In some embodiments, the hanging strand is a second hanging strand.

In some embodiments, the second hanging strand of the DNA construct according to this invention is as described under the title “hanging strand” hereinbelow.

In some embodiments, the second hanging strand is complementary to the first hanging strand comprised in the second compound as described hereinbelow. In some embodiments, the labeling moiety is a fluorescent dye. In some embodiments, the third compound of the DNA construct according to this invention comprises a DNA duplex (dsDNA), a hanging strand, and at least two labeling moieties.

In some embodiments, the third compound of the DNA construct according to this invention comprises a DNA duplex (dsDNA), a hanging strand, and at least two fluorescent dyes.

In some embodiments, the DNA construct according to this invention comprises a DNA duplex (dsDNA). In some embodiments, the dsDNA is as described hereinbelow under the title “DNA Duplex (dsDNA)”.

In some embodiments, the dsDNA comprises two oligonucleotides, a longer oligonucleotide comprising the second hanging strand (ssDNA-long), and a shorter oligonucleotide (ssDNA-short), wherein ssDNA-long is complementary to ssDNA-short.

In some embodiments, the shorter oligonucleotide (ssDNA-short) is attached to the longer oligonucleotide (ssDNA-long) via the hybridization of the shorter oligonucleotide (ssDNA-short) to the longer oligonucleotide (ssDNA-long).

In some embodiments, the shorter oligonucleotide (ssDNA-short) is 1-1000; 3-500; 4-250; 5-100; 10-80; 10-50; 15-35; 5-45; 15-25; 20-40; 15-50; 25-35; 10; 15; 16; 17; 18; 19; 20; 21; 22; 23; 24; 25; 26; 27; 28; 29; 30; 31; 32; 33; 34; 35; 36; 37; 38; 39; 40 bases long; each represents a separate embodiment according to this invention. In some embodiments, the shorter oligonucleotide (ssDNA-short) comprises a sequence comprising at least 80% homology to SEQ ID NO.: 24. In some embodiments, the shorter oligonucleotide (ssDNA-short) sequence is represented by SEQ ID NO.: 24.

In some embodiments, the longer oligonucleotide (ssDNA-long) is 1-1000; 3-500; 4-250; 5-100; 10-80; 10-50; 15-45; 5-50; 25-55; 20-45; 15-50; 30-45; 10; 15; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50 bases long; each represents a separate embodiment according to this invention. In some embodiments, the longer oligonucleotide (ssDNA-long) comprises a sequence comprising at least 80% homology to SEQ ID NO.: 22 or 23. In some embodiments, the longer oligonucleotide (ssDNA-long) sequence is represented by SEQ ID NO.: 22 or 23.

In some embodiments, the longer oligonucleotide (ssDNA-long) comprises a hanging strand. In some embodiments, the longer oligonucleotide (ssDNA-long) comprises a second hanging strand, which is complementary to the first hanging strand comprised in the second compound described hereinbelow. In some embodiments, the second hanging strand of the DNA duplex (dsDNA) is 1-1000; 3-500; 4-250; 5-100; 1-100; 3-50; 4-25; 5-50; 10-80; 10-50; 5-20; 5-15; 7-18; 5-45; 7-35; 15-30; 15-35; 20-30; 2; 4; 5; 6; 7; 8; 9; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the second hanging strand of the DNA duplex (dsDNA) is at least 2, at least 4, at least 8, at least 12, at least 16, at least 20, or at least 30 nucleotides in length; each is a separate embodiment according to this invention. In some embodiments, the second hanging strand of the DNA duplex (dsDNA) is no more than about 50 nucleotides in length.

In some embodiments, the DNA duplex (dsDNA) comprises at least 2; 3; 4; 5; 6; 7; 8; 9 or 10 fluorescent dyes; each represents a separate embodiment according to this invention. In some embodiments, the DNA duplex (dsDNA) comprises 2; 3; 4; 5; 6; 7; 8; 9 or 10 fluorescent dyes; each represents a separate embodiment according to this invention. In some embodiments, the DNA duplex (dsDNA) comprises no more than 2; 3; 4; 5; 6; 7; 8; 9 or 10 fluorescent dyes; each represents a separate embodiment according to this invention.

In some embodiments, the fluorescent dyes of the DNA duplex (dsDNA) are located on the longer oligonucleotide strand (ssDNA-long) that comprises the hanging strand. In some embodiments, the fluorescent dyes of the DNA duplex (dsDNA) are located on the shorter oligonucleotide strand of the dsDNA (ssDNA-short). In some embodiments, the fluorescent dyes of the DNA duplex (dsDNA) are located on both ssDNA-short and ssDNA-long.

In some embodiments, the fluorescent dyes of said DNA duplex (dsDNA) are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the fluorescent dyes of said DNA duplex (dsDNA) are located 4-6 bases apart from each other. In some embodiments, the fluorescent dyes of said DNA duplex (dsDNA) are located 5 bases apart from each other.

In some embodiments, the fluorescent dyes are covalently attached to the dsDNA. In some embodiments, the fluorescent dyes are covalently attached to a certain nucleotide of ssDNA-long oligonucleotide. In some embodiments, the fluorescent dyes are covalently attached to a Thymine nucleotide of the ssDNA-long oligonucleotide.

In some embodiments, the His-tag specific binder comprised in the first compounds of the DNA construct according to this invention, is capable of binding to an affinity tag comprising a poly-histidine peptide.

In some embodiments, the His-tag specific binder of the DNA construct according to this invention is as described for His-tag binder under the title “Binder (Y1)” hereinbelow.

In some embodiments, the His-tag specific binder of the DNA construct according to this invention comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) as described hereinbelow; each represents a separate embodiment according to this invention.

In some embodiments, the fluorescent dyes, which are comprised in the DNA duplex (dsDNA) of the DNA construct, are as described herein below for “fluorescent dye”. In some embodiments, the fluorescent dyes are selected from a group comprising dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof, each represents a separate embodiment according to this invention.

In some embodiments, the synthetic agent of the second compound is bound to the 3′ end of the second oligonucleotide. In some embodiments, the synthetic agent of the second compound is bound to the 5′ end of the second oligonucleotide. In some embodiments, the synthetic agent of the second compound is bound to the 3′ end or to the 5′ end of the second oligonucleotide.

In some embodiments, the synthetic agent comprises a molecular marker, a labeling moiety, a fluorescent dye, an adhesion molecule, a cancer cell binder, a cell surface protein (CSP) binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention. In some embodiments, the synthetic agent comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof, each represents a separate embodiment according to this invention.

In some embodiments, the synthetic agent comprises a protein binder or derivative thereof. In some embodiments, the protein binder comprises a biotin, a folate, an anisamide, a glutamate urea, an antibody; each represents a separate embodiment according to this invention. In some embodiments, the protein binder comprises a biotin derivative, a folate derivative, an anisamide derivative, a glutamate urea derivative, an antibody derivative; each represents a separate embodiment according to this invention.

In some embodiments, the synthetic agent comprises a cancer cell binder or derivative thereof. In some embodiments, the cancer cell binder comprises a biotin, a folate, an anisamide, a glutamate urea, an antibody; each represents a separate embodiment according to this invention. In some embodiments, the cancer cell binder comprises a biotin derivative, a folate derivative, an anisamide derivative, a glutamate urea derivative, an antibody derivative; each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention.

In some embodiments, the synthetic agent can interact with a specific CSP on a cancer cell. In some embodiments, the CSP binder comprises a biotin, a folate, an anisamide, a glutamate urea, an antibody or derivative thereof, each represents a separate embodiment according to this invention. In some embodiments, the CSP binder comprises a biotin derivative, a folate derivative, an anisamide derivative, a glutamate urea derivative, or an antibody derivative; each represents a separate embodiment according to this invention.

In some embodiments, the DNA construct according to this invention comprises a first linker (L₁) and/or a second linker (L₂) as described hereinbelow under the title(s) “A First Linker (L₁)” or “The second linker (L₂)”.

In some embodiments, the first and/or the second linker comprises at least one oligoethyleneglycol (OEG) moiety, at least one phosphate moiety, at least one thioalkyl moiety or any combination thereof, each represents a separate embodiment according to this invention. In some embodiments, the first and/or the second linker comprise two; three; four; five; six; seven or eight sequentially arranged oligoethylene glycol (OEG) spacers; each represents a separate embodiment according to this invention. In some embodiments, the first and/or the second linker comprises the following monomer:

—[(CH₂O)ₖ—PO₃H]ₗ—
wherein
   k and l are each independently an integer number between 0 and 10; and
   w is an integer number between 1 and 10.

In some embodiments, the first and/or the second linker comprises one; two; three; four; five; six; seven; eight; nine; or ten monomeric units; each represents a separate embodiment according to this invention. In some embodiments, the first and/or the second linker comprises two monomeric units. In some embodiments, the first and/or the second linker comprises five monomeric units.

In some embodiments, the first and/or the second linker is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

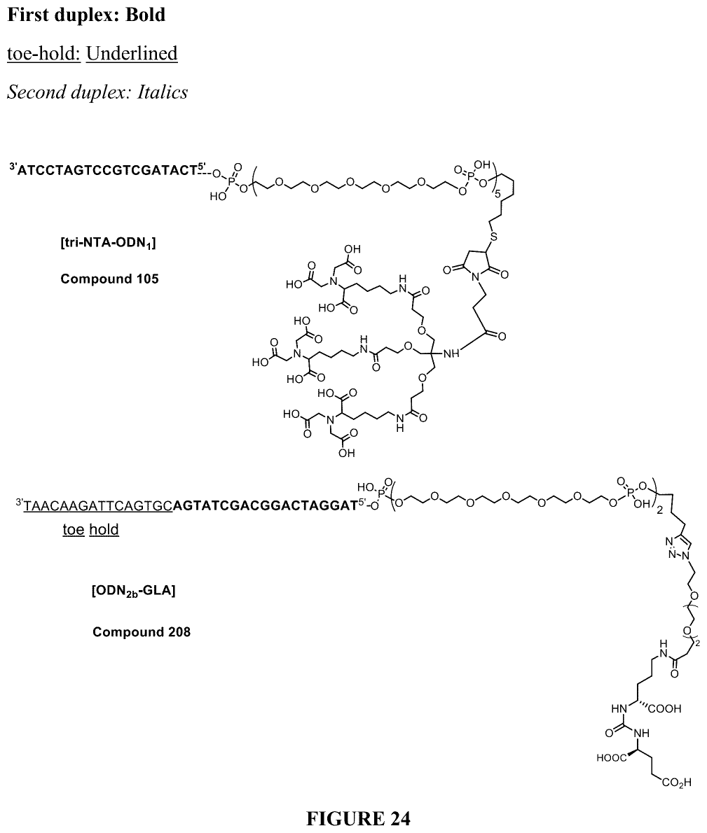

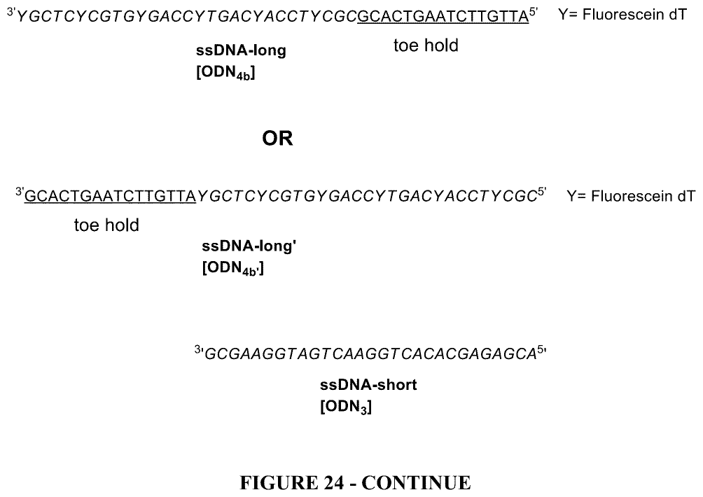

In some embodiments, the DNA construct according to this invention is prepared by hybridization of the oligonucleotide comprising building blocks, as disclosed in FIG. 24.

### System for Decorating Cell Membranes (1^{st} Generation)

In some embodiments, disclosed herein is a system comprising:
a. a recombinant cell ectopically expressing a polypeptide, wherein said polypeptide comprises a membranal anchoring domain and an extracellular binding domain,
b. a first compound comprising a first oligonucleotide (ODN-1) covalently bound to a binder, either directly or through a first linker, said binder comprising affinity to said extracellular binding domain,
c. a second compound comprising a second oligonucleotide (ODN-2) covalently bound to a synthetic agent, either directly or through a second linker, wherein said second oligonucleotide is complementary to said first oligonucleotide.

In some embodiments, the polypeptide is bound to the first compound, the second compound is bound to the first compound, or combination thereof, each represents a separate embodiment according to the invention. In some embodiments, when incubated together, the polypeptide, the first compound, and the second compound, form a complex, in which the polypeptide is attached to the first compound and the first compound is attached to the second compound. In some embodiments, the first compound is attached to the second compound via the hybridization of the first oligonucleotide to the second oligonucleotide. In some embodiments, the first compound is attached to the polypeptide via coordination of said binder to said extracellular binding domain of said polypeptide. In some embodiments, the first compound is attached to the polypeptide via coordination of said binder to an affinity tag comprised in said extracellular binding domain of said polypeptide. In some embodiments, the polypeptide is a cell surface proteins (CSPs). In some embodiments, the polypeptide is an outer membrane protein C (OmpC). In some embodiments, the polypeptide is a receptor tyrosine kinase (RTK).

In some embodiments, the system does not perturb said cell's function. In some embodiments, the system can be reversibly modified. In some embodiments, the recombinant cell is selected from: eukaryotes, prokaryotes, mammalian cells, plant cells, human cells, and bacteria. In some embodiments, the bacteria comprise E. coli. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex). In some embodiments, the first compound is represented by the structure of formula J, H, H(complex), H(a), H(a)(complex), H(b) and H(b)(complex) and compounds 100-106. In some embodiments, the second compound is represented by the structure of formula K and compounds 200-214, 220, 230, 240 and 250. In some embodiments, the first linker comprises at least one oligoethyleneglycol (OEG) moiety, at least one phosphate moiety, at least one thioalkyl moiety or any combination thereof. In some embodiments, the first linker comprises two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof, each represents a separate embodiment according to this invention. In some embodiments, the second compound further comprises a second labeling moiety. In some embodiments, the second labeling moiety comprises a fluorescent dye. In some embodiments, the system further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide.

In some embodiments, the second compound further comprises a labeling moiety. In some embodiments, the second compound further comprises at least 2; 3; 4; 5; 6; 7; 8; 9; or 10 labeling moieties; each represents a separate embodiment according to this invention. In some embodiments, the labeling moiety comprises a fluorescent dye. In some embodiments, the second oligonucleotide comprises a hanging strand (a toehold region). In some embodiments, the hanging strand is further appended with at least two; three; four; five; six; seven; eight; nine; ten fluorescent dyes; each represents a separate embodiment according to this invention. In some embodiments, the first compound is bound to the second compound through hybridization of ODN-1 and ODN-2. In some embodiments, the second compound, second oligonucleotide or the hanging strand, is as described hereinabove for the DNA construct (monoODN) according to this invention.

In some embodiments, the second oligonucleotide comprises a first hanging strand (a first toehold region). In some embodiments, the system further comprises a third compound comprising a DNA duplex (dsDNA) appended with a second hanging strand complementary to said first hanging strand, and further appended with at least two; three; four; five; six; seven; eight; nine; ten fluorescent dyes; each represents a separate embodiment according to this invention. In some embodiments, the first compound is bound to the second compound through hybridization of ODN-1 and ODN-2 and the third compound is bound to the second compound through hybridization of the first hanging strand and the second hanging strand. In some embodiments, the first hanging strand, the second hanging strand and the DNA duplex (dsDNA) are as described hereinabove for the DNA construct (2^{nd} generation) according to this invention.

### System for Decorating Cell Membranes (2^{nd} Generation)

In some embodiments, disclosed herein is a system comprising:
a. a recombinant cell ectopically expressing a polypeptide, wherein said polypeptide comprises a membranal anchoring domain and an extracellular binding domain,
b. a DNA construct according to this invention;
wherein the His-tag specific binder of the DNA construct, comprises affinity to said extracellular binding domain of said polypeptide.

In some embodiments, the DNA construct is bound to the recombinant cell in the presence of Ni²⁺ ions. In some embodiments, the first compound of said DNA construct is bound to the second compound through hybridization of ODN-1 and ODN-2. In some embodiments, the third compound of said DNA construct is bound to the second compound through hybridization of the first hanging strand and the second hanging strand. In some embodiments, the first compound is bound to the second compound through hybridization of ODN-1 and ODN-2, and the third compound is bound to the second compound through hybridization of the first hanging strand and the second hanging strand, thereby forming a DNA construct according to this invention. In some embodiments, the His-tag specific binder of said DNA construct, comprises affinity to said extracellular binding domain of said polypeptide.

In some embodiments, disclosed herein is a system comprising:
a. a recombinant cell ectopically expressing a polypeptide, wherein said polypeptide comprises a membranal anchoring domain and an extracellular binding domain,
b. a first compound comprising a first oligonucleotide (ODN-1) covalently bound to a His-tag specific binder, either directly or through a first linker;
c. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and
d. a third compound comprising a DNA duplex (dsDNA) appended with a second hanging strand complementary to said first hanging strand, and further appended with at least two fluorescent dyes;
wherein the first compound is bound to the second compound through hybridization of ODN-1 and ODN-2, and the third compound is bound to the second compound through hybridization of the first hanging strand and the second hanging strand; and the His-tag specific binder, comprises affinity to said extracellular binding domain of said polypeptide.

In some embodiments, the His-tag specific binder is bound to the recombinant cell in the presence of Ni²⁺ ions.

In some embodiments, the polypeptide is bound to the DNA construct through interaction between a histidine tag and the His-tag specific binder.

In some embodiments, when incubated together, the recombinant cell and the DNA construct according to this invention, form a complex, in which the polypeptide of the recombinant cell is bound to the DNA construct. In some embodiments, the first compound of the DNA construct is attached to the polypeptide via coordination of said binder to said extracellular binding domain of said polypeptide.

In some embodiments, the first compound is attached to the polypeptide via coordination of said binder to an affinity tag comprised in said extracellular binding domain of said polypeptide. In some embodiments, the polypeptide is a cell surface proteins (CSPs). In some embodiments, the CSP comprises: an outer membrane protein C (OmpC), G protein-coupled receptors (GPCRs), Receptor tyrosine kinases (RTKs), Programmed Cell Death protein 1 (PD1), Ion channel receptors, Adhesion proteins (e.g., Cadherins, Integrins, Selectins), Transporters and channels (e.g., Sodium-potassium pumps, Aquaporins (water channels), Glucose transporters (e.g., GLUT1), Major histocompatibility complex (MHC) proteins (e.g., MHC class I proteins, MHC class II proteins), Cell adhesion molecules (CAMs) (e.g., Neural cell adhesion molecule (NCAM), Epithelial cadherin (E-cadherin), Immunoglobulin superfamily CAMs (IgCAMs)), Enzymes (e.g., Adenylyl cyclase, Phospholipases, Receptor-associated kinases (e.g., insulin receptor kinase)), Antigenic proteins (e.g., CD antigens (cluster of differentiation), Blood group antigens (e.g., ABO blood group antigens)), Signal transduction proteins (e.g., Protein kinases, Protein phosphatases, or Second messenger receptors (e.g., cyclic AMP receptors)); each represents a separate embodiment according to this invention. In some embodiments, the polypeptide is an outer membrane protein C (OmpC). In some embodiments, the polypeptide is a receptor tyrosine kinase (RTK).

In some embodiments, the system does not perturb the recombinant cell's function. In some embodiments, the system can be reversibly modified. In some embodiments, the recombinant cell is selected from: eukaryotes, prokaryotes, mammalian cells, plant cells, human cells, and bacteria; each represents a separate embodiment according to this invention. In some embodiments, the recombinant cell is bacteria. In some embodiments, the recombinant cell is bacteria, and the complex formed by coordination of said DNA construct and the recombinant cell, comprises a bacterial probe (i.e., B-probe).

In some embodiments, the bacteria comprise E. coli. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the His-tag specific binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex).

In some embodiments, the first compound of the system according to this invention is as described under the title “The first Compound (X-ODN-1)” hereinbelow. In some embodiments, the first compound is represented by the structure of formula J, H, H(complex), H(a), H(a)(complex), H(b) and H(b)(complex) and compounds 100-106. In some embodiments, the first compound further comprises a labeling moiety. In some embodiments, the labeling moiety is a fluorescent dye.

In some embodiments, the second compound of the system according to this invention is as described under the title “The second Compound (Y-ODN-2)” hereinbelow.

In some embodiments, the second compound is represented by the structure of formula K and compounds 200-214. In some embodiments, the second compound is represented by the structure of any one of compounds 204, 205 or 208; each represents a separate embodiment according to this invention. In some embodiments, the second compound is represented by the structure of compound 208.

In some embodiments, the system according to this invention comprises a first linker (L₁) and/or a second linker (L₂) as described hereinbelow under the title(s) “A First Linker (L₁)” or “The second linker (L₂)”.

In some embodiments, the first linker comprises at least one oligoethyleneglycol (OEG) moiety, at least one phosphate moiety, at least one thioalkyl moiety or any combination thereof. In some embodiments, the first comprises two; three; four; five; six; seven or eight sequentially arranged oligoethylene glycol (OEG) spacers; each represents a separate embodiment according to this invention.

In some embodiments, the synthetic agent of said second compound is as described under the title “synthetic agent” hereinbelow, and/or as described hereinabove for DNA construct according to this invention.

In some embodiments, the system further comprises a fourth compound comprising a third oligonucleotide (ODN-3). In some embodiments, the third oligonucleotide (ODN-3) is complementary to the second oligonucleotide (ODN-2) of the DNA construct and/or system of the invention. In some embodiments, ODN-3 comprises higher affinity to said ODN-2 than the affinity of ODN-2 to ODN-1.

### A Bacterial Probe (B-Probe)—a Recombinant Cell Bound to the DNA Construct of the Invention (2^{nd} Generation)

In some embodiments, disclosed herein is a system comprising:
a. a recombinant cell ectopically expressing a polypeptide, wherein said polypeptide comprises a membranal anchoring domain and an extracellular binding domain comprising a poly-histidine affinity tag, and
b. a DNA construct according to this invention.

In some embodiments, the DNA construct is bound to the recombinant cell in the presence of Ni²⁺ ions.

In some embodiments, this invention relates to a recombinant cell bound to the DNA construct according to this invention, wherein the recombinant cell is ectopically expressing a polypeptide, which comprises a membranal anchoring domain and an extracellular binding domain, wherein the extracellular binding domain of the polypeptide comprises a poly-histidine affinity tag, which is bound to the DNA construct in the presence of Ni²⁺ ions.

In some embodiments, disclosed herein is a system comprising:
a. a recombinant cell ectopically expressing a polypeptide, wherein said polypeptide comprises a membranal anchoring domain and an extracellular binding domain comprising a poly-histidine affinity tag,
b. a first compound comprising a first oligonucleotide (ODN-1) covalently bound to a His-tag specific binder, either directly or through a first linker;
c. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and
d. a third compound comprising a DNA duplex (dsDNA) appended with a second hanging strand complementary to said first hanging strand, and further appended with at least two fluorescent dyes;
wherein the first compound is bound to the second compound through hybridization of ODN-1 and ODN-2, and the third compound is bound to the second compound through hybridization of the first hanging strand and the second hanging strand; and wherein the first compound is bound to the recombinant cell via the binding of the His-tag specific binder to the poly histidine affinity tag of the polypeptide, in the presence of Ni²⁺ ions.

In some embodiments, the recombinant cell is a bacteria as defined hereinbelow. In some embodiments, the membranal anchoring domain of said polypeptide comprises a transmembranal protein or a part of it or an artificial polypeptide; each represents a separate embodiment according to this invention. In some embodiments, the polypeptide is a cell surface protein (CSP). In some embodiments, the polypeptide comprises an outer membrane protein C (OmpC); receptor tyrosine kinases (RTKs); Ion channel linked receptors; Enzyme-linked receptors; G protein-coupled receptors; each represents a separate embodiment according to this invention. In some embodiments, the CSP is a histidine tagged Outer Membrane Protein C (His-OmpC). In some embodiments, the bacteria is a His-OmpC expressing bacteria.

In some embodiments, the first compound is bound to the second compound through hybridization of ODN-1 and ODN-2. In some embodiments, the third compound is bound to the second compound through hybridization of the first hanging strand and the second hanging strand. In some embodiments, the first compound is bound to the second compound through hybridization of ODN-1 and ODN-2, and the third compound is bound to the second compound through hybridization of the first hanging strand and the second hanging strand, thereby forming a DNA construct according to this invention.

In some embodiments, the polypeptide is bound to the DNA construct through interaction between a poly histidine affinity tag and the His-tag specific binder.

In some embodiments, when incubated together, the bacteria cell and the DNA construct according to this invention, form a complex, in which the polypeptide of the bacteria cell is bound to the DNA construct. In some embodiments, the first compound of the DNA construct is attached to the bacteria via coordination of said binder to said poly-histidine affinity tag in the extracellular binding domain of said polypeptide. In some embodiments, the polypeptide is a cell surface protein (CSP) comprising a poly-histidine affinity tag. In some embodiments, the polypeptide is an outer membrane protein C (OmpC). In some embodiments, the polypeptide is a receptor tyrosine kinase (RTK).

In some embodiments, the cell is a living cell. In some embodiments, the cell can be reversibly modified. In some embodiments, the recombinant cell is selected from: eukaryotes, prokaryotes, mammalian cells, plant cells, human cells, and bacteria; each represents a separate embodiment according to this invention. In some embodiments, the recombinant cell is bacteria. In some embodiments, the recombinant cell is bacteria, and the complex formed by coordination of said DNA construct and the recombinant cell, comprises a bacterial probe (i.e., B-probe). In some embodiments, the complex is formed in the presence of metal ions. In some embodiments, the complex is formed in the presence of Nickel ions.

In some embodiments, the bacteria of the bacterial probe (B-probe) according to this invention comprise E. coli. In some embodiments, the membranal anchoring domain comprises a transmembranal protein or a part of it, an artificial polypeptide, or a combination thereof; each represents a separate embodiment according to this invention. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the His-tag specific binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex).

In some embodiments, the first compound of the bacterial probe (B-probe) according to this invention is as described under the title “The first Compound (X-ODN-1)” hereinbelow. In some embodiments, the first compound is represented by the structure of J, H, H(complex), H(a), H(a)(complex), H(b) and H(b)(complex) and compounds 100-106. In some embodiments, the first compound further comprises a labeling moiety. In some embodiments, the labeling moiety is a fluorescent dye.

In some embodiments, the second compound of the bacterial probe (B-probe) according to this invention is as described under the title “The second Compound (Y-ODN-2)” hereinbelow.

In some embodiments, the second compound is represented by the structure of formula K and compounds 200-214. In some embodiments, the second compound is represented by the structure of any one of compounds 204, 205 or 208; each represents a separate embodiment according to this invention. In some embodiments, the second compound is represented by the structure of compound 208. In some embodiments, the second compound further comprises a labeling moiety. In some embodiments, the second compound further comprises at least 2; 3; 4; 5; 6; 7; 8; 9; or 10 labeling moieties; each represents a separate embodiment according to this invention. In some embodiments, the labeling moiety comprises a fluorescent dye.

In some embodiments, the bacterial probe (B-probe) according to this invention comprises a first linker (L₁) and/or a second linker (L₂) as described hereinbelow under the title(s) “A First Linker (L₁)” or “The second linker (L₂)”.

In some embodiments, the first linker comprises at least one oligoethyleneglycol (OEG) moiety, at least one phosphate moiety, at least one thioalkyl moiety or any combination thereof. In some embodiments, the first linker comprises two; three; four; five; six; seven or eight sequentially arranged oligoethylene glycol (OEG) spacers; each represents a separate embodiment according to this invention.

In some embodiments, the synthetic agent of said second compound is as described under the title “synthetic agent” hereinbelow, and/or as described hereinabove for DNA construct according to this invention.

In some embodiments, the bacterial probe (B-probe) further comprises a fourth compound comprising a third oligonucleotide (ODN-3). In some embodiments, the third oligonucleotide (ODN-3) is complementary to the second oligonucleotide (ODN-2) of the DNA construct and/or system of the invention. In some embodiments, ODN-3 comprises higher affinity to said ODN-2 than the affinity of ODN-2 to ODN-1.

### A Kit for Decorating Cell Membranes

In some embodiments, this invention relates to a kit comprising:
a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain, and
c. a second compound according to this invention, comprising a second oligonucleotide (ODN-2) covalently bound to a synthetic agent, either directly or through a second linker, wherein said second oligonucleotide is complementary to said first oligonucleotide

In some embodiments, the polypeptide is bound to the first compound, the second compound is bound to the first compound, or combination thereof; each represents a separate embodiment according to the invention. In some embodiments, when incubated together, the polypeptide, the first compound, and the second compound, form a complex, in which the polypeptide is attached to the first compound and the first compound is attached to the second compound. In some embodiments, the complex can be reversibly modified. In some embodiments, the first compound is attached to the second compound via the hybridization of the first oligonucleotide to the second oligonucleotide. In some embodiments, the first compound is attached to the polypeptide via coordination of said binder to said extracellular binding domain of said polypeptide. In some embodiments, the first compound is attached to the polypeptide via coordination of said binder to an affinity tag comprised in said extracellular binding domain of said polypeptide. In some embodiments, the polypeptide is a cell surface proteins (CSPs). In some embodiments, the polypeptide is an outer membrane protein C (OmpC). In some embodiments, the polypeptide is a receptor tyrosine kinase (RTK). In some embodiments, the polypeptide is an ion channel linked receptor. In some embodiments, the polypeptide is an enzyme-linked receptor. In some embodiments, the polypeptide is a G protein-coupled receptor.

In some embodiments, the kit further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide. In some embodiments, the recombinant cell is selected from: eukaryotes, prokaryotes, mammalian cells, plant cells, human cells, and bacteria. In some embodiments, the bacteria comprise E. coli. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex). In some embodiments, the first compound is represented by the structure of J, H, H(complex), H(a), H(a)(complex), H(b) and H(b)(complex) and compounds 100-106. In some embodiments, the second compound is represented by the structure of formula K and compounds 200-214. In some embodiments, the first linker comprises at least one oligoethyleneglycol (OEG) moiety, at least one phosphate moiety, at least one thioalkyl moiety or any combination thereof. In some embodiments, the first linker comprises two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof, each represents a separate embodiment according to this invention. In some embodiments, the second compound further comprises a second labeling moiety. In some embodiments, the second labeling moiety comprises a fluorescent dye.

### Artificial Receptor

In some embodiments, disclosed herein is an artificial receptor, capable of binding a His-tagged protein, comprising:
a. a first compound comprising a first oligonucleotide (ODN-1) bound to a His-tag binder, either directly or through a first linker, said His-tag binder comprises a moiety represented by the structure of formula E:

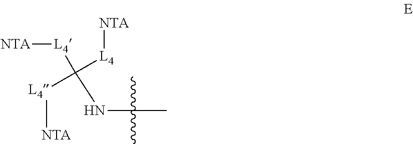

wherein
L₄, L₄′, and L₄″ is each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof, and
b. a second compound comprising a second oligonucleotide (ODN-2) covalently bound to a synthetic agent, either directly or through a second linker, said second oligonucleotide is complementary to said first oligonucleotide.

In some embodiments, the artificial receptor does not perturb the function of a living cell. In some embodiments, the receptor can be reversibly modified. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) as described herein below; each represents a separate embodiment according to this invention. In some embodiments, the first compound is represented by the structure of J, H, H(complex), H(a), H(a)(complex), H(b) and H(b)(complex) and compounds 100-106. In some embodiments, the second compound is represented by the structure of formula K and compounds 200-214, 220, 230, 240 and 250. In some embodiments, the first linker comprises at least one oligoethyleneglycol (OEG) moiety, at least one phosphate moiety, at least one thioalkyl moiety or any combination thereof. In some embodiments, the first linker comprises two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof, each represents a separate embodiment according to this invention. In some embodiments, the second compound further comprises a second labeling moiety. In some embodiments, the second labeling moiety comprises a fluorescent dye. In some embodiments, the artificial receptor further comprises a third compound comprising a third oligonucleotide (ODN-3), wherein said third oligonucleotide is complementary to said second oligonucleotide. In some embodiments, the third oligonucleotide comprises higher affinity to said second oligonucleotide than the affinity of said second oligonucleotide to said first oligonucleotide.

In some embodiments, the first compound is further attached to a polypeptide comprising a His-tag affinity tag, via the binding of said His-tag binder of the first compound, to the His-tag affinity tag of the polypeptide.

In some embodiments, the second compound is bound to the first compound. In some embodiments, when incubated together, the first compound, and the second compound, form a double helix complex, in which the first oligonucleotide is bound to the second oligonucleotide.

In some embodiments, a complex comprising the polypeptide, the first compound, and the second compound, wherein the polypeptide is attached to the first compound and the first compound is attached to the second compound, is termed herein an “artificial receptor”, “synthetic receptor”, “artificial receptor system”, or “synthetic receptor system”. In some embodiments, expressing a polypeptide in a cell and attaching to it a first compound, and in some embodiments, a second compound, is termed “decorating” a cell. In some embodiments, the terms “decorating”, “modifying” and “coating” are used herein interchangeably, having all the same meanings.

### Recombinant Cells

In some embodiments, disclosed herein is a recombinant cell ectopically expressing a polypeptide, wherein said polypeptide comprises a membranal anchoring domain and an extracellular binding domain, said extracellular binding domain bound to
a. a first compound comprising a first oligonucleotide (ODN-1) covalently bound to a binder, either directly or through a first linker, said binder comprising affinity to said extracellular binding domain,
b. a second compound comprising a second oligonucleotide (ODN-2) covalently bound to a synthetic agent, either directly or through a second linker, wherein said second oligonucleotide is complementary to said first oligonucleotide.

In some embodiments, disclosed herein is a recombinant cell ectopically expressing a polypeptide, wherein said polypeptide comprises a membranal anchoring domain and an extracellular binding domain, said extracellular binding domain bound to a DNA construct according to this invention as described hereinabove.

In some embodiments, the recombinant cell is selected from a group comprising eukaryotes, prokaryotes, mammalian cells, plant cells, human cells, and bacteria. In some embodiments, a mammalian or a human cell is selected from a group comprising epithelial cells, Brunner's gland cells in duodenum, insulated goblet cells of respiratory and digestive tracts, stomach, foveolar cells, chief cells, parietal cells, pancreatic acinar cells, Paneth cells of small intestine, Type II pneumocyte of lung, club cells of lung, barrier cells, type i pneumocytes, gall bladder epithelial cells, centroacinar cells, intercalated duct cells, intestinal brush border cells, hormone-secreting cells, enteroendocrine cells, K cells, L cells, I cells, G cells, enterochromaffin cells, enterochromaffin-like cells, N cells, S cells, D cells, Mo cells, thyroid gland cells, thyroid epithelial cells, parafollicular cells, parathyroid gland cells, parathyroid chief cells, oxyphil cells, pancreatic islets, alpha cells, beta cells, delta cells, epsilon cells, PP cells, salivary gland mucous cells, salivary gland serous cells, Von Ebner's gland cells in tongue, mammary gland cells, lacrimal gland cells, ceruminous gland cells in ear, eccrine sweat gland dark cells, eccrine sweat gland clear cells, apocrine sweat gland cells, gland of moll cells in eyelid, sebaceous gland cells, Bowman's gland cells in nose, hormone-secreting cells, anterior/intermediate pituitary cells, corticotropes, gonadotropes, lactotropes, melanotropes, somatotropes, thyrotropes, magnocellsular neurosecretory cells, parvocellsular neurosecretory cells, chromaffin cells, keratinocytes, epidermal basal cells, melanocytes, trichocytes, medullary hair shaft cells, cortical hair shaft cells, cuticular hair shaft cells, huxley's layer hair root sheath cells, Henle's layer hair root sheath cells, outer root sheath hair cells, surface epithelial cells of cornea, tongue, mouth, nasal cavity, distal anal canal, distal urethra, and distal vagina, basal cells, intercalated duct cells, striated duct cells, lactiferous duct cells, ameloblast, auditory inner hair cells of organ of Corti, auditory outer hair cells of organ of Corti, basal cells of olfactory epithelium, primary sensory neurons, Merkel cells of epidermis, olfactory receptor neuron, pain-sensitive primary sensory neurons, photoreceptor cells of retina in eye, proprioceptive primary sensory neurons, touch-sensitive primary sensory neurons, chemoreceptor glomus cells of carotid body cells, outer hair cells of vestibular system of ear, inner hair cells of vestibular system of ear, taste receptor cells of taste bud, neuron cells, interneurons, basket cells, cartwheel cells, Stellate cells, Golgi cells, granule cells, Lugaro cells, unipolar brush cells, Martinotti cells, chandelier cells, Cajal-Retzius cells, double-bouquet cells, neurogliaform cells, retina horizontal cells, amacrine cells, spinal interneuron, renshaw cells, spindle neurons, fork neurons, pyramidal cells, place cells, grid cells, speed cells, head direction cells, Betz cells, stellate cells, boundary cells, bushy cells, Purkinje cells, medium spiny neurons, astrocytes, oligodendrocytes, ependymal cells, tanycytes, pituicytes, adipocytes, white fat cells, brown fat cells, liver lipocytes, cells of the adrenal cortex, cells of the zona glomerulosa, cells of the zona fasciculata, cells of the zona reticularis, theca intema cells of ovarian follicle, granulosa lutein cells, theca lutein cells, leydig cells of testes, seminal vesicle cells, prostate gland cells, bulbourethral gland cells, Bartholin's gland cells, gland of littre cells, uterus endometrium cells, juxtaglomerular cells, macula densa cells of kidney, peripolar cells of kidney, mesangial cells of kidney, parietal epithelial cells, podocytes, proximal tubule brush border cells, loop of Henle thin segment cells, kidney distal tubule cells, kidney collecting duct cells, principal cells, intercalated cells, transitional epithelium, duct cells, efferent ducts cells, epididymal principal cells, epididymal basal cells, endothelial cells, planum semilunatum epithelial cells of vestibular system of ear, organ of Corti interdental epithelial cells, loose connective tissue fibroblasts, comeal fibroblasts, tendon fibroblasts, bone marrow reticular tissue fibroblasts, other nonepithelial fibroblasts, pericytes, hepatic stellate cells, nucleus pulposus cells of intervertebral disc, hyaline cartilage chondrocytes, fibrocartilage chondrocytes, elastic cartilage chondrocytes, osteoblast/osteocytes, osteoprogenitor cells, hyalocyte of vitreous body of eye, stellate cells of perilymphatic space of ear, pancreatic stellate cells, red skeletal muscle cells, white skeletal muscle cells, intermediate skeletal muscle cells, nuclear bag cells of muscle spindle, nuclear chain cells of muscle spindle, myosatellite cells, cardiac muscle cells, cardiac muscle cells, node cells, Purkinje fiber cells, smooth muscle cells, myoepithelial cells of iris, myoepithelial cells of exocrine glands, erythrocytes, megakaryocytes, platelets, monocytes, connective tissue macrophage, epidermal Langerhans cells, osteoclast, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells and committed progenitors for the blood and immune system, oogonium/oocytes, spermatids, spermatocytes, spermatogonium cells, spermatozoon, and interstitial kidney cells.

In some embodiments, a prokaryote comprises a microbial cell such as bacteria, e.g., Gram-positive or Gram-negative bacteria. In some embodiments, the bacteria comprise Gram-negative bacteria or Negativicutes that stain negative in Gram stain. In some embodiments, the bacteria comprise gram-positive bacteria, gram-negative bacteria, or archaea.

In some embodiments, Gram-negative bacteria comprise Acinetobacter calcoaceticus, Actinobacillus actinomycetemcomitans, Aeromonas hydrophila, Alcaligenes xylosoxidans, Bacteroides, Bacteroides fragilis, Bartonella bacilliformis, Bordetella spp., Borrelia burgdorferi, Branhamella catarrhalis, Brucella spp., Campylobacter spp., Chalmydia pneumoniae, Chlamydia psittaci, Chlamydia trachomatis, Chromobacterium violaceum, Citrobacter spp., Eikenella corrodens, Enterobacter aerogenes, Escherichia coli, Flavobacterium meningosepticum, Fusobacterium spp., Haemophilus influenzae, Haemophilus spp., Helicobacter pylori, Klebsiella spp., Legionella spp., Leptospira spp., Moraxella catarrhalis, Morganella morganii, Mycoplasma pneumoniae, Neisseria gonorrhoeae, Neisseria meningitidis, Pasteurella multocida, Plesiomonas shigelloides, Prevotella spp., Proteus spp., Providencia rettgeri, Pseudomonas aeruginosa, Pseudomonas spp., Rickettsia prowazekii, Rickettsia rickettsii, Rochalimaea spp., Salmonella spp., Salmonella typhi, Serratia marcescens, Shigella spp., Treponema carateum, Treponema pallidum, Treponema pallidum endemicum, Treponema pertenue, Veillonella spp., Vibrio cholerae, Vibrio vulnficus, Yersinia enterocolitica, Yersinia pestis.

In some embodiments, the bacteria comprise gammaproteobacteria (e.g. Escherichia coli, pseudomonas, vibrio and klebsiella) or Firmicutes (belonging to class Negativicutes that stain negative in Gram stain).

In some embodiments, Gram-positive bacteria comprise Actinomyces spp., Bacillus anthracis, Bifidobacterium spp., Clostridium botulinum, Clostridium perfringens, Clostridium spp., Clostridium tetani, Corynebacterium diphtheriae, Corynebacterium jeikeium, Enterococcus faecalis, Enterococcus faecium, Erysipelothrix rhusiopathiae, Eubacterium spp., Gardnerella vaginalis, Gemella morbillorum, Leuconostoc spp., Mycobacterium abcessus, Mycobacterium avium complex, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, Nocardia spp., Peptococcus niger, Peptostreptococcus spp., Proprionibacterium spp., Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae (group B streptococcus), Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes (group A streptococcus), Streptococcus salivarius, Streptococcus sanguis.

In some embodiments the bacteria is a species selected from the group consisting of Escherichia, Shigella, Salmonella, Erwinia, Yersinia, Bacillus, Vibrio, Legionella, Pseudomonas, Neisseria, Bordetella, Helicobacter, Listeria, Agrobacterium, Staphylococcus, Streptococcus, Enterococcus, Clostridium, Corynebacterium, Mycobacterium, Treponema, Borrelia, Francisella, Brucella, Campylobacter, Klebsiella, Frankia, Bartonella, Rickettsia, Shewanella, Serratia, Enterobacter, Proteus, Providencia, Brochothrix, and Brevibacterium.

In some embodiments, an oligonucleotide encoding the polypeptide is incorporated in an expression vector. In some embodiments, an oligonucleotide encoding the polypeptide is incorporated in a viral vector. An expression or viral vector can be introduced to the cell by any of the following: transfection, electroporation, infection, or transduction. In other embodiments, the polypeptide is encoded by an mRNA polynucleotide which is delivered for example by electroporation. In one embodiment, methods of electroporation comprise flow electroporation technology.

A skilled artisan would appreciate that the term “vector” encompasses a nucleic acid molecule capable of transporting another nucleic acid to which it has been linked. One type of vector is a “plasmid”, which encompasses a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. A skilled artisan would appreciate that the terms “plasmid” and “vector” may be used interchangeably having all the same qualities and meanings. In one embodiment, the term “plasmid” is the most commonly used form of vector. However, the disclosure presented herein is intended to include such other forms of expression vectors, such as viral vectors (e.g., replication defective retroviruses, lentivirus, adenoviruses and adeno-associated viruses), which serve equivalent functions. Additionally, some viral vectors are capable of targeting a particular cell type either specifically or non-specifically.

The recombinant expression vectors disclosed herein comprise a nucleic acid in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, a skilled artisan would appreciate that the term “operably linked” may encompass nucleotide sequences of interest linked to the regulatory sequence(s) in a manner that allows for expression of the nucleotide sequence (e.g., in an in vitro transcription/translation system or in a host cell when the vector is introduced into the host cell). A skilled artisan would appreciate that term “regulatory sequence” may encompass promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif (1990). Regulatory sequences include those that direct constitutive expression of a nucleotide sequence in many types of host cell and those that direct expression of the nucleotide sequence only in certain host cells (e.g., tissue-specific regulatory sequences).

It will be appreciated by those skilled in the art that the design of the expression vector can depend on such factors as the choice of the host cell to be transformed, the level of expression of protein desired, etc. The expression vectors disclosed here may be introduced into host cells to thereby produce proteins or peptides, including fusion proteins or peptides, encoded by nucleic acids as described herein. For example, an expression vector comprises a nucleic acid encoding a polypeptide comprising a membranal anchoring domain and an extracellular binding domain.

Another embodiment disclosed herein pertains to host cells into which a recombinant expression vector disclosed here has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

For stable transfection of mammalian cells, it is known that, depending upon the expression vector and transfection technique used, only a small fraction of cells may integrate the foreign DNA into their genome the remainder of the DNA remains episomal. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the polypeptide or can be introduced on a separate vector. Cells stably transfected with the introduced nucleic acid can be identified by drug selection (e.g., cells that have incorporated the selectable marker gene will survive, while the other cells die). In another embodiment the transfected cells are identified by the induction of expression of an endogenous reporter gene. In another embodiment the transfected cells are identified by the expression of the polypeptide.

A skilled artisan would appreciate that there are several methods in the art to identify recombinant cells expressing the polypeptide. In some embodiments, the expression of the mRNA encoding the polypeptide can be measured by RT-PCR. In some embodiments, the insertion of a DNA encoding the polypeptide can be identified by DNA gene sequencing. In some embodiments, expression of the polypeptide can be detected by an antibody, for example by Western blotting or ELISA. In some embodiments, the expression of a His-tag on the cell membrane can be detected by a labeled His-tag binder, for example by any of the binders disclosed herein, or by any other His-tag binder available.

In some embodiments, the cell's function is not disturbed by the presence of the polypeptide, the first, and the second compound on its surface. In some embodiments, the cell can be reversibly modified. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) as described herein below; each is a separate embodiment. In some embodiments, the first compound is represented by the structure of J, H, H(complex), H(a), H(a)(complex), H(b) and H(b)(complex) and compounds 100-106. In some embodiments, the second compound is represented by the structure of formula K and compounds 200-214, 220, 230, 240 and 250. In some embodiments, the first linker comprises at least one oligoethyleneglycol (OEG) moiety, at least one phosphate moiety, at least one thioalkyl moiety or any combination thereof, each is a separate embodiment. In some embodiments, the first linker comprises two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the synthetic agent of said second compound comprises a cancer cell binder, a CSP binder, antibody, a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof, each represents a separate embodiment according to this invention. In some embodiments, the second compound further comprises a second labeling moiety. In some embodiments, the second labeling moiety comprises a fluorescent dye.

In some embodiments, the first compound of the system according to this invention is as described under the title “The first Compound (X-ODN-1)” hereinbelow. In some embodiments, the second compound of the system according to this invention is as described under the title “The second Compound (Y-ODN-2)” hereinbelow. In some embodiments, the second compound is represented by the structure of formula K and compounds 200-214, 220, 230, 240 and 250. In some embodiments, the second compound further comprises a labeling moiety. In some embodiments, the second compound further comprises at least 2; 3; 4; 5; 6; 7; 8; 9; or 10 labeling moieties; each represents a separate embodiment according to this invention. In some embodiments, the labeling moiety comprises a fluorescent dye.

### Membranal Anchoring Domain

In some embodiments, the polypeptide according to this invention is a Cell Surface Protein (CSP). Examples of CSPs include but are not limited to: outer membrane protein C (OmpC), G protein-coupled receptors (GPCRs), Receptor tyrosine kinases (RTKs), Programmed Cell Death protein 1 (PD1), Ion channel receptors, Adhesion proteins (e.g., Cadherins, Integrins, Selectins), Transporters and channels (e.g., Sodium-potassium pumps, Aquaporins (water channels), Glucose transporters (e.g., GLUT1), Major histocompatibility complex (MHC) proteins (e.g., MHC class I proteins, MHC class II proteins), Cell adhesion molecules (CAMs) (e.g., Neural cell adhesion molecule (NCAM), Epithelial cadherin (E-cadherin), Immunoglobulin superfamily CAMs (IgCAMs)), Enzymes (e.g., Adenylyl cyclase, Phospholipases, Receptor-associated kinases (e.g., insulin receptor kinase)), Antigenic proteins (e.g., CD antigens (cluster of differentiation), Blood group antigens (e.g., ABO blood group antigens)), Signal transduction proteins (e.g., Protein kinases, Protein phosphatases, Second messenger receptors (e.g., cyclic AMP receptors)). In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention.

In some embodiments, the polypeptide comprises a membranal anchoring domain. In some embodiments, a membranal anchoring domain comprises a polypeptide that, when expressed in a cell, it attaches to the cell membrane. In some embodiments, a membranal anchoring domain comprises at least one end emerging to the extracellular side. In some embodiments, the membranal anchoring domain comprises a transmembranal protein. In some embodiments, the membranal anchoring domain comprises a transmembranal fragment of a protein. In some embodiments, the protein comprises a protein expressed in the recombinant cell. In some embodiments, the protein comprises a cell not expressed in the recombinant cell. In some embodiments, the anchoring domain comprises an artificial polypeptide.

A skilled artisan would appreciate that a membrane anchoring can be selected to be stably expressed in the recombinant cell. For example, the membrane anchoring domain can comprise a protein that is abundantly expressed in the recombinant cell. In some embodiments, the membrane anchoring comprises a protein or a part of it, known to be abundantly expressed on the membrane of the recombinant cell. Thus, a membrane anchoring can be chosen to be a protein abundantly expressed on the recombinant cell membrane.

In some embodiments, a membrane anchoring comprises outer membrane protein C (OmpC) or a part thereof In some embodiments, the transmembranal protein comprises an outer membrane protein C (OmpC); receptor tyrosine kinases (RTKs); Ion channel linked receptors; Enzyme-linked receptors; G protein-coupled receptors, a part thereof or any combination thereof, each represents a separate embodiment according to this invention. In some embodiments, a membrane anchoring comprises a polypeptide comprising at least 80% homology to any of SEQ ID NO.: 13, 16, or 21.

### Extracellular Binding Domain

In some embodiments, the extracellular domain comprised in the recombinant polypeptide comprises an affinity tag. In some embodiments, the binder comprises affinity to a specific affinity tag in the extracellular binding domain.

In some embodiments, an affinity tag comprises a protein tag. In some embodiments, an affinity tag comprises an epitope tag. In some embodiments, an affinity tag comprises a peptide tag. In some embodiments, an affinity tag comprises a combination of a number of tags.

In some embodiments, affinity tags are enzymatically modified, for example they are biotinylatated by biotin ligase. In some embodiments, affinity tags are chemically modified. In some embodiments, expression of a tag does not interfere with the cell functions. In some embodiments, an affinity tag can be removed by specific proteolysis. In some embodiments, tags are removed by TEV protease, Thrombin, Factor Xa or Enteropeptidase.

In some embodiments, an affinity tag is selected from a group comprising AviTag, C-tag, Calmodulin-tag, polyglutamate tag, E-tag, FLAG-tag, HA-tag, His-tag, 5-10 histidines bound by a nickel or cobalt chelate (HHHHHH), Myc-tag, NE-tag, Rho1D4-tag, S-tag, SBP-tag, Softag 1, Softag 3, Spot-tag, Strep-tag, TC tag, Ty tag, V5 tag, VSV-tag, Xpress tag, Isopeptag, SpyTag, SnoopTag, SnoopTagJr, DogTag, SdyTag, BCCP (Biotin Carboxyl Carrier Protein), Glutathione-S-transferase-tag, Green fluorescent protein-tag, HaloTag, SNAP-tag, CLIP-tag, Maltose binding protein-tag, Nus-tag, Thioredoxin-tag, Fc-tag, Designed Intrinsically Disordered tags containing disorder promoting amino acids (P,E,S,T,A,Q,G,.), and Carbohydrate Recognition Domain or CRDSAT-tag; each represents a separate embodiment.

In some embodiments, an affinity tag comprises a poly-histidine peptide comprising 6 histidine residues (6×-His-tag). In some embodiments, an affinity tag comprises a poly-histidine peptide comprising 10 histidine residues (10×-His-tag). In some embodiments, an affinity tag comprises a tetra cysteine peptide (CCPGCC, TC tag).

In some embodiments, more than one type of extracellular binding domain or affinity tag is used. A skilled artisan would recognize using more than one type of extracellular binding domain allows decorating the cell with more than one type of receptor. For example, a first extracellular binding domain and a second extracellular binding domain can be co-expressed in a recombinant cell. The recombinant cell is then incubated with a first and a second binder, wherein the first binder binds the first extracellular binding domain and the second binder binds the second extracellular binding domain. Thus, the first and the second binders will be bound to the same recombinant cell.

### The First Compound (X-ODN-1)

In some embodiments, the first compound (i.e., X-ODN-1) of the DNA construct, system, bacterial probe (B-probe), the artificial receptor, the recombinant cell, and the methods according to this invention comprises:
a. a first oligonucleotide (ODN-1),
b. a binder which comprises affinity to a tagged polypeptide,
c. optionally a first linker which links the first oligonucleotide with the binder,
d. optionally a labeling moiety; and
e. optionally a third linker which links the first oligonucleotide with the labeling moiety.

In some embodiments, the first compound (i.e., X-ODN-1), of the DNA construct, system, bacterial probe (B-probe), the artificial receptor, the recombinant cell, and the methods according to this invention comprises:
a. a first oligonucleotide (ODN-1),
b. a binder which comprises affinity to a tagged polypeptide, and
c. optionally a first linker which links the first oligonucleotide with the binder,

In some embodiments, the first oligonucleotide is directly bound to the binder. In other embodiments, the first oligonucleotide is bound to the binder through a first linker. In some embodiments, the first oligonucleotide is directly bound to the labeling moiety. In other embodiments, the first oligonucleotide is bound to the labeling moiety through a third linker.

In some embodiments, the first compound (i.e., X-ODN-1) of the DNA construct, system, bacterial probe (B-probe), the artificial receptor, the recombinant cell, and the methods according to this invention is represented by the structure of formula J:

F-L₃-ODN1-L₁-Y₁ (J)
wherein
   F is a labeling moiety (e.g., a dye or a dye derivative) or absent;
   L₃ is a third linker or absent;
   ODN1 is a first oligonucleotide sequence;
   L₁ is a first linker or absent; and
   Y₁ is a binder.

In some embodiments, Y₁ of compound of formula J is a His-tag binder. In some embodiments, F of compound of formula J is absent. In some embodiments, L₃ is absent. In some embodiments, both F and L₃ are absent.

In some embodiments, the first compound (i.e., X-ODN-1) of the DNA construct, system, bacterial probe (B-probe), the artificial receptor, the recombinant cell, and the methods according to this invention comprises:
a. a first oligonucleotide (ODN-1),
b. a His-tag binder, and
c. optionally a first linker which links the first oligonucleotide with the His-tag binder.

In some embodiments, the first compound (i.e., X-ODN-1) of the DNA construct, system, bacterial probe (B-probe), the artificial receptor, the recombinant cell, and the methods according to this invention is represented by the structure of formula H:

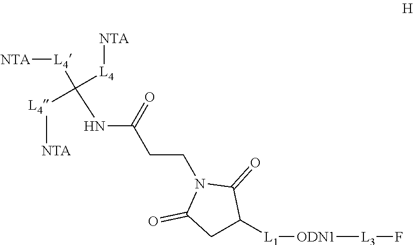

wherein
   F is a labeling moiety or absent (e.g., a dye or a dye derivative);
   L₃ is a third linker or absent;
   ODN1 is a first oligonucleotide sequence;
   L₁ is a first linker or absent;
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms, or any combination thereof.

In some embodiments, the first compound (i.e., X-ODN-1) of the DNA construct, system, bacterial probe (B-probe), the artificial receptor, the recombinant cell, and the methods according to this invention is represented by the structure of the nickel complex of formula H:

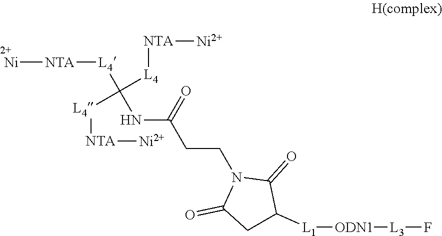

wherein
   F is a labeling moiety or absent (e.g., a dye or a dye derivative);
   L₃ is a third linker or absent;
   ODN1 is a first oligonucleotide sequence;
   L₁ is a first linker or absent;
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms, or any combination thereof.

In some embodiments, the first compound (i.e., X-ODN-1) of the DNA construct, system, bacterial probe (B-probe), the artificial receptor, the recombinant cell, and the methods according to this invention is represented by the structure of formula H(a):

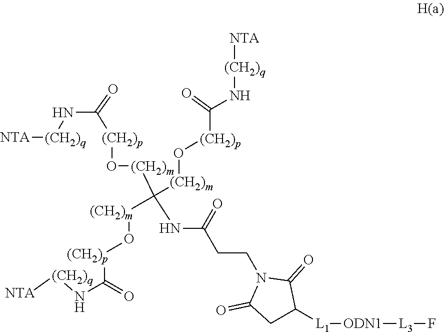

wherein
   F is a labeling moiety or absent (e.g., a dye or a dye derivative);
   L₃ is a third linker or absent;
   ODN1 is a first oligonucleotide sequence;
   L₁ is a first linker or absent;
   m, p and q are each independently an integer number between 1 and 8.

In some embodiments, the first compound (i.e., X-ODN-1) of the DNA construct, system, bacterial probe (B-probe), the artificial receptor, the recombinant cell, and the methods according to this invention is represented by the structure of the nickel complex of formula H(a):

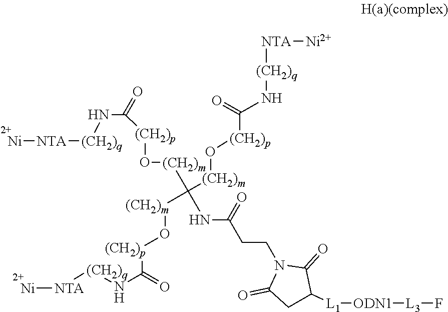

wherein
   F is a labeling moiety or absent (e.g., a dye or a dye derivative);
   L₃ is a third linker or absent;
   ODN1 is a first oligonucleotide sequence;
   L₁ is a first linker or absent;
   m, p and q are each independently an integer number between 1 and 8.

In some embodiments, the first compound (i.e., X-ODN-1) of the DNA construct, system, bacterial probe (B-probe), the artificial receptor, the recombinant cell, and the methods according to this invention is represented by the structure of formula H(b):

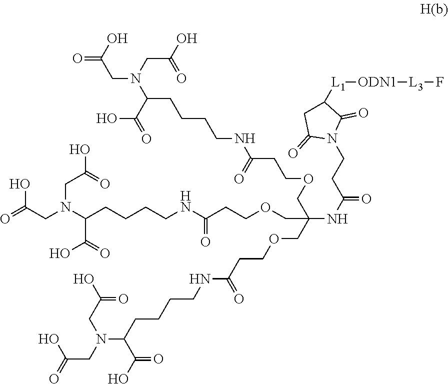

wherein
   F is a labeling moiety or absent (e.g., a dye or a dye derivative);
   L₃ is a third linker or absent;
   ODN1 is a first oligonucleotide sequence; and
   L₁ is a first linker or absent.

In some embodiments, the first compound (i.e., X-ODN-1) of the DNA construct, system, bacterial probe (B-probe), the artificial receptor, the recombinant cell, and the methods according to this invention is represented by the structure of the nickel complex of formula H(b):

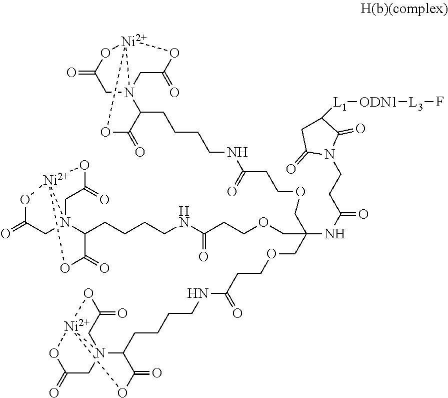

wherein
   F is a labeling moiety or absent (e.g., a dye or a dye derivative);
   L₃ is a third linker or absent;
   ODN1 is a first oligonucleotide sequence; and
   L₁ is a first linker or absent.

In some embodiments, the first compound (i.e., X-ODN-1) of the DNA construct, system, bacterial probe (B-probe), the artificial receptor, the recombinant cell, and the methods according to this invention is represented by the structure of the following compounds and/or their nickel complexes:

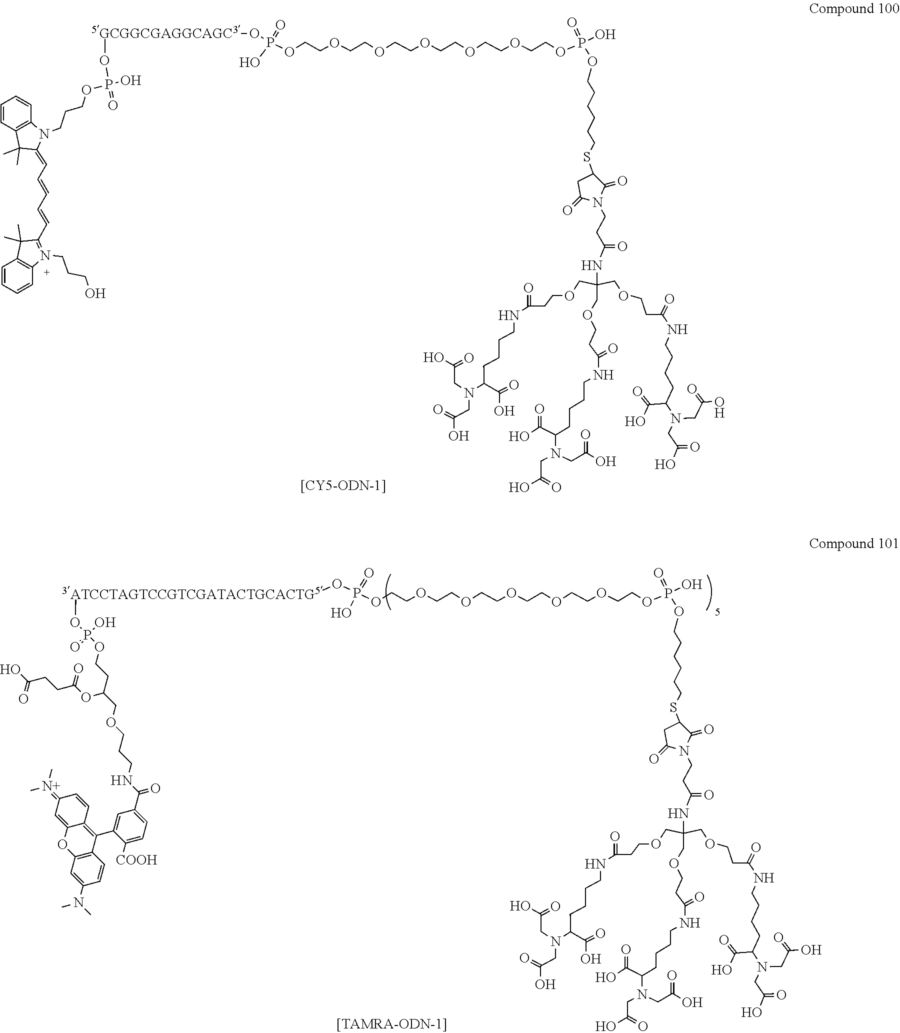

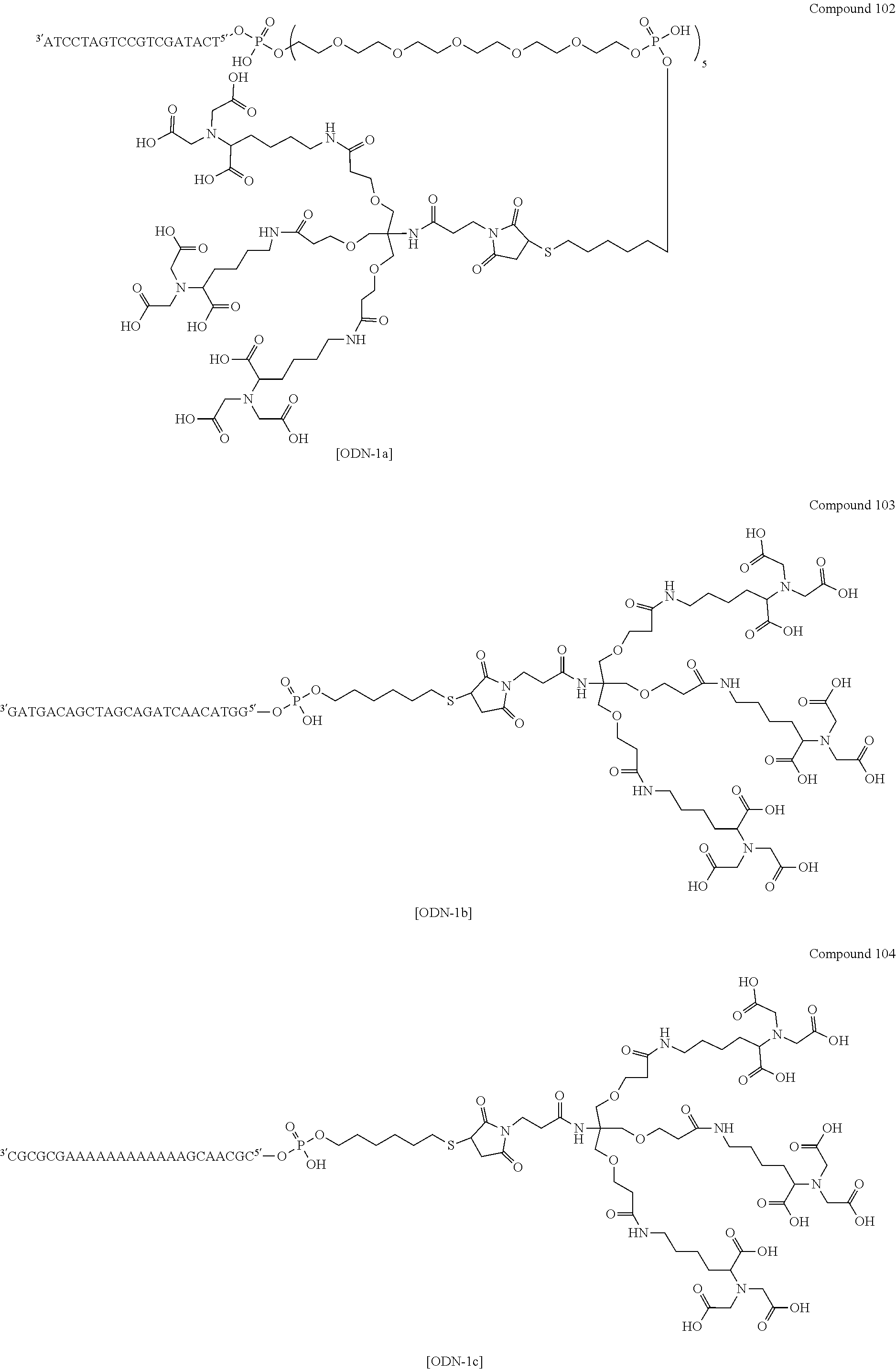

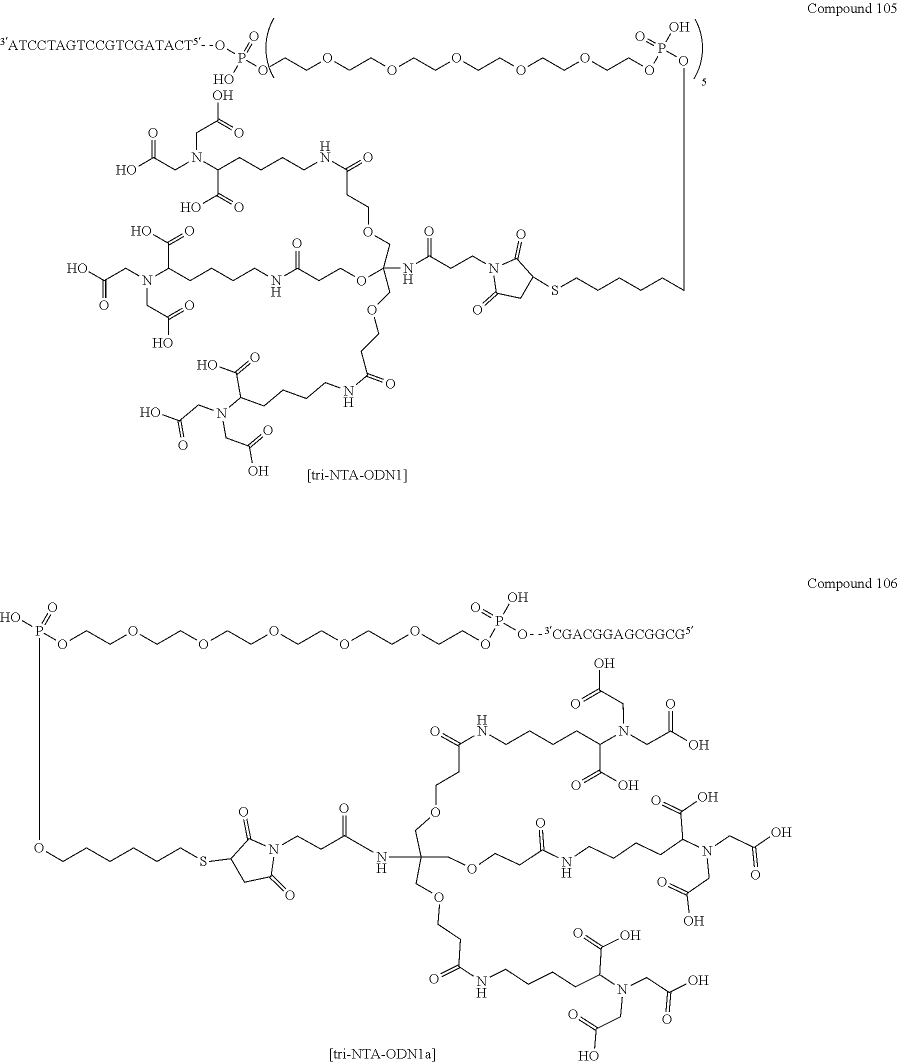

In some embodiments, Y₁ of formulas J is a binder. In some embodiments, Y₁ is an aptamer, a natural ligand, a synthetic group, or a peptide which binds a specific protein with high affinity and selectivity. In some embodiments, Y₁ comprises any selective protein binder known in the art. In another embodiment, Y₁ comprises marimastat, ethacrynic acid, bisethacrynic acid, complexed nitrilotriacetic acid (NTA), complexed bis NTA, complexed tris-NTA, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA), or a peptide binder. In another embodiment, the complexed NTA, complexed bis-NTA, complexed tris NTA is a nickel or cobalt complex. In some embodiments, Y₁ comprises a Tag-binding region. In some embodiments, Y₁ comprises any molecule that can target different type of affinity tags, such as poly-histidine peptide (HHHHHH, His-tag), or tetra cysteine peptide (CCPGCC, TC tag). In another embodiment, Y₁ comprises FlAsH probe. In another embodiment, Y₁ comprises ReAsH probe. In some embodiments, Y₁ comprises a His-tag binder. In some embodiments, Y₁ is a His-tag binder. In some embodiments, Y₁ comprises Ni-nitrilotriacetic acid (Ni-NTA), bis-Ni-NTA, or tris-Ni-NTA. In some embodiments, Y₁ comprises a derivative of Ni-nitrilotriacetic acid (Ni-NTA), bis-Ni-NTA, or tris-Ni-NTA, wherein the term “derivative” includes but not limited to alkyl derivatives, amide derivatives, amine derivatives, carboxy derivatives, and the like. In some embodiments, Y₁ comprises a derivative of tris-Ni-nitrilotriacetic acid (tris-Ni-NTA), a derivative of bis-Ni-nitrilotriacetic acid (bis-Ni-NTA), a derivative of mono-Ni-nitrilotriacetic acid (Ni-NTA); each represents a separate embodiment according to this invention. In some embodiments, Y₁ comprises any monomolecular compound which comprises three Ni-NTA moieties (i.e., tris-Ni-NTA). In some embodiments, Y₁ is represented by the structure of formulas D, D(a), D(b), G, G(a), G(b) as described herein below. In some embodiments, Y₁ comprises the structure of formulas D, D(a), D(b), G, G(a), G(b) as described herein below.

In some embodiments, L₁ of formulas J, H, H(complex), H(a), H(a)(complex), H(b) and H(b)(complex) is a first linker. In some embodiments, L₁ is absent. In some embodiments, L₁ is bound to the 3′ end of ODN1. In some embodiments, L₁ is bound to the 5′ end of ODN1. In some embodiments, L₁ is bound to Y₁ through an amide bond, an ester bond, a phosphate bond, an ether bond, a thioether bond, each represents a separate embodiment according to this invention. In some embodiments, L₁ is as defined for the “first linker” hereinbelow.

In some embodiments, ODN1 of formulas J, H, H(complex), H(a), H(a)(complex), H(b) and H(b)(complex) is a first oligonucleotide sequence.

In some embodiments, the first oligonucleotide (ODN-1 or ODN1) is 1-1000; 3-500; 4-250; 5-100; 10-80; 10-50; 15-35; 5-25; 15-25; 17-23; 15-20; 20-30; 10; 11; 12; 13; 14; 15; 16; 17; 18; 19; 20; 21; 22; 23; 24; 25; 26; 27; 28; 29; 30 bases long; each represents a separate embodiment according to this invention. In some embodiments, the first oligonucleotide is as described hereinbelow.

In some embodiments, ODN1 is directly bound to Y₁, through an amide bond, an ester bond, a phosphate bond, an ether bond, each represents a separate embodiment according to this invention. In some embodiments, ODN1 is directly bound to F, through an amide bond, an ester bond, a phosphate bond, an ether bond, a thioether bond, each represents a separate embodiment according to this invention. In some embodiments, ODN1 is directly bound to F, through a phosphate moiety.

In some embodiments, L₃ of formulas J, H, H(complex), H(a), H(a)(complex), H(b) and H(b)(complex) is a third linker. In some embodiments, L₃ is absent. In some embodiments, L₃ is bound to the 3′ end of ODN1. In some embodiments, L₃ is bound to the 5′ end of ODN1. In some embodiments, L₃ is bound to F through an amide bond, an ester bond, a phosphate bond, an ether bond, a thioether bond, each represents a separate embodiment according to this invention. In some embodiments, L₃ is as defined for the “third linker” hereinbelow.

In some embodiments, F of formulas J, H, H(complex), H(a), H(a)(complex), H(b) and H(b)(complex) is a labeling moiety. In some embodiments, F is absent. In some embodiments, F is a dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof. In some embodiments, F is a dye derivative. In some embodiments, a labeling moiety is bound to ODN1 through an amide bond, an ester bond, a phosphate bond, an ether bond, a thioether bond; each represents a separate embodiment according to this invention. In some embodiments, a labeling moiety F is bound to L₃ through an amide bond, an ester bond, a phosphate bond, an ether bond, a thioether bond; each represents a separate embodiment according to this invention.

### Linkers (L₁ and L₃)

In some embodiments, the first compound (i.e., X-ODN-1) of the DNA construct, system, bacterial probe (B-probe), the artificial receptor, the recombinant cell, and the methods according to this invention comprises:
a. a first oligonucleotide (ODN-1)
b. a binder which comprises affinity to the extracellular binding domain of said polypeptide,
c. optionally a first linker which links the first oligonucleotide with the binder
d. optionally a labeling moiety, and
e. optionally a third linker which links the first oligonucleotide with the labeling moiety.

In some embodiments, the first compound (i.e., X-ODN-1) of the DNA construct, system, bacterial probe (B-probe), the artificial receptor, the recombinant cell, and the methods according to this invention comprises:
a. a first oligonucleotide (ODN-1),
b. a binder which comprises affinity to a tagged polypeptide, and
c. optionally a first linker which links the first oligonucleotide with the binder,

The terms “linker” or “spacer” are used interchangeably and refer to a chemical fragment that connects between the 5′ or the 3′ end of an oligonucleotide according to this invention, and other chemical moieties of the system of the invention (e.g., binder, labeling moiety or a dye, synthetic agent, etc). In some embodiments, the linker is covalently bound to the oligonucleotide through a phosphate moiety.

### A First Linker (L1)

In some embodiments, the first compound (X-ODN-1) of the DNA construct, system, bacterial probe (B-probe), the artificial receptor, the recombinant cell, and the methods according to the invention, comprises a first linker, which links the first oligonucleotide with the binder. In some embodiments, the first linker is covalently bound to the 3′ end of the first oligonucleotide (ODN-1). In some embodiments, the first linker is covalently bound to the 5′ end of the first oligonucleotide. In some embodiments, the first linker is covalently bound to the binder through an amide bond, an ester bond, a phosphate bond, an ether bond, a thioether bond; each represents a separate embodiment according to this invention. In some embodiments, the first linker is covalently bound to the first oligonucleotide through an amide bond, an ester bond, a phosphate bond, an ether bond, a thioether bond, each represents a separate embodiment according to this invention. In some embodiments, the first linker is covalently bound to the first oligonucleotide through a phosphate moiety.

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invetion, is any chemical fragment which comprises at least one segment of a commercially available phosphoramidite spacer derivative. Phosphoramidite compounds are used as reactive agents for linking oligonucleotides according to this invention with other moieties, e.g., the binder of this invention, the labeling moiety, the synthetic agents, etc. Non limiting examples of such phosphoramidite derivatives, useful for linking oligonucleotides with other moieties include:

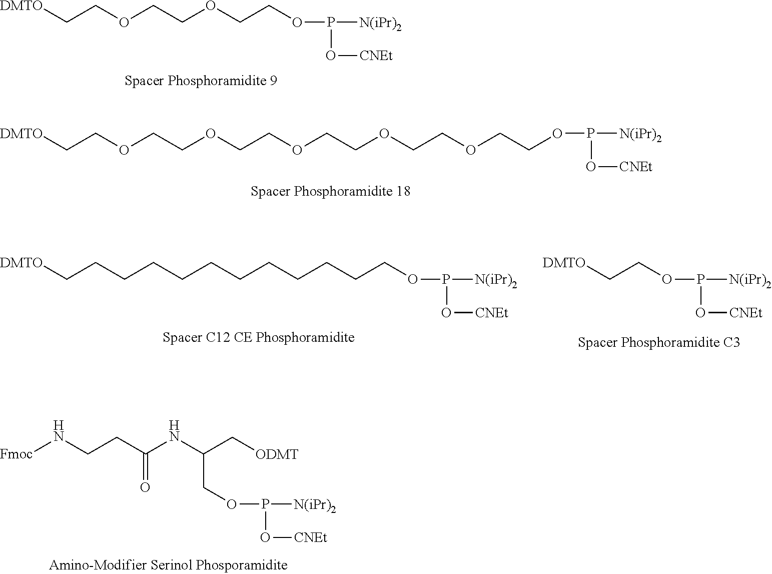

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invention, is a substituted or unsubstituted linear or branched alkyl chain of 1-20 carbon atoms, substituted or unsubstituted linear or branched alkyl ether chain of 1-20 carbon atoms, oligoethylene glycol (OEG), polyethylene glycol (PEG), oligopropylene glycol, polypropylene glycol (PPG), substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 2-10 carbon atoms, substituted or unsubstituted linear or branched alkyl triazole of 1-20 carbon atoms, substituted or unsubstituted linear or branched alkyl amine chain of 1-20 carbon atoms or any combination thereof; each represents a separate embodiment according to this invention.

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises at least one oligoethyleneglycol (OEG) moiety. In some embodiments, the first linker and/or L₁ comprises two; three; four; five; six; seven; eight; nine or ten sequentially arranged oligoethylene glycol (OEG) spacers; each represents a separate embodiment according to this invention. In some embodiments, the first linker, and/or L₁ comprises at least one phosphate moiety. In some embodiments, the first linker, and/or L₁ comprises at least one alkyl ether moiety. In some embodiments, the first linker, and/or L₁ comprises at least one alkyl diamide moiety. In some embodiments, the first linker, and/or L₁ comprises at least one alkyl moiety. In some embodiments, the first linker, and/or L₁ comprises at least one thioalkyl moiety. In some embodiments, the first linker, and/or L₁ comprises at least one oligoethyleneglycol (OEG) moiety and at least one phosphate moiety. In some embodiments, the first linker, and/or L₁ comprises at least one oligoethyleneglycol (OEG) moiety, at least one phosphate moiety, at least one thioalkyl moiety, at least one alkyl moiety, or any combination thereof.

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises the following monomer:

—[(CH₂O)ₖ—PO₃H]ₗ—
wherein
   k and l are each independently an integer number between 0 and 10; and
   w is an integer number between 1 and 10.

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises one; two; three; four; five; six; seven; eight; nine; or ten monomeric units; each represents a separate embodiment according to this invention. In some embodiments, the first and/or the second linker comprises two monomeric units. In some embodiments, the first linker comprises five monomeric units.

In some embodiments, the first linker and/or L₁ according to formula J, H, H(complex), H(a), H(a)(complex), H(b) and H(b)(complex) of the DNA construct, system, the bacterial probe, (B-probe), the artificial receptor, the recombinant cell, and the methods according to this invention, is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, l is 0. In some embodiments, 1 is 1. In some embodiments, 1 is 5. In some embodiments, 1 is 2. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### A Third Linker (L₃)

In some embodiments, the first compound (X-ODN-1) of the DNA construct, system, the bacterial probe, (B-probe), the artificial receptor, the recombinant cell, and the methods according to this invention comprises a third linker, which links the first oligonucleotide with the labeling moiety. In some embodiments, the third linker is absent. In some embodiments, the third linker is bound to the 3′ end of ODN-1. In some embodiments, the third linker is bound to the 5′ end of ODN-1. In some embodiments, the third linker is a part of a commercially available phosphoramidite dye derivative. In some embodiments, the third linker is bound to the labeling moiety through an amide bond, an ester bond, a phosphate bond, an ether bond, a thioether bond; each represents a separate embodiment according to this invention. In some embodiments, the third linker is bound to ODN-1 through an amide bond, an ester bond, a phosphate bond, an ether bond, a thioether bond; each represents a separate embodiment according to this invention. In some embodiments, the third linker is covalently bound to the first oligonucleotide through a phosphate moiety.

In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention is a substituted or unsubstituted linear or branched alkyl chain of 1-20 carbon atoms, substituted or unsubstituted linear or branched alkyl ether chain of 1-20 carbon atoms, oligoethylene glycol (OEG), polyethylene glycol (PEG), oligopropylene glycol, polypropylene glycol (PPG), substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 2-10 carbon atoms, substituted or unsubstituted linear or branched alkyl triazole of 1-20 carbon atoms, substituted or unsubstituted linear or branched alkyl amine chain of 1-20 carbon atoms or any combination thereof; each is a separate embodiment according to this invention.

In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises at least one oligoethyleneglycol (OEG) moiety. In some embodiments, the third linker and/or L₃ comprise two; three; four; five; six; seven; eight; nine or ten sequentially arranged oligoethylene glycol (OEG) spacers; each represents a separate embodiment according to this invention. In some embodiments, the third linker, and/or L₃ comprises at least one phosphate moiety. In some embodiments, the third linker, and/or L₃ comprises at least one alkyl ether moiety. In some embodiments, the third linker, and/or L₃ comprises at least one alkyl diamide moiety. In some embodiments, the third linker, and/or L₃ comprises at least one alkyl moiety. In some embodiments, the third linker, and/or L₃ comprises at least one thioalkyl moiety. In some embodiments, the third linker, and/or L₃ comprises at least one oligoethyleneglycol (OEG) moiety and at least one phosphate moiety. In some embodiments, the third linker, and/or L₃ comprises at least one oligoethyleneglycol (OEG) moiety, at least one phosphate moiety, at least one thioalkyl moiety, at least one alkyl moiety, or any combination thereof.

In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises the following monomer:

[(CH₂O)ₖ—PO₃H]ₗ—
wherein
   k and l are each independently an integer number between 0 and 10; and
   w is an integer number between 1 and 10.

In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises one; two; three; four; five; six; seven; eight; nine; or ten monomeric units; each represents a separate embodiment according to this invention. In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises two monomeric units. In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, comprises five monomeric units.

In some embodiments, the third linker and/or L₃ according to formula J, H, H(complex), H(a), H(a)(complex), H(b) and H(b)(complex) of the DNA construct, system, the bacterial probe, (B-probe), the artificial receptor, the recombinant cell, and the methods according to this invention, is represented by the following formula:

—[(CH₂O)ₖ—PO₃H]—(CH₂)_{w}—S—
wherein
   k and l are each independently an integer number between 0 and 10; and
   w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, l is 0. In some embodiments, 1 is 1. In some embodiments, 1 is 2. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### Binder (Y1)

In some embodiments, a binder of the DNA construct, system, bacterial probe (B-probe), the artificial receptor, the recombinant cell, and the methods according to this invention is an aptamer, a natural ligand, a synthetic group, or a peptide, which binds a specific protein with high affinity and selectivity.

In some embodiments, the binder of the DNA construct, system, bacterial probe (B-probe), the artificial receptor, the recombinant cell, and the methods of this invention is any selective protein binder known in the art. In another embodiment, the selective protein binder comprises marimastat, ethacrynic acid, bisethacrynic acid, complexed nitrilotriacetic acid (NTA), complexed bis NTA, complexed tris-NTA, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA), a peptide binder, or derivative thereof, each represents a separate embodiment according to this invention. In another embodiment, the complexed NTA, complexed bis-NTA, complexed tris NTA is a nickel or cobalt complex.

In some embodiments, the binder comprises a Tag-binding region.

In some embodiments, the binder is any molecule that can target different type of affinity tags, such as poly-histidine peptide (HHHHHH, His-tag), or tetra cysteine peptide (CCPGCC, TC tag). In another embodiment, the binder is FlAsH probe. In another embodiment, the binder is ReAsH probe.

In some embodiments, the selective binder is a His-tag specific binder. In some embodiments, the binder of this invention comprises nitrilotriacetic acid (NTA), bis-NTA, ortris-NTA moieties, which in the presence of Nickel ions bind a Histidine affinity tag. In some embodiments, the binder of this invention comprises Ni-nitrilotriacetic acid (Ni-NTA), bis-Ni-NTA, or tris-Ni-NTA. In some embodiments, the binder of this invention comprises a derivative of Ni-nitrilotriacetic acid (Ni-NTA), bis-Ni-NTA, or tris-Ni-NTA, wherein the term “derivative” includes but not limited to alkyl derivatives, amide derivatives, amine derivatives, carboxy derivatives, and the like. In some embodiments, the His-Tag binder comprises a derivative of tris-nitrilotriacetic acid (tris-NTA), a derivative of bis-nitrilotriacetic acid (bis-NTA), a derivative of mono-nitrilotriacetic acid (NTA), which in the presence of Nickel ions can bind a histidine affinity tag; each represents a separate embodiment according to this invention. In some embodiments, the His-Tag binder comprises a derivative of tris-Ni-nitrilotriacetic acid (tris-Ni-NTA), a derivative of bis-Ni-nitrilotriacetic acid (bis-Ni-NTA), a derivative of mono-Ni-nitrilotriacetic acid (Ni-NTA); each represents a separate embodiment according to this invention. In some embodiments, the His-tag binder is any monomolecular compound which comprises three Ni-NTA moieties (i.e., tris-Ni-NTA).

In some embodiments, the binder according to this invention is a His-tag specific binder. In some embodiments, the His-tag specific binder binds a histidine affinity tag in the presence of nickel ions.

In some embodiments, the His-tag specific binder comprised in the DNA construct, system, bacterial probe (B-probe), the artificial receptor, the recombinant cell, and the methods of the invention comprises a moiety represented by the structure of Formula C:

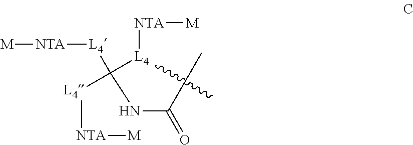

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof; and
   M-NTA is a metal complex of nitrilotriacetic acid.

In some embodiments, M is a metal ion. In some embodiments, M is cobalt (Co). In some embodiments, M is nickel (Ni). In some embodiments, M is Ni(II). In some embodiments, M is Co(II). In some embodiments, M is Co(III).

In some embodiments, the His-tag specific binder comprised in the DNA construct, system, bacterial probe (B-probe), the artificial receptor, the recombinant cell, and the methods of the invention comprises a moiety represented by the structure of formula D:

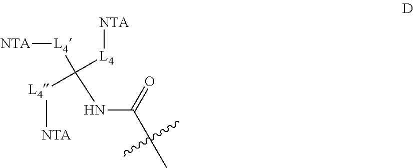

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In some embodiments, the His-tag specific binder comprised in the DNA construct, system, bacterial probe (B-probe), the artificial receptor, the recombinant cell, and the methods of the invention comprises a moiety represented by the structure of the nickel complex of formula D:

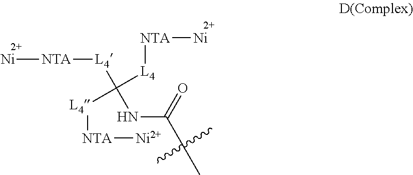

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag specific binder comprised in the DNA construct, system, bacterial probe (B-probe), the artificial receptor, the recombinant cell, and the methods of the invention comprises a moiety represented by the structure of formula D(a):

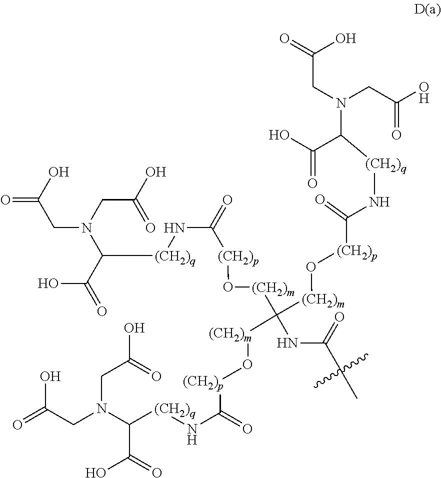

wherein
m, p and q are each independently an integer number between 1 and 8.

In another embodiment, the His-tag specific binder comprised in the DNA construct, system, bacterial probe (B-probe), the artificial receptor, the recombinant cell, and the methods of the invention comprises a moiety represented by the structure of the nickel complex of formula D(a):

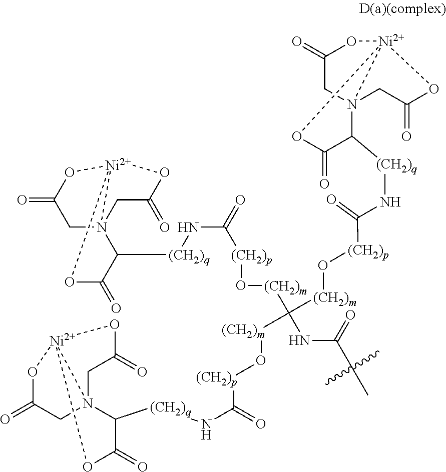

wherein
m, p and q are each independently an integer number between 1 and 8.

In another embodiment, the His-tag specific binder comprised in the DNA construct, system, bacterial probe (B-probe), the artificial receptor, the recombinant cell, and the methods of the invention comprises a moiety represented by the structure of formula D(b):

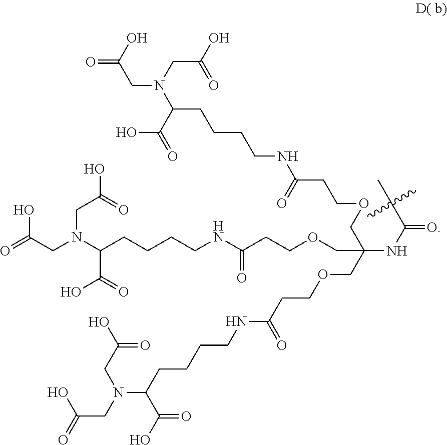 [SMILES: *C(=O)NC(COCCC(=O)NCCCCC(C(=O)O)N(CC(=O)O)CC(=O)O)(COCCC(=O)NCCCCC(C(=O)O)N(CC(=O)O)CC(=O)O)COCCC(=O)NCCCCC(C(=O)O)N(CC(=O)O)CC(=O)O]

In another embodiment, the His-tag specific binder comprised in the DNA construct, system, bacterial probe (B-probe), the artificial receptor, the recombinant cell, and the methods of the invention comprises a moiety represented by the structure of the nickel complex of formula D(b):

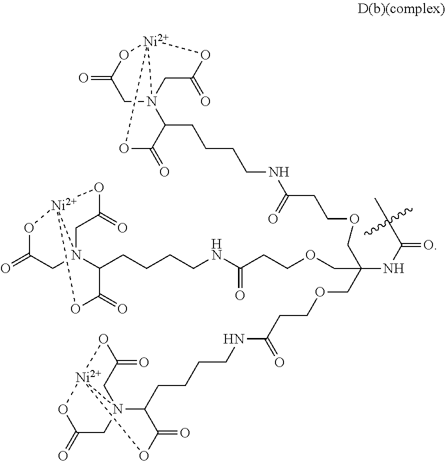

In some embodiments, the His-tag specific binder comprised in the DNA construct, system, bacterial probe (B-probe), the artificial receptor, the recombinant cell, and the methods of the invention comprises a moiety represented by the structure of formula E:

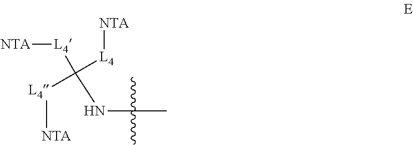

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In some embodiments, the His-tag specific binder comprised in the DNA construct, system, bacterial probe (B-probe), the artificial receptor, recombinant cell, and the methods of the invention comprises a moiety represented by the structure of the nickel complex of formula E:

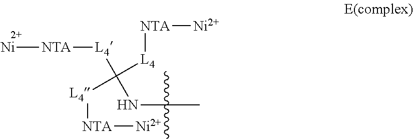

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag specific binder comprised in the DNA construct, system, bacterial probe (B-probe), the artificial receptor, the recombinant cell, and the methods of the invention comprises a moiety represented by the structure of formula E(a):

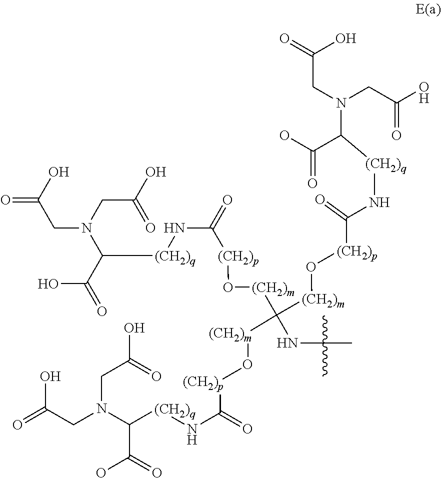

wherein
m, p and q are each independently an integer number between 1 and 8.

In another embodiment, the His-tag specific binder comprised in the DNA construct, system, bacterial probe (B-probe), the artificial receptor, the recombinant cell, and the methods of the invention comprises a moiety represented by the structure of the nickel complex of formula E(a):

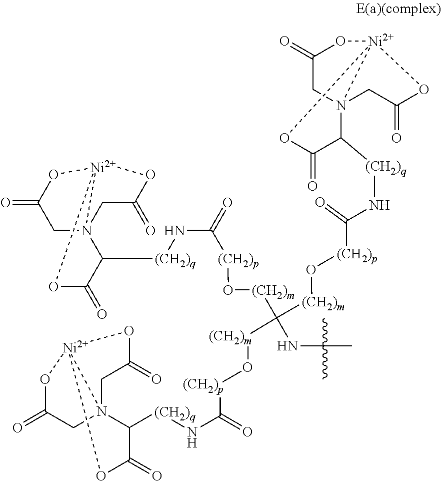

wherein
m, p and q are each independently an integer number between 1 and 8.

In some embodiments, the His-tag specific binder comprised in the DNA construct, system, bacterial probe (B-probe), the artificial receptor, the recombinant cell, and the methods of the invention comprises a moiety represented by the structure of formula E(b):

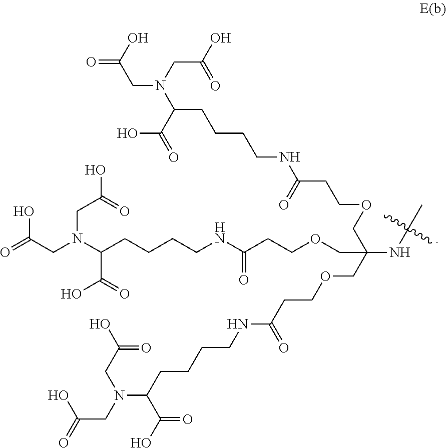 [SMILES: *NC(COCCC(=O)NCCCCC(C(=O)O)N(CC(=O)O)CC(=O)O)(COCCC(=O)NCCCCC(C(=O)O)N(CC(=O)O)CC(=O)O)COCCC(=O)NCCCCC(C(=O)O)N(CC(=O)O)CC(=O)O]

In some embodiments, the His-tag specific binder comprised in the DNA construct, system, bacterial probe (B-probe), the artificial receptor, the recombinant cell, and the methods of the invention comprises a moiety represented by the structure of the nickel complex of formula E(b):

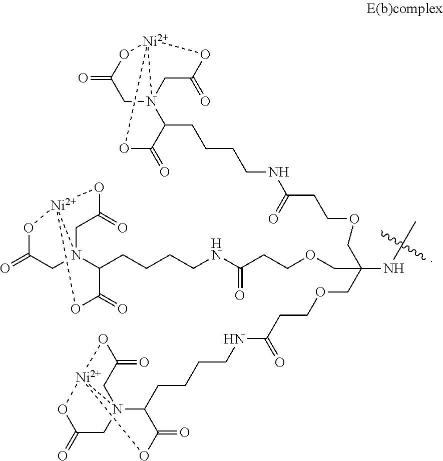

In some embodiments, the His-tag specific binder comprised in the DNA construct, system, bacterial probe (B-probe), the artificial receptor, the recombinant cell, and the methods of the invention comprises a moiety represented by the structure of formula G:

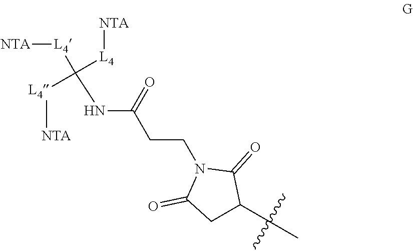

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In some embodiments, the His-tag specific binder comprised in the DNA construct, system, bacterial probe (B-probe), the artificial receptor, the recombinant cell, and the methods of the invention comprises a moiety represented by the structure of the nickel complex of formula G:

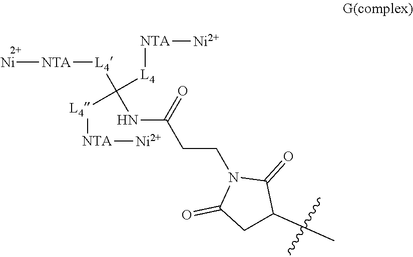

wherein
   L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof.

In another embodiment, the His-tag specific binder comprised in the of the DNA construct, system, the bacterial probe, (B-probe), the artificial receptor, the recombinant cell, and the methods according to this invention comprises a moiety represented by the structure of formula G(a):

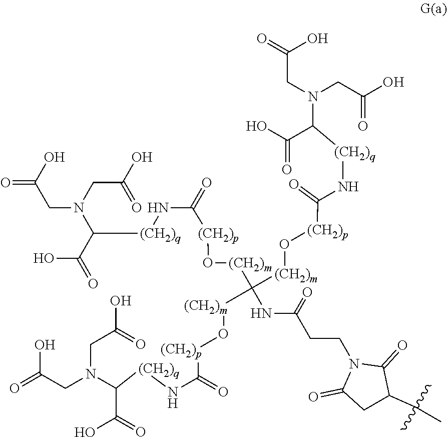

wherein
m, p and q are each independently an integer number between 1 and 8.

In another embodiment, the His-tag specific binder comprised in the of the DNA construct, system, the bacterial probe, (B-probe), the artificial receptor, the recombinant cell, and the methods according to this invention comprises a moiety represented by the structure of the nickel complex of formula G(a):

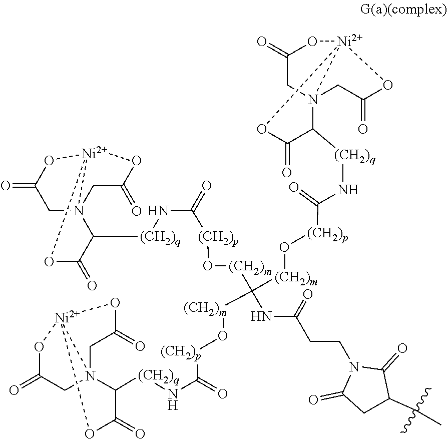

wherein
m, p and q are each independently an integer number between 1 and 8.

In some embodiments, the His-tag specific binder comprised in the DNA construct, system, bacterial probe (B-probe), the artificial receptor, the recombinant cell, and the methods of the invention comprises a moiety represented by the structure of formula G(b):

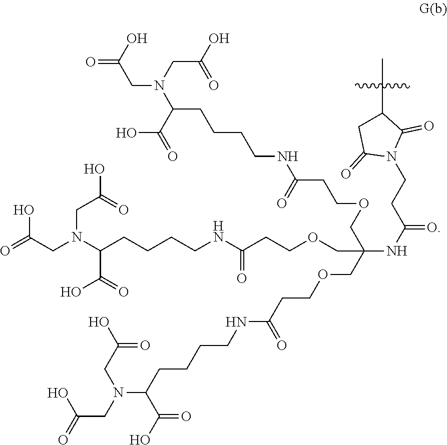 [SMILES: *C1CC(=O)N(CCC(=O)NC(COCCC(=O)NCCCCC(C(=O)O)N(CC(=O)O)CC(=O)O)(COCCC(=O)NCCCCC(C(=O)O)N(CC(=O)O)CC(=O)O)COCCC(=O)NCCCCC(C(=O)O)N(CC(=O)O)CC(=O)O)C1=O]

In some embodiments, the His-tag specific binder comprised in the DNA construct, system, bacterial probe (B-probe), the artificial receptor, the recombinant cell, and the methods of the invention comprises a moiety represented by the structure of the nickel complex of formula G(b):

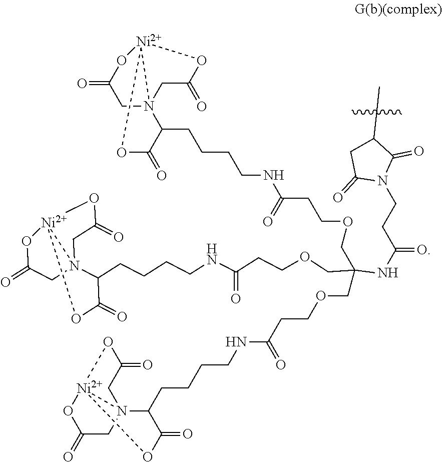

In some embodiments, each of L₄, L₄′, and L₄″ of the structures of formulas D, D(complex), E, E(complex), G, G(complex), H and/or H(complex), is independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof, each represents a separate embodiment according to this invention. In some embodiments, each of L₄, L₄′, and L₄″ is a combination of alkyl ether and alkyl amide (i.e., alkylether-alkylamide). In another embodiment, each of L₄, L₄′, and L₄″ is independently —(CH₂)_{q}—NHCO—(CH₂)ₚ—O—(CH₂)ₘ—, wherein q, p and m are each independently an integer between 1 and 8. In another embodiment, q is 4, p is 2 and m is 1. In another embodiment, each of L₄, L₄′, and L₄″ is —(CH₂)4—NHCO—(CH₂)₂O—CH₂—. In another embodiment, each of L₄, L₄′, and L₄″ is represented by the following structure:

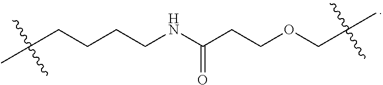 [SMILES: *CCCCNC(=O)CCOC*]

In another embodiment, m of the structures of formulas D(a), D(a)(complex), E(a), E(a)(complex), G(a), G(a)(complex), H(a) and/or H(a)(complex) is 1. In another embodiment, m is 2. In another embodiment, m is 3. In another embodiment, m is 4.

In another embodiment, p of the structures of formulas D(a), D(a)(complex), E(a), E(a)(complex), G(a), G(a)(complex), H(a) and/or H(a)(complex) is 1. In another embodiment, p is 2. In another embodiment, p is 3. In another embodiment, p is 4.

In another embodiment, q of the structures of formulas D(a), D(a)(complex), E(a), E(a)(complex), G(a), G(a)(complex), H(a) and/or H(a)(complex) is 1. In another embodiment, q is 2. In another embodiment, q is 3. In another embodiment, q is 4. In another embodiment, q is 5. In another embodiment, q is 6.

In another embodiment, m is 1, p is 2 and q is 4.

### ODN Sequences

As used herein, “oligonucleotide sequence,” “oligonucleotide” or “nucleic acid sequence” refer to a nucleotide, oligonucleotide, polynucleotide, or any fragment thereof and to naturally occurring or synthetic molecules, such as L-DNA, phosphorothioates, locked nucleic acids, etc.

As used herein, an “oligonucleotide”, “ODN” or “oligonucleotide sequence” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group. An oligonucleotide is a nucleic acid that includes at least two nucleotides.

One oligonucleotide sequence may be “complementary” to a second oligonucleotide sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete”, “full” or “total” complementarity between the nucleic acids. The degree of complementarity between the oligonucleotide strands has effects on the efficiency and strength of hybridization between the nucleic acid strands.

Oligonucleotides as described herein may be capable of forming hydrogen bonds with oligonucleotides having a complementary base sequence. These bases may include the natural bases such as A, G, C, T and U, as well as artificial bases. An oligonucleotide may include nucleotide substitutions. For example, an artificial or modified base may be used in place of a natural base such that the artificial base exhibits a specific interaction that is similar to the natural base.

An oligonucleotide that is complementary to another nucleic acid will “hybridize” to the nucleic acid under suitable conditions (described below). As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15M NaCl, 0.015M sodium citrate, pH 7.2)

In some embodiments, the oligonucleotide sequences of the DNA construct, system, bacterial probe (B-probe), the artificial receptor, the recombinant cell, and the methods according to the invention may each be at least 4, at least 8, at least 12, at least 16, at least 20, or at least 30 nucleotides in length; each is a separate embodiment according to this invention. In illustrative embodiments, oligonucleotide sequences may each be no more than about 200 nucleotides in length. In illustrative embodiments, oligonucleotide sequences may each be no more than about 50 nucleotides in length. In one embodiment, the oligonucleotide sequences, may be partially complementary to a third oligonucleotide, which binds the oligonucleotide sequences for the formation of larger molecular assemblies.

### ODN-1 (or ODN1)

In some embodiments, the first oligonucleotide (ODN1) of the DNA construct, system, bacterial probe (B-probe), the artificial receptor, the recombinant cell, and the methods according to the invention is at least 4, at least 8, at least 12, at least 16, at least 20, or at least 30 nucleotides in length; each is a separate embodiment according to this invention. In some embodiments, the first oligonucleotide of the DNA construct, system, bacterial probe (B-probe), the artificial receptor, the recombinant cell, and the methods according to the invention is no more than about 50 nucleotides in length. In some embodiments, the first oligonucleotide is at least 2, at least 4, at least 8, at least 12, at least 16, or at least 20 nucleotides shorter than the second oligonucleotide; each is a separate embodiment according to this invention.

In some embodiments, the first oligonucleotide (ODN-1) of the DNA construct, system, bacterial probe (B-probe), the artificial receptor, the recombinant cell, and the methods according to the invention is 1-1000; 3-500; 4-250; 5-100; 10-80; 10-50; 15-35; 5-25; 15-25; 17-23; 15-20; 20-30; 10; 11; 12; 13; 14; 15; 16; 17; 18; 19; 20; 21; 22; 23; 24; 25; 26; 27; 28; 29; 30 bases long; each represents a separate embodiment according to this invention.

In some embodiments, the first oligonucleotide comprises a sequence comprising at least 80% homology to any of SEQ ID NOs.: 1-5. In some embodiments, the first oligonucleotide sequence is represented by any one of SEQ ID NOs.: 1-5. In some embodiments, the first oligonucleotide comprises a sequence comprising at least 80% homology to any of SEQ ID NOs.: 3. In some embodiments, the first oligonucleotide sequence is represented by any one of SEQ ID NOs.: 3.

### ODN-2 (or ODN2)

In some embodiments, the second oligonucleotide (ODN2) of the DNA construct, system, bacterial probe (B-probe), the artificial receptor, the recombinant cell, and the methods according to the invention is at least 4, at least 8, at least 12, at least 16, at least 20, or at least 30 nucleotides in length; each is a separate embodiment according to this invention. In some embodiments, the second oligonucleotide of the DNA construct, system, bacterial probe (B-probe), the artificial receptor, the recombinant cell, and the methods according to the invention is no more than about 50 nucleotides in length. In some embodiments, the second oligonucleotide is at least 2, at least 4, at least 8, at least 12, at least 16, or at least 20 nucleotides longer than the first oligonucleotide; each is a separate embodiment according to this invention. In some embodiments, the second oligonucleotide comprises a toehold region.

In some embodiments, the second oligonucleotide (ODN-2) of the DNA construct, system, bacterial probe (B-probe), the artificial receptor, the recombinant cell, and the methods according to the invention is 1-1000; 3-500; 4-250; 5-100; 10-80; 10-50; 15-35; 5-45; 15-55; 17-23; 15-20; 20-40; 30-40; 30-35; 10; 15; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50 bases long; each represents a separate embodiment according to this invention.

In some embodiments, the second oligonucleotide comprises a sequence comprising at least 80% homology to any of SEQ ID NOs.: 6-9, 22, 23, and 25. In some embodiments, the second oligonucleotide sequence is represented by any one of SEQ ID NOs.: 6-9, 22, 23, and 25.

In some embodiments, the second oligonucleotide (ODN-2) is longer than the first oligonucleotide (ODN-1). In some embodiments, the second oligonucleotide (ODN-2) is shorter than the first oligonucleotide (ODN-1). In some embodiments, the second oligonucleotide (ODN-2) and the first oligonucleotide (ODN-1) are of the same length.

In some embodiments, the second oligonucleotide (ODN-2) comprises a hanging strand (a toehold region).

### Hanging Strand

In some embodiments, the second oligonucleotide (ODN-2) of the DNA construct, system, bacterial probe (B-probe), the artificial receptor, the recombinant cell, and the methods according to the invention, further comprises a hanging strand (a toehold region).

In some embodiments, the hanging strand of the second oligonucleotide (ODN-2) is appended with at least one labeling moiety. In some embodiments, the hanging strand of the second oligonucleotide (ODN-2) is appended with at least two; three; four; five; six; seven; eight; nine; or ten labeling moieties; each represents a separate embodiment according to this invention. In some embodiments, the hanging strand of the second oligonucleotide (ODN-2) is appended with two; three; four; five; six; seven; eight; nine; or ten labeling moieties; each represents a separate embodiment according to this invention. In some embodiments, the hanging strand of the second oligonucleotide (ODN-2) is appended with six labeling moieties.

In some embodiments, the labeling moieties attached to the hanging strand of the second oligonucleotide (ODN-2) are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the labeling moieties attached to said hanging strand are located 4-6 bases apart from each other. In some embodiments, the labeling moieties attached to said hanging strand are located 5 bases apart from each other. In some embodiments, the labeling moieties are covalently attached to the hanging strand. In some embodiments, the labeling moieties are covalently attached to a certain nucleotide of the hanging strand. In some embodiments, the labeling moieties are covalently attached to a Thymine nucleotide of the hanging strand.

In some embodiments, the hanging strand of the second oligonucleotide (ODN-2) is not appended with a labeling moiety.

In some embodiments, the hanging strand of the second oligonucleotide (ODN-2) is appended with at least one fluorescent dye. In some embodiments, the hanging strand of the second oligonucleotide (ODN-2) is appended with at least two; three; four; five; six; seven; eight; nine; or ten fluorescent dyes; each represents a separate embodiment according to this invention. In some embodiments, the hanging strand of the second oligonucleotide (ODN-2) is appended with two; three; four; five; six; seven; eight; nine; or ten fluorescent dyes; each represents a separate embodiment according to this invention. In some embodiments, the hanging strand of the second oligonucleotide (ODN-2) is appended with six fluorescent dyes.

In some embodiments, the fluorescent dyes attached to the hanging strand of the second oligonucleotide (ODN-2) are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the fluorescent dyes attached to said hanging strand are located 4-6 bases apart from each other. In some embodiments, the fluorescent dyes attached to said hanging strand are located 5 bases apart from each other. In some embodiments, the fluorescent dyes are covalently attached to the hanging strand. In some embodiments, the fluorescent dyes are covalently attached to a certain nucleotide of the hanging strand. In some embodiments, the fluorescent dyes are covalently attached to a Thymine nucleotide of the hanging strand.

In some embodiments, the hanging strand of the second oligonucleotide (ODN-2) is not appended with fluorescent dye.

In some embodiments, the second oligonucleotide (ODN-2) of the second compound of the DNA construct, system, bacterial probe (B-probe), the artificial receptor, the recombinant cell, and the methods according to the invention, further comprises a hanging strand (a toehold region). In some embodiments, the hanging strand is a first hanging strand. In some embodiments, the first hanging strand is complementary to a second hanging strand comprised in the DNA duplex (dsDNA) as described hereinbelow.

In some embodiments, the DNA duplex of the third compound of the DNA construct, system, bacterial probe (B-probe), the artificial receptor, the recombinant cell, and the methods according to the invention, further comprise a hanging strand (a toehold region). In some embodiments, the hanging strand is a second hanging strand. In some embodiments, the second hanging strand is comprised in the longer oligonucleotide comprising the DNA duplex (ssDNA-long). In some embodiments, the second hanging strand is complementary to a first hanging strand comprised in the second oligonucleotide (ODN2) of the second compound as described hereinbelow.

In some embodiments, the hanging strand of the second oligonucleotide (ODN2) and/or the hanging strand of the DNA duplex (dsDNA) of the DNA construct, system, bacterial probe (B-probe), the artificial receptor, the recombinant cell, and the methods according to the invention is at least 2, at least 4, at least 8, at least 12, at least 16, at least 20, or at least 30 nucleotides in length; each is a separate embodiment according to this invention. In some embodiments, the hanging strand of the second oligonucleotide (ODN2) and/or the hanging strand of the DNA duplex (dsDNA) of the DNA construct, system, bacterial probe (B-probe), the artificial receptor, the recombinant cell, and the methods according to the invention is no more than about 50 nucleotides in length.

In some embodiments, the hanging strand of the second oligonucleotide (ODN2) and/or the hanging strand of the DNA duplex (dsDNA) of the DNA construct, system, bacterial probe (B-probe), the artificial receptor, the recombinant cell, and the methods according to the invention is 1-1000; 3-500; 4-250; 5-100; 1-100; 3-50; 4-25; 5-50; 10-80; 10-50; 5-20; 5-15; 7-18; 5-45; 7-35; 15-30; 15-35; 20-30; 2; 4; 5; 6; 7; 8; 9; 10; 11; 12; 13; 14; 15; 16; 17; 18; 19;20; 21;22; 23; 24;25; 26; 27; 28; 29; 30 bases long; each represents a separate embodiment according to this invention.

### ODN-3 (or ODN3)

In some embodiments, the DNA construct, system and/or the bacterial probe (B-probe) according to this invention further comprises a fourth compound comprising a third oligonucleotide (ODN-3).

In some embodiments, the DNA construct, system and/or the bacterial probe (B-probe) according to this invention, further comprises a third oligonucleotide (ODN-3).

In some embodiments, the third oligonucleotide is fully complementary to the second oligonucleotide.

In some embodiments, ODN-3 is capable of detaching ODN-2 from ODN-1, thereby detaching the second compound according to this invention from the cell of the invention.

In some embodiments, the third oligonucleotide (ODN3) of the DNA construct, system and/or the bacterial probe (B-probe) according to the invention is at least 4, at least 8, at least 12, at least 16, at least 20, at least 25, or at least 30 nucleotides in length. In some embodiments, the third oligonucleotide of the system according to the invention is no more than about 50 nucleotides in length. In some embodiments, the third oligonucleotide is at least 2, at least 4, at least 8, at least 12, at least 16, or at least 20 nucleotides longer than the second oligonucleotide; each is a separate embodiment according to this invention. In some embodiments, the third oligonucleotide has the same length as the second oligonucleotide. In some embodiments, the third oligonucleotide is at least 2, at least 4, at least 8, at least 12, at least 16, or at least 20 nucleotides longer than the first oligonucleotide; each is a separate embodiment according to this invention.

In some embodiments, the third oligonucleotide comprises a sequence comprising at least 80% homology to SEQ ID NO.: 10. In some embodiments, the third oligonucleotide sequence is represented by SEQ ID NO.: 10. In some embodiments, the third oligonucleotide comprises a sequence comprising at least 80% homology to SEQ ID NO.: 24. In some embodiments, the third oligonucleotide sequence is represented by SEQ ID NO.: 24.

In some embodiments, ODN-3 of the system according to the invention is capable of detaching ODN-2 from ODN-1 by a toehold mechanism.

In some embodiments, ODN-2 comprises a toehold region (a hanging strand) complementary to a fragment of ODN-3. A “toehold region” refers to an oligonucleotide segment that comprises a single-stranded overhang that allows detaching two complementary oligonucleotides. In some embodiments, ODN-2 is hybridized to ODN-1, and ODN-2 further comprises a toehold region, which is a single-stranded overhang not complementary of ODN-1. In some embodiments, ODN-2's toehold region is complementary to a fragment of ODN-3. Therefore, in some embodiments, when ODN-3 is added, it binds to ODN-2 toehold region. Once ODN-3 is bound to the toehold region, ODN-3 will compete with ODN-1 for binding the rest of ODN-2's bases. As ODN-1 and ODN-3 exchange base pairs with ODN-2, the branch point of the three-stranded complex moves back and forth. This 'three-way branch migration′ is an unbiased random walk, as each step causes no net change in base pairing. Eventually, ODN-1 will fully dissociate, and ODN-2 will become fully bound to ODN-3. Thus, in some embodiments, ODN-3 can be used to detach the second compound, ODN-2, or the synthetic agent from the recombinant cell.

In some embodiments, ODN-2 comprises a toehold region (a hanging strand) complementary to the full length of ODN-3. Thus, in some embodiments, ODN-3 is not used to detach the second compound, ODN-2, or the synthetic agent from the recombinant cell.

In some embodiments, the third oligonucleotide (ODN-3) is complementary to the hanging strand. In some embodiments, the third oligonucleotide is 1-1000; 3-500; 4-250; 5-100; 10-80; 10-50; 15-35; 5-25; 15-25; 17-30; 15-30; 20-35; 10; 15; 16; 17; 18; 19; 20; 21; 22; 23; 24; 25; 26; 27; 28; 29; 30; 31; 32; 33; 34; 35; 36; 37; 38; 39; 40 bases long; each represents a separate embodiment according to this invention. In some embodiments, the third oligonucleotide comprises a sequence comprising at least 80% homology to SEQ ID NO.: 24. In some embodiments, the third oligonucleotide sequence is represented by SEQ ID NO.: 24.

In some embodiments, the third oligonucleotide is fully complementary to the hanging strand of the second oligonucleotide.

### The Second Compound (Y-ODN-2)

In some embodiments, the DNA construct, system, bacterial probe (B-probe), the artificial receptor, the recombinant cell, and the methods of this invention, comprise a second compound comprising a second oligonucleotide (ODN-2) covalently bound to a synthetic agent, either directly or through a second linker, wherein said second oligonucleotide is complementary to said first oligonucleotide.

In some embodiments, the first compound of the DNA construct, system, bacterial probe (B-probe), the artificial receptor, the recombinant cell, and the methods of this invention, is attached to the second compound via the hybridization of the first oligonucleotide (ODN-1) to the second oligonucleotide (ODN-2).

In some embodiments, the second compound (i.e., Y-ODN-2) of the DNA construct, system, bacterial probe (B-probe), the artificial receptor, the recombinant cell, and the methods according to this invention comprises:
a. a second oligonucleotide (ODN-2), which is complementary to said first oligonucleotide;
b. a synthetic agent,
c. optionally a second linker, which links the second oligonucleotide with the synthetic agent;
d. optionally a second labeling moiety;
e. optionally a fourth linker which links the second oligonucleotide with the second labeling moiety.

In some embodiments, the second compound (i.e., Y-ODN-2) of the DNA construct, system, bacterial probe (B-probe), the artificial receptor, the recombinant cell, and the methods according to this invention comprises:
a. a second oligonucleotide (ODN-2), which is complementary to said first oligonucleotide;
b. a synthetic agent, and
c. optionally a second linker which links the second oligonucleotide with the synthetic agent.

In some embodiments, the second oligonucleotide is bound directly to the synthetic agent. In some embodiments, the second oligonucleotide is bound to the synthetic agent through a second linker.

In some embodiments, the second oligonucleotide comprises a hanging strand (a toehold region). In some embodiments, the hanging strand is appended with at least one labeling moiety. In some embodiments, the hanging strand is appended with at least two labeling moieties. In some embodiments, the hanging strand is appended with at least two; three; four; five; six; seven; eight; nine; or ten labeling moieties; each represents a separate embodiment according to this invention.

In some embodiments, the hanging strand is appended with at least one fluorescent dye. In some embodiments, the hanging strand is appended with at least two fluorescent dyes. In some embodiments, the hanging strand is appended with at least two; three; four; five; six; seven; eight; nine; or ten fluorescent dyes; each represents a separate embodiment according to this invention. In some embodiments, the hanging strand is appended with at least three fluorescent dyes. In some embodiments, the hanging strand is appended with six fluorescent dyes.

In some embodiments, the second compound (i.e., Y-ODN-2) of the DNA construct, system, bacterial probe (B-probe), the artificial receptor, the recombinant cell, and the methods according to this invention comprises:
a. a second oligonucleotide (ODN-2), which is complementary to said first oligonucleotide, and wherein said second oligonucleotide comprises a hanging strand appended with at least two fluorescent dyes;
b. a synthetic agent, and
c. optionally a second linker which links the second oligonucleotide with the synthetic agent.

In some embodiments, the second oligonucleotide comprises a first hanging strand (a first toehold region). In some embodiments, the first hanging strand is complementary to a second hanging strand comprised in a DNA duplex as described hereinbelow. In some embodiments, the first hanging strand is appended with at least one labeling moiety. In some embodiments, the hanging strand is appended with at least two labeling moieties. In some embodiments, the hanging strand is appended with at least three labeling moieties. In some embodiments, the hanging strand is appended with at least two; three; four; five; six; seven; eight; nine; or ten labeling moieties; each represents a separate embodiment according to this invention. In some embodiments, the hanging strand is appended with at least one fluorescent dye. In some embodiments, the hanging strand is appended with at least two fluorescent dyes. In some embodiments, the hanging strand is appended with at least three fluorescent dyes. In some embodiments, the hanging strand is appended with at least two; three; four; five; six; seven; eight; nine; or ten fluorescent dyes; each represents a separate embodiment according to this invention.

In some embodiments, the second oligonucleotide comprises a hanging strand (a toehold region) appended with at least two fluorescent dyes.

In some embodiments, the second compound (i.e., Y-ODN-2) of the DNA construct, system, bacterial probe (B-probe), the artificial receptor, the recombinant cell, and the methods according to this invention comprises a second oligonucleotide (ODN-2), which comprises a hanging strand appended with at least two fluorescent dyes.

In some embodiments, the second oligonucleotide is directly bound to the synthetic agent. In other embodiments, the second oligonucleotide is bound to the synthetic agent through a second linker. In some embodiments, the second oligonucleotide is directly bound to at least one labeling moiety. In other embodiments, the second oligonucleotide is bound to the second labeling moiety through a fourth linker. In other embodiments, the labeling moiety is a fluorescent dye. In some embodiments, the second oligonucleotide comprises a hanging strand (a toehold region) appended with at least two fluorescent dyes.

In some embodiments, the second compound (i.e., Y-ODN-2) of the DNA construct, system, bacterial probe (B-probe), the artificial receptor, the recombinant cell, and the methods according to this invention is represented by the structure of formula K:

F₂-L₄-ODN2-L₂-X (K)
wherein
   X is a synthetic agent;
   L₂ is a second linker or absent;
   ODN2 is a second oligonucleotide sequence;
   L₄ is a fourth linker or absent; and
   F₂ is a second labeling moiety or absent.

In some embodiments, F₂ of the compound of formula K is absent. In some embodiments, L₄ is absent. In some embodiments, both F₂ and L₄ are absent.

In some embodiments, the second compound according to this invention (i.e., Y-ODN-2) is represented by the structure of the following compounds:

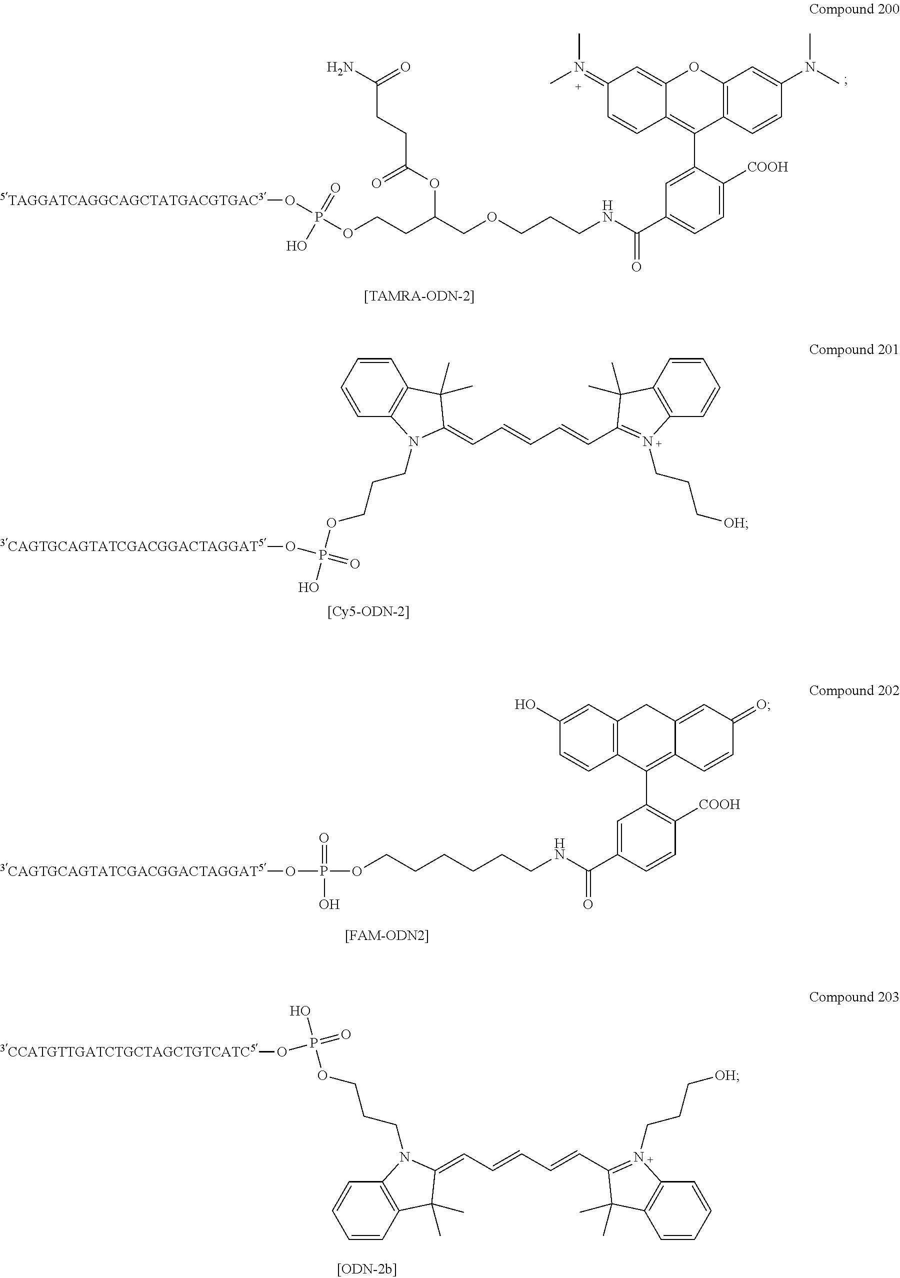

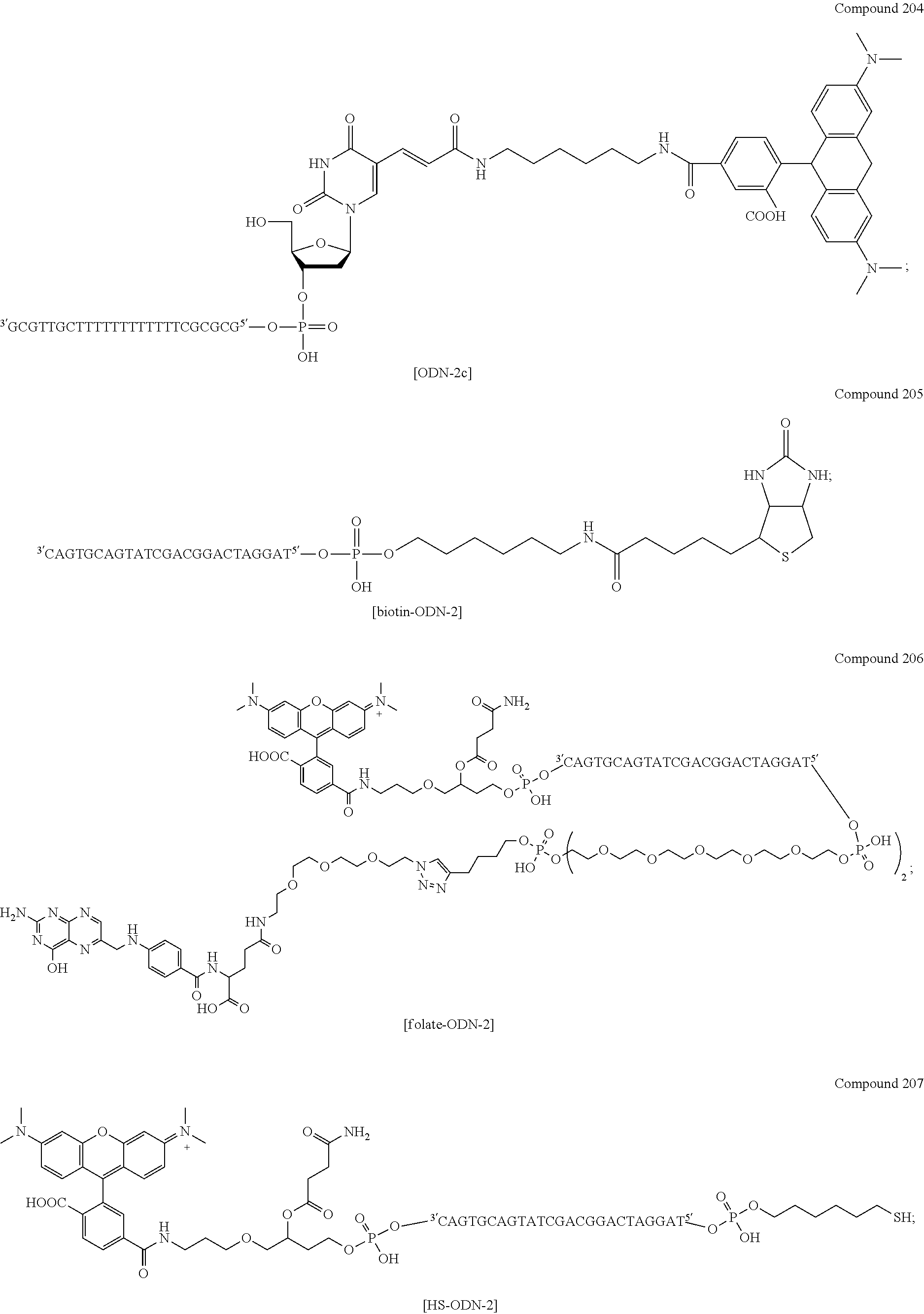

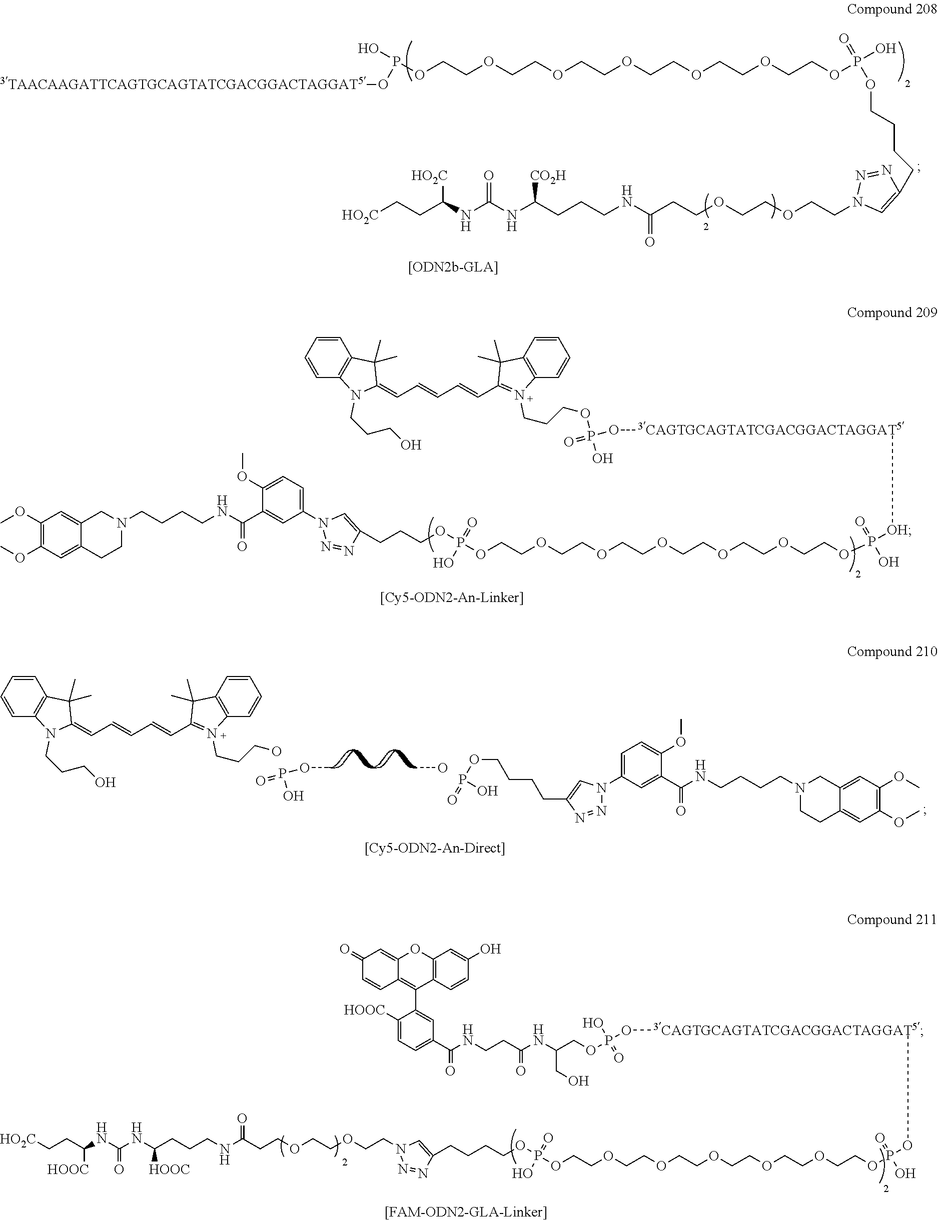

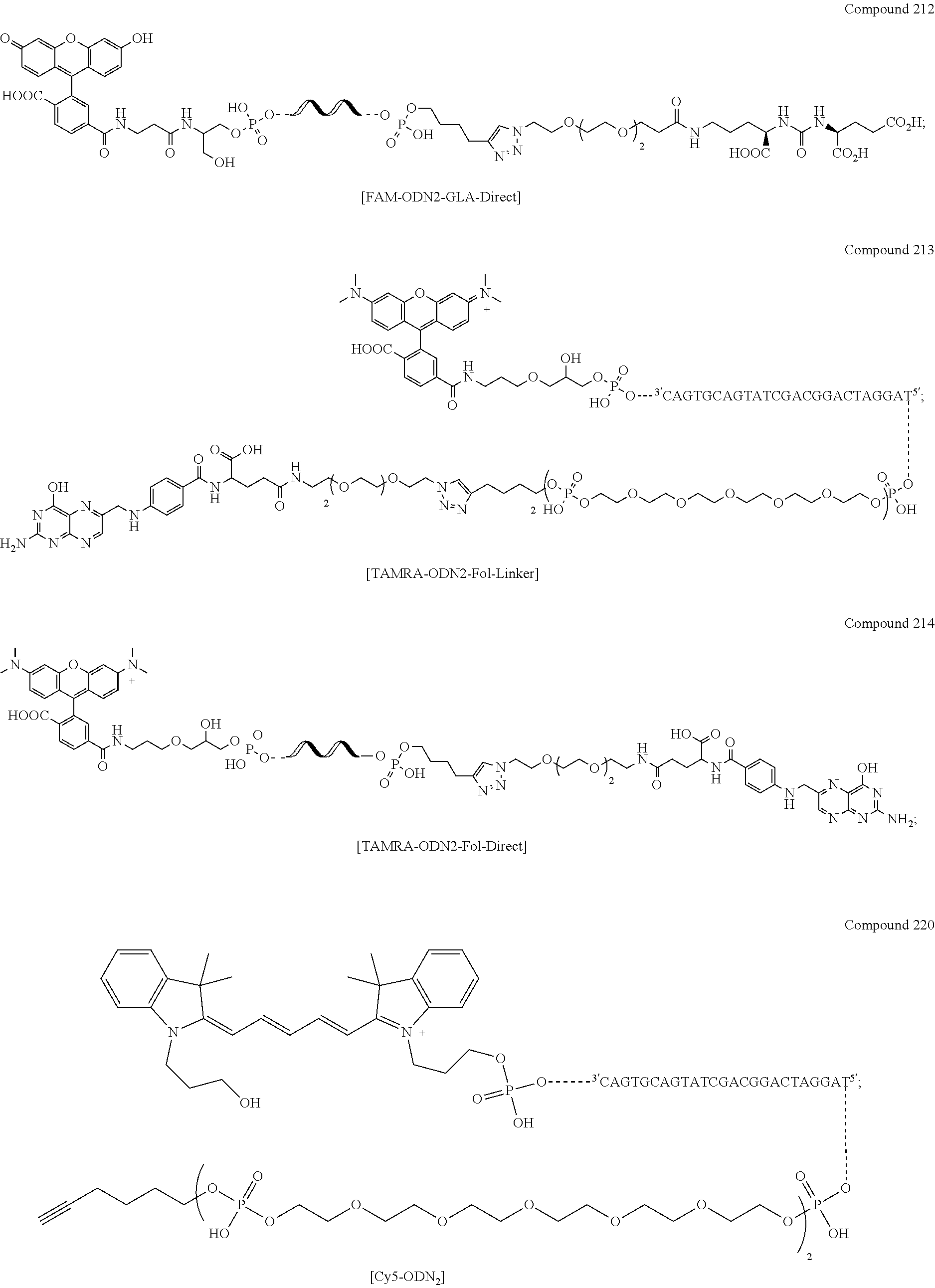

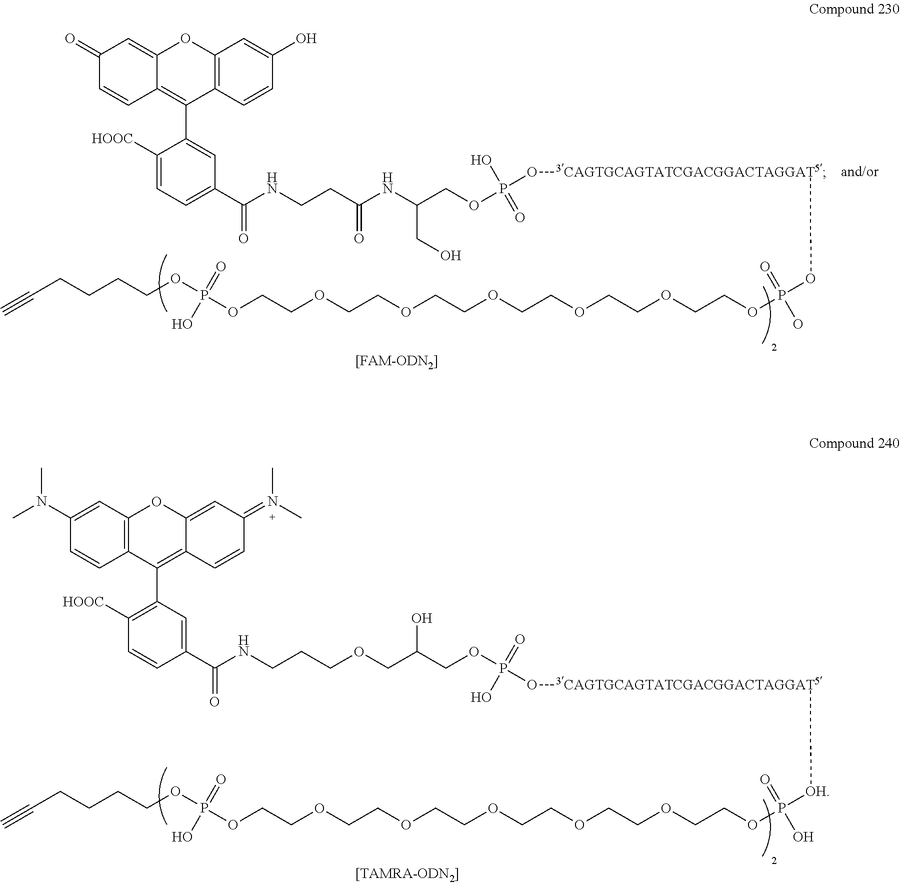

In some embodiments, X of formula K is a synthetic agent. In some embodiments, X of formula K comprises a synthetic agent. In some embodiments, X of formula K comprises a synthetic agent's derivative. In some embodiments, X is a selective protein binder. In some embodiments, X comprises a selective protein binder. In some embodiments, X comprises a selective protein binder's derivative. In some embodiments, X comprises a cancer cell binder. In some embodiments, X comprises a cancer cell binder's derivative. In some embodiments, X comprises a CSP binder. In some embodiments, X comprises a CSP binder's derivative. In some embodiments, X is a folate. In some embodiments, X is a folate derivative (Fol). In some embodiments, X is an anisamide. In some embodiments, X is an anisamide derivative (An). In some embodiments, X is a glutamate urea. In some embodiments, X is a glutamate urea derivative (GLA). In some embodiments, X is a biotin. In some embodiments, X is a biotin derivative. In some embodiments, X comprises an adhesion molecule. In some embodiments, X comprises a surface binder. In some embodiments, X comprises an abiotic surface binder. In some embodiments, X comprises an —SH functional group. In some embodiments, X is a thioalkyl. In some embodiments, X is a labeling moiety. In some embodiments, X is a dye. In some embodiments, X is a fluorescent dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof. In some embodiments, X is bound to ODN2 through an amide bond, an ester bond, a phosphate bond, an ether bond, a thiolether bond, each represents a separate embodiment according to this invention. In some embodiments, X is covalently bound to ODN2 through a phosphate moiety. In some embodiments, X is bound to L₂ through an amide bond, an ester bond, a phosphate bond, an ether bond, a thioether bond, each represents a separate embodiment according to this invention. In some embodiments, X is as described herein below in the definition of a synthetic agent. In some embodiments, X is a dye derivative. In some embodiments, X is a derivative of a commercially available phosphoramidite dye agent. Non limiting examples of such phosphoramidite dye agents include:

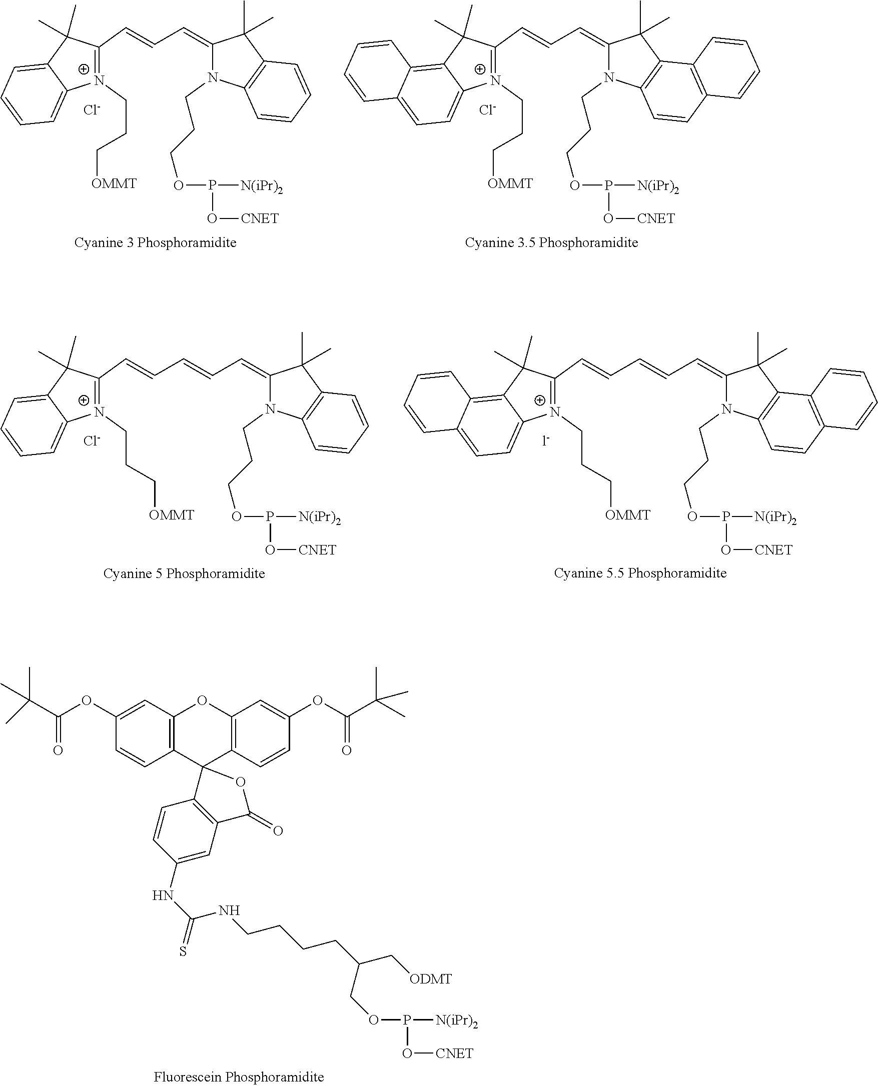

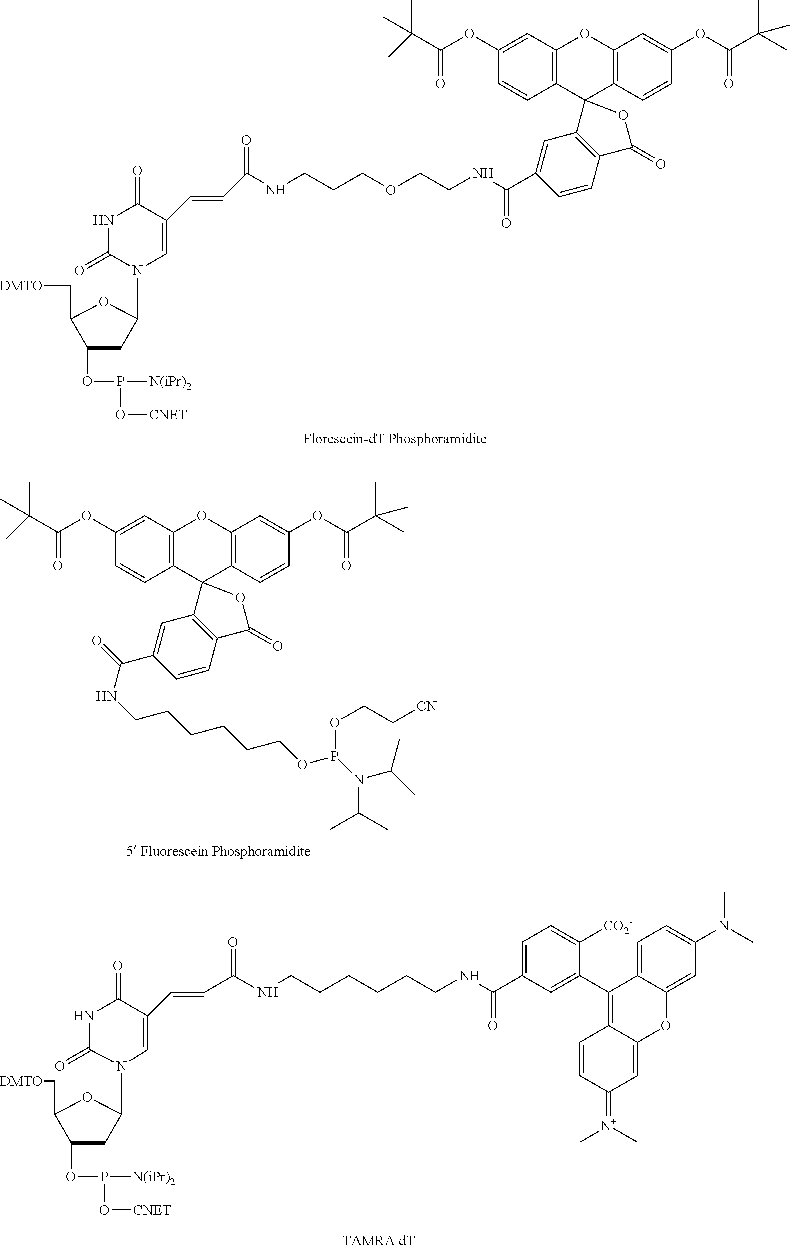

In some embodiments, L₂ of formula K is a second linker. In some embodiments, L₂ is absent. In some embodiments, L₂ is bound to the 3′ end of ODN2. In some embodiments, L₂ is bound to the 5′ end of ODN2. In some embodiments, L₂ is bound to X through an amide bond, an ester bond, a phosphate bond, an ether bond, a thioether bond, each represents a separate embodiment according to this invention. In some embodiments, L₂ is bound to ODN2 through an amide bond, an ester bond, a phosphate bond, an ether bond, a thioether bond, each represents a separate embodiment according to this invention. In some embodiments, L₂ is bound to ODN2 through a phosphate bond. In some embodiments, L₂ is defined for the “second linker” hereinbelow.

In some embodiments, ODN2 of formulas K is a second oligonucleotide sequence as described hereinabove. In some embodiments, ODN2 is directly bound to X, through an amide bond, an ester bond, a phosphate bond, an ether bond, a thioether bond, each represents a separate embodiment according to this invention. In some embodiments, ODN2 is directly bound to F₂, through an amide bond, an ester bond, a phosphate bond, an ether bond, a thioether bond, each represents a separate embodiment according to this invention. In some embodiments, ODN2 is directly bound to F₂, through a phosphate moiety.

In some embodiments, L₄ of formulas K is a fourth linker. In some embodiments, L₄ is absent. In some embodiments, L₄ is bound to the 3′ end of ODN2. In some embodiments, L₄ is bound to the 5′ end of ODN2. In some embodiments, L₄ is bound to F₂ through an amide bond, an ester bond, a phosphate bond, an ether bond, a thioether bond, each represents a separate embodiment according to this invention. In some embodiments, L₄ is bound to ODN2 through an amide bond, an ester bond, a phosphate bond, an ether bond, a thioether bond, each represents a separate embodiment according to this invention. In some embodiments, L₄ is bound to ODN2 through a phosphate moiety. In some embodiments, L₄ is as defined for the “fourth linker” hereinbelow.

In some embodiments, F₂ of formulas K is a second labeling moiety. In some embodiments, F₂ is absent. In some embodiments, F₂ is a dye. In some embodiments, F₂ is a fluorescent dye. Examples of dyes include but are not limited to: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or derivative thereof. In some embodiments, F₂ is a dye derivative. In some embodiments, F₂ is bound to ODN2 through an amide bond, an ester bond, a phosphate bond, an ether bond, a thiolether bond, each represents a separate embodiment according to this invention. In some embodiments, F₂ is bound to ODN2 through a phosphate bond. In some embodiments, F₂ is bound to L₄ through an amide bond, an ester bond, a phosphate bond, an ether bond, a thioether bond, each represents a separate embodiment according to this invention. In some embodiments, F₂ is as defined for the “labeling moiety” hereinbelow.

### Linkers (L₂ and L₄)

In some embodiments, the second compound of the DNA construct, system, bacterial probe (B-probe), the artificial receptor, the recombinant cell, and the methods according to this invention (i.e., Y-ODN-2) comprises:
a. a second oligonucleotide (ODN-2), which is complementary to said first oligonucleotide;
b. a synthetic agent,
c. optionally a second linker which links the second oligonucleotide with the synthetic agent;
d. optionally a second labeling moiety;
e. optionally a fourth linker which links the second oligonucleotide with the second labeling moiety.

### A Second Linker (L₂)

In some embodiments, the second compound (Y-ODN-2) of the DNA construct, system, bacterial probe (B-probe), the artificial receptor, the recombinant cell, and the methods of this invention, comprises a second linker, which links the second oligonucleotide with the synthetic agent. In some embodiments, the second linker is absent. In some embodiments, the second oligonucleotide is directly bound to the synthetic agent. In some embodiments, the second linker is bound to the 3′ end of the second oligonucleotide (ODN2). In some embodiments, the second linker is bound to the 5′ end of ODN2. In some embodiments, the second linker is bound to the synthetic agent through an amide bond, an ester bond, a phosphate bond, an ether bond, a thioether bond; each represents a separate embodiment according to this invention. In some embodiments, the second linker is bound to ODN2 through an amide bond, an ester bond, a phosphate bond, an ether bond, a thioether bond; each represents a separate embodiment according to this invention. In some embodiments, the second linker is covalently bound to the second oligonucleotide through a phosphate moiety.

In some embodiments, the second linker of the DNA construct, system, bacterial probe (B-probe), the artificial receptor, the recombinant cell, and the methods according to this invention and/or L₂ according to formula K is any chemical fragment which comprises at least one segment of a commercially available phosphoramidite spacer derivative as described hereinabove for the “first linker”.

In some embodiments, the second linker of the DNA construct, system, bacterial probe (B-probe), the artificial receptor, the recombinant cell, and the methods according to this invention and/or L₂ according to formula K is a substituted or unsubstituted linear or branched alkyl chain of 1-20 carbon atoms, substituted or unsubstituted linear or branched alkyl ether chain of 1-20 carbon atoms, substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 1-20 carbon atoms, substituted or unsubstituted linear or branched alkyl triazole of 1-20 carbon atoms, substituted or unsubstituted linear or branched alkyl amine chain of 1-20 carbon atoms or any combination thereof, each is a separate embodiment according to this invention.

In some embodiments, the second linker of the DNA construct, system, bacterial probe (B-probe), the artificial receptor, the recombinant cell, and the methods according to this invention and/or L₂ according to formula K comprises the following moieties:

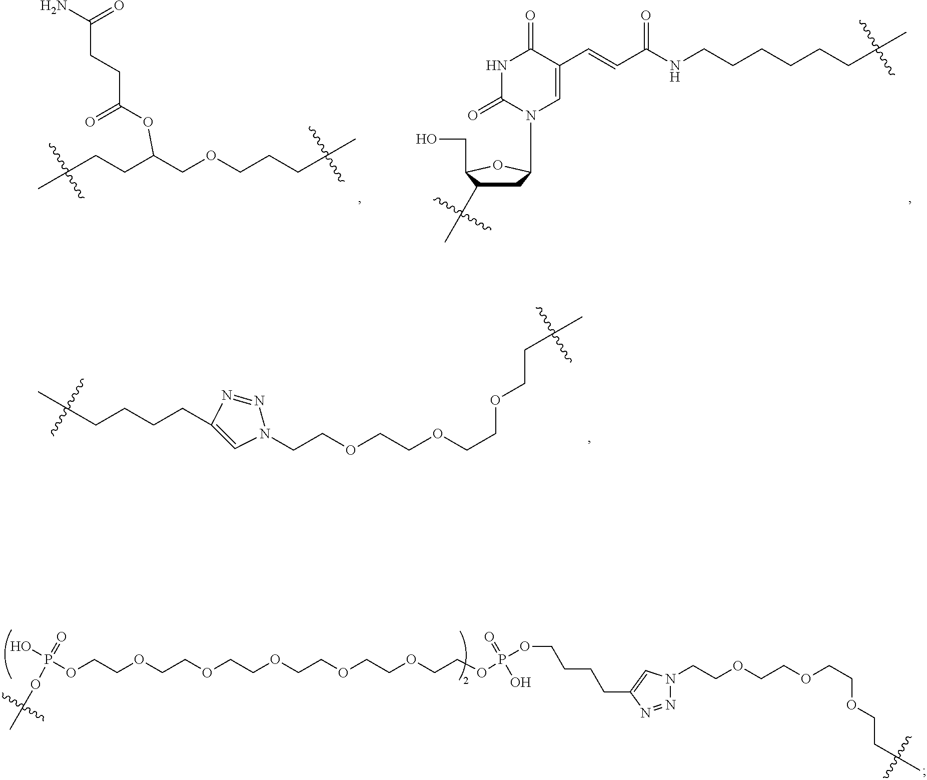

each represent a separate embodiment according to this invention.

In some embodiments, the second linker of the DNA construct, system, bacterial probe (B-probe), the artificial receptor, the recombinant cell, and the methods according to this invention and/or L₂ according to formula K comprises at least one oligoethyleneglycol (OEG) moiety, at least one phosphate moiety, at least one thioalkyl moiety; each represents a separate embodiment according to this invention. In some embodiments, the second linker and/or L₂ comprises at least one oligoethyleneglycol (OEG) moiety, at least one phosphate moiety, at least one thioalkyl moiety, or any combination thereof. In some embodiments, the second linker and/or L₂ comprise two; three; four; five; six; seven or eight sequentially arranged oligoethylene glycol (OEG) spacers; each represents a separate embodiment according to this invention.

In some embodiments, the second linker of the DNA construct, system, bacterial probe (B-probe), the artificial receptor, the recombinant cell, and the methods according to this invention and/or L₂ according to formula K comprises the following monomer:

—[(CH₂O)ₖ—PO₃H]ₗ—
wherein
   k and l are each independently an integer number between 0 and 10; and
   w is an integer number between 1 and 10.

In some embodiments, the second linker of the DNA construct, system, bacterial probe (B-probe), the artificial receptor, the recombinant cell, and the methods according to this invention and/or L₂ according to formula K comprises one; two; three; four; five; six; seven; eight; nine; or ten monomeric units; each represents a separate embodiment according to this invention. In some embodiments, the second linker comprises two monomeric units. In some embodiments, the second linker comprises five monomeric units.

In some embodiments, the second linker of the DNA construct, system, bacterial probe (B-probe), the artificial receptor, the recombinant cell, and the methods according to this invention and/or L₂ according to formula K is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, l is 0. In some embodiments, 1 is 1. In some embodiments, 1 is 2. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

### A Fourth Linker (L)

In some embodiments, the second compound (Y-ODN-2) of the system, the artificial receptor, the recombinant cell, and the methods according to this invention, comprises a fourth linker, which links the second oligonucleotide with the second labeling moiety. In some embodiments, the second oligonucleotide is directly (covalently) bound to the second labeling moiety. In other embodiments, the second oligonucleotide is covalently bound to the second labeling moiety through a fourth linker. In some embodiments, the fourth linker is absent. In some embodiments, the fourth linker is covalently bound to the 3′ end of ODN-2. In some embodiments, the fourth linker is covalently bound to the 5′ end of ODN-2. In some embodiments, the third linker is a part of a commercially available phosphoramidite dye derivative. In some embodiments, the fourth linker is covalently bound to the second labeling moiety through an amide bond, an ester bond, a phosphate bond, an ether bond, a thioether bond; each represents a separate embodiment according to this invention. In some embodiments, the fourth linker is covalently bound to ODN-2 through an amide bond, an ester bond, a phosphate bond, an ether bond, a thioether bond; each represents a separate embodiment according to this invention. In some embodiments, the fourth linker is covalently bound to the second oligonucleotide through a phosphate moiety.

In some embodiments, the fourth linker of the system, the artificial receptor, the recombinant cell, and the methods according to this invention, and/or L₄ according to formula K is any chemical fragment which comprises at least one segment of a commercially available phosphoramidite spacer derivative as described hereinabove for the “first linker”.

In some embodiments, the fourth linker of the system, the artificial receptor, the recombinant cell, and the methods according to this invention and/or L₄ according to formula K is a substituted or unsubstituted linear or branched alkyl chain of 1-20 carbon atoms, substituted or unsubstituted linear or branched alkyl ether chain of 1-20 carbon atoms, substituted or unsubstituted linear or branched thioalkyl chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl ester of 1-20 carbon atoms, substituted or unsubstituted linear or branched alkyl diamide chain of 1-20 carbon atoms, substituted or unsubstituted linear or branched alkyl triazole of 1-20 carbon atoms, substituted or unsubstituted linear or branched alkyl amine chain of 1-20 carbon atoms or any combination thereof; each is a separate embodiment according to this invention.

In some embodiments, the fourth linker of the system, the artificial receptor, the recombinant cell, and the methods according to this invention and/or L₄ according to formula K comprises the following moieties:

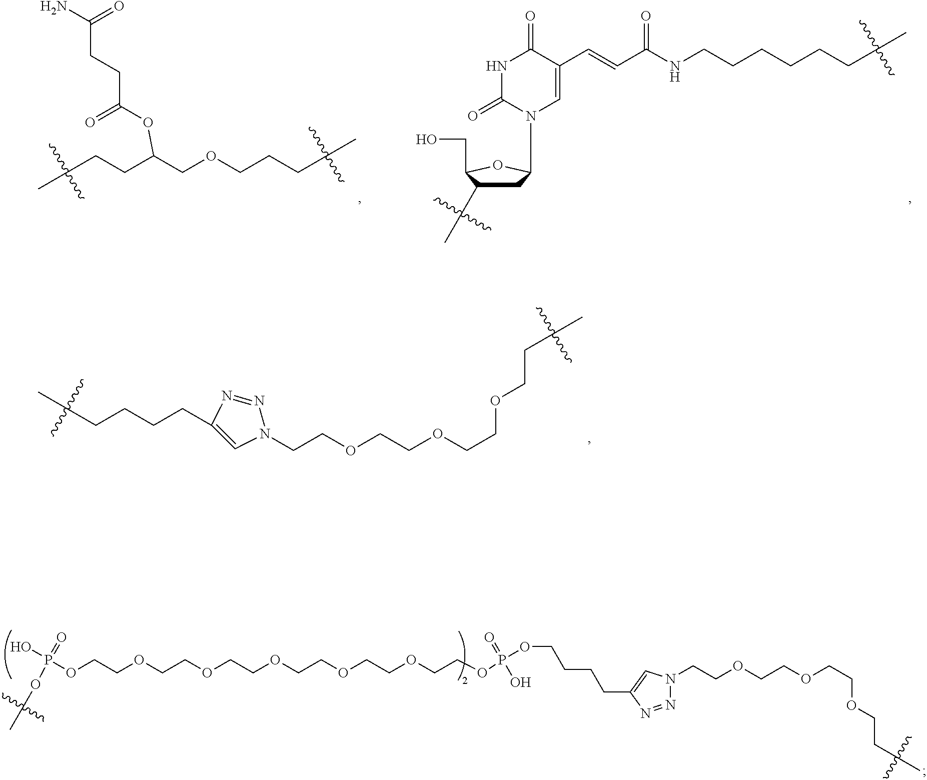

each represent a separate embodiment according to this invention.

In some embodiments, the fourth linker of the system, the artificial receptor, the recombinant cell, and the methods according to this invention and/or L₄ according to formula K comprises at least one oligoethyleneglycol (OEG) moiety, at least one phosphate moiety, at least one thioalkyl moiety; each represents a separate embodiment according to this invention. In some embodiments, the fourth linker and/or L₄ comprises at least one oligoethyleneglycol (OEG) moiety, at least one phosphate moiety, at least one thioalkyl moiety, or any combination thereof. In some embodiments, the fourth linker and/or L₄ comprise two; three; four; five; six; seven or eight sequentially arranged oligoethylene glycol (OEG) spacers; each represents a separate embodiment according to this invention.

In some embodiments, the fourth linker of the system, the artificial receptor, the recombinant cell, and the methods according to this invention and/or L₄ according to formula K comprises the following monomer:

—[(CH₂O)ₖ—PO₃H]ₗ—
wherein
   k and l are each independently an integer number between 0 and 10; and
   w is an integer number between 1 and 10.

In some embodiments, the fourth linker of the system, the artificial receptor, the recombinant cell, and the methods according to this invention and/or L₄ according to formula K comprises one; two; three; four; five; six; seven; eight; nine; or ten monomeric units; each represents a separate embodiment according to this invention. In some embodiments, the fourth linker comprises two monomeric units. In some embodiments, the fourth linker comprises five monomeric units.

In some embodiments, the fourth linker of the system, the artificial receptor, the recombinant cell, and the methods according to this invention and/or L₄ according to formula K is represented by the following formula:

—[(CH₂O)ₖ—PO₃H]ₗ—(CH₂)_{w}—S—
wherein
   k and l are each independently an integer number between 0 and 10; and
   w is an integer number between 1 and 10.

In some embodiments, k is 0. In some embodiments, k is 6. In some embodiments, k is 1, 2, 3, 4, 5, 7, 8, 9, 10; each is a separate embodiment according to this invention.

In some embodiments, l is 0. In some embodiments, 1 is 1. In some embodiments, 1 is 2. In some embodiments, 1 is 5. In some embodiments, 1 is 2, 3, 4, 6, 7, 8, 9, 10; each is a separate embodiment according to this invention.

In some embodiments, w is 6. In some embodiments, w is 1, 2, 3, 4, 5, 7, 8, 9, 10; each is a separate embodiment according to this invention.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent or it can be a component of a larger substituent, such as in an alkoxy, alkoxyalkyl, haloalkyl, arylalkyl, alkylamino, dialkylamino, alkylamido, alkylurea, thioalkyl, alkyldiamide, alkylamide, alkylphosphate, alkylether, alkyltriazole, alkylester, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

### The Third Compound

In some embodiments, the DNA construct, system, bacterial probe (B-probe), the artificial receptor, the recombinant cell, and the methods of this invention, comprises a third compound.

In some embodiments, the third compound comprises a DNA duplex (dsDNA).

In some embodiments, the dsDNA comprises a shorter oligonucleotide (ssDNA-short) and a longer oligonucleotide (ssDNA-long), which is complementary to said shorter oligonucleotide (ssDNA-short). In some embodiments, the longer oligonucleotide (ssDNA-long) comprises a second hanging strand (a second toehold region).

In some embodiments, the DNA duplex (dsDNA) is appended with a hanging strand (a second hanging strand), which is complementary to the first hanging strand comprised in the second oligonucleotide (ODN2) of the second compound as described hereinbelow.

In some embodiments, the DNA duplex (dsDNA) is further appended with at least one; two; three; four; five; six; seven; eight; nine; or ten labeling moieties; each represents a separate embodiment according to this invention. In some embodiments, the DNA duplex (dsDNA) is further appended with at least one; two; three; four; five; six; seven; eight; nine; or ten fluorescent dyes; each represents a separate embodiment according to this invention. In some embodiments, the DNA duplex (dsDNA) is further appended with at least two fluorescent dyes.

In some embodiments, the DNA construct, system, bacterial probe (B-probe), the artificial receptor, the recombinant cell, and the methods of this invention, comprise a third compound comprising a DNA duplex (dsDNA) appended with a second hanging strand complementary to the first hanging strand comprised in the second oligonucleotide (ODN2) as described hereinbelow, and further appended with at least two fluorescent dyes.

In some embodiments, the third compound of the DNA construct, system, bacterial probe (B-probe), the artificial receptor, the recombinant cell, and the methods of this invention, is attached to the second compound via the hybridization of the first hanging strand (of ODN2) to the second hanging strand (of dsDNA).

In some embodiments, the third compound of the DNA construct, system, bacterial probe (B-probe), the artificial receptor, the recombinant cell, and the methods according to this invention comprises:
a. a DNA duplex (dsDNA), said dsDNA comprises a shorter oligonucleotide (ssDNA-short) and a longer oligonucleotide (ssDNA-long) which is complementary to said shorter oligonucleotide (ssDNA-short), wherein said ssDNA-long comprises a second hanging strand which is complimentary to a first hanging strand comprised in a second oligonucleotide (ODN-2) of a second compound;
b. at least two labeling moieties,
c. optionally linkers, which link each of said labeling moieties to one of said oligonucleotides (i.e., ssDNA-long or ssDNA-short);

In some embodiments, the longer oligonucleotide (ssDNA-long) is bound directly to each of the labeling moieties. In some embodiments, the longer oligonucleotide (ssDNA-long) is bound to the labeling moieties through linkers.

In some embodiments, the labeling moieties are all bound to the longer oligonucleotide (ssDNA-long). In some embodiments, the labeling moieties are all bound to the shorter oligonucleotide (ssDNA-short). In some embodiments, some of the labeling moieties are bound to the longer oligonucleotide (ssDNA-long) and some to the shorter oligonucleotide (ssDNA-short). In some embodiments, the labeling moieties are as described hereinbelow. In some embodiments, the labeling moieties are fluorescent dyes as described herein below.

In some embodiments, the longer oligonucleotide (ssDNA-long) is appended with at least two labeling moieties. In some embodiments, the longer oligonucleotide (ssDNA-long) is appended with at least two; three; four; five; six; or seven labeling moieties; each represents a separate embodiment according to this invention. In some embodiments, the longer oligonucleotide (ssDNA-long) is appended with at least two fluorescent dyes. In some embodiments, the longer oligonucleotide (ssDNA-long) is appended with at least three fluorescent dyes. In some embodiments, the longer oligonucleotide (ssDNA-long) is appended with at least two; three; four; five; six; or seven fluorescent dyes; each represents a separate embodiment according to this invention.

In some embodiments, the fluorescent dyes attached to said longer oligonucleotide (ssDNA-long) are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the fluorescent dyes attached to said longer oligonucleotide (ssDNA-long) are located 4-6 bases apart from each other. In some embodiments, the fluorescent dyes attached to said longer oligonucleotide (ssDNA-long) are located 5 bases apart from each other.

In some embodiments, the shorter oligonucleotide (ssDNA-short) is appended with at least two labeling moieties. In some embodiments, the shorter oligonucleotide (ssDNA-short) is appended with at least two; three; four; five; six; or seven labeling moieties; each represents a separate embodiment according to this invention. In some embodiments, the shorter oligonucleotide (ssDNA-short) is appended with at least two fluorescent dyes. In some embodiments, the shorter oligonucleotide (ssDNA-short) is appended with at least two fluorescent dyes. In some embodiments, the shorter oligonucleotide (ssDNA-short) is appended with at least two; three; four; five; six; or seven fluorescent dyes; each represents a separate embodiment according to this invention.

In some embodiments, the fluorescent dyes attached to said shorter oligonucleotide (ssDNA-short) are located 2-10; 3-9; 4-8; 4-7; 3-7; 4-6; 3-6; 3-5; 3; 4; 5; 6; 7; 8; 9 bases apart from each other; each represents a separate embodiment according to this invention. In some embodiments, the fluorescent dyes attached to said shorter oligonucleotide (ssDNA-short) are located 4-6 bases apart from each other. In some embodiments, the fluorescent dyes attached to said shorter oligonucleotide (ssDNA-short)) are located 5 bases apart from each other.

In some embodiments, the shorter oligonucleotide (ssDNA-short) is appended with at least one labeling moiety and the longer oligonucleotide (ssDNA-long) is appended with at least one labeling moiety. In some embodiments, the shorter oligonucleotide (ssDNA-short) is appended with at least one; two; three; four; five; six; or seven labeling moieties; each represents a separate embodiment according to this invention, and the longer oligonucleotide (ssDNA-long) is appended with at least one; two; three; four; five; six; or seven labeling moieties; each represents a separate embodiment according to this invention. In some embodiments, the shorter oligonucleotide (ssDNA-short) is appended with at least two fluorescent dyes and the longer oligonucleotide (ssDNA-long) is appended with at least two fluorescent dyes.

### DNA Duplex (dsDNA)

In some embodiments, the second oligonucleotide (ODN2) of the DNA construct, system, bacterial probe (B-probe), the artificial receptor, the recombinant cell, and the methods according to the invention, further comprises a first hanging strand (a first toehold region).

In some embodiments, the DNA construct, system, bacterial probe (B-probe), the artificial receptor, the recombinant cell, and the methods according to the invention, comprise a third compound, which comprises a DNA duplex (dsDNA). In some embodiments, the DNA duplex (dsDNA) comprises a second hanging strand, which is complementary to the first hanging strand comprised in the second oligonucleotide (ODN2) as described hereinabove.

In some embodiments, the dsDNA comprises two oligonucleotides, a longer oligonucleotide comprising the second hanging strand (ssDNA-long), and a shorter oligonucleotide (ssDNA-short), wherein ssDNA-long is complementary to ssDNA-short.

In some embodiments, the shorter oligonucleotide of the DNA duplex (ssDNA-short) of the DNA construct, system, bacterial probe (B-probe), the artificial receptor, the recombinant cell, and the methods according to the invention is at least 4, at least 8, at least 12, at least 16, at least 20, or at least 30 nucleotides in length; each is a separate embodiment according to this invention. In some embodiments, the shorter oligonucleotide of the DNA duplex (ssDNA-short) of the DNA construct, system, bacterial probe (B-probe), the artificial receptor, the recombinant cell, and the methods according to the invention is no more than about 50 nucleotides in length. In some embodiments, the shorter oligonucleotide of the DNA duplex (ssDNA-short) is at least 2, at least 4, at least 8, at least 12, at least 16, or at least 20 nucleotides shorter than the longer oligonucleotide of the DNA duplex (ssDNA-long); each is a separate embodiment according to this invention.

In some embodiments, the shorter oligonucleotide of the DNA duplex (ssDNA-short) of the DNA construct, system, bacterial probe (B-probe), the artificial receptor, the recombinant cell, and the methods according to the invention is 1-1000; 3-500; 4-250; 5-100; 10-80; 10-50; 15-35; 5-45; 15-25; 20-40; 15-50; 25-35; 10; 15; 16; 17; 18; 19; 20; 21; 22; 23; 24; 25; 26; 27; 28; 29; 30; 31; 32; 33; 34; 35; 36; 37; 38; 39; 40 bases long; each represents a separate embodiment according to this invention. In some embodiments, the shorter oligonucleotide (ssDNA-short) comprises a sequence comprising at least 80% homology to SEQ ID NO.: 24. In some embodiments, the shorter oligonucleotide (ssDNA-short) sequence is represented by SEQ ID NO.: 24.

In some embodiments, the longer oligonucleotide of the DNA duplex (ssDNA-long) of the DNA construct, system, bacterial probe (B-probe), the artificial receptor, the recombinant cell, and the methods according to the invention is at least 4, at least 8, at least 12, at least 16, at least 20, or at least 30 nucleotides in length; each is a separate embodiment according to this invention. In some embodiments, the longer oligonucleotide of the DNA duplex (ssDNA-long) of the DNA construct, system, bacterial probe (B-probe), the artificial receptor, the recombinant cell, and the methods according to the invention is no more than about 50 nucleotides in length. In some embodiments, the longer oligonucleotide of the DNA duplex (ssDNA-long) is at least 2, at least 4, at least 8, at least 12, at least 16, or at least 20 nucleotides longer than the shorter oligonucleotide of the DNA duplex (ssDNA-short); each is a separate embodiment according to this invention. In some embodiments, the longer oligonucleotide of the DNA duplex (ssDNA-long) comprises a hanging strand (toehold region). In some embodiments, the longer oligonucleotide of the DNA duplex (ssDNA-long) comprises the second hanging strand (second toehold region) as described herein above.

In some embodiments, the longer oligonucleotide of the DNA duplex (ssDNA-long) of the DNA construct, system, bacterial probe (B-probe), the artificial receptor, the recombinant cell, and the methods according to the invention is 1-1000; 3-500; 4-250; 5-100; 10-80; 10-50; 15-45; 5-50; 25-55; 20-45; 15-50; 30-45; 10; 15; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; 50 bases long; each represents a separate embodiment according to this invention. In some embodiments, the longer oligonucleotide (ssDNA-long) comprises a sequence comprising at least 80% homology to SEQ ID No.: 22 or 23. In some embodiments, the longer oligonucleotide (ssDNA-long) sequence is represented by SEQ ID NO.: 22 or 23.

In some embodiments, the longer oligonucleotide (ssDNA-long) comprises a hanging strand. In some embodiments, the hanging strand comprised in the longer oligonucleotide is a second hanging strand. In some embodiments, the longer oligonucleotide (ssDNA-long) comprises a second hanging strand, which is complementary to the first hanging strand comprised in the second compound. In some embodiments, the DNA duplex is appended with at least one; two; three; four; five; six; seven; eight; nine; or ten labeling moieties; each represents a separate embodiment according to this invention. In some embodiments, the DNA duplex is appended with at least one; two; three; four; five; six; seven; eight; nine; or ten fluorescent dyes; each represents a separate embodiment according to this invention. In some embodiments, the DNA duplex is appended with at least two fluorescent dyes. In some embodiments, the DNA duplex is appended with at least three fluorescent dyes. In some embodiments, the DNA duplex is appended with at least four fluorescent dyes.

In some embodiments, the DNA duplex (dsDNA) is comprised in a third compound as described hereinabove.

In some embodiments, the fluorescent dyes of the DNA duplex (dsDNA) are located 1-10; 2-8; 3-7; 4-6; 4-5; 5-6; 5-7; 3-8 bases apart from each other; each represents a separate embodiment according to this invention. In some embodiments, the fluorescent dyes of the DNA duplex (dsDNA) are located 4-6 bases apart from each other. In some embodiments, the fluorescent dyes of the DNA duplex (dsDNA) are located 5 bases apart from each other.

In some embodiments, the fluorescent dyes of the DNA duplex (dsDNA) are located on the longer oligonucleotide strand that comprises the second hanging strand (ssDNA-long). In some embodiments, the fluorescent dyes of the DNA duplex (dsDNA) are located on the shorter oligonucleotide strand (ssDNA-short). In some embodiments, the fluorescent dyes of the DNA duplex (dsDNA) are located on both the shorter (ssDNA-short) and the longer (ssDNA-long) oligonucleotide strands.

In some embodiments, the longer oligonucleotide of the DNA duplex (ssDNA-long) of the DNA construct, system, bacterial probe (B-probe), the artificial receptor, the recombinant cell, and the methods according to the invention is appended with at least one labeling moiety. In some embodiments, the longer oligonucleotide of the DNA duplex (ssDNA-long) is appended with at least two; three; four; five; six; seven; eight; nine; or ten labeling moieties; each represents a separate embodiment according to this invention. In some embodiments, the longer oligonucleotide of the DNA duplex (ssDNA-long) is appended with one; two; three; four; five; six; seven; eight; nine; or ten labeling moieties; each represents a separate embodiment according to this invention.

In some embodiments, the longer oligonucleotide of the DNA duplex (ssDNA-long) of the DNA construct, system, bacterial probe (B-probe), the artificial receptor, the recombinant cell, and the methods according to the invention is appended with at least one fluorescent dye. In some embodiments, the longer oligonucleotide of the DNA duplex (ssDNA-long) is appended with at least two; three; four; five; six; seven; eight; nine; or ten fluorescent dyes; each represents a separate embodiment according to this invention. In some embodiments, the longer oligonucleotide of the DNA duplex (ssDNA-long) is appended with one; two; three; four; five; six; seven; eight; nine; or ten fluorescent dyes; each represents a separate embodiment according to this invention.

In some embodiments, the longer oligonucleotide of the DNA duplex (ssDNA-long) of the DNA construct, system, bacterial probe (B-probe), the artificial receptor, the recombinant cell, and the methods according to the invention is complementary to the shorter oligonucleotide of the DNA duplex (ssDNA-short) comprised in the DNA duplex (dsDNA), which comprised in the third compound as described hereinbelow.

### Labeling Moiety (F and F₂)

In accordance with the DNA construct, system, bacterial probe (B-probe), the artificial receptor, the recombinant cell, and the methods disclosed herein, the compounds may comprise one or more labeling moieties, which are attached to the oligonucleotides. Oligonucleotides can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

As used herein, “labeling moieties” or “labels” are chemical or biochemical moieties useful for labeling a compound. Such labeling moieties include fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and may be covalently or noncovalently joined to an oligonucleotide or nucleotide.

In some embodiments, the labeling moieties are covalently bound to the oligonucleotides of the invention. In some embodiments, the labeling moieties are covalently bound to the oligonucleotides of the invention through a linker or a spacer.

In illustrative embodiments, the compounds according to this invention, may be labeled with a “fluorescent dye” or a “fluorophore.” As used herein, a “fluorescent dye” or a “fluorophore” is a chemical group that can be excited by light to emit fluorescence. Some fluorophores may be excited by light to emit phosphorescence. Dyes may include acceptor dyes that are capable of quenching a fluorescent signal from a fluorescent donor dye. In some embodiments, the dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red phycoerythrin (PE) or a derivative thereof. In some embodiments, the dye is selected from: dansyl, fluorescein (6-FAM), FAM, cyanine dyes (e.g. Cy3, Cy5, Cy7, etc), sulfoindocyanine, nile red, Rhodamine dyes (e.g., Rhodamine 123, Rhodamine Red-X, etc.), perylene, fluorenyl, coumarin, 7-methoxycoumarin (Mca), dabcyl, NBD, Nile blue, TAMRA, BODIPY dyes, FITC (Fluorescein isothiocyanate), Thiazole orange, Quinoline blue, Thiazole red, phycoerythrin (PE), Acridine Orange, Alexa Fluor dyes (e.g., Alexa Fluor 488, Alexa Fluor 555, Alexa Fluor 647, etc.), Cascade Blue, DAPI (4′,6-diamidino-2-phenylindole), DiI (1,1′-Dioctadecyl-3,3,3′,3′-tetramethylindocarbocyanine perchlorate), Ethidium Bromide, GFP (Green Fluorescent Protein), Hoechst dyes (e.g., Hoechst 33342, Hoechst 33258, etc.), Indo-1, Lucifer Yellow, MitoTracker dyes (e.g., MitoTracker Green, MitoTracker Red, etc.), Oregon Green, Propidium Iodide, SYBR Green, Texas Red, YOYO-1, ZsGreen or derivative thereof. Further non limiting examples of Dyes that may be used in the disclosed compounds, system and methods include, but are not limited to, the following dyes and/or dyes sold under the following trade names: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™_1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue™; Calcium Crimson™; Calcium Green; Calcium Orange; Calcofluor White; Cascade Blue™; Cascade Yellow; Catecholamine; CCF2 (GeneBlazer); CFDA; CFP-Cyan Fluorescent Protein; CFP/YFP FRET; Chlorophyll; Chromomycin A; CL-NERF (Ratio Dye, pH); CMFDA; Coelenterazine f, Coelenterazine fcp; Coelenterazine h; Coelenterazine hcp; Coelenterazine ip; Coelenterazine n; Coelenterazine 0; Coumarin Phalloidin; C-phycocyanine; CPM Methylcoumarin; CTC; CTC Formazan; Cy2™; Cy3. 1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof, each is a separate embodiment according to this invention.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

In some embodiments, the labeling moiety on the oligonucleotides and the compounds of the DNA construct, system, bacterial probe (B-probe), the artificial receptor, the recombinant cell, and the methods according to the invention, is a quencher. Quenching may include dynamic quenching (e.g., by FRET), static quenching, or both. Illustrative quenchers may include Dabcyl. Illustrative quenchers may also include dark quenchers, which may include black hole quenchers sold under the tradename “BHQ” (e.g., BHQ-0, BHQ-1, BHQ-2, and BHQ-3, Biosearch Technologies, Novato, Calif). Dark quenchers also may include quenchers sold under the tradename “QXL™” (Anaspec, San Jose, Calif.). Dark quenchers also may include DNP-type non-fluorophores that include a 2,4-dinitrophenyl group.

In some situations, it may be useful to include interactive labels on two oligonucleotides with due consideration given for maintaining an appropriate spacing of the labels on the oligonucleotides to permit the separation of the labels during conformational changes. One type of interactive label pair is a quencher-dye pair, which may include a fluorophore and a quencher. The ordinarily skilled artisan can select a suitable quencher moiety that will quench the emission of the particular fluorophore. In an illustrative embodiment, the Dabcyl quencher absorbs the emission of fluorescence from the fluorophore moiety.

Alternatively, the proximity of the two labels can be detected using fluorescence resonance energy transfer (FRET) or fluorescence polarization. FRET is a distance-dependent interaction between the electronic excited states of two dye molecules in which excitation is transferred from a donor molecule to an acceptor molecule without emission of a photon. Examples of donor/acceptor dye pairs for FRET are known in the art and may include fluorophores and quenchers described herein such as Fluorescein/Tetramethylrhodamine, IAEDANS/Fluorescein (Molecular Probes, Eugene, Oreg.), EDANS/Dabcyl, Fluorescein/Fluorescein (Molecular Probes, Eugene, Oreg.), BODIPY FL/BODIPY FL (Molecular Probes, Eugene, Oreg.), BODIPY TMR/ALEXA 647, ALEXA-488/ALEXA-647, and Fluorescein/QSY7™

The labels can be conjugated to the oligonucleotides directly, or indirectly through linkers or spacers, by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. According to various embodiments, the labeling moiety is attached to the 5′ or 3′ end of the first and/or the second oligonucleotide; each is a separate embodiment. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite.

Oligonucleotides may also incorporate oligonucleotide functionalizing reagents having one or more sulfhydryl, amino or hydroxyl moieties into the oligonucleotide sequence. For example, biotin can be added to the 5′ end by reacting an aminothymidine residue, introduced during synthesis, with an N-hydroxysuccinimide ester of biotin. Labels at the 3′ terminus, for example, can employ polynucleotide terminal transferase to add the desired moiety, such as for example, cordycepin, ³⁵S-dATP, and biotinylated dUTP.

In some embodiments, the first and/or the second compound of the DNA construct, system, bacterial probe (B-probe), the artificial receptor, the recombinant cell, and the methods according to the invention, comprises one or more labeling moieties (e.g., F of formula H, H(complex), H(a), H(a)(complex), H(b) and H(b)(complex); F₂ of formula K). In some embodiments, the first oligonucleotide (ODN-1) is bound to a labeling moiety in its 3′ or 5′ end. In some embodiments, the labeling moiety is bound to the first oligonucleotide directly. In some embodiments, the labeling moiety is bound to first oligonucleotide through a third linker. In some embodiments, the second oligonucleotide (ODN-2) is bound to a second labeling moiety in its 3′ or 5′ end. In some embodiments, the second labeling moiety is bound to the second oligonucleotide directly. In some embodiments, the second labeling moiety is bound to second oligonucleotide through a fourth linker. In some embodiments, the labeling moieties are covalently attached to the oligonucleotide. In some embodiments, the labeling moieties are covalently attached to a certain nucleotide of ODN2. In some embodiments, the labeling moieties are covalently attached to Thymine nucleotides of ODN2. In some embodiments, the labeling moieties are covalently attached to a certain nucleotide of ssDNA-long. In some embodiments, the labeling moieties are covalently attached to Thymine nucleotides of ss-DNAlong.

### Synthetic Agent

In some embodiments, the second compound of the DNA construct, system, bacterial probe (B-probe), the artificial receptor, the recombinant cell, and the methods according to this invention, comprises a synthetic agent. In some embodiments, the second oligonucleotide (ODN-2) is bound to a synthetic agent in its 3′ or 5′ end. In some embodiments, the synthetic agent is bound to ODN-2 directly. In some embodiments, the synthetic agent is bound to ODN-2 through a second linker.

In some embodiments, the second compound comprises a synthetic agent and a second labeling moiety. In some embodiments, the second compound does not comprise a second labeling moiety.

According to this invention, the term “synthetic agent” refers to any chemical or biological moiety, which provides a chemical or biological function to the system, or to the cell, to which it is attached. In some embodiments, synthetic agent refers to any chemical moiety, which is capable of binding to various extracellular signals such as ions, small molecules, proteins, and cells, and can control the response of cells to their surroundings. In some embodiments, a synthetic agent refers to any chemical moiety, which has a chemical, physical or biological effect on the cell to which it is attached.

In some embodiments, a synthetic agent refers to any chemical and/or biological moiety, which has a biological effect on a living organism, a tissue or a cell (also referred herein as “a bioactive moiety”). In some embodiments, a biological effect comprises affecting the growth, the survival, the replication, the differentiation, the transcriptome, the proteome, or the function of a cell. Bioactive and/or biological moieties refer to a broad category of compounds that have specific effects on living organisms. These molecules can be naturally occurring or synthetic and often have therapeutic or biological activity. In some embodiments, the biological moiety may be any bioactive moiety known in the art, some of which are listed hereinbelow as illustrative examples: Adenosine triphosphate (ATP), Insulin, Dopamine, Serotonin, Acetylcholine, Epinephrine (adrenaline), Histamine, Prostaglandins, Endorphins, Antibiotics, Steroids (e.g., cortisol, estrogen, and testosterone), Caffeine, Curcumin, Resveratrol, Quercetin, Cannabinoids (e.g., THC (tetrahydrocannabinol) and CBD (cannabidiol)), Alkaloids, Vitamin C (ascorbic acid), Omega-3 fatty acids, Glutathione, as well as antibodies and various peptides.

In some embodiments, synthetic agent refers to any chemical moiety, which can bind, either covalently or non-covalently, to a solid support, and/or to an abiotic surface (also referred herein as “a surface binder”). In some embodiments, a synthetic agent refers an artificial receptor appended with a specific functionality. In some embodiments, a synthetic agent refers to any chemical or biological moiety, which provides the cell, system or compound to which it is attached, with a specific functionality (e.g., fluorescence, therapeutic effect, solid surface binding capability, specific cell targeting, etc.).

In some embodiments, the synthetic agent is a labeling moiety as described herein above.

In some embodiments, the synthetic agent is a therapeutically active agent. In some embodiments, the therapeutically active agent is a drug. In some embodiments, the therapeutically active agent is selected from: anticancer agents, DNA-interacting molecules, cholesterol-lowering compounds, antibiotics, anti-AIDS molecules, each represents a separate embodiment according to the invention.

In some embodiments, the synthetic agent is a is an oligonucleotide, a nucleic acid construct, an antisense, a plasmid, a polynucleotide, an amino acid, a peptide, a polypeptide, a hormone, a steroid, an antibody, an antigen, a radioisotope, a chemotherapeutic agent, a toxin, an anti-inflammatory agent, a growth factor or any combination thereof, each represents a separate embodiment according to the invention.

In some embodiments, the synthetic agent is a molecular marker. In some embodiments, the synthetic agent is an adhesion molecule. In some embodiments, synthetic agent is a cancer cell binder. In some embodiments, “cancer cell binder” refers to any chemical moiety capable of interacting with proteins expressed by cancer cells. In some embodiments, “cancer cell binder” refers to a protein binder capable of interacting with proteins expressed by cancer cells. In some embodiments, the cancer cell binder is cervical cancer cells, melanoma cells or prostate cancer cells; each represents a separate embodiment according to this invention. In some embodiments, the synthetic agent is a protein ligand. In some embodiments, the synthetic agent is a protein binder. In some embodiments, the synthetic agent is a protein receptor. In some embodiments, the synthetic agent is a CSP binder. In some embodiments, the synthetic agent is an antibody. In some embodiments, the synthetic agent is a drug. In some embodiments, the synthetic agent is an anticancer agent. In some embodiments, the synthetic agent is a growth factor. In some embodiments, the synthetic agent is a surface binder. In some embodiments, the synthetic agent is an abiotic surface binder. In some embodiments, the surface binder is a functional group capable of binding a solid surface or a solid support.

In some embodiments, the synthetic agent is a protein binder. In some embodiments, a “protein binder” refers to any biological agent which binds to a specific target protein. Non limiting examples of protein binders groups known in the art include: Antibodies: (i.e., immunoglobulins), Enzymes, Hormones, (e.g., insulin, estrogen, and testosterone), Receptors, Lectins, Carrier proteins (e.g., hemoglobin and albumin), DNA-binding proteins (e.g., transcription factors and DNA polymerases), RNA-binding proteins (e.g., splicing factors and ribosomal proteins), Chaperones, Metal-binding proteins (e.g. metalloproteins), Drugs (e.g., antibiotics, antiviral drugs, anticancer agents), Inhibitors (e.g., statins, protease inhibitors), Agonists, antagonists, Natural products (e.g., resveratrol), Ligands, Metabolites (e.g., adenosine triphosphate (ATP)), Toxins (e.g., botulinum toxin, ricin), Dyes and fluorescent probes. Non limiting examples of more specific protein binders known in the art include: folate, anisamide (An), or glutamate urea (GLA), biotin, marimastat, ethacrynic acid, bisethacrynic acid, Ni-nitrilotriacetic acid (Ni-NTA), bis Ni-NTA, tris-Ni-NTA, PDGF-BB, heparin, FGF aptamer, estrogen, DNA aptamer, RNA aptamer, peptide aldehyde, estrogen, suberoylanilidehydroxamic acid (SAHA); each represents a separate embodiment according to this invention.

In some embodiments, the synthetic agent is a cancer cell binder. In some embodiments, the cancer cell binder is a folate receptor binder or derivative thereof. In some embodiments, the cancer cell binder is a folate or derivative thereof. In some embodiments, the cancer cell binder is a sigma receptor binder or derivative thereof. In some embodiments, the cancer cell binder is an anisamide or derivative thereof. In some embodiments, the cancer cell binder is a prostate-specific membrane antigen (PSMA) receptor binder or derivative thereof. In some embodiments, the cancer cell binder is a glutamate urea or derivative thereof.

In some embodiments, the synthetic agent is a molecular marker. In some embodiments, the synthetic agent is an angiogenic factor. In some embodiments, the synthetic agent is a cytokine. In some embodiments, the synthetic agent is a hormone. In some embodiments, the synthetic agent is a DNA molecule. In some embodiments, the synthetic agent is a siRNA molecule. In some embodiments, the synthetic agent is an oligosaccharide.

In some embodiments, the synthetic agent is a protein receptor. In some embodiments, the synthetic agent is an immune activator. In some embodiments, the synthetic agent is an immune suppressor. In some embodiments, the synthetic agent is a small molecule. In some embodiments, the small molecule is a drug.

In some embodiments, the synthetic agent is a labeling moiety as described herein above. In some embodiments, the labeling moiety is a dye. In some embodiments, the dye is a fluorescent dye. In some embodiments, the dye is selected from a group consisting of: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) or a derivative thereof.

In some embodiments, the synthetic agent is a protein receptor. In some embodiments, the synthetic agent is a protein binder. In some embodiments, the synthetic agent is a biotin.

In some embodiments, the synthetic agent is a surface binder. In some embodiments, the synthetic agent is an abiotic surface binder. In some embodiments, the synthetic agent is a binder for abiotic surfaces. In some embodiments, the synthetic agent is an agent capable of binding to solid support. In some embodiments, the surface binder is capable of binding a surface. According to this invention, a “surface binder” is any chemical moiety, or functional group, that is capable of binding solid surfaces. In some embodiments, the binding is covalent, electrostatic, van der Waals or any combination thereof; each is a separate embodiment. In some embodiments, attachment of the surface binder to the surface comprises covalent bond, coordination bond, polar bond, van der Waals bond or any combination thereof. In some embodiments, the surface binder comprises a functional moiety capable of binding a surface. According to this aspect and in some embodiments, the surface binder comprises a thiol end group (SH) or an end group comprising a sulfur-sulfur bond (—S—S—). Such bonds are capable of binding to a noble metal. For example, thiol or —S—S— moieties binds strongly to gold surfaces and to other noble metal surface including but not limited to silver, platinum and palladium. Thiols and —S—S— bonds also bind to semiconductor surfaces such as GaAs etc. In some embodiments, the surface binder comprises a thiol group (HS). In some embodiments, the surface binder is a C₁C₂₀ thioalkyl. In some embodiments, the surface binder is a C₂-C₈ thioalkyl. In some embodiments, the surface binder is a thiohexyl. In some embodiments, attachment of the surface binder to a surface comprise silicon chemistry. According to this aspect and in some embodiments, the surface is or comprises silicon. In some embodiments, the surface comprises silicon oxide. In some embodiments, the silicon oxide surface comprises glass or quartz. In some embodiments, the surface comprises silicon coated by a silicon oxide layer. According to this aspect and in some embodiments, the surface binder comprises a functional group capable of binding to silicon oxide. In some embodiments, the functional group comprises silicon atom. In some embodiments, the functional group comprises silicon bonded to a halogen atom. In some embodiments, the halogen atom is Cl, Br, F or n. In one embodiment the silicon-halogen functional group comprise Si-trichloride, Si-tribromide, Si-dichloride, Si dibromide. In some embodiments, the functional group comprises Si bonded to oxygen atom. In some embodiments, the functional group comprises Si bonded to two or three oxygen atoms. In some embodiments, the functional group of the surface binder comprises Si-halogen bond and upon reaction with the surface, the halogen atom is replaced by an oxygen atom, and bonding to the surface occurs. In some embodiments, the surface binder comprises a pyridine moiety.

In some embodiments, the surface is an abiotic surface. In some embodiments, the surface is a passivated. In some embodiments, surfaces of this invention are inorganic (e.g. silicon oxide, gold). In some embodiments, surfaces of this invention are organic (e.g. an organic polymer). In some embodiments, surfaces of this invention are metals (e.g., gold). In some embodiments, surfaces of this invention comprise both organic and inorganic materials. In some embodiments, the surface is a material selected from gold, glass, a doped glass, indium tin oxide (ITO)-coated glass, silicon, a doped silicon, Si(100), Si(111), SiO₂, SiH, silicon carbide mirror, quartz, a metal, metal oxide, a mixture of metal and metal oxide, group IV elements, mica, a polymer such as polyacrylamide and polystyrene, a plastic, a zeolite, a clay, wood, a membrane, an optical fiber, a ceramic, a metalized ceramic, an alumina, an electrically-conductive material, a semiconductor, steel or a stainless steel; each is a separate embodiment according to the invention. In some embodiments, the surface is a gold surface. In some embodiments, the surface is a passivated gold surface. In some embodiments, surfaces of this invention are flat. In some embodiments, the surfaces are curved. In some embodiments, the surface is macroscopically flat and microscopically curved or vice-versa. In some embodiments, the surface is the surface of a particle. In some embodiments, the surface is the surface of a nanoparticle.

### Methods for Decorating a Cell

In some embodiments, this invention is directed to a method for decorating a cell with a synthetic agent, said method comprises:
a. ectopically expressing in said cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a synthetic agent, either directly or through a second linker, wherein said second oligonucleotide is complementary to said first oligonucleotide thereby generating a DNA construct;
c. incubating said cell of (a) with said DNA construct of (b);
   thereby decorating said cell with said synthetic agent.

In some embodiments, the binder of said first compound has affinity to said extracellular binding domain of said cell. In some embodiments, the binder is a His-tag binder according to this invention. In some embodiments, the extracellular binding domain of said cell comprises a histidine-tag. In some embodiments, the DNA construct is incubated with the cell in the presence of metal ions. In some embodiments, the metal is nickel. In some embodiments, the DNA construct is incubated with the cell in the presence of Ni²⁺ ions.

In some embodiments, this invention is directed to a method for modifying a cell with a synthetic agent, said method comprises:
a. ectopically expressing in said cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a synthetic agent, either directly or through a second linker, wherein said second oligonucleotide is complementary to said first oligonucleotide thereby generating a DNA construct;
c. incubating said cell of (a) with said DNA construct of (b);
   thereby modifying said cell with said synthetic agent.

In some embodiments, the binder of said first compound has affinity to said extracellular binding domain of said cell. In some embodiments, the binder is a His-tag binder according to this invention. In some embodiments, the extracellular binding domain of said cell comprises a histidine-tag. In some embodiments, the DNA construct is incubated with the cell in the presence of metal ions. In some embodiments, the metal is nickel. In some embodiments, the DNA construct is incubated with the cell in the presence of Ni²⁺ ions.

A skilled artisan would appreciate that “decorating” a cell with a compound or a molecule comprises attaching a number of such molecules to the cell surface. In some embodiments the cell surface is a cell membrane. In some embodiments, the terms “decorating”, “modifying”, “attaching”, “incorporating”, and “binding” are used herein interchangeably, having all the same meanings.

In some embodiments, the methods disclosed herein are applicable to any type of cells. In some embodiments, the cell is an eukaryote cell, a prokaryote cell, a mammalian cell, a plant cell, a human cell, and a bacteria cell. In some embodiments, the cell is E. coli.

In some embodiments, the cell is a living cell. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex). In some embodiments, the first compound is represented by the structure of J, H, H(complex), H(a), H(a)(complex), H(b) and H(b)(complex) and compounds 100-106. In some embodiments, the second compound is represented by the structure of formula K and compounds 200-214, 220, 230, 240 and 250. In some embodiments, the first linker comprises at least one oligoethyleneglycol (OEG) moiety, at least one phosphate moiety, at least one thioalkyl moiety or any combination thereof. In some embodiments, the first linker comprise two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the synthetic agent of said second compound comprises a molecular marker, a labeling moiety, a fluorescent dye, an adhesion molecule, a cancer cell binder, a protein binder, a protein ligand, an anticancer agent, a surface binder (e.g., an abiotic surface binder), a growth factor, an angiogenic factor, a cytokine, a hormone, a DNA molecule, a siRNA molecule, an oligosaccharide, a protein receptor, an immune activator, an immune suppressor, a small molecule, a drug, or a derivative therefore, or any combination thereof; each represents a separate embodiment according to this invention. In some embodiments, the second compound further comprises a second labeling moiety. In some embodiments, the second labeling moiety comprises a fluorescent dye. In some embodiments, the second compound further comprises at least two fluorescent dyes. In some embodiments, the second compound further comprises at least three fluorescent dyes.

In some embodiments, the method is for decorating a cell surface. In some embodiments, the method is for decorating a cell membrane. In some embodiments, the method is for modifying a cell surface. In some embodiments, the method is for modifying a cell membrane. In some embodiments, the synthetic agent is a labeling moiety. In some embodiments, the synthetic agent is a fluorescent dye. In some embodiments, the synthetic agent is a surface binder. In some embodiments, the synthetic agent is an abiotic surface binder. In some embodiments, the synthetic agent is a thioalkyl. In some embodiments, the synthetic agent is a protein binder. In some embodiments, the synthetic agent is a biotin. In some embodiments, the synthetic agent is a cancer cell binder. In some embodiments, the synthetic agent is a folate. In some embodiments, the synthetic agent is an anisamide (An). In some embodiments, the synthetic agent is a glutamate urea (GLA). In some embodiments, the synthetic agent selectively binds folate receptors (FRα). In some embodiments, the synthetic agent selectively bind sigma receptors (SRs). In some embodiments, the synthetic agent selectively bind prostate-specific membrane antigen (PSMA) receptor. In some embodiments, the synthetic agent comprises a cancer cell binder or derivative thereof. In some embodiments, the synthetic agent comprises a folate or derivative thereof. In some embodiments, the synthetic agent comprises an anisamide (An) or derivative thereof. In some embodiments, the synthetic agent comprises a glutamate urea (GLA) or derivative thereof. In some embodiments, the synthetic agent selectively binds folate receptors (FRa). In some embodiments, the synthetic agent selectively bind sigma receptors (SRs). In some embodiments, the synthetic agent selectively bind prostate-specific membrane antigen (PSMA) receptor. In some embodiments, the binder is a His-tag binder. In some embodiments, the His-tag binder is represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex) described hereinabove.

In some embodiments, cells are transformed with a construct encoding a polypeptide comprising a membranal anchoring domain and an extracellular binding domain. In some embodiments, said anchoring domain comprises OmpC, and said binding domain comprises a Histidine tag as described herein above. In some embodiments, transformed cells are cultured to saturation in a growth medium, such as LB supplemented with antibiotics at 30° C. In some embodiments, cells are incubated until the OD₆₀₀ reaches about 0.6, then the expression of the polypeptide is induced by addition of an inducer, such as Rhamnose or isopropyl-b-D-1-thiogalactopyranoside (IPTG), letting cultures to grow further.

Recombinant cells expressing the polypeptide are then collected, in some embodiments, by centrifugation at 6,000 g for 4 min, washed, and resuspended in the same buffer to an OD₆₀₀ of 0.3. A preincubated sample of a first molecule comprising a first oligonucleotide (ODN-1) can be added to a sample of the cell suspension. In some embodiments, 500 nM of ODN-1 and 2.5 μM of NiCl₂ can be added to the cells, which can then be incubated in some embodiments for 1 hour.

After a first compound is bound to the cell membrane, cells can be incubated with a second compound comprising a second oligonucleotide (ODN-2), wherein ODN-2 is complementary to ODN-1. Cells ODN-2 can be added in some embodiments at a concentration of 500 nM and incubated in some embodiments for 30 min.

In some embodiments, a second oligonucleotide ODN-2 can be detached from ODN-1 and from the recombinant cells by adding a third compound comprising a third oligonucleotide ODN-3, wherein ODN-3 is complementary to ODN-2. In some embodiments, ODN-3 can be added at a concentration of 2 μM and incubated for 2 h.

In some embodiments, a first, a second, or a third compound comprising a first, a second, or a third oligonucleotide, respectively, is added at a concentration lower than about 5 nM, between about 5 nM and 50 nM, between about 50 nM and 500 nM, between about 500 nM and 5 μM, between about 5M and 50 μM, between about 50 μM and 500 μM, or higher than 500 μM.

In some embodiments, the first compound comprising the first oligonucleotide (ODN-1) can in some embodiments be removed from the cell surface by incubating the cells with EDTA. In some embodiments, incubating the cells with about 5 mM or about 10 mM EDTA for 1 hour detaches the first compound from the cell surface. Cells can then be collected by centrifugation and washed.

### Applications of the DNA Construct, System, Recombinant Cells and B-Probes of the Invention

In some embodiments, the methods of this invention as described hereinbelow, make use of the DNA construct as described hereinabove under the titles: “DNA Construct for decorating cell membranes (1^{st} generation)”, “DNA Construct for decorating cell membranes (2^{nd} generation)” and “DNA Construct for decorating cell membranes (monoODN)”; each represents a separate embodiment according to this invention.

In some embodiments, this invention is directed to a method for labeling a cancer cell and/or carcinogenic tissues, said method comprises incubating a system, recombinant cell, or a B-probe according to this invention, with a cancer cell;
wherein the cancer cell comprises a CSP; and wherein the synthetic agent of said system, recombinant cell, or a B-probe, is a CSP binder or cancer cell binder, which comprises binding affinity to the CSP of said cancer cell.

In some embodiments, this invention is directed to a method for labeling a cancer cell and/or carcinogenic tissues, said method comprises:
a. incubating a recombinant cell ectopically expressing a polypeptide, which comprises a membranal anchoring domain and an extracellular binding domain, wherein said extracellular binding domain comprises a poly-histidine affinity tag, with a DNA construct according to this invention, wherein said synthetic agent of said DNA construct, is a CSP binder or cancer cell binder;
b. incubating said recombinant cell of (a) with a cancer cell;
   wherein the cancer cell comprises a cell surface protein (CSP); and
   wherein said CSP binder of said DNA construct, comprises binding affinity to the CSP of said cancer cell.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the recombinant cell is a bacterial probe according to this invention (B-probe). In some embodiments, the CSP binder is a cancer cell binder. In some embodiments, the CSP binder targets a small-molecule binding site in the cancer cell CSP. In some embodiments, the CSP is selectively expressed in said cancer cell. In some embodiments, the CSP is overexpressed in said cancer cell. In some embodiments, the method is taking place in a cellular environment. In some embodiments, the CSP binder or a cancer cell binder comprises a biotin, a folate, an anisamide, a glutamate urea, an antibody or derivative thereof, each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention. In some embodiments, the interaction between the recombinant cell and the cancer cell is multivalent.

In some embodiments, this invention is directed to a method for binding a first cell to a second cell, said method comprises incubating a system, recombinant cell, or a B-probe according to this invention (a first cell) with a second cell;
wherein the second cell comprises a CSP; and wherein the synthetic agent of said system, recombinant cell, or a B-probe is a CSP binder, which comprises binding affinity to the CSP of said second cell.

In some embodiments, this invention is directed to a method for binding a first cell to a second cell, said method comprises:
a. incubating a recombinant cell ectopically expressing a polypeptide, which comprises a membranal anchoring domain and an extracellular binding domain, wherein said extracellular binding domain comprises a poly-histidine affinity tag (a first cell), with a DNA construct according to this invention, wherein said synthetic agent of said DNA construct, is a CSP binder, thereby generating a DNA construct bound to a first cell (e.g., B-probe);
b. incubating said first cell of (a) with a second cell;
   wherein the second cell comprises a cell surface protein (CSP); and
   wherein said CSP binder of said DNA construct, comprises binding affinity to the CSP of said second cell.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the first cell is a bacterial probe according to this invention (B-probe). In some embodiments, the second cell is a cancer cell. In some embodiments, the CSP binder is a cancer cell binder. In some embodiments, the CSP is selectively expressed in said second cell. In some embodiments, the CSP is overexpressed in said second cell. In some embodiments, the method is taking place in a cellular environment. In some embodiments, the CSP binder or a cancer cell binder comprises a biotin, a folate, an anisamide, a glutamate urea or an antibody; each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention. In some embodiments, the interaction between the first cell and the second cell is multivalent.

In some embodiments, this invention is directed to a method for binding a cell to a protein of interest (POI), said method comprises incubating a system, recombinant cell, or a B-probe according to this invention (a cell) with a sample comprising a POI;
wherein the synthetic agent of said system, recombinant cell, or a B-probe is a protein binder, which comprises binding affinity to said POI.

In some embodiments, this invention is directed to a method for binding a cell to a protein of interest (POI), said method comprises:
a. incubating a recombinant cell ectopically expressing a polypeptide, which comprises a membranal anchoring domain and an extracellular binding domain, wherein said extracellular binding domain comprises a poly-histidine affinity tag (a cell), with a DNA construct according to this invention, wherein said synthetic agent of said DNA construct, is a protein binder, thereby generating a DNA construct bound to a cell (e.g., B-probe);
b. incubating said cell of (a) with a sample comprising a POI;
   wherein said protein binder of said DNA construct, comprises binding affinity to the POI.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the cell is a bacterial probe according to this invention (B-probe). In some embodiments, the method is taking place in a cellular environment. In some embodiments, the POI is a cell surface protein (CSP). In some embodiments, the CSP is a polypeptide or a protein, which is selectively expressed on the surface of a cell, preferably a cancer cell. In some embodiments, the CSP is a polypeptide or a protein, which is overexpressed on the surface of a cell, preferably a cancer cell. In some embodiments, the protein binder is selective to said POI. In some embodiments, the protein binder is a cell surface protein (CSP) binder, a small molecule ligand, an antibody, a peptide, a polypeptide, a protein or a part thereof; each represents a separate embodiment according to this invention. In some embodiments, the protein binder comprises a biotin, a folate, an anisamide, a glutamate urea or an antibody; each represents a separate embodiment according to this invention.

In some embodiments, this invention is directed to a method for detecting and/or labeling a protein of interest (POI) in a cellular environment, said method comprises:
a. imaging a sample comprising a protein of interest (POI) in cellular environment;
b. incubating the sample of (a) with a system, recombinant cell or a B-probe according to this invention, wherein said synthetic agent of said system, recombinant cell or a B-probe, is a protein binder, which comprises affinity to said POI;
c. optionally washing the sample of (b) from excess of said system, recombinant cell or a B-probe; and
d. imaging the fluorescence of said sample;
   wherein an increase in the fluorescence signal is indicative of the presence of said POI in said cellular environment, thereby detecting and/or labeling said protein of interest (POI) in said cellular environment.

In some embodiments, this invention is directed to a method for detecting and/or labeling a protein of interest (POI) in a cellular environment, said method comprises:
a. incubating a recombinant cell ectopically expressing a polypeptide, which comprises a membranal anchoring domain and an extracellular binding domain, wherein said extracellular binding domain comprises a poly-histidine affinity tag (a cell), with a DNA construct according to this invention, wherein said synthetic agent of said DNA construct, is a protein binder, which comprises affinity to said POI, thereby generating a DNA construct bound to a cell (e.g., B-probe);
b. imaging a sample comprising a protein of interest (POI) in cellular environment;
c. incubating the sample of (b) with the cell of (a);
d. optionally washing the sample of (c) from excess of said cell; and
e. imaging the fluorescence of said sample;
   wherein an increase in the fluorescence signal is indicative of the presence of said POI in said cellular environment, thereby detecting and/or labeling said protein of interest (POI) in said cellular environment.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the cell is a bacterial probe according to this invention (B-probe). In some embodiments, the cellular environment comprises living cells. In some embodiments, the POI is a cell surface protein (CSP). In some embodiments, the CSP is a polypeptide or a protein, which is selectively expressed on the surface of a cell, preferably a cancer cell. In some embodiments, the CSP is a polypeptide or a protein, which is overexpressed on the surface of a cell, preferably a cancer cell. In some embodiments, the protein binder is selective to said POI. In some embodiments, the protein binder is a cell surface protein (CSP) binder, a small molecule ligand, an antibody, a peptide, a polypeptide, a protein or a part thereof; each represents a separate embodiment according to this invention. In some embodiments, the protein binder comprises a biotin, a folate, an anisamide, a glutamate urea or an antibody; each represents a separate embodiment according to this invention. In some embodiments, the fluorescence signal is measured by a fluorescence microscope or by recording the emission with a spectrophotometer at a particular wavelength; each represents a separate embodiment according to this invention.

In some embodiments, this invention is directed to a method for measuring the interaction between a protein of interest (POI) and a potential ligand for said POI, said method comprises: a. imaging a sample comprising a protein of interest (POI);
b. incubating the sample of (a) with a system, recombinant cell or a B-probe according to this invention, and with a potential ligand, wherein said synthetic agent of said system, recombinant cell or a B-probe, is a protein binder, which comprises affinity to said POI;
c. optionally washing the sample of (b) from excess of said cell and said potential ligand;
d. measuring the fluorescence imaging of said sample;
e. comparing the measured fluorescence of the sample of (d) with the fluorescence measured from incubating a control sample comprising a protein of interest (POI) with a system, recombinant cell or a B-probe according to this invention, followed by washing excess of the system, recombinant cell or a B-probe according to this invention (i.e. a control);
   wherein reduction in the fluorescence signal with respect to the control is indicative of the interaction between said POI and said potential ligand, thereby measuring the interaction between a protein of interest (POI) and a potential ligand for said POI.

In some embodiments, this invention is directed to a method for measuring the interaction between a protein of interest (POI) and a potential ligand for said POI, said method comprises: a. incubating a recombinant cell ectopically expressing a polypeptide, which comprises a membranal anchoring domain and an extracellular binding domain, wherein said extracellular binding domain comprises a poly-histidine affinity tag (a cell), with a DNA construct according to this invention, wherein said synthetic agent of said DNA construct, is a protein binder, which comprises affinity to said POI, thereby generating a DNA construct bound to a cell (e.g., B-probe);
b. imaging a sample comprising a protein of interest (POI);
c. incubating the sample of (b) with the sample of (a) and with the potential ligand;
d. washing the sample of (c) from excess of said cell and said potential ligand;
e. measuring the fluorescence imaging of said sample;
f. comparing the measured fluorescence of the sample of (e) with the fluorescence measured from incubating a control sample comprising a protein of interest (POI) with the cell of (a), followed by washing excess of the cell (i.e. a control);
wherein reduction in the fluorescence signal with respect to the control is indicative of the interaction between said POI and said potential ligand, thereby measuring the interaction between a protein of interest (POI) and a potential ligand for said POI.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the cell is a bacterial probe according to this invention (B-probe). In some embodiments, the cellular environment comprises living cells. In some embodiments, the POI is a cell surface protein (CSP). In some embodiments, the CSP is a polypeptide or a protein, which is selectively expressed on the surface of a cell, preferably a cancer cell. In some embodiments, the CSP is a polypeptide or a protein, which is overexpressed on the surface of a cell, preferably a cancer cell. In some embodiments, the protein binder is selective to said POI. In some embodiments, the protein binder is a cell surface protein (CSP) binder, a small molecule ligand, an antibody, a peptide, a polypeptide, a protein or a part thereof; each represents a separate embodiment according to this invention. In some embodiments, the protein binder comprises a biotin, a folate, an anisamide, a glutamate urea or an antibody; each represents a separate embodiment according to this invention. In some embodiments, the fluorescence signal is measured by a fluorescence microscope or by recording the emission with a spectrophotometer at a particular wavelength; each represents a separate embodiment according to this invention.

In some embodiments, this invention is directed to a method for cell-based screening for potential ligands for a protein of interest (POI), said method comprises:
a. imaging a sample comprising a protein of interest (POI);
b. incubating the sample of (a) with a system, recombinant cell or a B-probe according to this invention, and with a potential ligand, wherein said synthetic agent of said system, recombinant cell or a B-probe, is a protein binder, which comprises affinity to said POI;
c. optionally washing the sample of (b) from excess of said cell and said potential ligand;
d. measuring the fluorescence imaging of said sample;
e. comparing the measured fluorescence of the sample of (d) with the fluorescence measured from incubating a control sample comprising a protein of interest (POI) with a system, recombinant cell or a B-probe according to this invention, followed by washing excess of the system, recombinant cell or a B-probe according to this invention (i.e. a control);
   wherein reduction in the fluorescence signal with respect to the control is indicative of the interaction between said POI and said potential ligand, thereby screening for potential ligands for said POI.

In some embodiments, this invention is directed to a method for cell-based screening for potential ligands for a protein of interest (POI), said method comprises:
a. incubating a recombinant cell ectopically expressing a polypeptide, which comprises a membranal anchoring domain and an extracellular binding domain, wherein said extracellular binding domain comprises a poly-histidine affinity tag (a cell), with a DNA construct according to this invention, wherein said synthetic agent of said DNA construct, is a protein binder, which comprises affinity to said POI, thereby generating a DNA construct bound to a cell (e.g., B-probe);
b. imaging a sample comprising a protein of interest (POI);
c. incubating the sample of (b) with the sample of (a);
d. washing the sample of (c) from excess of said cell and said potential ligand;
e. measuring the fluorescence imaging of said sample;
f. comparing the measured fluorescence of the sample of (e) with the fluorescence measured from incubating a control sample comprising a protein of interest (POI) with the cell of (a), followed by washing excess of the cell (i.e. a control);
wherein reduction in the fluorescence signal with respect to the control is indicative of the interaction between said POI and said potential ligand, thereby screening for potential ligands for said POI.

In some embodiments, step (a) is carried out in the presence of Ni²⁺ ions. In some embodiments, the binding of said DNA construct to said recombinant cell is obtained via the binding of the His-tag specific binder of the DNA construct, to the poly-histidine affinity tag of the polypeptide of said recombinant cell, in the presence of Ni²⁺ ions.

In some embodiments, the cell is a bacterial probe according to this invention (B-probe). In some embodiments, the cellular environment comprises living cells. In some embodiments, the POI is a cell surface protein (CSP). In some embodiments, the CSP is a polypeptide or a protein, which is selectively expressed on the surface of a cell, preferably a cancer cell. In some embodiments, the CSP is a polypeptide or a protein, which is overexpressed on the surface of a cell, preferably a cancer cell. In some embodiments, the protein binder is selective to said POI. In some embodiments, the protein binder is a cell surface protein (CSP) binder, a small molecule ligand, an antibody, a peptide, a polypeptide, a protein or a part thereof; each represents a separate embodiment according to this invention. In some embodiments, the protein binder comprises a biotin, a folate, an anisamide, a glutamate urea or an antibody; each represents a separate embodiment according to this invention. In some embodiments, the fluorescence signal is measured by a fluorescence microscope or by recording the emission with a spectrophotometer at a particular wavelength; each represents a separate embodiment according to this invention.

### Methods for Adhering a First Cell to a Second Cell

In some embodiments, this invention is directed to a method for adhering a first cell to a second cell, said method comprises incubating a system, B-probe, or recombinant cell according to this invention, with a second cell, wherein the synthetic agent is an adhesion molecule, a cancer cell binder, a protein binder, a CSP binder; each represents a separate embodiment according to this invention.

In some embodiments, this invention is directed to a method for binding a first cell to a second cell, said method comprises incubating a system, B-probe, or recombinant cell according to this invention, with a second cell, wherein the synthetic agent is an adhesion molecule, a cancer cell binder, a protein binder, a CSP binder; each represents a separate embodiment according to this invention. In another embodiment, the synthetic agent is a protein binder. In another embodiment, the synthetic agent is a cancer cell binder. In another embodiment, the second cell is a cancer cell. In some embodiments, the interaction between the first cell and the second cell is multivalent.

In some embodiments, this invention is directed to a method for adhering a first cell to a second cell, said method comprises incubating a cell ectopically expressing a polypeptide according to this invention, wherein said polypeptide comprises a membranal anchoring domain and an extracellular binding domain, with a DNA construct according to this invention, wherein the synthetic agent is an adhesion molecule, a cancer cell binder, a protein binder, a CSP binder; each represents a separate embodiment according to this invention, thereby forming a complex according to this invention, following by incubating the formed complex with a second cell, thereby adhering a first cell to a second cell.

In some embodiments, this invention is directed to a method for binding a first cell to a second cell, said method comprises incubating a cell ectopically expressing a polypeptide according to this invention, wherein said polypeptide comprises a membranal anchoring domain and an extracellular binding domain, with a DNA construct according to this invention, wherein the synthetic agent is a protein binder, thereby forming a complex according to this invention, following by incubating the formed complex with a second cell, thereby binding a first cell to a second cell.

In some embodiments, this invention is directed to a method for binding a first cell to a cancer cell, said method comprises incubating a cell ectopically expressing a polypeptide according to this invention, wherein said polypeptide comprises a membranal anchoring domain and an extracellular binding domain, with a DNA construct according to this invention, wherein the synthetic agent is a cancer cell binder, thereby forming a complex according to this invention, following by incubating the formed complex with a cancer cell, thereby binding a first cell to a cancer cell. In some embodiments, the cancer cell binder is a folate (Fo), an anisamide (An), a glutamate urea (GLA); each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention. In some embodiments, the interaction between the first cell and the second cell is multivalent.

In some embodiments, this invention is directed to a method for adhering a first cell to a second cell, said method comprises:
a. ectopically expressing in the first cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a synthetic agent, either directly or through a second linker, wherein said second oligonucleotide is complementary to said first oligonucleotide thereby generating a DNA construct according to this invention and wherein said synthetic agent comprises affinity to a compound present on the surface of said second cell;
c. incubating said DNA construct with said first cell thereby forming a DNA construct bound to the recombinant cell of (a);
d. incubating said cell of (c) with a second cell,
   wherein said synthetic agent is an adhesion molecule, a protein binder, a cancer cell binder, or a CSP binder; each represents a separate embodiment according to this invention.
   thereby adhering said first cell to said second cell.

In some embodiments, this invention is directed to a method for adhering a first cell to a second cell, said method comprises:
a. ectopically expressing in the first cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain;
   with a second compound comprising a second oligonucleotide (ODN-2) covalently bound to a synthetic agent, either directly or through a second linker, wherein said synthetic agent comprises affinity to a compound present on the surface of said second cell, and wherein said second oligonucleotide is complementary to said first oligonucleotide, and wherein said second oligonucleotide comprises a first hanging strand (a first toehold region);
   and with a third compound comprising a DNA duplex (dsDNA) appended with a second hanging strand complementary to said first hanging strand, and further appended with at least two fluorescent dyes,
thereby generating a DNA construct according to this invention;
c. incubating said DNA construct with said first cell thereby forming a DNA construct bound to the recombinant cell of (a);
d. incubating said cell of (c) with a second cell,
   wherein said synthetic agent is an adhesion molecule, a protein binder, a cancer cell binder, or a CSP binder; each represents a separate embodiment according to this invention.
   thereby adhering said first cell to said second cell.

In some embodiments, this invention is directed to a method for binding a first cell to a second cell, said method comprises:
a. ectopically expressing in the first cell a polypeptide, wherein said polypeptide comprises a membranal anchoring domain and an extracellular binding domain;
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a synthetic agent, either directly or through a second linker, wherein said second oligonucleotide is complementary to said first oligonucleotide, thereby generating a DNA construct according to this invention;
and wherein said synthetic agent comprises affinity to a compound present on the surface of said second cell,
c. incubating said DNA construct with said first cell thereby forming a DNA construct bound to the first cell of (a);
d. incubating said DNA construct bound first cell of (c) with a second cell, thereby binding said first cell to said second cell.

In some embodiments, this invention is directed to a method for binding a first cell to a second cell, said method comprises:
a. ectopically expressing in the first cell a polypeptide, wherein said polypeptide comprises a membranal anchoring domain and an extracellular binding domain;
b. incubating a first compound comprising a first oligonucleotide (ODN-1) covalently bound to a binder, either directly or through a first linker, said binder comprising affinity to said extracellular binding domain;
   with a second compound comprising a second oligonucleotide (ODN-2) covalently bound to a synthetic agent, either directly or through a second linker, wherein said synthetic agent comprises affinity to a compound present on the surface of said second cell, and wherein said second oligonucleotide is complementary to said first oligonucleotide, and wherein said second oligonucleotide comprises a first hanging strand (a first toehold region);
   and with a third compound comprising a DNA duplex (dsDNA) appended with a second hanging strand complementary to said first hanging strand, and further appended with at least two fluorescent dyes,
   thereby generating a DNA construct according to this invention;
c. incubating said DNA construct with said first cell thereby forming a DNA construct bound to the first cell of (a);
d. incubating said DNA construct bound first cell of (c) with a second cell, thereby binding said first cell to said second cell.

In some embodiments, synthetic agent is a protein binder. In some embodiments, incubation of the DNA construct with the first cell is carried out in the presence of metal ions. In some embodiments, the metal is nickel. In some embodiments, incubation of the DNA construct with the first cell is carried out in the presence of Ni²⁺ ions.

In some embodiments, the interaction between the first cell and the second cell is multivalent.

In some embodiments, the recombinant cell of the system, the B-probe, and/or the methods according to this invention, is selected from a group comprising eukaryotes, prokaryotes, mammalian cells, plant cells, human cells, and bacteria. In some embodiments, a mammalian or a human cell is selected from a group comprising epithelial cells, Brunner's gland cells in duodenum, insulated goblet cells of respiratory and digestive tracts, stomach, foveolar cells, chief cells, parietal cells, pancreatic acinar cells, Paneth cells of small intestine, Type II pneumocyte of lung, club cells of lung, barrier cells, type i pneumocytes, gall bladder epithelial cells, centroacinar cells, intercalated duct cells, intestinal brush border cells, hormone-secreting cells, enteroendocrine cells, K cells, L cells, I cells, G cells, enterochromaffin cells, enterochromaffin-like cells, N cells, S cells, D cells, Mo cells, thyroid gland cells, thyroid epithelial cells, parafollicular cells, parathyroid gland cells, parathyroid chief cells, oxyphil cells, pancreatic islets, alpha cells, beta cells, delta cells, epsilon cells, PP cells, salivary gland mucous cells, salivary gland serous cells, Von Ebner's gland cells in tongue, mammary gland cells, lacrimal gland cells, ceruminous gland cells in ear, eccrine sweat gland dark cells, eccrine sweat gland clear cells, apocrine sweat gland cells, gland of moll cells in eyelid, sebaceous gland cells, Bowman's gland cells in nose, hormone-secreting cells, anterior/intermediate pituitary cells, corticotropes, gonadotropes, lactotropes, melanotropes, somatotropes, thyrotropes, magnocellsular neurosecretory cells, parvocellsular neurosecretory cells, chromaffin cells, keratinocytes, epidermal basal cells, melanocytes, trichocytes, medullary hair shaft cells, cortical hair shaft cells, cuticular hair shaft cells, huxley's layer hair root sheath cells, Henle's layer hair root sheath cells, outer root sheath hair cells, surface epithelial cells of cornea, tongue, mouth, nasal cavity, distal anal canal, distal urethra, and distal vagina, basal cells, intercalated duct cells, striated duct cells, lactiferous duct cells, ameloblast, auditory inner hair cells of organ of Corti, auditory outer hair cells of organ of Corti, basal cells of olfactory epithelium, primary sensory neurons, Merkel cells of epidermis, olfactory receptor neuron, pain-sensitive primary sensory neurons, photoreceptor cells of retina in eye, proprioceptive primary sensory neurons, touch-sensitive primary sensory neurons, chemoreceptor glomus cells of carotid body cells, outer hair cells of vestibular system of ear, inner hair cells of vestibular system of ear, taste receptor cells of taste bud, neuron cells, interneurons, basket cells, cartwheel cells, Stellate cells, Golgi cells, granule cells, Lugaro cells, unipolar brush cells, Martinotti cells, chandelier cells, Cajal-Retzius cells, double-bouquet cells, neurogliaform cells, retina horizontal cells, amacrine cells, spinal interneuron, renshaw cells, spindle neurons, fork neurons, pyramidal cells, place cells, grid cells, speed cells, head direction cells, Betz cells, stellate cells, boundary cells, bushy cells, Purkinje cells, medium spiny neurons, astrocytes, oligodendrocytes, ependymal cells, tanycytes, pituicytes, adipocytes, white fat cells, brown fat cells, liver lipocytes, cells of the adrenal cortex, cells of the zona glomerulosa, cells of the zona fasciculata, cells of the zona reticularis, theca interna cells of ovarian follicle, granulosa lutein cells, theca lutein cells, leydig cells of testes, seminal vesicle cells, prostate gland cells, bulbourethral gland cells, Bartholin's gland cells, gland of littre cells, uterus endometrium cells, juxtaglomerular cells, macula densa cells of kidney, peripolar cells of kidney, mesangial cells of kidney, parietal epithelial cells, podocytes, proximal tubule brush border cells, loop of Henle thin segment cells, kidney distal tubule cells, kidney collecting duct cells, principal cells, intercalated cells, transitional epithelium, duct cells, efferent ducts cells, epididymal principal cells, epididymal basal cells, endothelial cells, planum semilunatum epithelial cells of vestibular system of ear, organ of Corti interdental epithelial cells, loose connective tissue fibroblasts, corneal fibroblasts, tendon fibroblasts, bone marrow reticular tissue fibroblasts, other nonepithelial fibroblasts, pericytes, hepatic stellate cells, nucleus pulposus cells of intervertebral disc, hyaline cartilage chondrocytes, fibrocartilage chondrocytes, elastic cartilage chondrocytes, osteoblast/osteocytes, osteoprogenitor cells, hyalocyte of vitreous body of eye, stellate cells of perilymphatic space of ear, pancreatic stellate cells, red skeletal muscle cells, white skeletal muscle cells, intermediate skeletal muscle cells, nuclear bag cells of muscle spindle, nuclear chain cells of muscle spindle, myosatellite cells, cardiac muscle cells, cardiac muscle cells, node cells, Purkinje fiber cells, smooth muscle cells, myoepithelial cells of iris, myoepithelial cells of exocrine glands, erythrocytes, megakaryocytes, platelets, monocytes, connective tissue macrophage, epidermal Langerhans cells, osteoclast, dendritic cells, microglial cells, neutrophil granulocytes, eosinophil granulocytes, basophil granulocytes, hybridoma cells, mast cells, helper T cells, suppressor T cells, cytotoxic T cells, natural killer T cells, B cells, natural killer cells, reticulocytes, hematopoietic stem cells and committed progenitors for the blood and immune system, oogonium/oocytes, spermatids, spermatocytes, spermatogonium cells, spermatozoon, and interstitial kidney cells.

In some embodiments, the second cell comprises a cellular pathology. In some embodiments, the second cell is a cancer cell. In some embodiments, the cancer is selected from: a carcinoma, a sarcoma, a lymphoma, leukemia, a germ cell tumor, a blastoma, chondrosarcoma, Ewing's sarcoma, malignant fibrous histiocytoma of bone/osteosarcoma, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoetic cancer, lymphoma, Non-hodgkin lymphoma, skin cancer, basal-cell carcinoma, melanoma, small cell lung cancer, non-small cell lung cancer, or any combination thereof. In some embodiments, the cancer is selected from: prostate cancer, cervical cancer, and melanoma; each represents a separate embodiment according to this invention.

In some embodiments, the adhesion molecule is any compound that comprises affinity to a compound present in the surface of a second cell. In some embodiments, the adhesion molecule is any compound that comprises affinity to a compound present in the membrane of a second cell. In some embodiments, the adhesion molecule is selected according to its binding potency to a molecule known to be expressed in a second cell. In some embodiments, the adhesion molecule is any adhesion molecule known in the art. In some embodiments, the adhesion molecule comprises an antibody, a biotin, a folate, an anisamide, a glutamate urea or derivative thereof, each represents a separate embodiment according to this invention.

In some embodiments, the adhesion molecule is a peptide, a polypeptide, a protein or a part thereof. In some embodiments, the adhesion molecule comprises an integrin or a fragment thereof. In some embodiments, the adhesion molecule comprises an immunoglobulin (Ig) or a fragment thereof. In some embodiments, the adhesion molecule comprises a cadherin, or a fragment thereof. In some embodiments, the adhesion molecule comprises a selectins, or a fragment thereof. In some embodiments, the adhesion molecule comprises a calcium-dependent cell adhesion molecule, or a fragment thereof. In some embodiments, the adhesion molecule comprises a proteoglycan, or a fragment thereof. A skilled artisan would appreciate that adhesion molecule recognizes a different ligand.

In some embodiments an adhesion molecule is selected from a group comprising VLA1, VLA2, VLA3, VLA4, VLA5, VLA6, FLJ25220, RLC, HsT18964, FLJ39841, HUMINAE, LFA1A, MAC-I, VNRA, MSK8, GPIIb, FNRB, MSK12, MDF2, LFA-1, MAC-I, MFI7, GP3A, GPIIIa, FLJ26658, fibronectin receptor, laminin receptor, LFA-1, CR3, fibrinogen receptor; gpIIbIIIa, vitronectin receptor, CDH1, CDH2, CDH12, CDH3, DSG1, DSG2, DSG3, DSG4, Desmocollin, DSC1, DSC2, DSC3, Protocadherins, IgSF CAMs, NCAMs, ICAM-1, CD2, CD58, CD48, CD150, CD229, CD244, E-selectin, L-selectin, P-selectin, any fragment thereof, or any combination thereof.

In some embodiments, the cell adhesion molecule comprises a folate or derivative thereof. In some embodiments, the second cell expresses an extracellular folate receptor on its surface. In some embodiments, the cell adhesion molecule comprises an anisamide or derivative thereof. In some embodiments, the second cell expresses an extracellular sigma receptor on its surface. In some embodiments, the cell adhesion molecule comprises a glutamate urea or derivative thereof. In some embodiments, the second cell expresses an extracellular prostate-specific membrane antigen (PSMA) receptor on its surface.

In some embodiments, the first cell is a living cell. In some embodiments, the second cell is a living cell. In some embodiments, the second cell is a cancer cell. In some embodiments, the second cell expresses an extracellular protein receptor on its surface. In some embodiments, the adhesion molecule is a protein binder. In some embodiments, the adhesion molecule is a folate or derivative thereof. In some embodiments, the adhesion molecule is an anisamide or derivative thereof. In some embodiments, the adhesion molecule is a glutamate urea or derivative thereof.

In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention.

In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof.

In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex).

In some embodiments, the first compound of the methods according to this invention is as described under the title “The first Compound (X-ODN-1)” hereinbelow. In some embodiments, the first compound is represented by the structure of J, H, H(complex), H(a), H(a)(complex), H(b) and H(b)(complex) and compounds 100-106. In some embodiments, the first compound further comprises a labeling moiety. In some embodiments, the labeling moiety is a fluorescent dye.

In some embodiments, the second compound of the methods according to this invention is as described under the title “The second Compound (Y-ODN-2)” hereinbelow. In some embodiments, the second compound is represented by the structure of formula K and compounds 200-214, 220, 230, 240 and 250. In some embodiments, the second compound further comprises a second labeling moiety. In some embodiments, the second labeling moiety comprises a fluorescent dye. In some embodiments, the second compound comprises at least two labeling moieties. In some embodiments, the second compound comprises at least two fluorescent dyes. In some embodiments, the second compound comprises a first hanging strand. In some embodiments, the DNA construct further comprises a third compound comprising a DNA duplex (dsDNA) appended with a second hanging strand complementary to said first hanging strand, and further appended with at least two fluorescent dyes.

In some embodiments, the first linker comprises at least one oligoethyleneglycol (OEG) moiety, at least one phosphate moiety, at least one thioalkyl moiety or any combination thereof. In some embodiments, the first linker comprise two; three; four; five; six; seven or eight sequentially arranged oligoethylene glycol (OEG) spacers; each represents a separate embodiment according to this invention.

### Methods for Adhering a Cell to a Surface

In some embodiments, this invention is directed to a method for adhering a cell to a surface, said method comprises incubating a recombinant cell according to the invention, with a first compound according to the invention, following by incubating the formed cell with a second compound according to this invention, wherein the synthetic agent is a surface binder.

In some embodiments, this invention is directed to a method for adhering a cell to a surface, said method comprises:
a. ectopically expressing in a cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a surface binder, either directly or through a second linker, wherein said second oligonucleotide is complementary to said first oligonucleotide thereby generating a DNA construct according to this invention and wherein said surface binder is capable of binding to said surface, and
c. incubating said DNA construct with said cell thereby forming a DNA construct bound to the recombinant cell of (a);
d. applying said cell to said surface under conditions sufficient for the binding of said surface binder to said surface, thereby adhering said cell to said surface.

In some embodiments, this invention is directed to a method for adhering a cell to a surface, said method comprises:
a. ectopically expressing in a cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain;
   with a second compound comprising a second oligonucleotide (ODN-2) covalently bound to a surface binder, either directly or through a second linker, wherein said surface binder is capable of binding to said surface, and wherein said second oligonucleotide is complementary to said first oligonucleotide, and wherein said second oligonucleotide comprises a first hanging strand (a first toehold region);
   and with a third compound comprising a DNA duplex (dsDNA) appended with a second hanging strand complementary to said first hanging strand, and further appended with at least two fluorescent dyes, thereby generating a DNA construct according to this invention;
c. incubating said DNA construct with said cell thereby forming a DNA construct bound to the recombinant cell of (a);
d. applying said cell to said surface under conditions sufficient for the binding of said surface binder to said surface, thereby adhering said cell to said surface.

In some embodiments, incubation of the DNA construct with the cell is carried out in the presence of metal ions. In some embodiments, the metal is nickel. In some embodiments, incubation of the DNA construct with the first cell is carried out in the presence of Ni²⁺ ions.

In some embodiments, the cell is a living cell. In some embodiments, the cell is a bacteria. In some embodiments, the bacteria binds to the surface in a multivalent fashion. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex).

In some embodiments, the first compound of the methods according to this invention is as described under the title “The first Compound (X-ODN-1)” hereinbelow. In some embodiments, the first compound is represented by the structure of J, H, H(complex), H(a), H(a)(complex), H(b) and H(b)(complex) and compounds 100-106. In some embodiments, the first compound further comprises a labeling moiety. In some embodiments, the labeling moiety is a fluorescent dye.

In some embodiments, the second compound of the methods according to this invention is as described under the title “The second Compound (Y-ODN-2)” hereinbelow. In some embodiments, the second compound is represented by the structure of formula K and compounds 200-214, 220, 230, 240 and 250. In some embodiments, the second compound further comprises a second labeling moiety. In some embodiments, the second labeling moiety comprises a fluorescent dye. In some embodiments, the second compound comprises at least two labeling moieties. In some embodiments, the second compound comprises at least two fluorescent dyes. In some embodiments, the second compound comprises a first hanging strand. In some embodiments, the DNA construct further comprises a third compound comprising a DNA duplex (dsDNA) appended with a second hanging strand complementary to said first hanging strand, and further appended with at least two fluorescent dyes.

In some embodiments, the first linker comprises at least one oligoethyleneglycol (OEG) moiety, at least one phosphate moiety, at least one thioalkyl moiety or any combination thereof. In some embodiments, the first linker comprise two; three; four; five; six; seven or eight sequentially arranged oligoethylene glycol (OEG) spacers; each represents a separate embodiment according to this invention.

In some embodiments, the surface binder is an abiotic surface binder. In some embodiments, the surface is a solid support. In some embodiments, the surface is a passivated. In some embodiments, the surface is a material selected from gold, glass, a doped glass, indium tin oxide (ITO)-coated glass, silicon, a doped silicon, Si(100), Si(111), SiO₂, SiH, silicon carbide mirror, quartz, a metal, metal oxide, a mixture of metal and metal oxide, group IV elements, mica, a polymer such as polyacrylamide and polystyrene, a plastic, a zeolite, a clay, wood, a membrane, an optical fiber, a ceramic, a metalized ceramic, an alumina, an electrically-conductive material, a semiconductor, steel or a stainless steel; each is a separate embodiment according to the invention. In some embodiments, the surface is a gold surface.

In some embodiments, the surface binder is a C₁-C₂₀ thioalkyl. In some embodiments, the surface binder is a C₂-C₈ thioalkyl. In some embodiments, the surface binder is a thiohexyl. In some embodiments, the surface binder is a pyridine-terminated moiety.

### Methods for Inducing Luminescent in a Cell

In some embodiments, this invention is directed to a method for inducing luminescence in a cell, said method comprises incubating a recombinant cell according to the invention, with a first compound according to the invention, following by incubating the formed cell with a second compound according to this invention, wherein the synthetic agent is a luminescent moiety.

In some embodiments, this invention is directed to a method for inducing luminescence in a cell, said method comprises:
a. ectopically expressing in a cell a first polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a luminescent molecule, either directly or through a second linker, wherein said second oligonucleotide is complementary to said first oligonucleotide thereby generating a DNA construct according to this invention, and
c. incubating said DNA construct with said cell of (a) thereby forming a DNA construct bound to said recombinant cell;
   thereby inducing luminescence in said cell.

Any luminescent molecule can be used in the methods disclosed herein. In some embodiments, the luminescent molecule is as described for a “labeling moiety” herein above. In some embodiments, the luminescent molecule is a fluorescent dye. Examples of fluorescent dyes are given herein above. In some embodiments, the dye is selected from: dansyl, fluorescein (6-FAM), FAM, cyanine dyes (e.g. Cy3, Cy5), sulfoindocyanine, nile red, rhodamine, perylene, fluorenyl, coumarin, 7-methoxycoumarin (Mca), dabcyl, NBD, Nile blue, TAMRA, BODIPY, FITC, Thiazole orange, Quinoline blue, Thiazole red, phycoerythrin (PE) and derivatives thereof.

In some embodiments, incubation of the DNA construct with the cell is carried out in the presence of metal ions. In some embodiments, the metal is nickel. In some embodiments, incubation of the DNA construct with the first cell is carried out in the presence of Ni²⁺ ions.

In some embodiments, this invention is directed to a method for inducing luminescence in a cell, said method comprises:
a. ectopically expressing in a cell a polypeptide, wherein said polypeptide comprises a membranal anchoring domain and an extracellular binding domain, wherein said extracellular binding domain of said polypeptide comprises an affinity tag;
b. incubating a DNA construct according to this invention with said cell of (a);
   thereby inducing luminescence in said cell.

In some embodiments, incubation of the DNA construct with the cell is carried out in the presence of metal ions. In some embodiments, the metal is nickel. In some embodiments, incubation of the DNA construct with the first cell is carried out in the presence of Ni²⁺ ions.

In some embodiments, the cell is a living cell. In some embodiments, the cell is a bacteria. In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof; each represents a separate embodiment according to this invention. In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof. In some embodiments, the binder of the DNA construct comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex). In some embodiments, the first compound is represented by the structure of J, H, H(complex), H(a), H(a)(complex), H(b) and H(b)(complex) and compounds 100-106. In some embodiments, the second compound is represented by the structure of formula K and compounds 200-214, 220, 230, 240 and 250. In some embodiments, the first linker comprises at least one oligoethyleneglycol (OEG) moiety, at least one phosphate moiety, at least one thioalkyl moiety or any combination thereof. In some embodiments, the first linker comprises two; three; four; five; six; seven or eight sequentially arranged oligoethylene glycol (OEG) spacers; each represents a separate embodiment according to this invention. In some embodiments, the first compound further comprises a labeling moiety. In some embodiments, the labeling moiety is a fluorescent dye. In some embodiments, the second compound further comprises a second labeling moiety. In some embodiments, the second labeling moiety comprises a fluorescent dye. In some embodiments, the second compound further comprises a hanging strand. In some embodiments, the hanging strand is appended with at least two fluorescent dyes. In some embodiments, the hanging strand is bound to a DNA duplex appended with at least two fluorescent dyes, as described hereinabove under the title “DNA duplex (dsDNA)”.

### Methods for Binding a Cell to a Protein

In some embodiments, this invention is directed to a method for binding a cell to a protein of interest (POI), said method comprises incubating a recombinant cell according to this invention, with said POI, wherein the synthetic agent is a protein binder.

In some embodiments, this invention is directed to a method for binding a cell to a protein of interest (POI), said method comprises incubating a cell ectopically expressing a polypeptide according to this invention, wherein said polypeptide comprises a membranal anchoring domain and an extracellular binding domain, with a first compound according to this invention and with a second compound according to this invention, thereby forming a complex according to this invention, following by incubating the formed complex with a POI, wherein the synthetic agent is a protein binder, thereby binding a cell to a protein of interest (POI).

In some embodiments, this invention is directed to a method for binding a cell to a protein of interest (POI), said method comprises:
a. ectopically expressing in a cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain, with a second compound comprising a second oligonucleotide (ODN-2) covalently bound to a protein binder, either directly or through a second linker, wherein said second oligonucleotide is complementary to said first oligonucleotide thereby generating a DNA construct according to this invention and wherein said protein binder is selective to said POI;
c. incubating said DNA construct with said recombinant cell of (a) thereby forming a DNA construct bound to said cell;
d. incubating said cell with said POI, thereby binding said cell to said POI.

In some embodiments, this invention is directed to a method for binding a cell to a protein of interest (POI), said method comprises:
a. ectopically expressing in a cell a polypeptide, wherein said polypeptide comprises a membranal anchoring domain and an extracellular binding domain,
b. incubating a first compound comprising a first oligonucleotide (ODN-1) covalently bound to a binder, either directly or through a first linker, said binder comprising affinity to said extracellular binding domain;
   with a second compound comprising a second oligonucleotide (ODN-2) covalently bound to a protein binder, either directly or through a second linker, wherein said protein binder is selective to said POI, and wherein said second oligonucleotide is complementary to said first oligonucleotide, and wherein said second oligonucleotide comprises a first hanging strand (a first toehold region);
   and with a third compound comprising a DNA duplex (dsDNA) appended with a second hanging strand complementary to said first hanging strand, and further appended with at least two fluorescent dyes, thereby generating a DNA construct according to this invention;
c. incubating said DNA construct with said recombinant cell of (a) thereby forming a DNA construct bound to said cell;
d. incubating said cell with said POI,
   thereby binding said cell to said POI.

In some embodiments, incubation of the DNA construct with the cell is carried out in the presence of metal ions. In some embodiments, the metal is nickel. In some embodiments, incubation of the DNA construct with the cell is carried out in the presence of Ni²⁺ ions.

In some embodiments, the cell is a living cell. In some embodiments, the cell is a bacteria. In some embodiments, the bacteria binds at least one, at least two, at least three, at least four POIs, each represents a separate embodiment according to this invention.

In some embodiments, the membranal anchoring domain comprises a transmembranal protein or a part of it, an artificial polypeptide, or a combination thereof. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention.

In some embodiments, the extracellular domain comprises an affinity tag. In some embodiments, the affinity tag comprises a poly-histidine peptide (6×-His-tag, 10×-His-tag, His-tag), a tetra cysteine peptide (CCPGCC, TC tag), or a combination thereof.

In some embodiments, the binder comprises a His-tag specific binder. In some embodiments, the binder comprises a moiety represented by the structure of formula C, D, D(complex), D(a), D(a)(complex), D(b), D(b)(complex), E, E(complex), E(a), E(a)(complex), E(b), E(b)(complex), G, G(complex), G(a), G(a)(complex), G(b) or G(b)(complex).

In some embodiments, the first compound of the methods according to this invention is as described under the title “The first Compound (X-ODN-1)” hereinbelow. In some embodiments, the first compound is represented by the structure of formula J, H, H(complex), H(a), H(a)(complex), H(b) and H(b)(complex) and compounds 100-106. In some embodiments, the first compound further comprises a labeling moiety. In some embodiments, the labeling moiety is a fluorescent dye.

In some embodiments, the second compound of the methods according to this invention is as described under the title “The second Compound (Y-ODN-2)” hereinbelow. In some embodiments, the second compound is represented by the structure of formula K and compounds 200-214, 220, 230, 240 and 250. In some embodiments, the second compound further comprises a second labeling moiety. In some embodiments, the second labeling moiety comprises a fluorescent dye. In some embodiments, the second compound comprises at least two labeling moieties. In some embodiments, the second compound comprises at least two fluorescent dyes. In some embodiments, the second compound comprises a first hanging strand. In some embodiments, the DNA construct further comprises a third compound comprising a DNA duplex (dsDNA) appended with a second hanging strand complementary to said first hanging strand, and further appended with at least two fluorescent dyes.

In some embodiments, the first linker comprises at least one oligoethyleneglycol (OEG) moiety, at least one phosphate moiety, at least one thioalkyl moiety or any combination thereof. In some embodiments, the first linker comprise two; three; four; five; six; seven or eight sequentially arranged oligoethylene glycol (OEG) spacers; each represents a separate embodiment according to this invention.

In some embodiments, the protein binder is a small molecule ligand. In some embodiments, the protein binder is a peptide, polypeptide a protein, or a part thereof, each is a separate embodiment. In some embodiments, the protein binder is a biotin or derivative thereof. In some embodiments, the protein binder is a folate or derivative thereof. In some embodiments, the protein binder is an anisamide or derivative thereof. In some embodiments, the protein binder is a glutamate urea or derivative thereof.

### Methods for Treating a Disease

In some embodiments, the recombinant cells disclosed herein comprise a therapeutic effect and are delivered to a patient in need thereof. When used therapeutically, the recombinant cells are referred to herein as “therapeutics”. Methods of administration of therapeutics include, but are not limited to, intravenal, intradermal, intraperitoneal, or surgical routes. The therapeutics of the disclosure presented herein may be administered by any convenient route, for example by infusion, by bolus injection, by surgical implantation and may be administered together with other biologically-active agents. Administration can be systemic or local. It may also be desirable to administer the therapeutic locally to the area in need of treatment; this may be achieved by, for example, and not by way of limitation, local infusion during surgery, by injection, by means of a catheter, or by means of an implant.

A skilled artisan would appreciate that a therapeutically effective amount of the cells may encompass total the amount of cells that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the a therapeutically effective amount refers to that ingredient alone. When applied to a combination, the term refers to combined amounts of the active ingredients that result in the therapeutic effect, whether administered in combination, serially or simultaneously.

In some embodiments, suitable dosage ranges of the therapeutics of the disclosure presented herein are generally between 1 million and 2 million recombinant cells. In some embodiments, suitable doses are between 2 million and 5 million recombinant cells. In some embodiments, suitable doses are between 5 million and 10 million recombinant cells. In some embodiments, suitable doses are between 10 million and 25 million recombinant cells. In some embodiments, suitable doses are between 25 million and 50 million recombinant cells. In some embodiments, suitable doses are between 50 million and 100 million recombinant cells. In some embodiments, suitable doses are between 100 million and 200 million recombinant cells. In some embodiments, suitable doses are between 200 million and 300 million recombinant cells. In some embodiments, suitable doses are between 300 million and 400 million recombinant cells. In some embodiments, suitable doses are between 400 million and 500 million recombinant cells. In some embodiments, suitable doses are between 500 million and 600 million recombinant cells. In some embodiments, suitable doses are between 600 million and 700 million recombinant cells. In some embodiments, suitable doses are between 700 million and 800 million recombinant cells. In some embodiments, suitable doses are between 800 million and 900 million recombinant cells. In some embodiments, suitable doses are between 900 million and 1 billion recombinant cells. In some embodiments, suitable doses are between 1 billion and 2 billion recombinant cells. In some embodiments, suitable doses are between 2 billion and 3 billion recombinant cells. In some embodiments, suitable doses are between 3 billion and 4 billion recombinant cells. In some embodiments, suitable doses are between 4 billion and 5 billion recombinant cells.

One skilled in the art would appreciate that effective doses may be extrapolated from dose-response curves derived from in vitro or animal model test systems.

In some embodiments, recombinant cells are decorated in vitro before delivering to a patient. In some embodiments, recombinant cells are decorated in vivo. In some embodiments, cells are decorated in vivo by first delivering to a patient the recombinant cells, then delivering a first compound that binds the extracellular binding domain of the cells, and then delivering a second compound that binds the first compound. In some embodiments, recombinant cells can proliferate after being delivered to a patient.

The herein-described recombinant cells, either decorated or not, can be incorporated into pharmaceutical compositions suitable for administration. Such compositions typically comprise a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition disclosed here is formulated to be compatible with its intended route of administration. Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, polyalcohols such as mannitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, the recombinant cells are prepared with carriers that will protect them against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers.

In some embodiments, this invention is directed to a kit comprising:
a. a recombinant cell ectopically expressing a polypeptide according to this invention, wherein said polypeptide comprises a membranal anchoring domain and an extracellular binding domain, said extracellular binding domain bound to
b. a first compound according to this invention, comprising a first oligonucleotide (ODN-1) covalently bound to a binder according to this invention, either directly or through a first linker, said binder comprises affinity to said extracellular binding domain,
c. a second compound according to this invention, comprising a second oligonucleotide (ODN-2) covalently bound to a synthetic agent, either directly or through a second linker, wherein said second oligonucleotide is complementary to said first oligonucleotide.

In some embodiments, this invention is directed to a kit comprising:
a. a recombinant cell ectopically expressing a polypeptide according to this invention, wherein said polypeptide comprises a membranal anchoring domain and an extracellular binding domain, said extracellular binding domain bound to
b. a first compound comprising a first oligonucleotide (ODN-1) covalently bound to a His-tag specific binder, either directly or through a first linker, said His-tag specific binder comprises affinity to said extracellular binding domain,
c. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and
d. a third compound comprising a DNA duplex (dsDNA) appended with a second hanging strand complementary to said first hanging strand, and further appended with at least two fluorescent dyes.

### Additional Embodiments of this Invention

In some embodiments, this invention is further directed to a DNA construct comprising:
a. a first compound comprising a first oligonucleotide (ODN-1) covalently bound to a His-tag specific binder, either directly or through a first linker;
b. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a hanging strand appended with at least two fluorescent dyes.

In some embodiments, the first compound of the DNA construct according to this invention is as described under the title “The first Compound (X-ODN-1)” hereinabove. In some embodiments, the first compound is represented by the structure of the nickel complexes of compounds 103-105 as described hereinabove.

In some embodiments, the second compound of the DNA construct according to this invention is as described under the title “The second Compound (Y-ODN-2)” hereinabove.

In some embodiments, the first oligonucleotide (ODN-1) of the DNA construct according to this invention is as described under the title “ODN-1 (or ODN1)” hereinabove.

In some embodiments, the second oligonucleotide (ODN-2) of the DNA construct according to this invention is as described under the title “ODN-2 (or ODN2)” hereinabove.

In some embodiments, the third oligonucleotide (ODN-3) of the DNA construct according to this invention is as described under the title “ODN-3 (or ODN3)” hereinabove.

In some embodiments, the hanging strand of the DNA construct according to this invention is as described under the title “hanging strand” hereinbelow.

In some embodiments, the His-tag specific binder of the DNA construct according to this invention is as described for His-tag binder under the title “Binder (Y1)” hereinabove. In some embodiments, the His-tag specific binder comprises a moiety represented by the structure of formula E, E(complex), E(a), E(a)(complex), E(b), E(b)(complex) as described hereinabove.

In some embodiments, the synthetic agent of said second compound is as described under the title “synthetic agent” hereinabove, and/or as described hereinabove for DNA construct according to this invention.

In some embodiments, the first linker (L₁) and/or a second linker (L₂) are as described hereinabove under the title(s) “A First Linker (L₁)” and/or “The second linker (L₂)”. In some embodiments, the first linker comprises at least one oligoethyleneglycol (OEG) moiety, at least one phosphate moiety, at least one thioalkyl moiety or any combination thereof, each represents a separate embodiment according to this invention. In some embodiments, the first linker comprises two; three; four; five; six; seven or eight sequentially arranged oligoethylene glycol (OEG) spacers; each represents a separate embodiment according to this invention. In some embodiments, the first linker comprises the following monomer:

—[(CH₂O)ₖ—PO₃H]ₗ—
wherein
   k and l are each independently an integer number between 0 and 10; and
   w is an integer number between 1 and 10.

In some embodiments, the first linker comprises one; two; three; four; five; six; seven; eight; nine; or ten monomeric units; each represents a separate embodiment according to this invention. In some embodiments, the first linker comprises two monomeric units. In some embodiments, the first linker comprises five monomeric units.

In some embodiments, the first linker is represented by the following formula:

—[(CH₂O)ₖ—PO₃H]r(CH₂)_{w}—S—
wherein
   k and l are each independently an integer number between 0 and 10; and
   w is an integer number between 1 and 10.

In some embodiments, the construct further comprises a third oligonucleotide (ODN-3) that is complementary to said hanging strand. In some embodiments, the His-tag specific binder is capable of binding to an affinity tag comprising a poly-histidine peptide. In some embodiments, ODN-1 is 5-100 bases long. In some embodiments, the ODN-1 is 5-25 bases long. In some embodiments, the hanging strand is 5-50 bases long. In some embodiments, the hanging strand is 15-35 bases long. In some embodiments, the hanging strand is 26 bases long. In some embodiments, the hanging strand comprises at least 4 fluorescent dyes. In some embodiments, the hanging strand comprises at least 5 fluorescent dyes. In some embodiments, the hanging strand comprises at least 6 fluorescent dyes. In some embodiments, the fluorescent dyes of said hanging strand are located 4-6 bases apart from each other. In some embodiments, the synthetic agent of said second compound is bound to the 3′ end or to the 5′ end of said second oligonucleotide. In some embodiments, the synthetic agent of said second compound is a chemical or a biological moiety. In some embodiments, the synthetic agent of said second compound is naturally occurring compound. In some embodiments, the synthetic agent of said second compound is a synthetic compound. In some embodiments, the synthetic agent of said second compound comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, an antibody, a small molecule, a drug, or a derivative thereof, each represents a separate embodiment according to this invention. In some embodiments, the synthetic agent of said second compound comprises a CSP binder, a cancer cell binder, or a protein binder. In some embodiments, the CSP binder, a cancer cell binder, or a protein binder comprises a biotin, a folate, an anisamide, or a glutamate urea. In some embodiments, the synthetic agent of said second compound can interact with a specific CSP on a cancer cell. In some embodiments, the CSP of said CSP binder is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof. In some embodiments, the protein binder comprises an antibody, a biotin, a folate, an anisamide, a glutamate urea or derivative thereof, each represents a separate embodiment according to this invention. In some embodiments, the cancer cell binder comprises an antibody, a biotin, a folate, an anisamide, a glutamate urea or derivative thereof, each represents a separate embodiment according to this invention. In some embodiments, the cancer cell of said cancer cell binder, is KB cell (cervical cancer cell), MDA-MB-435 (melanoma cell), or LNCaP (prostate cancer cell); each represents a separate embodiment according to this invention.

In some embodiments, the fluorescent dyes are selected from a group comprising dansyl, fluorescein (6-FAM), FAM, cyanine dyes (e.g. Cy3, Cy5, Cy7, etc), sulfoindocyanine, nile red, Rhodamine dyes (e.g., Rhodamine 123, Rhodamine Red-X, etc.), perylene, fluorenyl, coumarin, 7-methoxycoumarin (Mca), dabcyl, NBD, Nile blue, TAMRA, BODIPY dyes, FITC (Fluorescein isothiocyanate), Thiazole orange, Quinoline blue, Thiazole red, phycoerythrin (PE), Acridine Orange, Alexa Fluor dyes (e.g., Alexa Fluor 488, Alexa Fluor 555, Alexa Fluor 647, etc.), Cascade Blue, DAPI (4′,6-diamidino-2-phenylindole), DiI (1,1′-Dioctadecyl-3,3,3′,3′-tetramethylindocarbocyanine perchlorate), Ethidium Bromide, GFP (Green Fluorescent Protein), Hoechst dyes (e.g., Hoechst 33342, Hoechst 33258, etc.), Indo-1, Lucifer Yellow, MitoTracker dyes (e.g., MitoTracker Green, MitoTracker Red, etc.), Oregon Green, Propidium Iodide, SYBR Green, Texas Red, YOYO-1, ZsGreen or derivative thereof, each represents a separate embodiment according to this invention.

In some embodiments, this invention is further directed to a system comprising:
a. a recombinant cell ectopically expressing a polypeptide, wherein said polypeptide comprises a membranal anchoring domain and an extracellular binding domain;
b. the DNA construct as described hereinabove;
   wherein the His-tag specific binder of said DNA construct, comprises affinity to said extracellular binding domain of said polypeptide; and
   wherein the DNA construct is bound to said recombinant cell in the presence of Ni²⁺ ions.

In some embodiments, the system does not perturb said cell's function. In some embodiments, the system can be reversibly modified. In some embodiments, the recombinant cell is a bacteria. In some embodiments, the polypeptide is a cell surface protein (CSP) comprising a histidine tag (e.g., His-OmpC). In some embodiments, the membranal anchoring domain of said polypeptide comprises a transmembranal protein or a part of it, an artificial polypeptide, or a combination thereof, each represents a separate embodiment according to this invention. In some embodiments, the extracellular binding domain of said polypeptide comprises a poly-histidine tag. In some embodiments, the transmembranal protein comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the bacteria is a His-OmpC expressing bacteria.

In some embodiments, this invention is further directed to a recombinant cell bound to the DNA construct of this invention, as described hereinabove. In some embodiments, the recombinant cell is ectopically expressing a polypeptide, said polypeptide comprises a membranal anchoring domain and an extracellular binding domain, said extracellular binding domain comprises a poly-histidine affinity tag, which is bound to said DNA construct in the presence ofNi²⁺ ions. In some embodiments, the polypeptide comprises an outer membrane protein C (OmpC); receptor tyrosine kinases (RTKs); Ion channel linked receptors; Enzyme-linked receptors; G protein-coupled receptors or any combination thereof, each represents a separate embodiment according to this invention. In some embodiments, the polypeptide is a cell surface protein (CSP) comprising a histidine tag. In some embodiments, the cell surface protein (CSP) comprising a histidine tag is a His-OmpC. In some embodiments, the cell is a bacteria. In some embodiments, the bacteria is a His-OmpC expressing bacteria. In some embodiments, the membranal anchoring domain of said polypeptide comprises a transmembranal protein or a part of it, an artificial polypeptide, or a combination thereof, each represents a separate embodiment according to this invention.

In some embodiments, this invention is further directed to a method for binding a first cell to a second cell, said method comprises incubating the recombinant cell as described hereinabove (a first cell) with a second cell, wherein the second cell comprises a CSP, and said synthetic agent of said DNA construct of said first cell, comprises binding affinity to said CSP of said second cell.

In some embodiments, the first cell is a native cell, a living cell or an engineered cell; each represents a separate embodiment according to this invention. In some embodiments, the first cell is a bacteria. In some embodiments, the second cell is a cancer cell. In some embodiments, the CSP is selectively expressed in said second cell. In some embodiments, the CSP is overexpressed in said second cell. In some embodiments, the CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof; each represents a separate embodiment according to this invention. In some embodiments, the synthetic agent of the DNA construct of the first cell, comprises a CSP binder, a protein binder, a cancer cell binder or derivative thereof; each represents a separate embodiment according to this invention. In some embodiments, the method is taking place in a cellular environment. In some embodiments, the protein binder, CSP binder or a cancer cell binder comprises an antibody, a biotin, a folate, an anisamide, a glutamate urea or a derivative thereof, each represents a separate embodiment according to this invention. In some embodiments, the cancer cell is KB cell (cervical cancer cell), MDA-MB-435 (melanoma cell), or LNCaP (prostate cancer cell); each represents a separate embodiment according to this invention. In some embodiments, the interaction between the first cell and the second cell is multivalent.

In some embodiments, this invention is further directed to a method for binding a cell to a protein of interest (POI), said method comprises incubating a sample comprising a POI with the recombinant cell as described hereinabove, wherein the synthetic agent of the DNA construct is a protein binder, which comprises binding affinity to said POI.

In some embodiments, the method is taking place in a cellular environment. In some embodiments, the POI is a cell surface protein (CSP). In some embodiments, the protein binder is selective to said POI. In some embodiments, the protein binder is a cell surface protein (CSP) binder, a small molecule ligand, an antibody, a peptide, a polypeptide, a protein or a part thereof, each represents a separate embodiment according to this invention. In some embodiments, the CSP is a polypeptide or a protein, which is overexpressed on the surface of a cell. In some embodiments, the CSP is a polypeptide or a protein, which is selectively expressed on the surface of a cell. In some embodiments, the CSP is a polypeptide or a protein, which is overexpressed on the surface of a cancer cell. In some embodiments, the CSP is a polypeptide or a protein, which is selectively overexpressed on the surface of a cancer cell.

In some embodiments, this invention is further directed to a method for detecting and/or labeling a protein of interest (POI) in a cellular environment, said method comprises a. imaging a sample comprising a protein of interest (POI) in cellular environment;
b. incubating the sample of (a) with the recombinant cell of this invention as described hereinabove, wherein said synthetic agent of said DNA construct of said cell is a protein binder, which comprises affinity to said POI;
c. optionally washing the sample of (b) from excess of said cell; and
d. imaging the fluorescence of said sample;
   wherein increase in the fluorescence signal is indicative of the presence of said POI in said cellular environment, thereby detecting and/or labeling said protein of interest (POI) in said cellular environment.

In some embodiments, the cellular environment comprises living cells. In some embodiments, the POI is a cell surface protein (CSP). In some embodiments, the protein binder is selective to said POI. In some embodiments, the synthetic agent is a protein binder. In some embodiments, the fluorescence signal is measured by a fluorescence microscope or by recording the emission with a spectrophotometer at a particular wavelength. In some embodiments, the CSP is a polypeptide or a protein, which is selectively expressed on the surface of a cell. In some embodiments, the CSP is a polypeptide or a protein, which is selectively expressed on the surface of a cancer cell. In some embodiments, the CSP is a polypeptide or a protein, which is overexpressed on the surface of a cell. In some embodiments, the CSP is a polypeptide or a protein, which is overexpressed on the surface of a cancer cell. In some embodiments, the CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof, each represents a separate embodiment according to this invention. In some embodiments, the protein binder is selective to said POI.

In some embodiments, this invention is further directed to a method for measuring the interaction between a protein of interest (POI) and a potential ligand, said method comprises: a. imaging a sample comprising a protein of interest (POI);
b. incubating the sample comprising a POI with the recombinant cell of this invention as described hereinabove and with a potential ligand, wherein said synthetic agent of said DNA construct of said cell is a protein binder, which comprises affinity to said POI;
c. optionally washing the sample of (b) from excess of said cell and said potential ligand;
d. measuring the fluorescence imaging of said sample;
e. comparing the measured fluorescence of the sample of (d) with the fluorescence measured from incubating a sample comprising a protein of interest (POI) with the recombinant cell of this invention, followed by washing excess of said cell (i.e. a control);
wherein reduction in the fluorescence signal with respect to the control is indicative of the interaction between said POI and said potential ligand, thereby measuring the interaction between a protein of interest (POI) and a potential ligand for said POI.

In some embodiments, the POI is a cell surface rotein (CSP). In some embodiments, the synthetic agent is a protein binder. In some embodiments, the protein binder is selective to said POI. In some embodiments, the potential ligand is a protein binder, a peptide, small molecule, modulator, agonist, antagonist, or any combination thereof, each represents a separate embodiment according to this invention. In some embodiments, the potential ligand is added after, before or concurrently with said cell in step (b); each represents a separate embodiment according to this invention. In some embodiments, the fluorescence signal is measured by a fluorescence microscope or by recording the emission with a spectrophotometer at a particular wavelength; each represents a separate embodiment according to this invention.

In some embodiments, this invention is further directed to a method for cell-based screening for potential ligands for a protein of interest (POI), said method comprises:
a. imaging a sample comprising a protein of interest (POI);
b. incubating the sample comprising a POI with a recombinant cell of this invention as described hereinabove, and with a potential ligand, wherein said synthetic agent of said DNA construct of said cell is a protein binder, which comprises affinity to said POI;
c. optionally washing the sample from excess of said cell and said potential ligand;
d. measuring the fluorescence imaging of said sample;
e. comparing the measured fluorescence of the sample of (d) with the fluorescence measured from incubating a control sample comprising a protein of interest (POI) with a cell of this invention, followed by washing excess of said cell (i.e. a control);
   wherein reduction in the fluorescence signal with respect to the control is indicative of the interaction between said POI and said potential ligand, thereby screening for potential ligands for said POI.

In some embodiments, cell-based screening is performed in living cells. In some embodiments, the synthetic agent is a protein binder, a drug, or a small molecule; each represents a separate embodiment according to this invention. In some embodiments, the protein binder, drug or small molecule is selective to said POI; each represents a separate embodiment according to this invention. In some embodiments, the potential ligand is a protein binder, a peptide, small molecule, modulator, agonist or antagonist; each represents a separate embodiment according to this invention. In some embodiments, the potential ligand is added after, before or concurrently with said cell in step (b); each represents a separate embodiment according to this invention. In some embodiments, the fluorescence signal is measured by a fluorescence microscope or by recording the emission with a spectrophotometer at a particular wavelength; each represents a separate embodiment according to this invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The following examples are presented in order to more fully illustrate the preferred embodiments of the invention. They should in no way be construed, however, as limiting the broad scope of the invention.

### Examples

### Example 1—Materials and Methods

All reagents and solvents were obtained from commercial suppliers. Dry solvents were purchased from Sigma Aldrich. Deuterated solvents were purchased from Cambridge Isotope Laboratories, Inc. (Andover, MA). Aluminium-backed silica plates (Merck silica gel 60 F254) were used for thin layer chromatography (TLC) to monitor solution-phase reactions. The ¹H NMR and ¹³C NMR spectra were recorded on a Bruker Advance 300, 400 or 500 MHz spectrometer. The chemical shifts are represented in ppm on the δ scale down field from TMS as the internal standard. The following abbreviations were used to describe the peaks: br-broad, s-singlet, d-doublet, t-triplet, td-triplet of doublets, q-quartet, quin-quintet and m-multiplet. Oligodeoxynucleotides (ODNs) were obtained from W. M. Keck Foundation Biotechnology at Yale University. The mass spectrum was recorded by Waters SYNAPT-XS Q-TOF High Resolution mass spectrometer (Manchester, UK) with an electrospray ionization (ESI) interface in the negative ion mode within a mass range from 400 to 5000 m/z. Matrix-assisted laser desorption ionization time-of-flight (MALDI-TOF) mass spectrometry was performed on an AB SCIEX 5800 system, equipped with an Nd: YAG (355 nm) laser with a 1 KHz pulse (Applied Biosystems), at the Weizmann Institute of Science mass spectrometry facility. For small molecules, the analytical reversed phase high-performance liquid chromatography (RP-HPLC) analysis was performed on an Agilent Technologies 1260 Infinity quaternary pump LC system, equipped with a diode-array detector using a C₁₈ column. Preparative HPLC was carried out using an Agilent 218 purification system, equipped with an autosampler, a UV-Vis dual wavelength detector, and a 440-LC fraction collector operating under OpenLab ChemStation software. The purification of oligodeoxynucleotides was carried out on a Waters 2695 separation module HPLC system with a 2994 photodiode array detector using either a Waters XBridge™ OST C₁₈ column (2.5 μM, 4.6 mm×50 mm) or an XBridge™ OST C18 column (2.5 μM, 10 mm×50 mm). Oligodeoxynucleotide samples were desalted using illustra MicroSpin G-25 Columns (GE Healthcare) according to the supplier's instructions. Concentrations of the oligodeoxynucleotides were quantified based on their respective electronic absorption at 260 nm and the molar extinction coefficient of the oligodeoxynucleotide at this wavelength. For gel electrophoresis, 6×DNA loading dye (Thermo Scientific) and PCR-25 bp or PCR-20 bp ladder (Sigma Aldrich) were used. MCherry-CaaX HRAS, was a kind gift from Professor Rob Parton, The University Of Queensland, Australia (Addgene plasmid #108886; http://n2t.net/addgene:108886). KB cell lines were obtained from Prof. Ronit Satchi-Fainaro's group (Tel Aviv University, Israel) while LNCaP and MDA-MB-435 were obtained from Stem Cell Core Facility and Advanced Cell Technologies, Life Sciences Core Facilities, Weizmann |Institute of Science. These cell lines were screened negative for mycoplasma using a PCR-based assay (EZ-PCR mycoplasma detection kit, Biological Industries). Nude female mice were procured from Veterinary Resources, Weizmann Institute of Science. Fluorophore conjugated antibodies were purchased from Bio Legend and Santa Cruz Biotechnology. Cell images were acquired using an Olympus IX51 fluorescent microscope equipped with a U-MNIBA3 fluorescence filter cube (excitation and emission filters of 470-495 nm, and 510-550 nm, respectively), a U-MNG2 fluorescence filter cube narrow-band (excitation and emission filters of 530-550 nm, and 590 nm, respectively) and a U-MF2 fluorescence filter cube (excitation and emission filters of 620-660 nm, and 700-775 nm, respectively). Tissue samples were imaged with Leica LAS X inverted microscope. Fluorescence was measured using a BioTek synergy H4 hybrid multiwall plate reader, in black flat-bottom polystyrene NBS 384-well microplates (Coming).

Abbreviations. Acetonitrile (ACN), N,N′-Dicyclohexylcarbodiimide (DCC), Dichloromethane (DCM), N,N′-Diisopropylethylamine (DIPEA), 1,2-Dimethylethylenediamine (DMEDA), N,N′-Dimethylformamide (DMF), Dimethyl sulfoxide (DMSO), 1-Ethyl-3-(3-dimethylaminopropyl) carbodiimide (EDC), 1-[Bis(dimethylamino)methylene]-1H-1,2,3-triazolo[4,5-b]pyridinium3-oxide hexafluorophosphate (HATU), Formalin-fixed paraffin-embedded (FFPE), Nitrilotriacetic acid (NTA), Paraformaldehyde (PFA), Polyacrylamide gel electrophoresis (PAGE), Phosphate buffer saline (PBS), Reverse phase high-performance liquid chromatography (RP-HPLC), Sodium dodecyl sulfate (SDS), 2-(1H-6-chlorobenzotriazole-1-yl)-1,1,3,3-tetramethyluroniumtetrafluoroborate (TCTU), Trifluorocetic acid (TFA), Thin layer chromatograpy (TLC).

Synthetic procedures

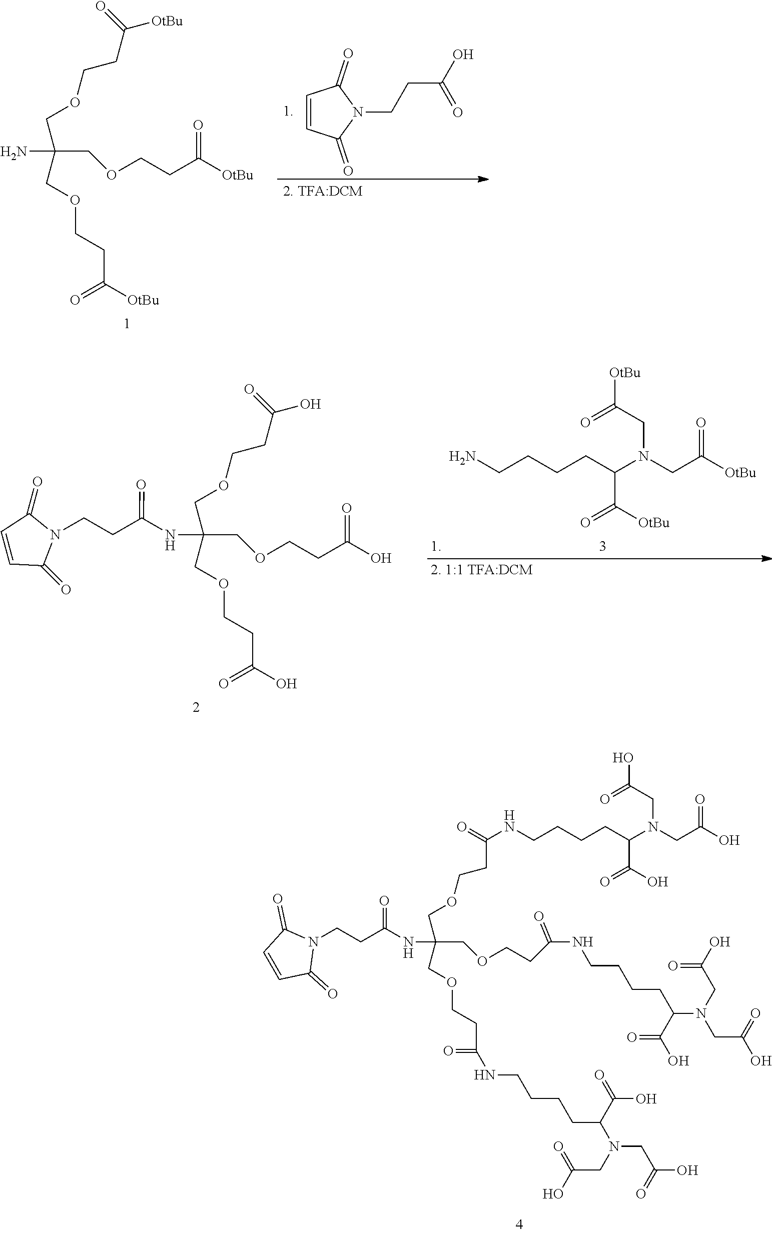 [SMILES: CC(C)(C)OC(=O)CCOCC(N)(COCCC(=O)OC(C)(C)C)COCCC(=O)OC(C)(C)C ; O=C(O)CCN1C(=O)C=CC1=O ; O=C(O)CCOCC(COCCC(=O)O)(COCCC(=O)O)NC(=O)CCN1C(=O)C=CC1=O ; CC(C)(C)OC(=O)CN(CC(=O)OC(C)(C)C)C(CCCCN)C(=O)OC(C)(C)C ; O=C(O)CN(CC(=O)O)C(CCCCNC(=O)CCOCC(COCCC(=O)NCCCCC(C(=O)O)N(CC(=O)O)CC(=O)O)(COCCC(=O)NCCCCC(C(=O)O)N(CC(=O)O)CC(=O)O)NC(=O)CCN1C(=O)C=CC1=O)C(=O)O]

Compounds 1 and 3 were synthesized according to previously reported procedures (Cardona, C.M. An improved synthesis of a trifurcated newkome-type monomer and orthogonally protected two-generation dendrons. J. Org. Chem. 67, 1411-1413 (2002); Huang, Z. Facile synthesis of multivalent nitrilotriacetic acid (nta) and nta conjugates for analytical and drug delivery applications. Bioconjugate Chem. 17, 1592-1600 (2006).

Compound 2: Compound 1 (600 mg, 1.18 mmol) was dissolved in dry DCM (30 ml) under argon and cooled to 0° C. Then, EDC (339 mg, 1.7 mmol) and DIPEA (413.7 μl, 2.32 mmol) were added and the reaction mixture was stirred for 30 min at room temperature. 3-Maleimidopropionic acid (240.1 mg, 1.4 mmol) was added, and the solution was stirred overnight. Then 40 ml DCM was added, and the solution was washed with water (10 ml), and brine (10 ml). The organic layer was dried with Na₂SO₄, filtered, and concentrated under high vacuum. Finally, the crude product was purified by column chromatography (DCM/MeOH, 97:3) to yield a yellow oil (501.6 mg, 64%). ¹H NMR (CDCl₃, 300 MHz): δ 1.44 (s, 27H); 2.44 (t, J=6 Hz, 6H); 2.51 (t, J=6 Hz, 2H); 3.63 (t, J=6 Hz, 6H); 3.67 (s, 6 H); 3.80 (t, J=6 Hz, 6H); 6.69 (s, 2H). ESI-MS (m/z): calcd. for (M+H): 657.35, found 657.44; calcd. for (M+Na): 679.35, found 679.31. The tert-butyl groups were then deprotected using a 1:1 (v/v) mixture of TFA: DCM for 2.5 h. After removing the solvents, the excess of TFA was co-evaporated 4 times with DCM and then the product was dried under high vacuum. ¹H NMR (D₂O, 300 MHz): δ 2.47 (t, J=6 Hz, 2H); 2.59 (t, J=6 Hz, 6H); 3.61 (s, 6H); 3.67-3.75 (m, 8H); 6.83 (s, 2H). ESI-MS (m/z): calcd. for (M+H): 489.16, found 489.18; calcd. for (M+Na): 511.16, found 511.12; calcd. for (2M+H): 977.32, found 977.03; calcd. for (2M+Na): 999.32, found 999.15 (2M+Na).

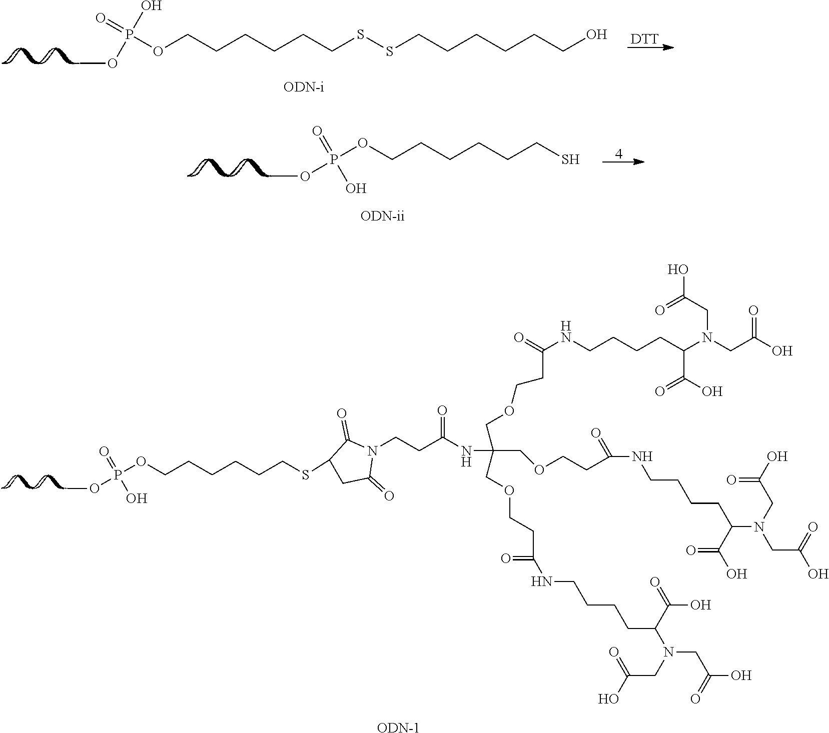

Compound 4: A solution of compound 2 (160 mg, 304.8 μmol) in dry DCM (10 ml) was cooled to 0° C. in an ice bath and DIPEA (212 μl, 1.2 mmol), EDC (191 mg, 1 mmol), and HOBt (41 mg, 304.8 μmol) were added consecutively. After 15 min, compound 3 (433 mg, 1 mmol) was added and the reaction was stirred overnight. Then DCM (40 ml) was added and the solution was washed with water (10 ml). The organic layer was dried with Na₂SO₄, filtered, and concentrated at high vacuum. Finally, the crude product was purified by column chromatography (DCM/MeOH, 96:4) to yield a colorless oil (96.6 mg, 18.3%). ¹H NMR (MeOD, 300 MHz): δ 1.50 (s, 54H); 1.55 (s, 27H); 1.71 (m, 18H); 2.42 (t, J=6 Hz, 6H); 2.49 (m, 2H); 3.20 (t, J=6 Hz, 6H); 3.31 (m, 12H); 3.55-3.74 (m, 17H); 6.84 (s, 2H). ESI-MS (m/z): calcd. for (M+Na): 1749.13, found 1748.72; calcd. for (M+2Na): 886.06, found 886.27; calcd. for (M+3Na): 598.37, found 598.52. The tert-butyl groups were then deprotected using a 1:1 (v/v) mixture of TFA: DCM for 2.5 h. After removing the solvents, the excess of TFA was co-evaporated 4 times with DCM and then the product was dried under high vacuum ¹H NMR (MeOD, 300 MHz): δ 1.47 (m, 6H); 1.53 (m, 6H); 1.91 (m, 6H); 2.43 (m, 8H); 3.17 (m, 6H); 3.58-3.65 (m, 15H); 4.1 (m, 14H); 6.82 (s, 2H). ESI-MS (m/z): calcd. for (M+H): 1221.48, found 1221.53; calcd. for (M+Na): 1243.48, found 1243.39. HRMS.

### General Procedure for the Synthesis of the ODN-1 Strands

ODN-i (200 nmol) was treated with 400 μl of a DTT solution (50 mM DTT in 50 mM Tris buffer, pH 8.3) for 1 hour. The reduced oligonucleotide (ODN-ii) was then desalted on Sephadex™ G-25 and dried under reduced pressure. ODN-ii was added to a solution of 4 (8 mg) in concentrated PBS×10, pH 7. The reaction was stirred overnight. The product was purified using RP-HPLC. MALDI-TOF MS (m/z): X-ODN-1: calcd. 6319.6, found 6334.2; ODN-1: calcd. 8876.1, found 8893.3; Compound 101: calcd. 11453.6, found 11454.3; Compound 103: calcd. 9139.8, found 9139.2; Compound 104: calcd. 9119.9, found 9115.9.

Compounds 100-106 where synthesized according to the general synthesis described hereinabove.

### Synthesis of Folate-ODN-2 (Compound 206)

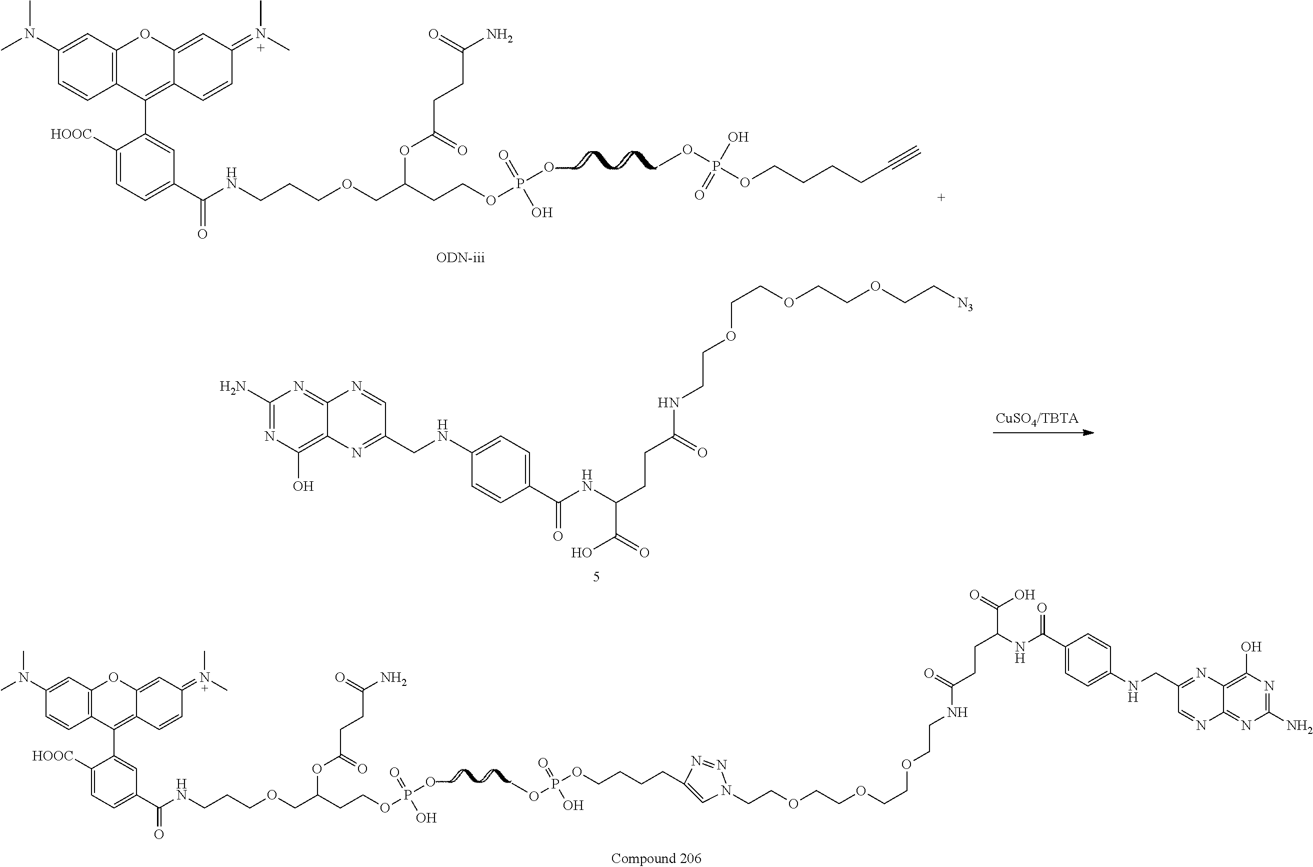

Folate azide 5 was prepared according to a previously published procedure.³ ODN-iii (150 nmol) was dissolved in 160 μl MQ water, followed by the addition of compound 5 (1.5 μmol), ascorbic acid (20 μl, 0.9 μmol), TEAA buffer (40 μl, 2 M, pH=7), and DMSO (200 μL). After degassing with argon, Cu-TBTA (80 μL, 0.9 μmol) was added, and the mixture was stirred for 12 h. The product was purified using RP-HPLC to afford Compound 206. MALDI-TOF MS (m/z): calcd. 9940, found 9941.

### OmpC Construction and Expression

OmpC construction. E. coli outer membrane protein C (OmpC) was isolated by PCR, amplified from E. coli ASKA library and cloned into pET21 using RF cloning OmpC_FpET21: TTTGTTTAACTTTAAGAAGGAGATATACATATGAAAGTTAAAGTACTGTCCCTC (SEQ ID NO.: 11) and OmpC_RpET21: TTCCTTTCGGGCTTTGTTAGCAGCCGGATCTTAGAACTGGT AAACCAGACCC (SEQ ID NO.: 12). The resulting plasmid was a His-tag less construct. Polyhistidine-linker sequences were inserted in the predicted 7^{th} loop of the OmpC. OmpC-(6His)₁ contains 11 amino acid (Aa) sequence: SAGHBHHIHGT (SEQ ID NO.: 13) was constructed by Inverse PCR using the following 2 primers: OmpC_His1 F: CATCATCACCATGGTACCTCTAAAGGTAAAAACCTGGGTCGTGGCTAC (SEQ ID NO.: 14), and OmpC_His1R: ATGGTGATGATGATGATGACCCGCGGAGGTACCATGGTGATGATG GTGATGACCCGCGGA (SEQ ID NO.: 15). The resulting plasmid served as a template for introducing a second His-linker to obtain OmpC-(6His)₂ 22 Aa sequence: SAGHHHHHHGTSAGHHIHHHHGT (SEQ ID NO.: 16) by using the following 2 primers: OmpC_His2FInverse: CACCATC ACGGTACCTCTAAAGGTAAAAACCTGGGTCGTG (SEQ ID NO.: 17) and OmpC_His2RInverse: GTGATGGTGACCCGCGGAGGTACCATGGTGATGATGGTGATG (SEQ ID NO.: 18). An additional third His-linker was introduced to OmpC-(6His)₂ by using the following 2 primers: OmpC_His3FInverse: CATCATCATGGTACCTCTAAAGGTAAAAACCTGGGTCGTG (SEQ ID NO.: 19) and OmpC_His3RInverse: ATGATGATGACCCGCGGAGGTACCGTGAT GGTGGTGATGGTG (SEQ ID NO.: 20). The resulting construct OmpC-(6His)₃ contains 33 Aa His-linker: SAGHHHHHHGTSAGHHHHHHGTSAGHHHHHHGT (SEQ ID NO.: 21) in the same position at the predicted 7^{th} loop of OmpC. For the Inverse PCR cloning reactions one primer of each set of primers had to be phosphorylated.

Purification of OmpC. The expression of OmpC was tested in the whole cell extracts (WCE) and in the membrane fraction. Cultures expressing OmpC, and His-OmpC were harvested, resuspended in Na₂HPO₄ (10 mM, pH 7.3) and lyzed by sonication. A sample from each culture was analyzed by SDS-PAGE for the expression of OmpC in the WCE. Following sonication, the supernatant was separated by centrifugation at 13800 g for 10 min. The membrane fraction was recovered by centrifugation of the supernatant at 13800 g for 30 min., resuspended in 10 mM Na₂HPO₄, pH 7.3, 2% Triton X-100 and incubated at 37° C. for 30 min. The insoluble fraction was recovered by centrifugation at 13800 g for 30 min., washed and resuspended in 10 mM Na₂HPO4 pH 7.3. Proteins from the membrane fractions were analyzed by SDS-PAGE.

### Oligonucleotides

The oligonucleotides used in the experiments are detailed in Table 1.

TABLE 1
Oligonucleotides (ODNs)

| | | SEQ |
|---|---|---|
| | | ID |
| Description | Sequence (5′->3′) | NO. |
| Compound 100 | GCGGCGAGGCAGC | 1 |
| ODNia | | |
| Compound 106 | | |
| duplex 5 | | |
| duplex 6 | | |
| Compound 101 | GTCACGTCATAGCTGCCTGATCCTA | 2 |
| ODN-1 | TCATAGCTGCCTGATCCTA | 3 |
| ODN-i | | |
| Compound 102 | | |
| Compound 105 | | |
| Compound 103 | GGTACAACTAGACGATCGACAGTAG | 4 |
| Compound 104 | CGCAACGAAAAAAAAAAAAGCGCGC | 5 |
| ODN₂ | TAGGATCAGGCAGCTATGACGTGAC | 6 |
| Compound 200 | | |
| Compound 201 | | |
| Compound 202 | | |
| Compound 205 | | |
| Compound 206 | | |
| Compound 207 | | |
| Compound 209 | | |
| Compound 211 | | |
| Compound 213 | | |
| Compound 220 | | |
| Compound 230 | | |
| Compound 240 | | |
| Compound 208 | TAGGATCAGGCAGCTATGACGTGACTTAGAACAAT | 7 |
| Compound 203 | CTACTGTCGATCGTCTAGTTGTACC | 8 |
| Compound 204 | GCGCGCTTTTTTTTTTTTCGTTGCG | 9 |
| ODN-3 | GTCACGTCATAGCTGCCTGATCCTA | 10 |
| ODN₄ | ATTGTTCTAAGTCACGCGCNTCCANCAGTNCCA | 22 |
| ssDNA-long | GNGTGCNCTCGN | |
| FAM₆-ODN_{2b} | N = Fluorescein dT | |
| Construct 1 | | |
| B-probe 4 | | |
| ssDNA-long′ | CGCNTCCANCAGTNCCAGNGTGCNCTCGNATTGT | 23 |
| Construct 2 | TCTAAGTCACG | |
| B-probe 5 | N = Fluorescein dT | |
| ODN₃ | ACGAGAGCACACTGGAACTGATGGAAGCG | 24 |
| ssDNA-short | | |
| ODN-vi | | |
| duplex 6 | | |
| ODN-v | GCTGCCTCGCCGCNTCCANCAGTNCCAGNGTGCN | 25 |
| FAM₆-ODN₂ | CTCGN | |
| (Compound | N = Fluorescein dT | |
| 250) | | |
| duplex 5 | | |
| duplex 6 | | |

### Bacterial Strains and Growth Conditions

E. coli K-12 strain KRX (Promega) was used for protein expression. Transformed bacteria with the different OmpC constructs (OmpC or His-OmpC) were cultured to saturation in LB medium supplemented with 100 μg/ml of ampicillin at 30° C. 40 μl of the pre-cultured cells were then diluted into 4 ml of fresh LB medium supplemented with ampicillin, and incubated until the OD₆₀₀ reaches 0.6. Protein expression was then induced by the addition of 0.1% Rhamnose and 20 μM isopropyl-b-D-1-thiogalactopyranoside (IPTG) and cultures were allowed to grow at 30° C. for 18 h. Then, the bacterial cells were harvested by centrifugation at 6,000 g for 4 min.

### General Procedure for Decorating Bacteria with the Oligonucleotides

The bacterial cells (OmpC or His-OmpC) were collected by centrifugation at 6000 g for 4 min. The pellet was washed twice with PBS x 1 buffer and resuspended in the same buffer to an OD₆₀₀ of 0.3. To a 100 μl sample of the bacteria suspension, a preincubated sample of DNA (500 nM) and NiCl₂ (2. μM) was added, and the cells were incubated at room temperature for 1 h. Then the bacterial sample were washed twice with PBS, resuspended in 100 μl PBS and placed on a glass-bottom dish (P35G-1.5-14-C; MatTek) precoated with poly−1-lysine (Sigma Aldrich) and left to adhere for 1 h. Finally, the wells were washed vigorously with PBS three times and imaged using an Olympus IX51 fluorescent microscope. The samples were imaged using 60× or 100× objective lenses.

### Treatment of the Modified Bacteria with EDTA

Bacterial samples decorated with Compound 100 were incubated with various concentration of EDTA (0, 5, 10 mM) for 1 h. Cells were then collected (6,000 g, 4 min) and washed twice with 200 μl PBS buffer. Cells were resuspended in 100 μl PBS buffer and added to poly−1-lysine-coated slides for imaging.

### Flow Cytometry

Bacteria were decorated with Compound 101 according to the procedure described above. The samples were analyzed using BD FACS Aria Fusion instrument (BD Biosciences, San Jose, CA, USA) equipped with 488 nm (blue), 561 nm (green), and 640 nm (red) lasers. Sorting was performed using a 100-μm nozzle equipped with BD FACS Diva software v8.0.1 (BD Biosciences). Data was analyzed using FlowJo software.

### Bacterial Cell Growth

His-OmpC bacteria decorated with Compound 101 was incubated for 30 min in M9 minimal medium containing 2% glucose. The sample was spun down at 6,000 g for 2 min and the supernatant was discarded. After washing the pellet with M9 minimal medium, the cells were diluted to OD₆₀₀=0.05 in M9 medium in a 96-well plate. Growth kinetics was monitored by recording OD₆₀₀ under shaking at 30° C. for 24 h. Bacteria expressing His-OmpC was used as a control. The ability of the modified His-tagged bacteria to grow and divide was also demonstrated using fluorescence microscopy. For these experiments, the bacteria were prepared using a similar procedure. After diluting the sample to OD₆₀₀=0.3, it was allowed to grow at 30° C. 100 μl samples were withdrawn at different time intervals and plated on poly−1-lysine-coated glass bottom dishes and imaged by fluorescent microscopy.

### Introducing Posttranslational Modifications' to the Bacteria

Bacterial cells were decorated with ODN-1 according to the procedure described above. After washing the sample with PBS, the following ODNs were added sequentially: Compound 200, ODN-3, Compound 201, ODN-3, Compound 202, and ODN-3. After each incubation step, cells were washed twice with PBS and a sample was taken for imaging before the addition of the subsequent strand. Fluorescently labeled ODN-2 strands were added at a concentration of 500 nM and incubated for 30 min, while ODN-3 strand was added at a concentration of 2 μM and incubated for 2 h.

### Mixed Population of Bacteria

Three samples of His-OmpC bacteria (100 μl each) were separately labeled with Compound 102, Compound 103, or Compound 104. Each sample was washed twice with PBS. Then, an equal ratio (30 μl each) of the three samples were combined and Compound 202, Compound 203 and Compound 204 (500 nM) were added to the mixture and incubated for 10 min. The bacterial cells were centrifuged at 6,000 g for 2 min, washed twice with PBS and imaged by fluorescent microscopy using 488, 561, and 647 nm excitation lasers and 488/50, 610/60, and 685/50 emission filters. For flow cytometry analysis, the samples were not washed after addition of ODN-2 strands.

### Bacteria-Streptavidin Interaction

His-tagged bacterial cells were decorated with a duplex consisting of ODN−1 and Compound 205 duplex according to a similar procedure described above. For binding with streptavidin, cells were incubated with Alexa-647 streptavidin conjugate (500 nM) in PBS x 1 for 1 h, and after washing twice with PBS were imaged by fluorescent microscopy. The fluorescent signal was abolished when bacterial cells were treated with ODN-3 (3 μM) for 1 h. The control experiment was performed similarly using bacteria decorated with a duplex containing ODN-1 and the complementary strand.

### Bacteria-KB Cell Interaction

KB cells were maintained in folate-depleted RPMI supplemented with 10% fetal bovine serum (FBS), 1% L-glutamine, and 1% penicillin/streptomycin. Cells (12,500 cells/well) were seeded onto glass bottom culture dishes (Mattek) and allowed to adhere overnight. Cells were then washed twice with PBS and incubated with 100 μl His-tagged bacteria decorated with ODN-1: Compound 206 duplex for 30 min. The medium was removed and cells were rinsed three times with PBS. Cells were then imaged using a fluorescence microscope and a 60×objective lens. A control experiment was performed similarly using bacteria decorated with a duplex lacking the folate moiety (ODN-1 and ODN-iii). To show the reversibility of interaction, the bacteria bound KB cells were incubated with ODN-3 (5 μM) for 15 min. After washing twice with PBS buffer, cells were imaged again.

### Adhesion to the Solid Support

The gold substrates were prepared by electron-beam evaporation of an adhesion layer of chromium (3 nm), followed by a 20 nm layer of gold (99.99% purity) onto high precision cover glasses (170±5 m, Marienfeld-Superior, Germany). A solution of (11-mercaptoundecyl)tetra(ethylene glycol)⁹ (2 mM in ethanol) were added to the gold coated substrates and incubated for 2 h. After removing the solution, the slides were washed four times with ethanol. Bacteria samples decorated with a duplex consisting of Compound 102 and Compound 207 were washed twice with PBS, resuspended in 100 μl phosphate buffer (pH=3.8), and then incubated on gold surfaces for 15 min. The solution containing bacteria was removed, and the slides were rinsed three times with PBS, and twice with water. Finally, they were imaged using an Olympus IX51 microscope.

### Super-Resolution Microscopy

Super-resolution images were collected on a Vutara SR200 STORM (Bruker) microscope based on the single-molecule localization biplane technology. His-tagged bacteria was decorated with ODN-1:Compound 201 duplex according to the procedure described in above. The bacteria were imaged using 647 nm excitation laser and 405 nm activation laser in an imaging buffer composed of 5 mM cysteamine, oxygen scavengers (7 μM glucose oxidase and 56 nM catalase) in 50 mM Tris, 10 mM NaCl and 10% glucose at pH 8.0. Images were recorded using a 60×NA 1.2 water immersion objective (Olympus) and Evolve 512 EMCCD camera (Photometrics) with gain set at 50, frame rate at 50 Hz, and maximal power of 647 and 405 nm lasers set at 6 and 0.05 kW/cm², respectively. Total number of frames acquired was 8000. Data was analyzed by the Vutara SRX software.

### Example 2—Design Principles of a Dynamic Artificial Receptor System

Objective: To produce an artificial receptor fulfilling the following requirements: (1) the artificial receptor is easily modifiable by molecular signals in their environment, (2) the artificial receptor is capable of attaching different bioactive molecules, labeling molecules, and synthetic agents, (3) the artificial receptor does not perturb desirable cell functions, (4) the artificial receptor can be reversibly modified.

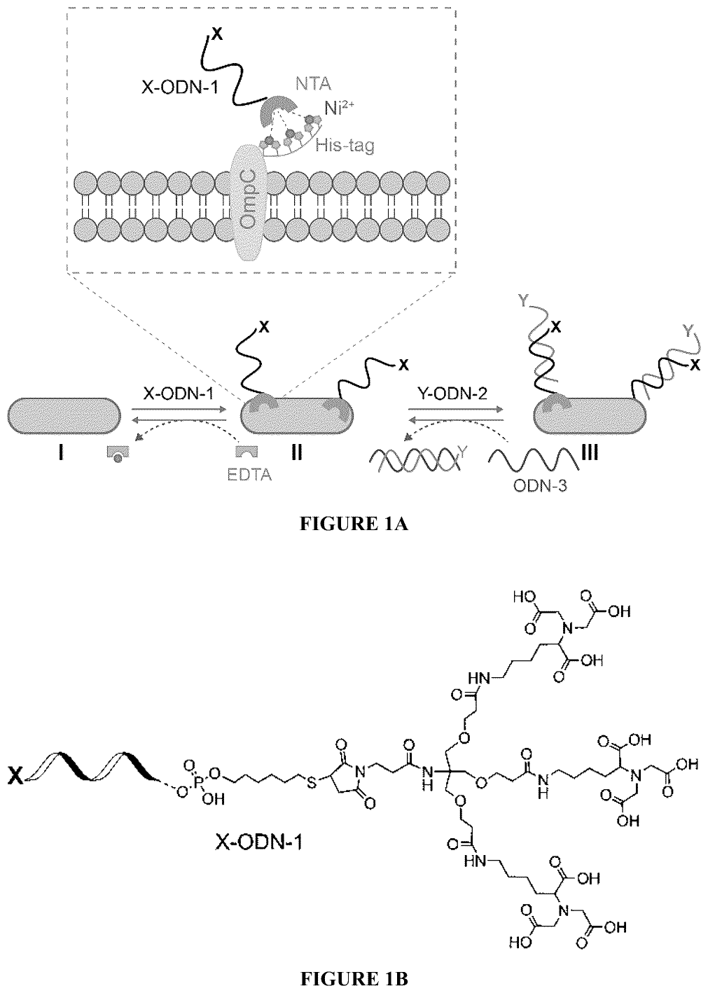

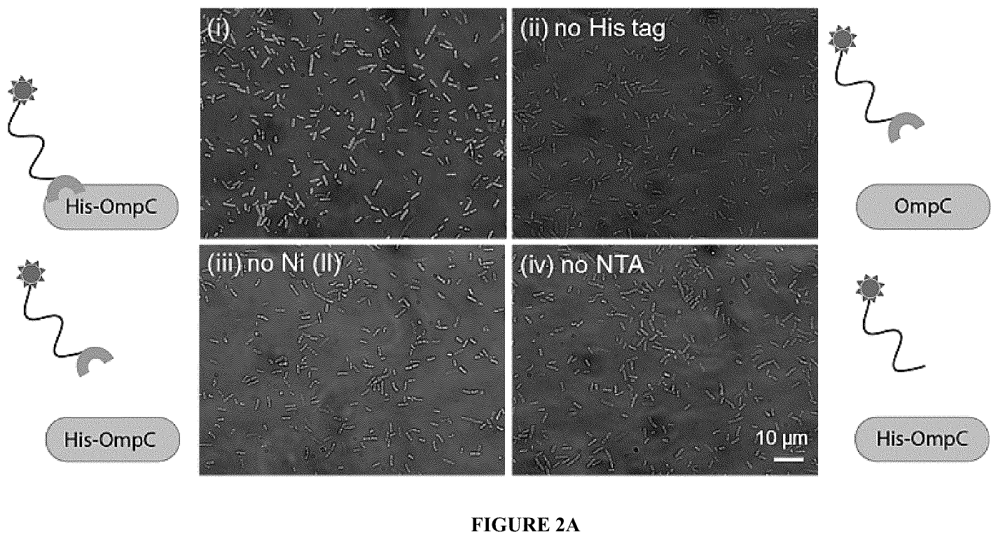

FIG. 1A shows the design and operation principles of an embodiment of the synthetic receptor system presented herein. The system comprises: A first polypeptide, said polypeptide comprising a membranal anchoring domain and an extracellular binding domain. In the examples shown herein, the membranal anchoring domain used is outer membrane protein C (OmpC) and the extracellular binding domain is hexa-histidine tag (His-tag). The first compound is sometimes termed His-OmpC in the Examples.

The first compound, comprises a first oligonucleotide (ODN-1) bound to a binder, said binder comprises affinity to said extracellular binding domain. The first compound is sometimes termed X-ODN-1 in the Examples, wherein ODN-1 denotes the first oligonucleotide, and X denotes an optional labeling moiety. In the examples shown herein, the binder is a three nitrilo acetic acid (Tri-NTA) conjugate, which binds His-tag. FIG. 1B shows an embodiment of X-ODN-1.

The second compound comprises a second oligonucleotide (ODN-2) bound to a synthetic agent on its end. The second compound is sometimes termed Y-ODN-2 in the Examples, wherein ODN-2 denotes the second oligonucleotide, and Y denotes the synthetic agent on its end. The oligonucleotide ODN-2 is complementary to the first oligonucleotide ODN-1. However, Y-ODN-2 bears also a short overhang region, termed a toe-hold region. Such toe-hold region can be used to initiate strand displacement and detachment of Y-ODN-2 from X-ODN-1 by an oligonucleotide complementary to the whole ODN-2 oligonucleotide.

The system optionally comprises a third compound, comprising a third oligonucleotide (ODN-3). The oligonucleotide ODN-3 is complementary to the whole ODN-2 sequence, i.e., both to the toe-hold region and to the region bound to ODN-1. Cells can be optionally incubated with ODN-3, which produces strand displacement. In a first step, ODN-3 binds to Y-ODN-2 toe hold region. In a second step, ODN-3 competes with ODN-1 for binding with ODN-2, until eventually it detaches Y-ODN-2 from X-ODN-1.

The artificial receptor system described above was used for decorating a cell surface according to at least two approaches. In the first approach, cells expressing His-OmpC were incubated with X-ODN-1 in the presence of Ni (II) (FIG. 1, steps I and II). X-ODN-1 was efficiently bound to His-OmpC in such conditions. The effect of the synthetic agent was terminated by detaching X-ODN-1 from His-OmpC, for example by incubating the cells with a Ni (II) chelator as EDTA.

In the second approach, cells expressing His-OmpC were first incubated with X-ODN-1 in the presence of Ni (II) (FIG. 1, steps I and II). Then, cells were incubated with Y-ODN-2, which bound to X-ODN-1 (FIG. 1, steps III). Optionally, addition of ODN-3 terminated the effect of the synthetic agent of Y-ODN-2 (FIG. 1, steps III and II).

The artificial receptor system developed and disclosed herein present a number of advantages. First, the receptors are non-covalently anchored to the cellular membrane. Such non-covalent anchoring allows controlling the number of receptors on the cell membrane and surface by external molecular signals (e.g., X-ODN-1, EDTA, Y-ODN-2, and ODN-3). Second, the anchoring domain of the receptors is stably inserted into the cell membrane, and an extracellular domain can bind different synthetic agents. Thus, different synthetic agents can be bound to the extracellular domain without re-engineering the cells. Third, the anchoring domain has a minimal size and is present only at specific locations on the bacteria membrane. Thus, the anchoring domain does not perturb cellular function. Fourth, the synthetic receptors can be to reversible modified. This allows dynamically altering their structure while they are attached to the bacterial membrane, resembling post-translational modifications that occur on natural receptors.

### Example 3—Decorating Bacteria with Artificial Receptors and Controlling the Receptors Functioning

Objective: To decorate bacterial membranes with an artificial receptor.

Methods: His-tagged OmpC was expressed in E. coli, which was then incubated with an X-ODN-1 appended either with a Cy5 dye or TAMRA (Compounds 100-101) in the presence of nickel ions and EDTA. Methods and protocols are detailed in Example 1.

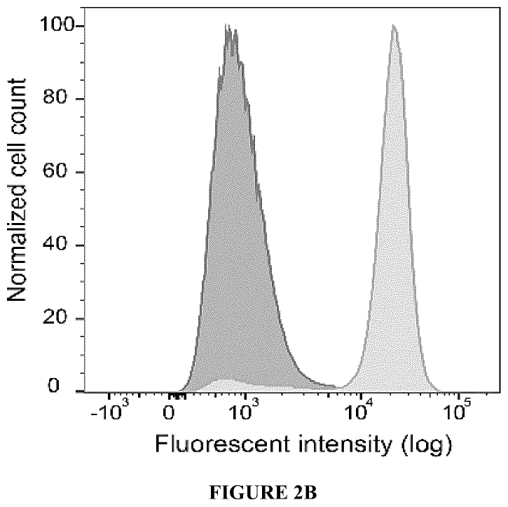

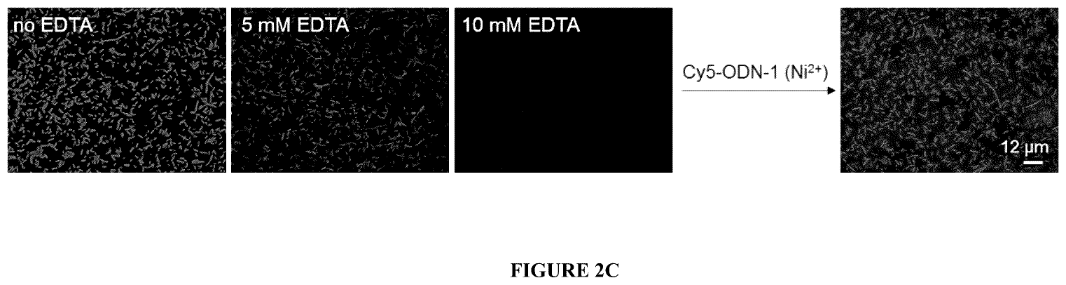

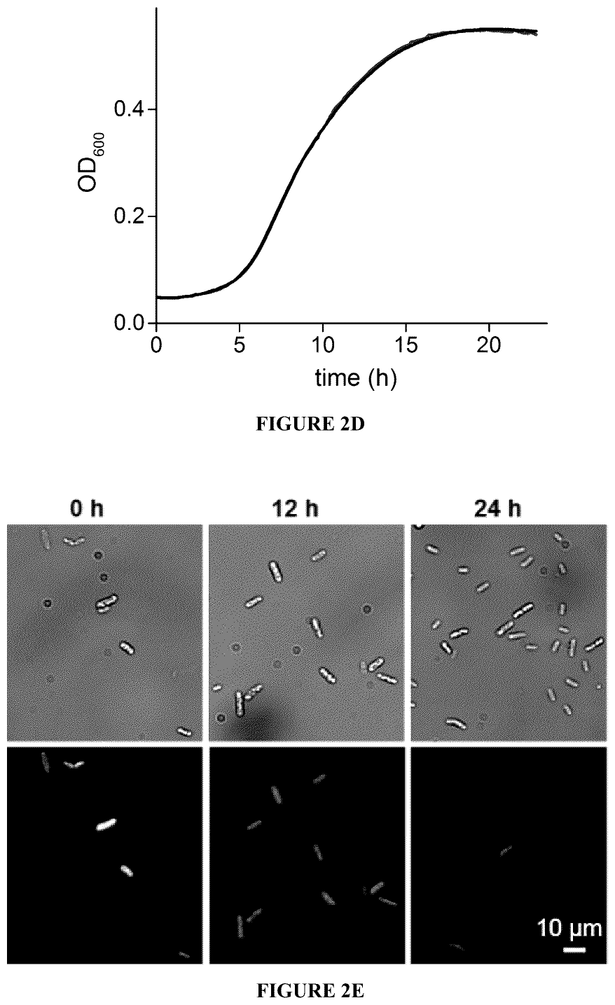

Results: Fluorescence imaging revealed that His-tagged OmpC engineered bacteria incubated with Compound 100 were successfully decorated with the Cy5 fluorophore (FIG. 2A, i). To confirm that the labeling did not result from a non-specific interaction between Compound 100 and the bacteria surface, Compound 100 was also incubated with native bacteria lacking His-OmpC (FIG. 2A, ii), as well as with the His-tagged bacteria in the absence of nickel ions (FIG. 2A, iii). Additionally, His-tagged bacteria was incubated with a Cy5-labeled ODN lacking a tri-NTA group (FIG. 2A, iv). No fluorescence was observed in any of these controls, confirming the selectivity of ODN-1 to membrane bound His-tags.

The selectivity and degree of labeling were further analyzed by flow cytometry. 90.9% of His-tagged modified bacteria and 1% of native bacteria were labeled by Cy5 (FIG. 2B).

The ability of the system to control the activity levels of the artificial receptors by external signals was further tested. Bacteria were exposed to increased concentrations of EDTA, which resulted in a decrease in surface coverage with Compound 100. 10 mM of EDTA completely removed Compound 100 from the cell surface. Detached Compound 100 could be washed from the medium and bacteria could be re-decorated with other molecules (FIG. 2C).

To confirm that attachment of X-ODN-1 does not affect the ability of the bacteria to grow and divide, the growth of TAMRA-ODN-1 (Compound 101) decorated bacteria was measured by optical density (OD) and compared to that of bare His-tagged bacteria. The growth kinetic curves were not affected by Compound 101 binding (FIG. 2D) indicating that the biomimetic cellular surface protein system does not affect cell division and survival.

The ability of Compound 101 decorated bacteria to grow and divide was further demonstrated using fluorescence microscopy. Fluorescence microscopy revealed that the number of Compound 101 labeled cells increased with time, but that the fluorescence recorded in each cell decreased (FIG. 2E). These results were interpreted as a consequence of the Compound 101 molecules being divided between the daughter cells in each division.

### Example 4—Reversible Modification of Membrane-Bound Synthetic Receptors Using Complementary Strands

Objective: To reversibly modify the synthetic receptors by external molecules.

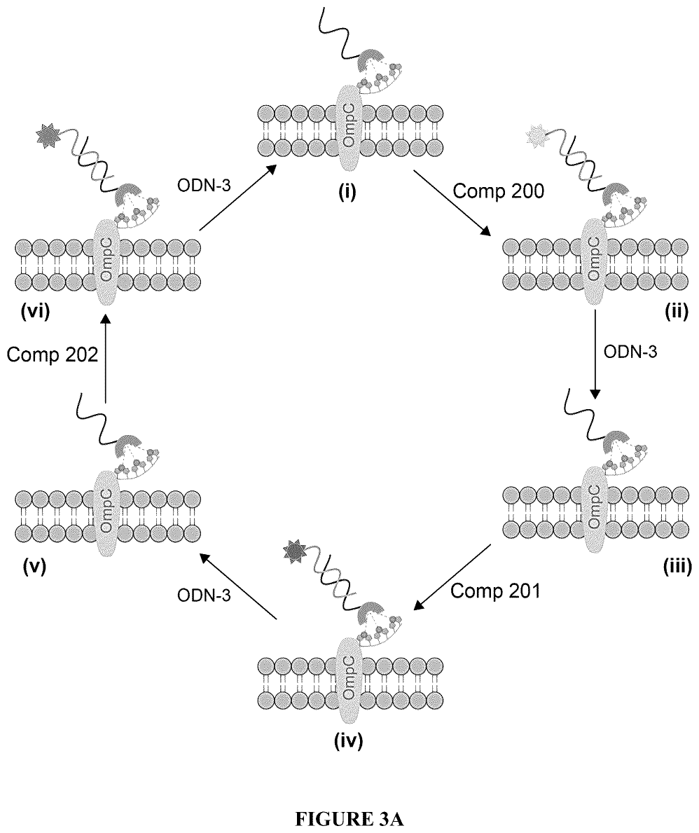

Methods: FIG. 3A schematically illustrates the experiments detailed herein. E. Co/i ectopically expressing His-OmpC were first incubated with oligonucleotide X-ODN-1 (FIG. 3A, step (i)). Afterwards cells were incubated with a Compound 200, wherein ODN-2 is an oligonucleotide complementary to ODN-1 (FIG. 3A, step (ii)). Cells were then incubated with an ODN-3 oligonucleotide complementary to ODN-2 (FIG. 3A, step (iii)). Then cells were incubated with a Compound 201 (FIG. 3A, step (iv)). Next, cells were again incubated with an ODN-3 oligonucleotide (FIG. 3A, step (v)). Cells were finally incubated with a Compound 202 (FIG. 3A, step (vi)). Fluorescence was measured in all steps assessing the binding of TAMRA, Cy5, and FAM to the cell membranes.

Results: Fluorescence microscopy revealed the presence of the corresponding dye (TAMRA, Cy5, and FAM) after bacteria were incubated with it. Further, the fluorescent emission disappeared after each time bacteria were incubated ODN-3 (FIG. 3B).

### Example 5—Decorating Populations of Heterogenous Bacteria with Different Artificial Receptors

Objective: To create a mixed population of bacteria, where each subpopulation bears a different sequences of ODN-1 and is modified by a different X-ODN-2 molecule.

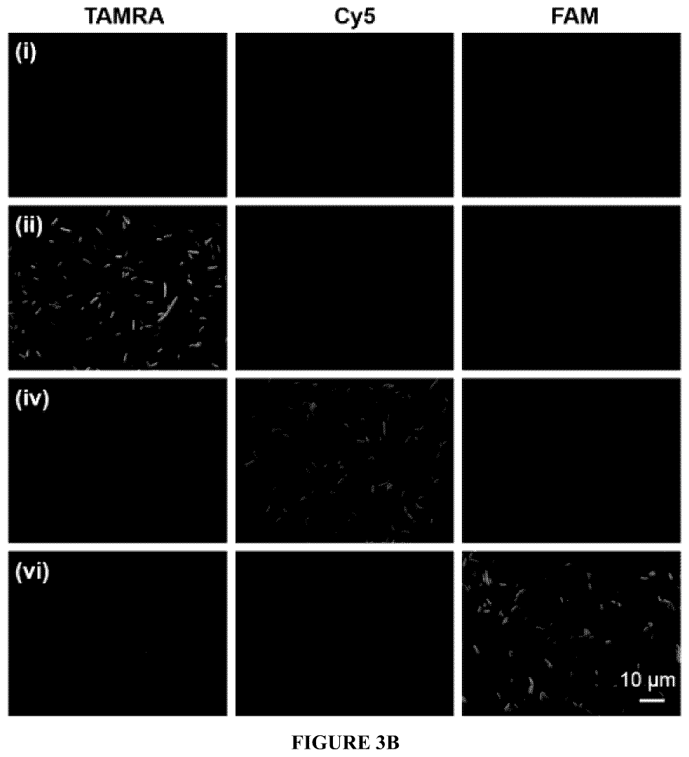

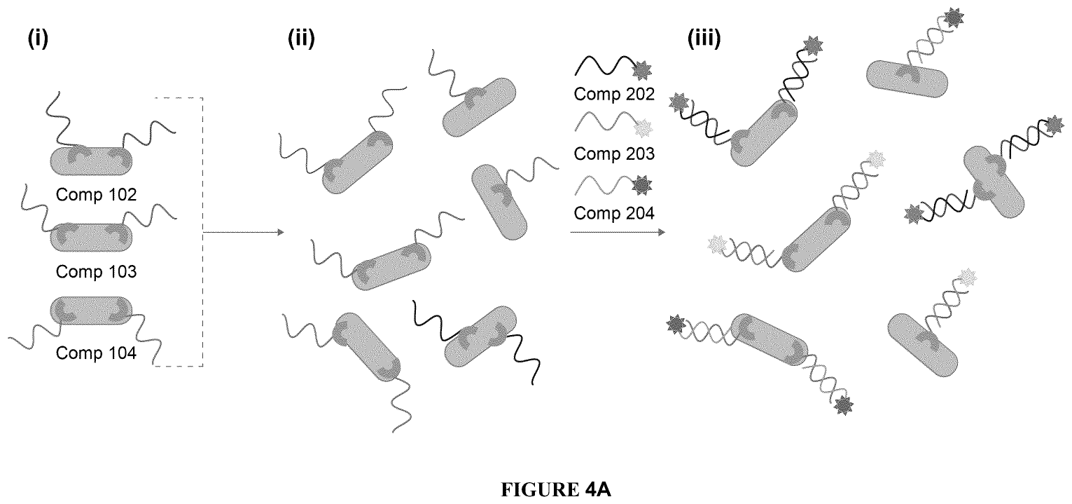

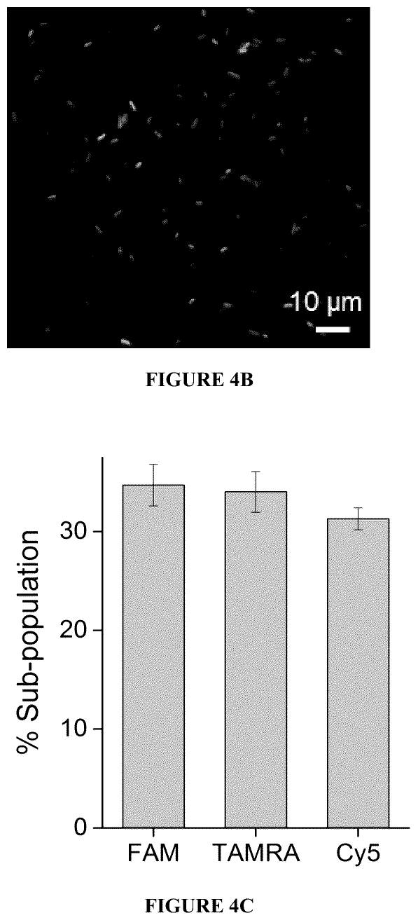

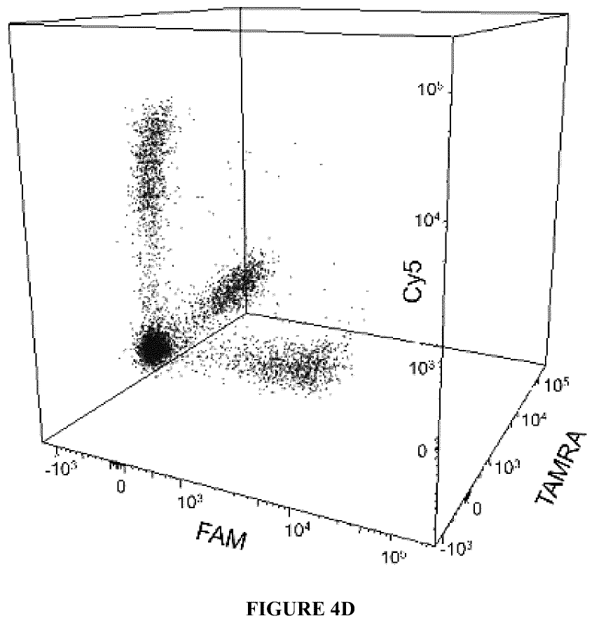

Methods: Three populations of His-tagged E. coli were incubated with three different types of ODN-1 (Compound 102, Compound 103, and Compound 104; Compound 102, 103 and 104 respectively), which bared the same tri-NTA types but differed in their oligonucleotide sequences. Then, the three samples were combined and incubated with a mixture of three types of dye-labeled ODN-2 (Compound 202, Compound 200, and Compound 201 respectively); each of which was complementary to only one of the bacteria-bound ODN-is (FIG. 4A). Bacteria were then analyzed by fluorescent microscopy and FACS.

Results: Fluorescence microscopy (FIG. 4B) and FACS analysis (FIG. 4C) revealed the presence of three distinct groups of bacteria, each labeled with only one dye. Calculating the percentage of each population out of the total number of bacteria revealed a 1:1:1 ratio between the three sub-populations. (FIG. 4D) indicating that there is no strand swap between the three populations and that the sub-population modification occurs with very high selectivity.

Discussion: This experiment demonstrates a means to selectively label His-tagged proteins with different colors. Hence, one practical application that can be achieved with this approach is using the synthetic receptors to image specific proteins or cellular compartments in living cells. The advantage of using this method, over using other fluorescent probes that can bind and label short fusion peptides in living cells is the simplicity by which the fluorescent dye can be changed. Specifically, when DNA duplex-based fluorescent probes are used for live cell imaging there is no need to synthetize a new probe for each application. Instead, various different fluorescent dyes can be used for imaging, simply by preparing a wide range of fluorescently labeled ODN-2s from commercially available phosphoramidites and by using an automated DNA synthesizer.

### Example 6—Endowment of New Properties to Bacteria by Artificial Receptors

Objective: To endow bacteria with unnatural and potentially useful properties by using the artificial receptor system.

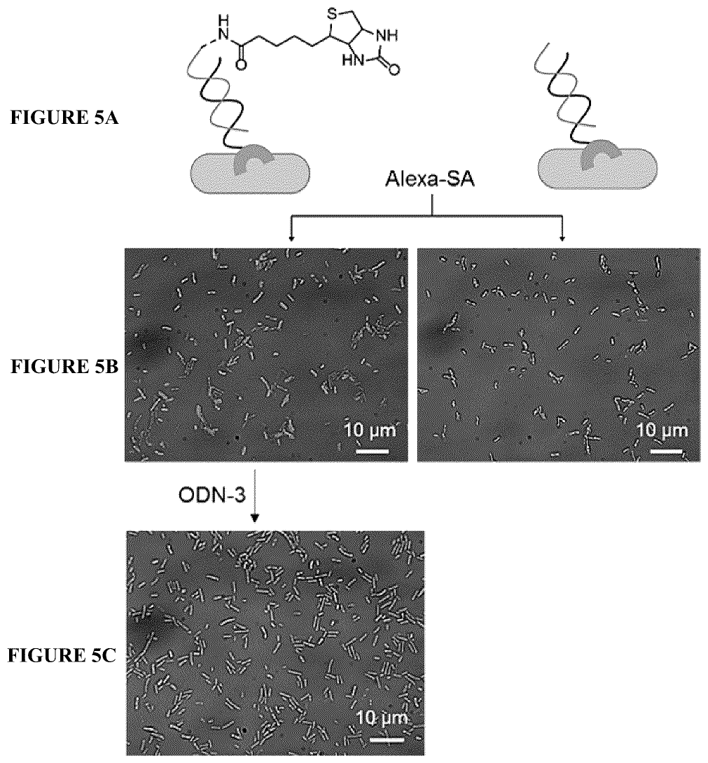

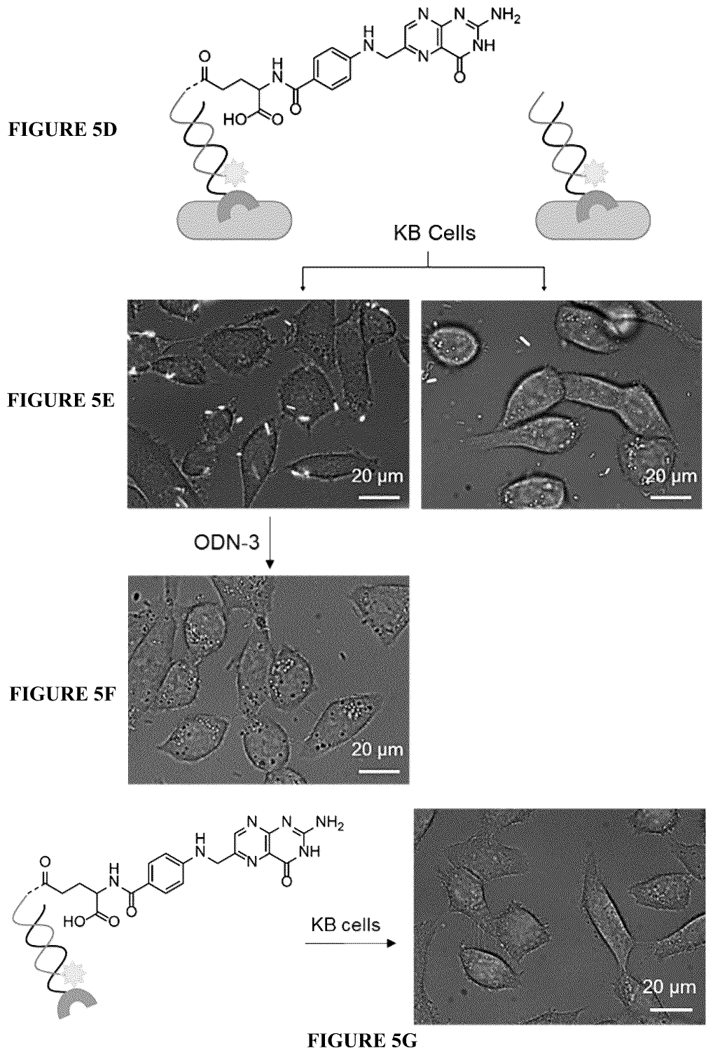

Methods: His-tagged E. coli were incubated with an ODN-1 molecule and afterwards with a biotin-ODN-2 molecule (Compound 205). Then, the cells were incubated with an Alexa 647-modified streptavidin (FIG. 5A). To verify specificity, the same experiment was performed with an ODN-2 molecule lacking biotin (FIG. 5A). Cells were then incubated with ODN-3 to detach ODN-2 from the cell membranes.

Results: Fluorescent microscopy revealed that bacteria became fluorescent only when Compound 205 was incorporated in the synthetic receptor (FIG. 5B), indicating specific binding of the protein to the bacterial membrane. The fluorescent signal disappeared when ODN-3 was added (FIG. 5C), indicating the reversibility of this process, and suggesting the possibility of regulating unnatural cell-protein-interactions using synthetic molecular signals as Compound 205 and ODN-3.

### Example 7—Induction of Unnatural Cell-Cell Interactions by Artificial Receptors

Objective: To test whether synthetic receptor-protein interactions can mediate unnatural cell-cell interactions in general, and interactions resembling bacterial-mammalian cell interactions in particular.

Methods: His-tagged bacteria were decorated with a DNA duplex containing Compound 101 and a folate-modified ODN-2 (compound 206). Then, bacteria were incubated with human epidermoid carcinoma KB cells overexpressing an extracellular folate receptor (FIG. 5D). As a control, KB cells were incubated with bacteria decorated with a similar TAMRA-labeled DNA duplex lacking the folate group (FIG. 5D). Cells were then incubated with ODN-3 to detach compound 206 from bacteria membranes.

Results: Fluorescent imaging revealed KB cells were labeled with glowing bacteria when incubated with compound 206 bound bacteria, but not with control bacteria (FIG. 5E). Incubation with ODN-3 fully detached compound 206 from the bacteria, thus releasing the bacteria from the KB cells (FIG. 5F).

Incubation of KB cells with the DNA duplex alone (without His-tagged bacteria) did not result in fluorescent cancer cell labeling (FIG. 5G). This observation indicates that the bacteria scaffold itself plays a critical role in the interaction of folate with the folate receptor. One contribution of the bacteria to effective cell labeling is an increased avidity, which results from multivalent interactions between natural folate receptors on the KB cell and the folate-modified DNA duplexes on the surface of E. Coli. The second contribution is that each bacterial cell is decorated with multiple fluorophores, leading to a bright fluorescent labeling and consequently, to sensitive detection.

Discussion: These experiments provide evidence that unnatural cell-cell interactions can be both induced and disrupted using a biomimetic receptor system that responds to external molecular signals, such as compound 206 and ODN-3, respectively.

These experiments also demonstrate the relevance of this study to cell-based therapy. Here it is shown the ability to program bacterial cells to target cancer cells with increased avidity and selectively, by using synthetic cell-surface receptors to guide therapeutic cells to their targets. Further, the disruption of bacteria-cancer cell interactions with ODN-3 suggests that this approach can be used as an antidote to this class of therapeutics.

### Example 8—Induction of Bacterial Adhesion to Abiotic Surfaces by Artificial Receptors

Objective: To test whether synthetic receptor can provide bacteria with the ability to interact selectively with solid substrates.

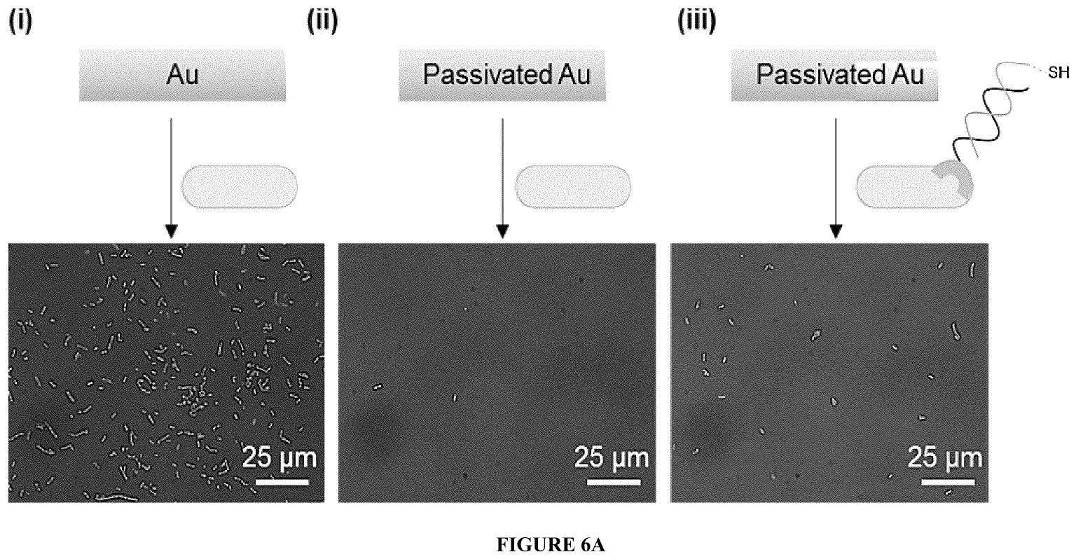

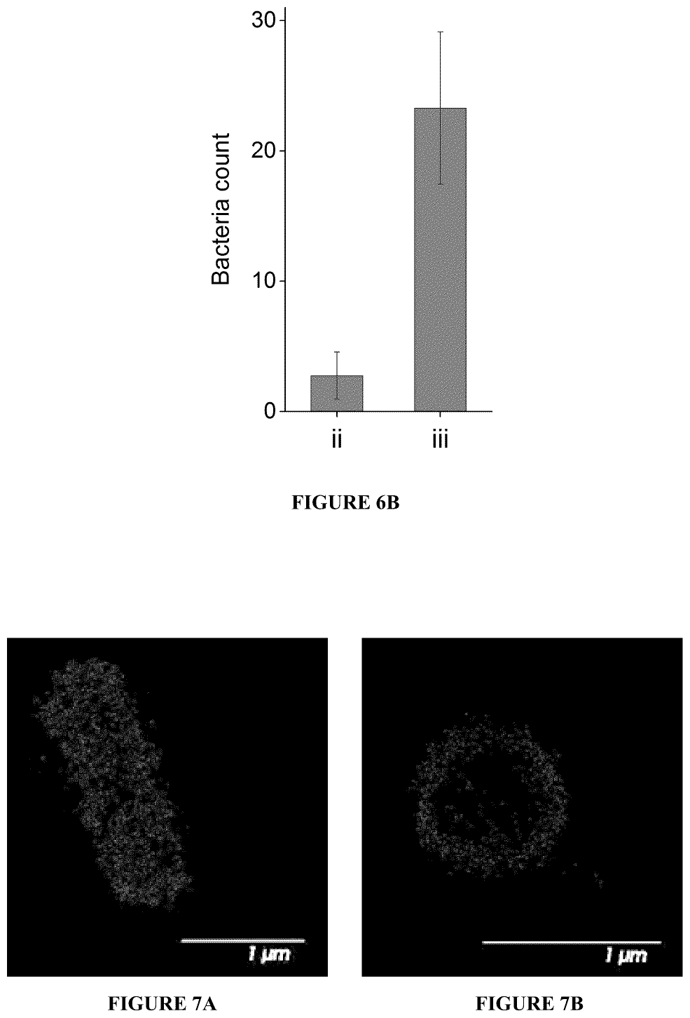

Methods: His-tagged bacteria were decorated with a duplex assembled from ODN-1 and HS—ODN-2 (Compound 207), namely, an ODN-2 that is appended with a thiol group. HS is known to have high affinity to gold. In the following step, unmodified His-tagged bacteria and thiol-modified His-tagged bacteria (FIG. 6A) were incubated with a gold substrate that was previously passivated with (11-mercaptoundecyl)tetra(ethylene glycol) to prevent non-specific bacterial adhesion. Gold surfaces were observed after 15 min incubation. Cells were then incubated with ODN-3 to detach Compound 207 from bacteria membranes.

Results: Microscopy revealed an increase of about 8.5-fold in the attachment of thiol-modified bacteria to the gold substrate compared with the control (FIG. 6B). This indicates that the ODN-1:Compound 207 duplex acts as an unnatural adhesin that can mediate specific binding of bacteria to solid support. The selectivity of these synthetic adhesin to gold was further demonstrated by incubating the thiol-modified bacteria with the gold substrate in the presence of ODN-3, which led to a significant decrease in the number of surface-bound His-tagged bacteria.

Discussion: In the context of biomimicry, disruption of adhesion owing to changes that occur on the synthetic receptors resembles the way post-translational modification of natural adhesins are used by bacteria to disrupt adhesion processes. The unnatural adhesins presented herein can be used to have a precise control of the way bacteria are attached to solid supports. For example, changing the length of the DNA linkers or attaching the modified bacteria to more complex DNA architectures (such as DNA Origami/nanotechnology type structures) on the surface may alter the binding properties of the bacteria. Further, the approach presented herein can be used to generate engineered living materials (ELMs) made of controlled bacterial assemblies.

### Example 9—Induction of Luminescence in Bacteria by Artificial Receptors

Background: Reversible switching of luminescence in response to the binding of cell surface proteins to extracellular molecular signals is a fundamental property of serval bacterial strains. A key principle underlying natural bacterial luminescence processes is the selective interaction between peptide autoinducers (AI) and their protein receptors, which enables them to trigger the emission of specific bacterial strains in complex biological mixtures. According to this invention, the ability to selectively label specific bacteria (modified with a unique ODN-1) in complex mixtures is described.

Objective: To control bacterial cell luminescence using biomimetic receptor systems (using super resolution microscopy).

Methods: Due to the small size of bacteria, super resolution (SR) microscopy was used to visualize E. Co/i's membrane with super resolution (SR). This was achieved by combining ODN-1 with a commercially available ODN-2 (Cy5-ODN-2; Compound 201) bearing a Cy5 dye, which is compatible with stochastic optical reconstruction microscopy (STORM). SR images of individual bacteria revealed that DNA duplex-based label clearly outlines the bacterial cell's borders (FIG. 7A). Imaging of the transverse cut of the bacteria confirms that only the outer membrane of the bacteria is labeled, namely, that the synthetic receptors are exposed on the bacterial surface and are not internalized (FIG. 7B).

### Example 10—Discussion

The Examples disclosed above show a number of unexpected advantages as shown in the following examples: 1) The His-OmpC molecule can be stably expressed in E. coli. 2) The hexa-histidine moiety does not perturb the function of cell or of the synthetic agent due to its small size. 3) The His-tag can be efficiently targeted by NTA-Ni (II) complexes, including complexes of ODN-NTA conjugates. 4) The binding of His-OmpC to X-ODN-1 can be efficiently released by incubating the cells with a Ni (II) chelator, as EDTA. 5) The use of Y-ODN-2 circumvents the complexity of synthesizing the oligonucleotide X-ODN-1 which is attached on one end to the Tri-NTA moiety, and on the other to a synthetic agent. 6) The activity of the synthetic agent of Y-ODN-2 can be effectively terminated by incubating the cells with ODN-3.

The advantages of using ODN-small molecule conjugates as synthetic protein binders include the ability to precisely control the orientation, distances and valency of their binding units, as well as the ability to dynamically change their structure, which provides a means to regulate protein functions in real time. The Examples provided herein show that when synthetic proteins binders of this class are attached to cell's surfaces, their regulatory effect can be extended from the protein level to the cellular level. Specifically, on the cell′ membrane such systems can act as artificial cell surface receptors that can be reversibly modified and hence, can provide the cells with ‘programmable’ properties. In this model system, metal coordination and DNA-hybridization were used to direct the formation of artificial receptors on a short peptide tag fused to an outer membrane protein on the surface of E. coli. Owing to the high selectivity and reversibility of the self-assembly processes, a biomimetic cell surface receptor system with unique features was obtained. For example, the ability to control reversibly the type of membrane-bound receptors and their local concentration levels with external molecular signals demonstrates the possibility of imitating dynamic processes that occur of cell surface proteins, such as changes in their expression level or post-translational modification. It was also shown that these changes can provide the bacteria with new properties such as an ability to glow with different colors, adhere to surfaces, and interact with proteins or cells; properties that may eventually be used in developing cell imaging methods, living materials and devices, or cell-based therapeutics, respectively. In light of these potential applications, the studies presented herein guide the development of additional biomimetic cell surface receptors, with which living cells could be ‘programed’ to preform diverse sets of functions.

### Example 11—Design of the ₁^{st} Generation B-Probes Intended to Selectively Label Distinct Cancer Cells

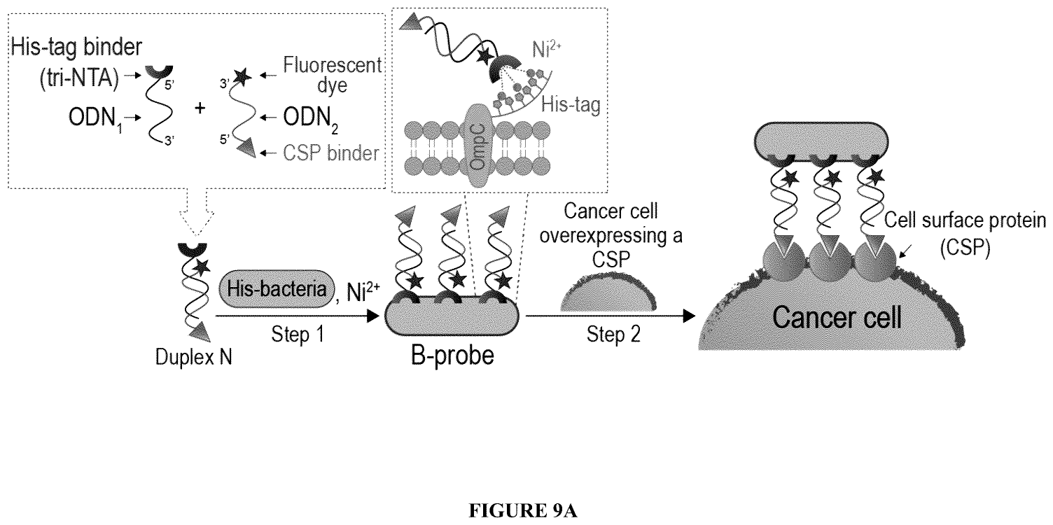

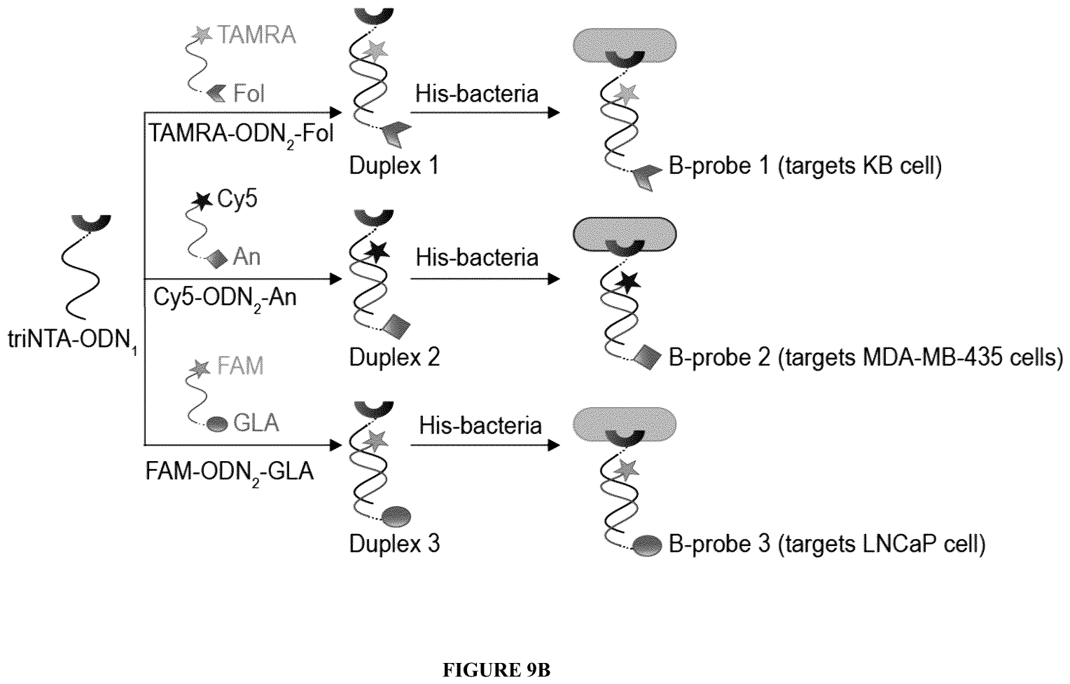

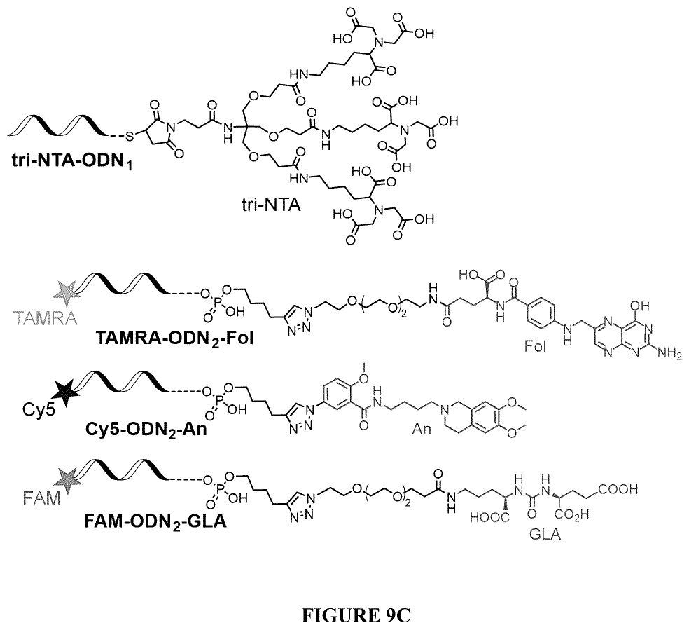

FIG. 9A schematically illustrates the approach of creating a new class of fluorescent probes that, similar to fluorescent Abs, are available in a wide range of colors and are able to selectively label diverse types of CSPs. These chemically modified bacterial probes (i.e., B-probes) were created by decorating His-OmpC expressing bacteria (His-bacteria) with modified DNA duplexes (duplex N). One of the ODNs constituting these duplexes is Compound 105 (tri-NTA-ODN₁), namely, a 19-base-long ODN (ODN₁) that is modified at its 5′ terminus with a tri-nitrilotriacetic acid (tri-NTA) unit. The tri-NTA group is connected to ODN₁ by a linker consisting of five sequentially arranged oligoethylene glycol (OEG) spacers (Scheme 1). In the presence of nickel ions, this tri-NTA group strongly and selectively interacts with the His-OmpC of E. coli. The other ODN is a complementary DNA strand (ODN₂) appended at its 3′ with a fluorescent dye and at its 5′ with a small-molecule-based ligand (i.e., a CSP binder) that can interact with a specific CSP on a cancer cell. The ligand is linked to ODN₂ via two consecutive OEG spacers. (Scheme 1)

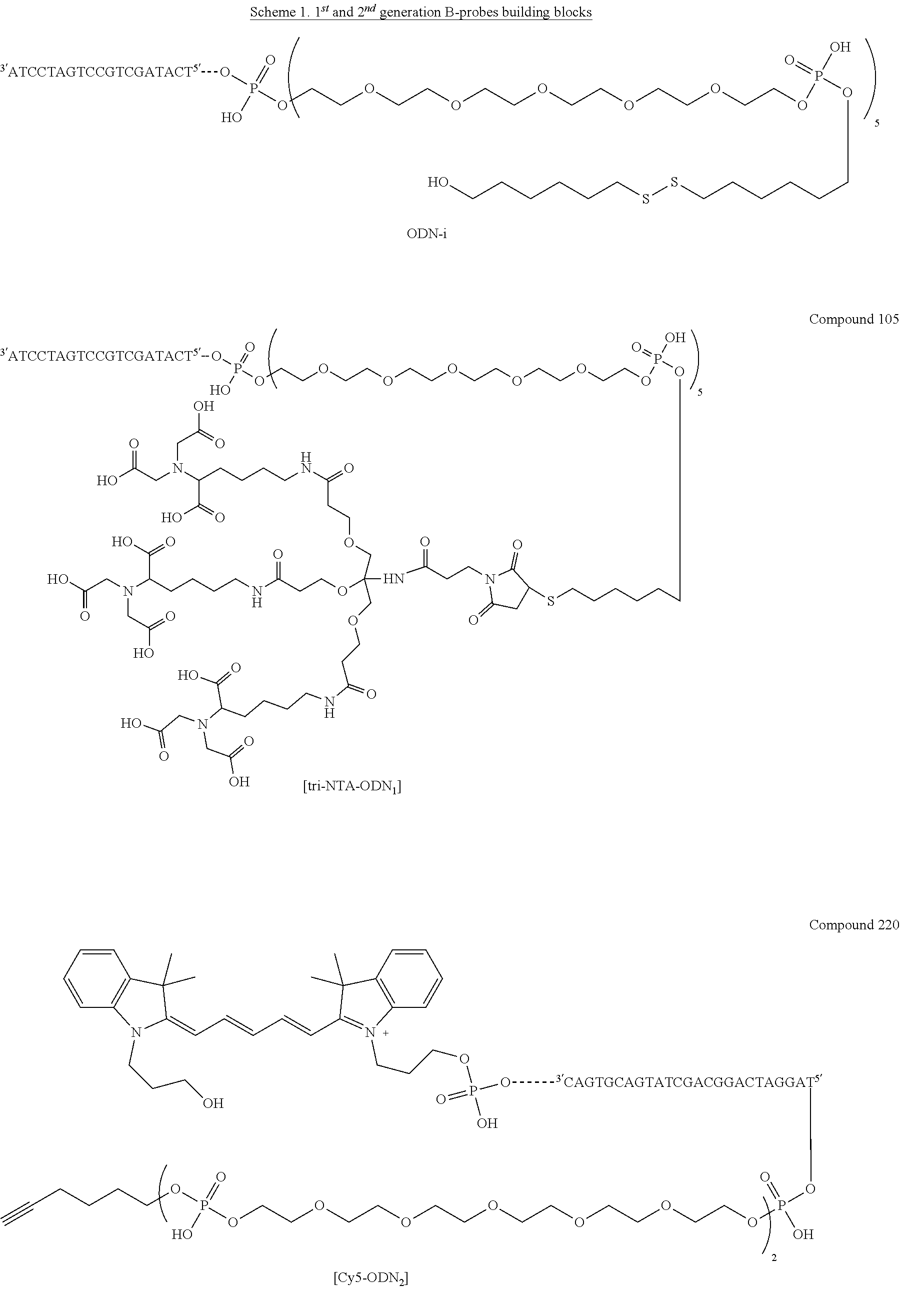

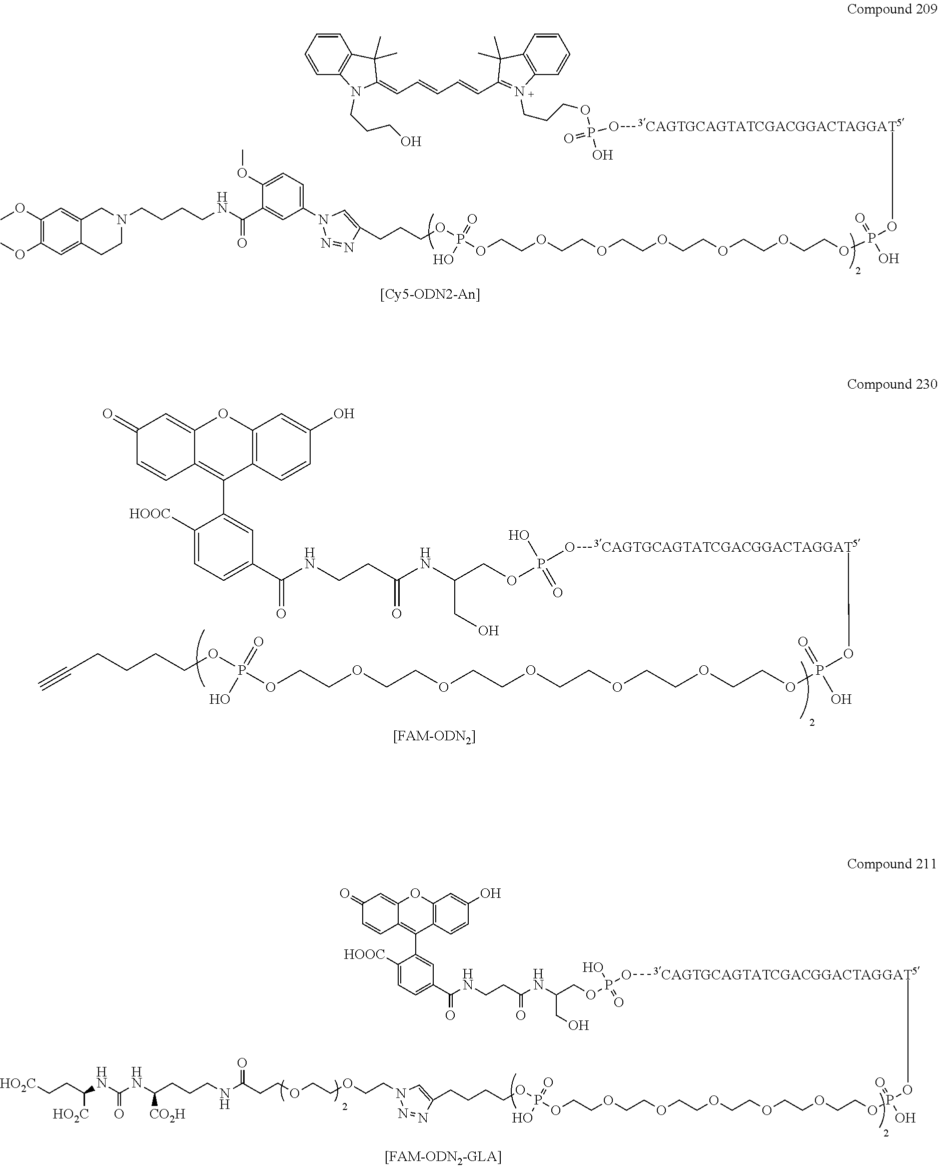

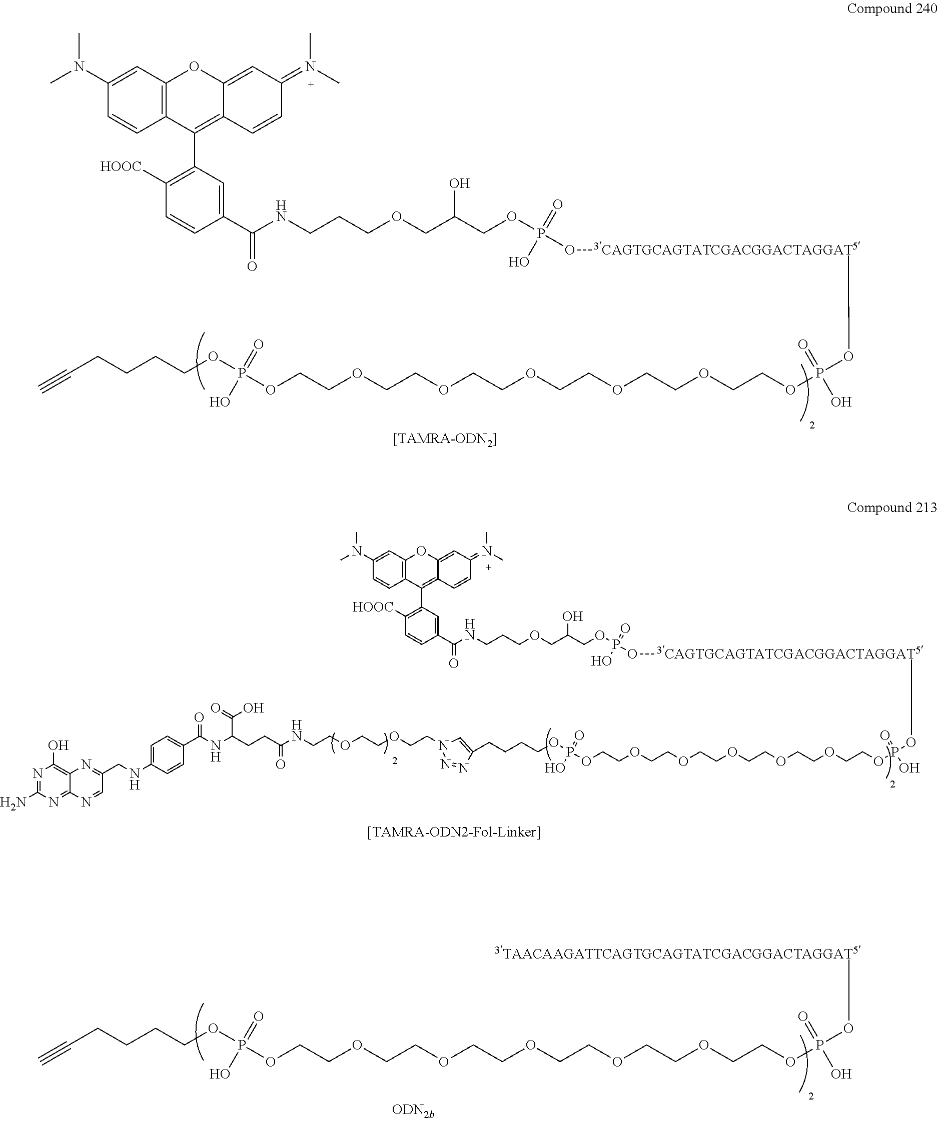

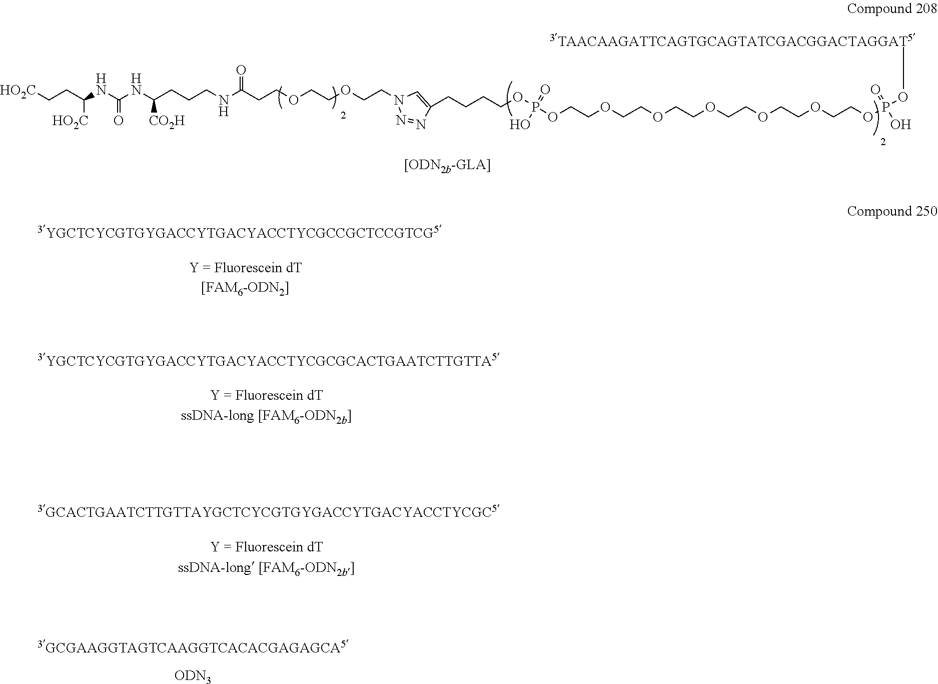

According to the design described herein, incubating the His-bacteria with the modified duplex and Ni²⁺ ions (FIG. 9A, step 1) afforded a B-probe that selectively labeled a specific type of cancer cells (FIG. 9A, step 2). His-OmpC expressing bacteria were selected to scaffold these probes owing to the high expression of His-OmpC, and the large surface area of bacterial cells. In nature, this combination (large surface area and high CSP density) enables the natural bacteria to bind a variety of host cells in a multivalent fashion. In the system described herein, similar design principles were applied to endow the B-probes with the ability to engage in unnatural bacteria-cancer cell interactions.

Based on these design principles, three modified DNA duplexes were created (FIG. 9B, duplexes 1-3) intended to afford three distinct B-probes (FIG. 9B, B-probes 1-3); each of which can label a specific cancer cell with a unique emission color. Duplexes 1-3 were generated by hybridizing the same His-tag binding strand (FIG. 9B, tri-NTA-ODN₁; Compound 105) with distinctly modified ODN₂ₛ, namely, FAM-ODN₂-GLA (Compound 212), TAMRA-ODN₂-Fol (Compound 213) or Cy5-ODN₂-An (Compound 210). The modified ODN₂, bear distinct fluorescent dyes: FAM (Ex/Em: 490/520 nm), TAMRA (Ex/Em: 545/580 nm) or Cy5 (Ex/Em: 630/670 nm). In addition they are appended with distinct CSP binding molecules: folate (Fol), anisamide (An), or glutamate urea (GLA), known to selectively bind the folate receptors (FRα), sigma receptor (SR), or the prostate-specific membrane antigen (PSMA) receptor, respectively. It was suggested that these CSP binders would enable B-probes 1, 2, and 3 to selectively label KB cells (cervical cancer cells), MDA-MB-435 (melanoma cells), and LNCaP (prostate cancer cells) overexpressing FRα, SRs and PSMA, correspondingly.

The design of B-probes 1-3 (FIG. 9B) highlights the high modularity of this technology, which should provide the means to create a wide range of B-probes from the same His-tag binding strand (Compound 105, tri-NTA-ODN₁) and His-OmpC expressing bacteria. Specifically, it shows that diversification of the B-probes' emission color and cancer cell targets can be achieved through a simple alteration of the fluorescent dyes and the small-molecule ligands that are linked to ODN₂. The fact that a wide range of dyes can be incorporated in ODN₂ during the automated DNA synthesis (by the proper selection of fluorescent phosphoramidites) further contributes to the high modularity of these probes. In this study, the TAMRA-ODN₂-Fol (Compound 213), Cy5-ODN₂-An (Compound 210), or FAM-ODN₂-GLA (Compound 212) was prepared simply by conjugating commercially available ODN₂ derivatives (fluorophore- and alkyne-modified ODN₂ₛ) to distinct azide-modified small-molecule ligands, using the copper catalyzed ‘click’ chemistry (Scheme 2).

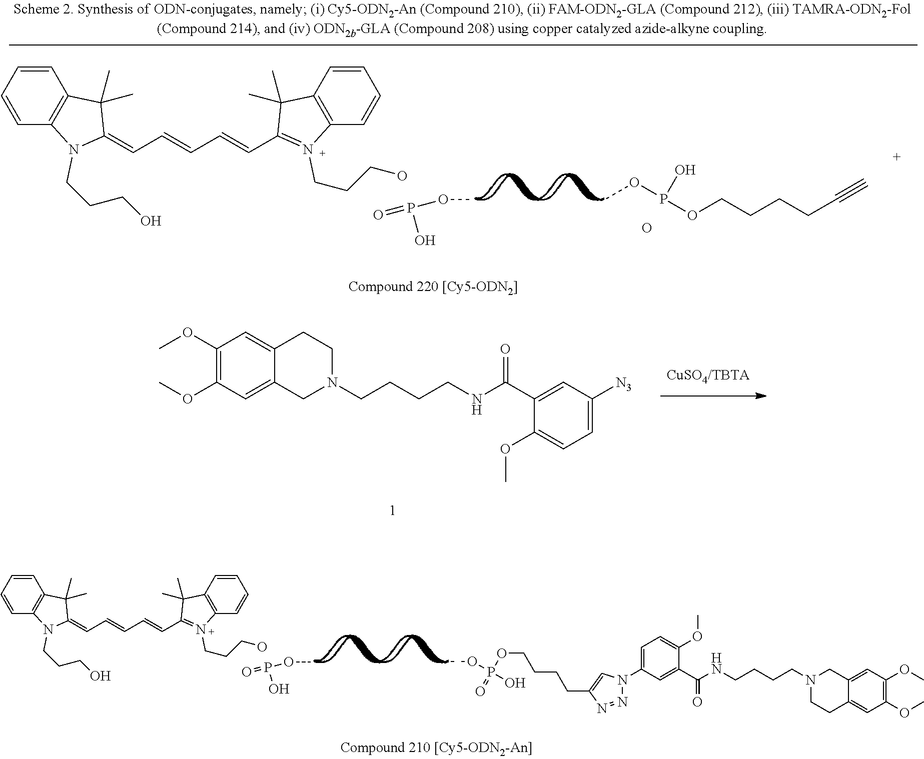

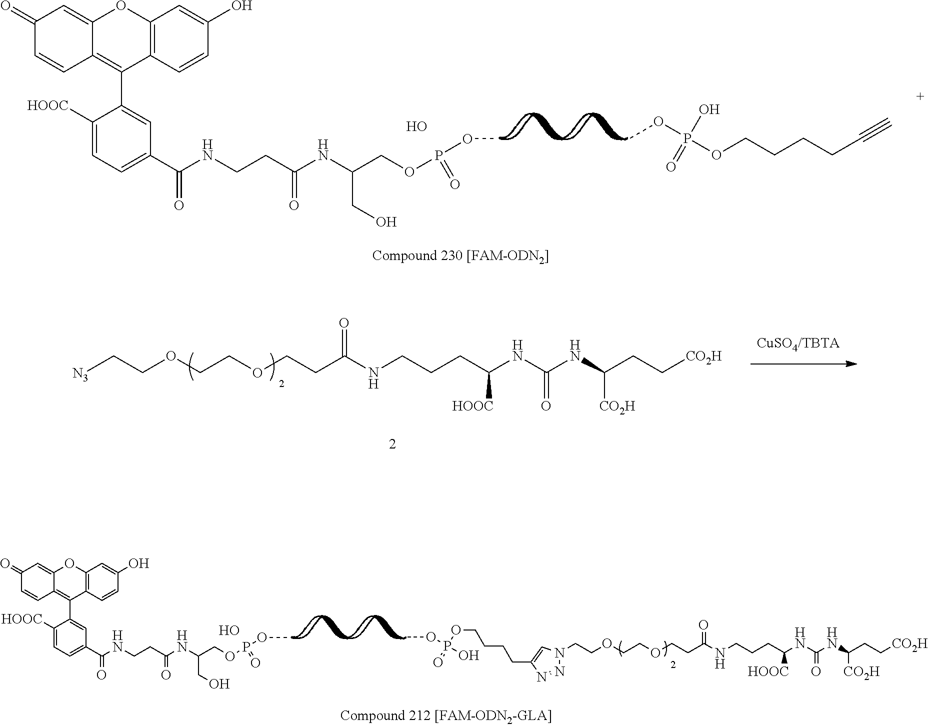

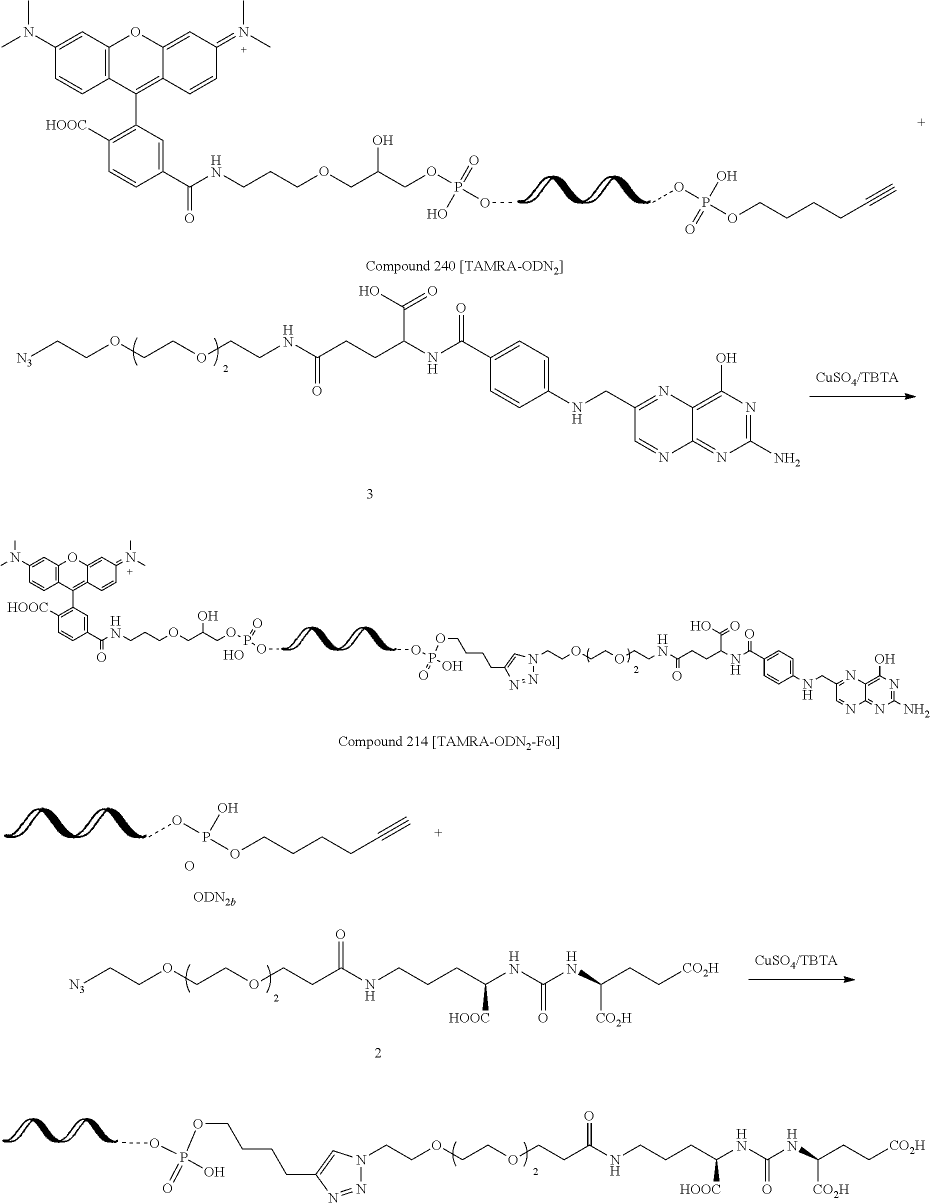

### Example 12—Fluorescent Labeling of Cancer Cells and Tissues with the 1^{St} Generation B-Probes

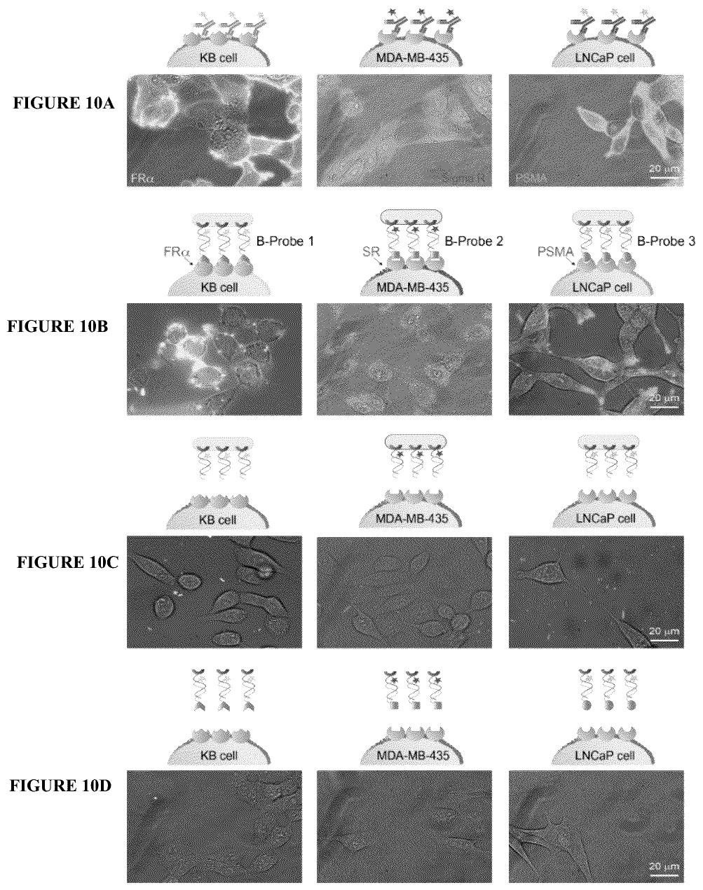

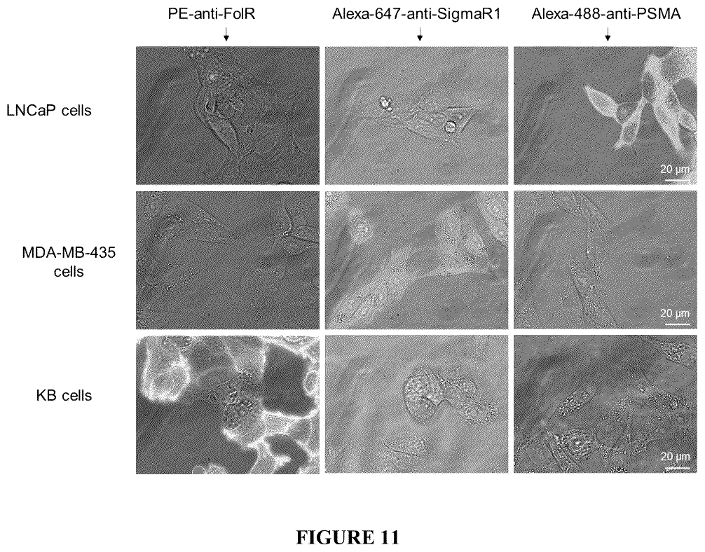

Before testing the ability of B-probes 1-3 to identify KB, MDA-MB-435, and LNCaP cells according to their unique CSP markers (FRα, SRs, and PSMA, respectively), these CSPs were first labeled with primary antibodies bearing different fluorophores (FIG. 10A and FIG. 11). As expected, the PE-anti-FolR Ab labeled the KB cells, the Alexafluor-647-anti-SigmaR Ab labeled MDA-MB-435 melanoma, and the Alexafluor-488-anti-PSMA Ab labeled the LNCaP cells. The results show, that each antibody selectively labeled one type of cancer cell (FIG. 11), indicating the low or no expression of each CSP target in the other two cell lines. Another reason for performing this IF labeling experiment was to demonstrate how efficient CSP labeling is.

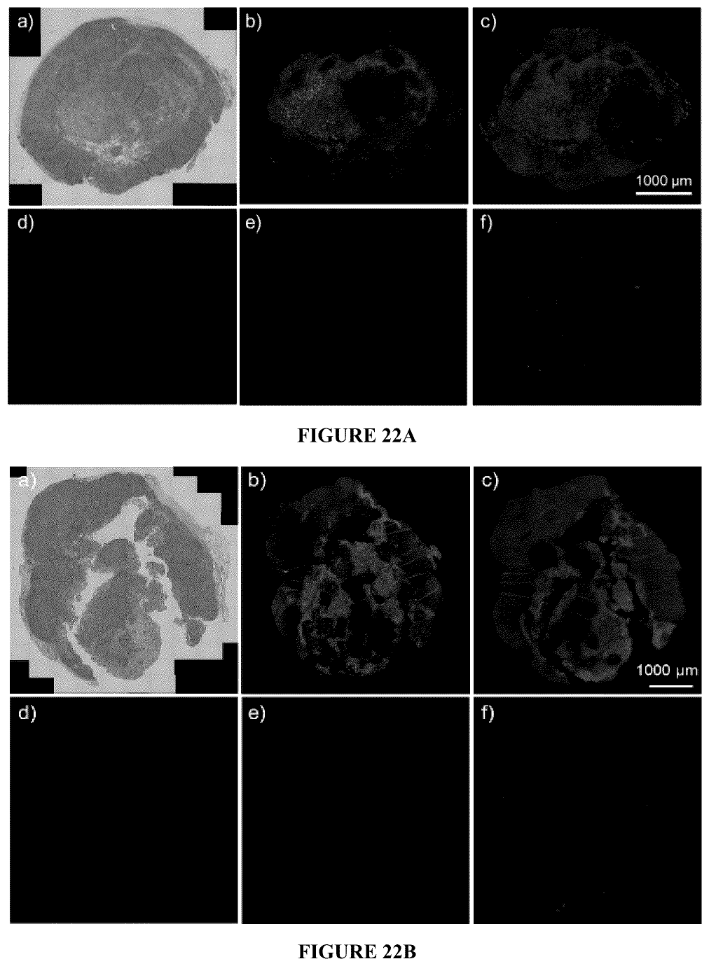

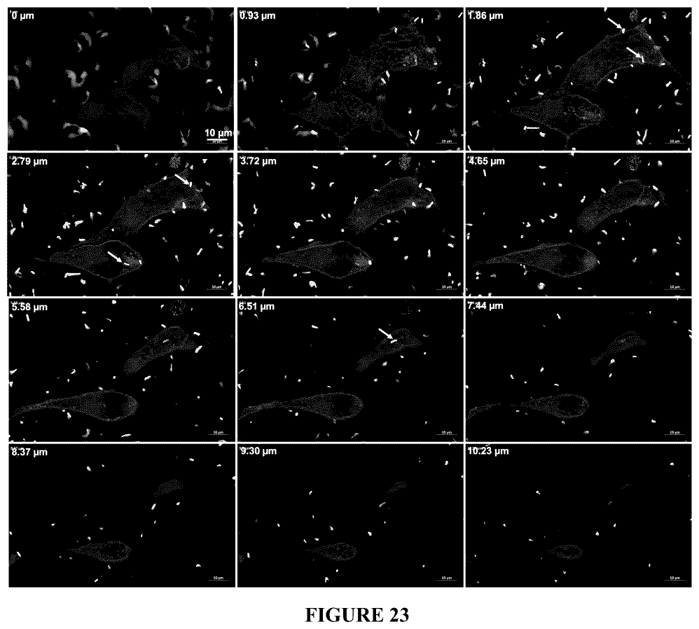

To determine whether efficient fluorescent cell labeling could also be obtained with the newly developed B-probes, the three cancer cell types were subjected to B-probes 1-3 (FIG. 10B). In this experiment, each cell type (KB, MDA-MB-435, or LNCaP) was incubated separately with each of the B-probes, washed, and imaged using a fluorescence microscope. As negative controls, the cancer cells were also imaged following incubation with bacteria linked to duplexes that lack the CSP binders, namely, duplexes generated from tri-NTA-ODN₁ (Compound 105) and TAMRA-ODN₂ (Compound 240), Cy5-ODN₂ (Compound 220) or FAM-ODN₂ (Compound 230) (FIG. 10C). In addition, the cancer cells were incubated with duplexes 1-3 in the absence of the His-bacteria (FIG. 10D). Inspecting the fluorescence images revealed that cancer cell labeling was only achieved when the cells were treated with the B-probes, namely, with His-bacteria decorated with fluorescent DNA duplexes bearing the specific CSP binders (i.e., Fol, An, or GLA) (FIG. 10B). The fact that no labeling was achieved in the absence CSP binders (FIG. 10C) indicates the selectivity of the system, namely, that the labeling does not result from non-specific B-probe-cancer cell interactions. Moreover, the inability of duplexes 1-3 to label the cancer cells in the absence of bacteria (FIG. 10D) shows that the bacterial scaffold is essential for obtaining multivalent interactions with the CSP targets, as well as for enhancing the emission signal by integrating a large number of fluorophores in a single probe. To confirm the fact that, unlike fluorescent Abs, B-probes bind CSPs at their small-molecule binding sites, it was also shown that the KB-cells are not labeled with B-probe 1 following incubation with folic acid (1 μM) (FIG. 21). This experiment, in which the B-probe was displaced by a small-molecule ligand, also indicates the potential of using the B-probe method to screen for small-molecule CSP agonists or antagonists, something that cannot be achieved with fluorescent Abs.

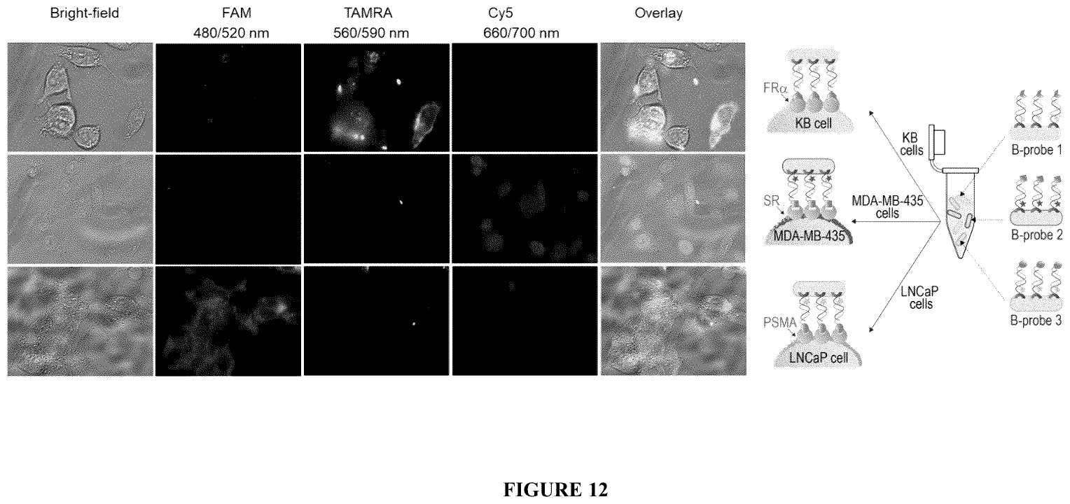

A more efficient way to differentiate between the cancer cell types can be achieved by subjecting the cells to a homogeneous mixture of B-probes 1-3 (FIG. 12, right). This may reduce incubation and washing steps, as well as minimize the number of samples needed for analysis. Because the fluorescent DNA duplexes are non-covalently bound to the His-bacteria, one may expect that combining different B-probes in a single solution would lead to an exchange of duplexes, which would lead to a mixed labeling of each cancer cell. The expectation that such mixed labeling will not occur was based on a previous analysis showing that mixing His-bacteria covered with distinct fluorescent duplexes did not lead to the formation of new populations of labeled bacteria. Fluorescent imaging of each cell type (under excitation and emission filters suitable for detecting FAM, TAMRA and Cy5) following its incubation with the B-probe mixture (FIG. 12, left), revealed that labeling with the mixture did not lead to cross-reactivity. For example, the melanoma cells were primarily labeled in far-red owing to the selective attachment of the Cy5-bearing B-probe (B-probe 2). In this case, the yellow (TAMRA) and green (FAM) emitting B-probes 1 and 3, were washed off Similarly, the KB cells and LNCaP were exclusively labeled with B-probes 1 and 3, respectively. The specific labeling by the mixture further indicates the selectivity of the B-probes toward their targets, as well as the potential to use them in multiplexed labeling; something that is currently achieved with mixtures of fluorescent Abs.

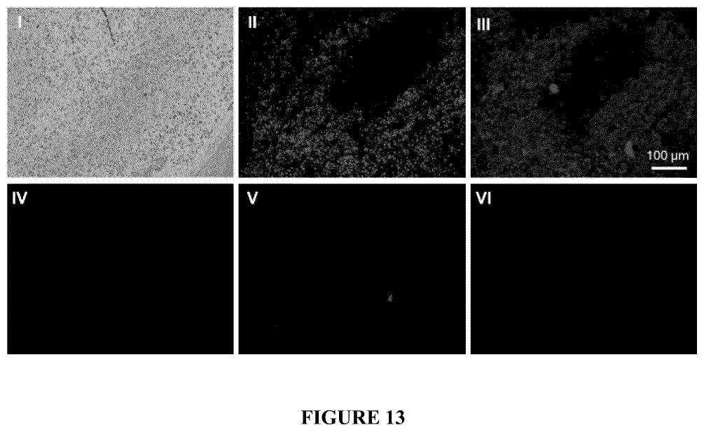

The selectivity, target variability, as well as the ability to combine distinct B-probes in a single solution (FIGS. 10 and 12), indicate some similarities between the B-probe labeling method and IF. In addition to labeling proteins in cells, an essential application of IF lies in analyzing tissues obtained from biopsies. The Ab-based fluorescence-staining patterns, which generally results from the labeling of overexpressed CSPs, can be used to diagnose the type of tumor, as well as determine to what extent it has spread. To further highlight the resemblance between the B-probe method and IF, a comparison of the ex-vivo tissue-labeling pattern obtained with fluorescent Abs to the B-probes was carried out (FIG. 13). To this end, MDA-MB-435 cell-derived xenografts (CDX) were induced in nude mice. After 4 weeks, the tumor (0.8 cm³) was extracted and processed into FFPE (Formalin-Fixed Paraffin-Embedded) blocks. Sections of 6 μm thickness from the FFPE tissue blocks were used for the staining experiments. One sample was stained with hematoxylin and eosin dye (FIG. 13, sample I), which is commonly used in histology to localize nuclei and other cytosolic and extracellular proteins. The other samples were labeled with Alexafluor647-anti-SigmaR1 Ab or B-probe-2 (FIG. 22) following the standard staining protocols.

Fluorescence imaging of the Ab-treated slides (FIG. 13, sample II) revealed that that, as expected, the fluorescent Ab clearly stained the regions bearing antigen. Remarkably, despite the notable structural differences between fluorescent Abs and B-probes, staining the tumor tissue sample with B-probe 2 led to the generation of an almost identical fluorescence pattern (FIG. 13, sample III). To validate the specificity of tissue labeling, it was also shown that the tissue was not labeled following incubation with Alexafluor-647 conjugated normal mouse IgG2b isotype control (FIG. 13, sample IV) or with a His-bacteria covered with a duplex that lacks the An unit (FIG. 13, sample V). Additionally, as was observed with cell labeling experiments (FIG. 10D) incubation of the cancer tissue with duplex 2 did not result in fluorescence labeling (FIG. 13, sample VI).

### Example 13—Development of 2^{nd} Generation B-Probes that can Produce Stronger Emission Signals

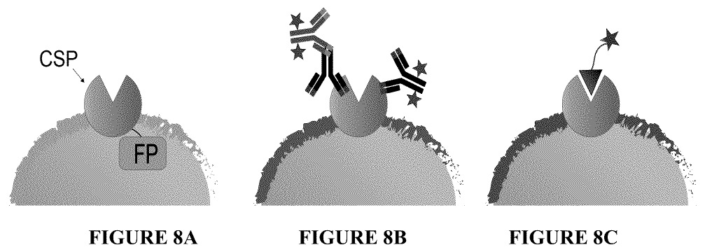

The efficiency by which fluorescent probe label their protein targets not only depends on their binding affinity, but also on the intensity of the emission signal they can produce. Because the fluorescence signal generally depends on the concentration of fluorophores in the medium, a rational approach to enhance the emission of CSP binding agents could be to link them to multiple fluorophores. Multi-dye conjugation, however, is generally unsuitable for improving the labeling efficiency of fluorescent Abs (FIG. 8B) or synthetic probes (FIG. 8C). The problem in using this method to enhance the emission of fluorescent Abs is that the fluorophores are generally non-specifically conjugated to lysine side chains on the Ab's scaffold (FIG. 8B). Hence, linking an Ab to more than a few dyes is expected to modify its antigen binding site, which will reduce its binding affinity. In contrast to Abs, synthetic probes (FIG. 8C) can be modified at well-defined positions, not at their protein-binding site. However, integrating several fluorophores in a single probe is synthetically challenging and it could also lead to sterically hindrance that would disrupt their interaction with the target protein. An additional problem that could be encountered when linking multiple dyes to both systems (i.e., fluorescent Abs and synthetic probes) is self-quenching that often occurs when fluorophores are located in proximity to each other.

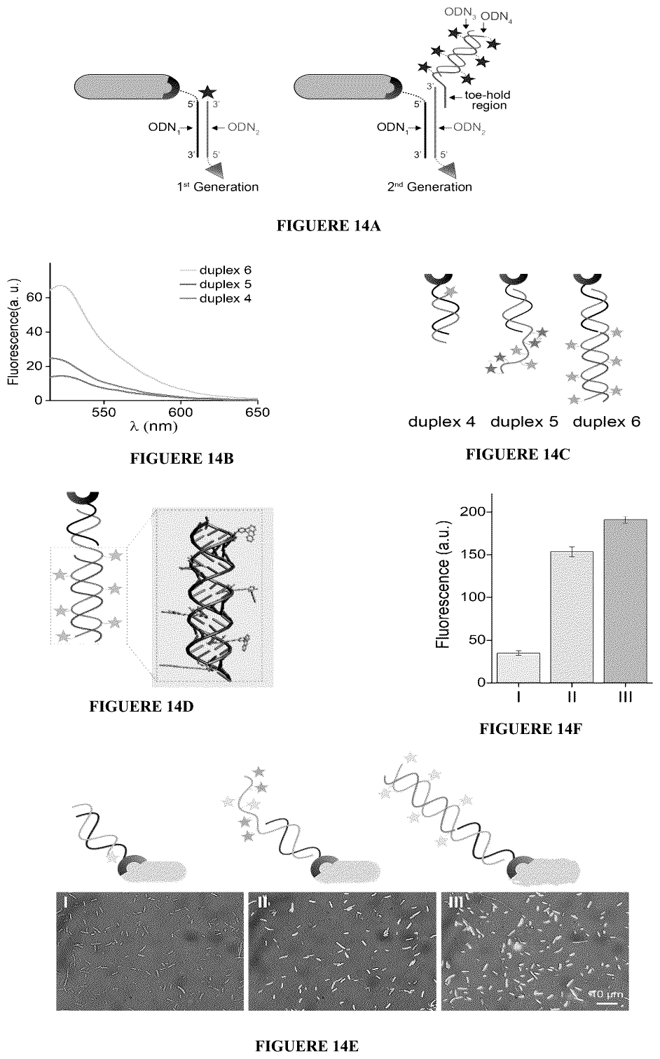

By using the structural programmability of the B-probes' DNA duplexes, it was possible to create brighter B-probes, in which the multiple fluorophores neither interact with each other nor hinder the probe-protein interactions (FIG. 14A, right). Specifically, it was suggested that brighter B-probes (FIG. 14A-right, 2^{nd} generation B-probes) could be obtained by replacing the single fluorescent dye incorporated in the duplexes constituting the 1^{st} generation probes (FIG. 14A, left) with a double stranded DNA (dsDNA) bearing multiple fluorophores (FIG. 14A, right). Notably, the fluorophores in this duplex are spatially separated; hence, they cannot self-quench. To facilitate the preparation of the 2^{nd} generation B-probes, the multi-dye-modified dsDNA is attached to ODN₂ via hybridization of two complementary hanging strands (or toeholds). This should enable one to change the type and number of dyes simply by preparing a new, multi-dye-modified dsDNA from ssDNA building blocks (ODN₃ and a multi-dye-modified ODN₄) that can be readily synthesized on an automated DNA synthesizer.

Prior to creating the proposed 2^{nd} generation B-probes (FIGS. 14A, right), a model system was used (FIGS. 14B-14F) to evaluate two key hypotheses underlying the design. The first hypothesis was that fluorescence quenching resulting from dye-dye contacts could be prevented by controlling the orientation of the fluorophores on a multi-dye modified DNA duplex. The second assumption was that His-bacteria decorated with such a duplex would emit more strongly than His-bacteria decorated with a duplex containing a single dye. To test the first hypothesis, the emission spectra of three modified DNA duplexes (FIG. 14B) whose structures are schematically depicted in FIG. 14C was recorded. Specifically, the emission generated by a duplex that bears a single FAM dye (FIG. 14C, duplex 4) was compared to the emission of two duplexes appended with six FAM units (FIG. 14C, duplexes 5 and 6). In duplex 5, ODN₂ was elongated with 26 base-long strand containing six FAMs dyes. Duplex 6 was generated by hybridizing duplex 5 with a DNA stand that is complementary to the elongated sequences (ODN₃). Because a right-handed R-helix has categorically ˜10 base pairs per turn, it was hypothesized that placing the dyes at positions i and i+5 would make the neighboring FAMs on duplex 6 project at distinct orientations. Molecular modeling (with BIOVIA Discovery Studio Visualizer) supported this assumption (FIG. 14D), as well as showed that FAM dyes located at positions i and i+10 should not interact with each other owing to the large distance between them. Inspecting the emission spectra generated by duplexes 4 and 5 (FIG. 14B) revealed that, despite having five additional FAMs, duplex 5 did not exhibit enhanced fluorescence when compared to duplex 4. In fact, a twofold reduction in emission was observed with respect to duplex 4, indicating a strong self-quenching effect. Duplex 6, on the other hand, in which the neighboring FAMs are separated, generated the strongest emission with a threefold enhancement in fluorescence over duplex 4.

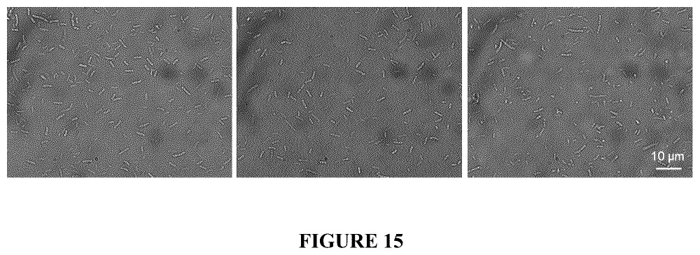

To determine whether the enhanced emission of duplex 6 would enable it to better label the His-bacteria, fluorescence images of the His-bacteria following incubation with 100 nM of duplexes 4-6 (in the presence of Ni²⁺) and washing were taken (FIG. 14E) and the average intensities of the fluorescence signals generated by individual bacterial cells were quantified (FIG. 14F). The results show that, as expected from the duplexes' emission spectra (FIG. 14C), the His-bacteria were most effectively labeled with duplex 6. Interestingly, although the emission intensity of duplex 5 in solution was lower than duplex 4, it better labeled the His-bacteria than did duplex 4 (FIG. 14E, middle vs. left). This indicates that the binding of duplex 5 to the His-bacteria disrupts the undesired dye-dye interaction, presumably due to non-specific interactions with the bacteria surface. The fact none of the duplexes labeled the His-bacteria in the absence of Ni²⁺ (FIG. 15) confirms that duplex elongation and modification with additional dyes does disrupt the specificity of such systems, namely, the binding of duplexes 5 and 6 to the bacterial His-OmpC is mediated by the highly selective interaction between the tri-NTA-Ni²⁺ complex and the His-tag.

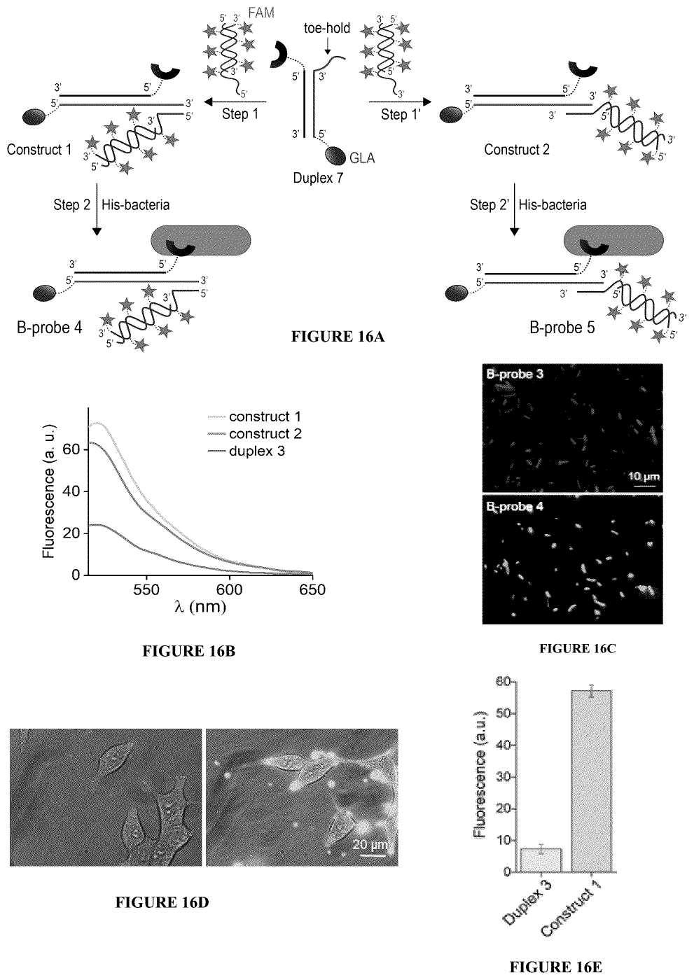

After demonstrating the feasibility of creating brighter chemically modified His-bacteria (FIGS. 14E and 14F), it was queried whether similar design principles could be used to develop the brighter, 2nd generation B-probes (FIG. 14A, right). Such probes should be able to label cancer cells more effectively than the 1P B-probe generation (FIG. 14A, left). To this end, B-probes 4 and 5 were created (FIG. 16A). These 2^{nd} generation B-probes were intended to label prostate cancer cells with greater efficiency than B-probe 3 (FIG. 9B) whose DNA duplexes are appended with a single FAM dye. B-probes 4 and 5 were prepared as follows (FIG. 16A): First, a DNA duplex integrating a tri-NTA unit, a GLA functionality and a toe-hold was created by (FIG. 16A, duplex 7); to this, another duplex appended with six FAM dyes and a complementary toe-hold was attached (FIG. 16A, step 1 or 1′) to afford DNA construct 1 (step 1) or 2 (step 1′) whose six FAM-appended duplex project to different orientations. The reason for altering the directionality of the multi-FAM duplex is to decrease the chance that it would interfere with the binding of the tri-NTA unit to His-OmpC. This change in directionality was achieved simply by altering the position of the hanging strand of the multi-FAM duplex from the 3′ to the 5′ terminus.

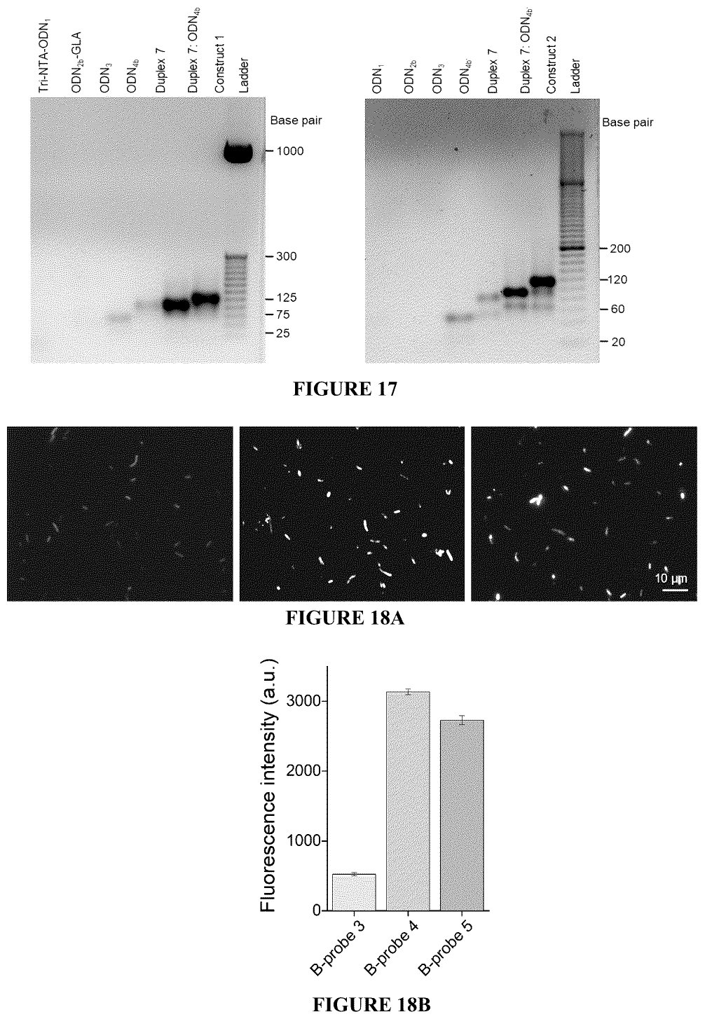

After confirming the efficient self-assembly of DNA constructs 1 and 2 by gel electrophoresis (FIG. 17), their emission spectra were recorded (FIG. 16B) and compared to the emission generated by single FAM-appended duplex 3 previously used to create the 1^{St} generation B-probe 3 (FIGS. 9B and 9C). The measurements (FIG. 16B) showed that the fluorescence generated by 100 nM of constructs 1 and 2 is 3-3.5 fold larger that the emission of duplex 3 under the same concentration. As expected from these emission differences, fluorescence imaging of B-probe 3 (FIG. 16C, top), B-probe 4 (FIG. 16C, bottom), and B-probe 5 (FIG. 18A-18B) showed that the 2nd generation B-probes (B-probes 4 and 5) are 5-6-fold brighter than the 1P generation B-probe 3. The fluorescence images also showed that constructs 1 and 2 afforded B-probes (B-probes 4 and 5) with similar brightness (FIG. 18A). Therefore, only one of them (B-probe 4) was used in the next experiments in which B-probe 4 was used to label prostate cancer cells (FIGS. 16D and 16E).

### Example 14—Using the 2^{nd} Generation B-Probes to Improve Cancer Cell Labeling and Track B-Probe Internalization

To determine whether the stronger fluorescence generated by the 2^{nd} a generation probes would enable them to label cancer cells more effectively than the 1^{st} generation probes, prostate cancer cells (LNCaP cells) were imaged following incubation with B-probe 3 (FIG. 16D, left) and B-probe 4 (FIG. 16D, right). Quantification of the emission signals (FIG. 16E) shows that, as expected from the stronger emission of the 2^{nd} generation B-probes, LNCaP cells subjected to B-probe 4 exhibited ˜6 fold enhancement in their emission when compared to LNCaP cells labeled with B-probe 3.

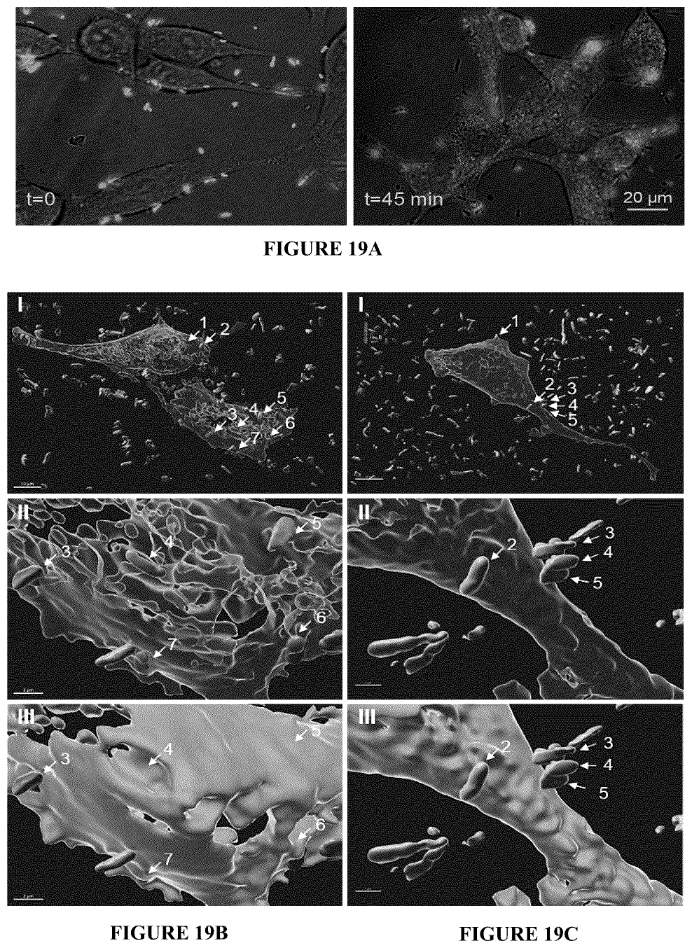

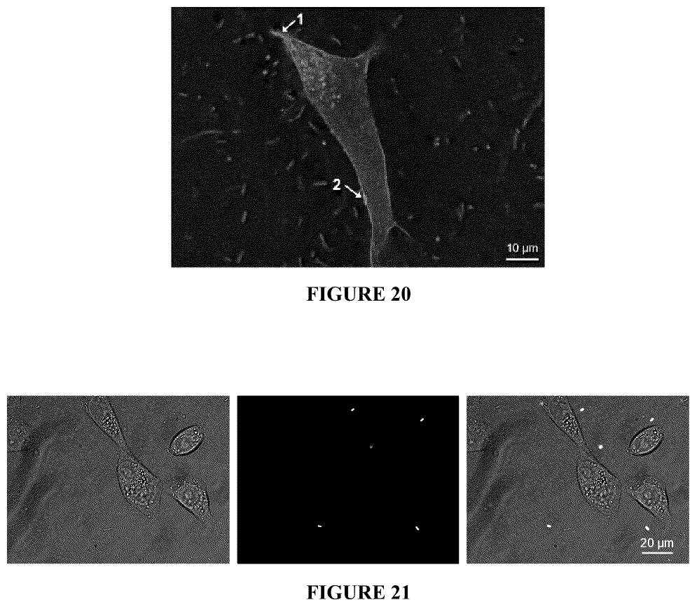

After demonstrating more efficient labeling of cancer cells with the 2^{nd} generation probe 4, the aim was to determine whether the multiple fluorophores incorporated in its DNA duplex would facilitate imaging of the chemically modified bacteria over time. An interesting phenomenon that was observed when imaging the 1P generation B-probes (B-probes 1-3) post incubation with cancer cells was that initially, intact fluorescently labeled bacteria (B-probes) were visualized on the surface of the cancer cells (FIG. 19A, left), whereas after ˜45 minutes large domains in the cancer cells became fluorescent (FIG. 19A, right). A probable explanation for this is a receptor-mediated endocytosis, which has been shown to induce the internalization of various small molecules and nano-carriers to these cells. However, when trying to follow the 1P generation B-probe-3 after binding to LNCaP cells, it was impossible to obtain clear images (FIG. 20). A plausible explanation for the loss of fluorescence signal is photobleaching that FAM dyes undergo under continuous illumination. Therefore, it was expected that a possible solution to this problem could be to repeat this imaging experiment with B-probe 4, which has a larger number of FAM dyes. This could make it less susceptible to photobleaching and thus, enable tracking the events that follow the initial binding of B-probe 4 to prostate cancer cells.

FIG. 19B shows the results of an experiment in which B-probe 4 was imaged in real time following incubation with LNCaP cells. To visualize the positioning of the bacterial probe (B-probe 4) with respect to the cancer cell membrane, LNCaP cells were transiently transfected with a plasmid encoding a mCherry CaaX-HRas protein, which resulted in the labeling of the boundary of the plasma membrane. Co-imaging of B-probe 4 and the cancer cells' membrane (FIG. 19B) shown in transparent (FIG. 19B, I, and II) and opaque membrane masks (FIG. 19B, III) under ambient conditions (37° C., 5% CO₂) indicates that the bacteria imaged over the cancer cells are either partially (bacteria 3 and 6) or entirely (bacteria 4, 5, and 7) engulfed in the cell. As a control, this experiment was repeated with a derivative of B-probe 4, which lacks the GLA unit (FIG. 19C). The fact that under these conditions bacterial internalization into the LNCaP cancer was not observed indicates that the targeting unit of B-probe 4 (GLA) mediates a selective interaction between B-probe 4 and prostate cancer cells, as well as the consequent receptor-mediated endocytosis. The ability to image both B-probe 4 (FIG. 19B) and its non-targeting derivative (FIG. 19C) under continuous irradiation is another important outcome of this experiment, showing how an increase in the number of fluorescent dyes provides a possible means to minimize undesired photobleaching effects.

### Example 15—Conclusion

Several innovations that were introduced to the bacterial-based fluorescent labeling strategy were described herein. This strategy enabled the creation of B-probes that can bind to different CSPs and that can emit at different colors with enhanced brightness. These features enabled the probes to identify three types of cancer cells, even when the B-probes were combined in a single solution. The results indicate the programmability, selectivity, and target versatility of B-probes, as well as the potential to use them to obtain rapid, multiplexed detection. The applicability of this technology was further demonstrated by using B-probes to fluorescently label ex-vivo tissue derived from a human tumor-bearing mouse model, as well as by developing brighter B-probes that can be used to label cancer cells with higher sensitivity and to follow the internalization of the chemically modified bacteria into cancer cells. The high selectivity of the unnatural bacterium-cancer cell interactions that were presented here and the fact that these interactions engage the small-molecule binding site of CSPs also indicate the potential to apply some design principles underlying B-probes in the future development of bacterial therapeutics or live cell-based inhibitor screening.

In nature, bacterial infections are primarily mediated by the multivalent interaction between bacterial and host cell CSPs. The high efficiency of this recognition event is attributed to the large surface area of the bacterial cells and the high density of the targeting CSPs (e.g., adhesins). This work has shown that these properties make living bacteria an excellent scaffold for creating fluorescent probes able to effectively label specific types of cancer cells and tissues. The bacterial probes (B-probes) were created by non-covalently linking a highly expressed bacterial CSP (e.g., His-OmpC) to DNA duplexes appended with a fluorophore and two protein binders. One binder interacts with His-OmpC, whereas the other binds to cancer cell CSP. The high efficiency by which the B-probes labeled the cancer cells can be attributed to two main factors: The first is the large number of small-molecule ligands (binders of cancer cell CSPs) covering the bacterial scaffolds. This enables the B-probes to engage in multivalent interactions with the cancer cells, resulting in high affinity binding. The second factor is the large number of fluorophores decorating each bacterium, enabling individual B-probes to generate a strong emission signal.

This combination of multiple targeting elements and multiple fluorescent dyes not only makes B-probes superior to most monovalent fluorescent probes (FIG. 8C)—it also enables using B-probes in applications generally achieved with IF. It has been shown herein, that similar to CSP imaging with fluorescent Abs, B-probes can label CSPs overexpressed cancer cells (KB, MDA-MB-435, and LNCaP cells). The ability to label these three cell types using a mixture of distinct B-probes, further demonstrates the resemblance of this technology to IF, since it indicates the high selectivity of such systems and the potential to apply them in multiplexed detection. It has also been shown that B-probes can be used to identify carcinogenic tissues, providing labeling patterns similar to the ones obtained with fluorescence Abs.

Although the above-mentioned experiments highlight some similarities between the B-probe labeling method and IF, the design, structure and operating principles underlying B-probes and fluorescent Abs are fundamentally different. One key difference is the way fluorescent Abs and B-probes are constructed. Whereas fluorescent Abs are generated through covalent conjugation of fluorophores to a few, randomly distributed amino acid side chains, B-probes are generated through self-assembly processes involving Ni²⁺ complexation and DNA hybridization. One advantage of self-assembly is that it makes B-probes more amenable to modification than fluorescent Abs. It has been shown, that brighter B-probes (the 2^{nd} generation B-probes) can be readily created by integrating multi-dye containing DNA duplexes into their structures. By controlling the orientation of the dyes, it has been ensured that adjacent dyes were spatially separated and consequently, the self-quenching of fluorescence was prevented. This enabled the 2^{nd} generation B-probes to better label cancer cells and has also made them less susceptible to photobleaching. The ability to place the multi-dye-modified duplexes far from the His-tag binder or the CSP ligand is another important advantage of the B-probes over Abs, because it ensures that the addition of dyes does not disrupt the binding of the B-probes to their protein targets. The observation that the B-probes undergo internalization following a highly selective interaction with cancer cells is another interesting aspect of this study because it indicates the potential to apply some design principles underlying B-probes in bacterial therapy. Specifically, it indicated the possibility of improving the targeting capabilities of therapeutic bacteria through systematic chemical modification of their membranes.

Another critical difference between B-probes and fluorescent Abs lies in the CSP domains that they target. Whereas B-probes target small-molecule binding sites of CSPs, Abs target the CSPs' surfaces. This difference may open the way to using B-probes to label CSPs that do not have suitable Abs. In addition, as was exemplified in the folate displacement experiment (FIG. 21), it may enable using B-probes to screen for CSP agonists or antagonists. Although optimizing such probes for these purposes may require additional modifications of their structures, this work has shown that ID probes are exceptionally simple to manufacture and modify. First, it has been shown that distinct B-probes can be formed from the same basic components. One is a self-replicating bacterial scaffold, whereas the other is a His-tag binding strand. Second, it has been shown that most structural modifications, such as changing the type and number of dyes, can be readily obtained through a rational choice of commercially available phosphoramidites used in the automated DNA synthesis. Considering this structural programmability, it can be reasonably assumed that new classes of probes for detecting additional types of cancer cells with higher efficiencies will be developed, which would extend the fluorescence toolbox currently used to detect CSPs in native (non-engineered) cells.

### Example 16—Experimental Details

### Synthetic Procedures of More Compounds of the Invention

### Synthesis of Anisamide Derivative

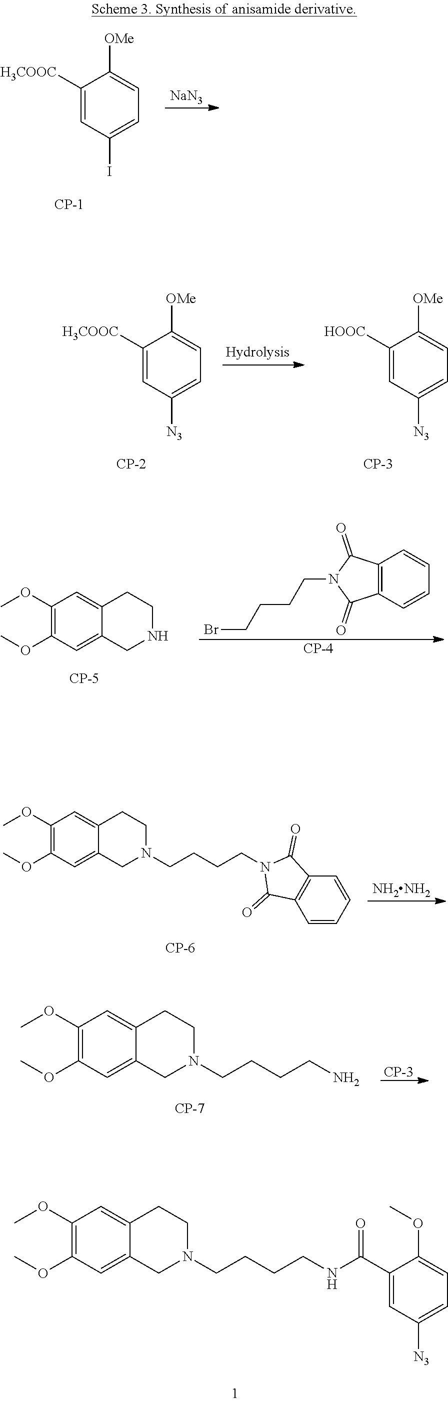

Synthesis of CP-2 (Scheme 3). To a stirred solution of CP-1 (1.0 g, 3.42 mmol) in 20 mL of dry DMF at room temperature, was added copper (I) iodide (650 mg, 3.42 mmol), DMEDA (45 mg, 0.51 mmol) and sodium ascorbate (34 mg, 0.17 mmol). This was followed by addition of sodium azide (450 mg, 6.84 mmol), the reaction mixture was allowed to stir at an elevated temperature of 80° C. On completion of the reaction (as monitored by TLC), DMF was evaporated under reduced pressure while the residue was re-suspended in H₂O/EtOAc (1:1). The aqueous layer was extracted with ethyl acetate (3×25 mL). The organic layers were combined, dried over anhydrous sodium sulphate and evaporated under reduced pressure. The resulting crude mixture was purified by flash column chromatography (EtOAc/hexane, 10:90) to yield an off-white solid (490 mg, 70%). ¹H NMR (400 MHz, CDCl₃): δ 3.89 (s, 3H), 3.90 (s, 3H), 6.97 (d, J=9.0 Hz, 1H), 7.11 (dd, J=9.0, 2.9 Hz, 1H), 7.47 (d, J=2.9 Hz, 1H). ¹³C NMR (100 MHz, CDCl₃): δ 52.2, 56.4, 113.6, 121.1, 121.9, 123.8, 132.2, 156.4, 165.7. ESI-MS (m/z): calcd. For [M+H]⁺ 208.07; found: 208.09.

Synthesis of CP-3 (Scheme 3). To a stirred solution of CP-2 (250 mg, 1.2 mmol) in 5 mL of THF: MeOH: H₂O (3:1:1) at room temperature, was added lithium hydroxide monohydrate (255 mg, 6.0 mmol). The reaction mixture was stirred at room temperature for 4 h. On completion of the reaction (as monitored by TLC), the solvents were evaporated under reduced pressure while the resulting crude mixture was purified by flash column chromatography (MeOH/DCM, 5:95) to yield a colorless powder (200 mg, 86%).¹H NMR (500 MHz, DMSO-d₆): δ 3.81 (s, 3H), 7.17 (d, J=8.9 Hz, 1H), 7.26 (dd, J=8.9, 3.0 Hz, 1H), 7.31 (d, J=3.0 Hz, 1H), 12.90 (br. s., 1H); ¹³C NMR (125 MHz, DMSO-d₆): δ 56.2, 114.3, 120.8, 122.7, 123.4, 131.2, 155.4, 166.4. ESI-MS (m/z): calcd. For [M+H]⁺ 194.06; found: 194.07.

Synthesis of CP-6 (Scheme 3). To a stirred solution of CP-5 (1.0 g, 4.36 mmol) in 70 mL of anhydrous ethanol at room temperature, was added 2-(4-bromobutyl)isoindoline-1,3-dione (1.35 g, 4.80 mmol), followed by addition of K₂CO₃ (660 mg, 4.80 mmol). The reaction mixture was refluxed at 80° C. overnight. After completion of the reaction, the mixture was filtered and the filtrate was evaporated under reduced pressure to afford the crude product. The crude mixture was purified by flash column chromatography (MeOH/DCM, 3:97) to yield an off-white solid (1.5 g, 87%). ¹H NMR (400 MHz, CDCl₃): δ 1.52-1.63 (m, 2H), 1.66-1.76 (m, 2H), 2.47 (t, J=7.3 Hz, 2H), 2.62 (t, J=5.6 Hz, 2H), 2.73 (t, J=5.6 Hz, 2H), 3.47 (s, 2H), 3.67 (t, J=6.9 Hz, 2H), 3.76 (br. s, 6H), 6.44 (s, 1H), 6.51 (s, 1H), 7.63 (dd, J=5.2, 3.2 Hz, 2H), 7.70-7.88 (m, 2H). ¹³C NMR (100 MHz, DMSO-d₆): δ 23.9, 25.9, 28.3, 37.4, 50.7, 55.1, 55.4, 55.4, 57.1, 109.9, 111.7, 122.9, 125.9, 126.6, 131.6, 134.3, 146.8, 147.1, 167.9. ESI-MS (m/z): calcd. For [M+H]⁺ 395.20; found: 395.19.

Synthesis of CP-7 (Scheme 3). To a stirred a solution of CP-6 (473 mg, 1.2 mmol) in 20 mL of anhydrous ethanol was added hydrazine monohydrate (750 mg, 15 mmol) and the solution was refluxed for 1 h at 80° C. The reaction mixture was cooled and treated with an additional 20 mL of ethanol and concentrated HCl (1.3 mL). The reaction mixture was then refluxed for another 4 h and left overnight in a refrigerator. It resulted in formation of fine crystals, the solution was filtered. The residue was extracted with n-hexane (20 mL) and NH₄OH (15 mL). The solution was extracted with CHCl₃ (3×15 mL), the organic layer was dried over anhydrous K₂CO₃, and the solvents were evaporated to give CP-7 (colourless powder, 250 mg, 79%) which was used without further purification. ¹H NMR (400 MHz, DMSO-d₆): δ 1.61-1.71 (m, 2H), 1.84-1.94 (m, 2H), 2.81 (t, J=7.2 Hz, 2H), 3.16 (t, J=7.2 Hz, 2H), 3.72 (s, 3H), 3.73 (s, 3H), 6.77 (s, 1H), 6.80 (s, 1H). ESI-MS (m/z): calcd. For [M+H]⁺ 265.19; found: 265.20.

Synthesis of 1 (Scheme 3). To a stirred solution of CP-4 (25 mg, 0.129 mmol) in 5 mL of DCM:THF (1:1) mixture, under argon atmosphere, was added DCC (40 mg, 0.1932 mmol), followed by the addition of Et₃N (15.6 mg, 0.1546 mmol). After 10 min., CP-7 (40 mg, 0.1546 mmol) was added. The reaction mixture was stirred under argon at room temperature for 24 h. After completion of the reaction, the solvent was evaporated under reduced pressure and the crude was purified by RP-HPLC to yield pure compound 1 (colourless oil, 42 mg, 74%). ¹H NMR (500 MHz, ACN-d₃): δ 1.67 (quin, J=7.0 Hz, 2H), 1.82-1.90 (m, 2H), 2.88-3.00 (m, 1H), 3.10-3.16 (m, 1H), 3.17-3.28 (m, 4H), 3.43 (q, J=6.5 Hz, 2H), 3.62-3.69 (m, 1H), 3.76 (s, 3H), 3.78 (s, 3H), 3.94 (s, 3H), 4.06 (dd, J=14.9, 4.7 Hz, 1H), 4.43 (d, J=14.9 Hz, 3H), 6.67 (s, 1H), 6.76 (s, 1H), 7.10-7.14 (m, 1H), 7.15-7.19 (m, 1H), 7.64 (d, J=2.8 Hz, 1H), 8.09 (br. s., 1H). ¹³C NMR (125 MHz, ACN-d₃): δ 22.0, 25.3, 27.5, 39.3, 50.5, 53.1, 56.1, 56.4, 56.5, 57.3, 110.6, 112.5, 114.7, 120.5, 122.4, 124.1, 133.8, 149.4, 150.1, 156.1, 165.5. ESI-MS (m/z): calcd. For [M+H]⁺ 440.23; found: 440.23.

### Synthesis of Glutamate Urea

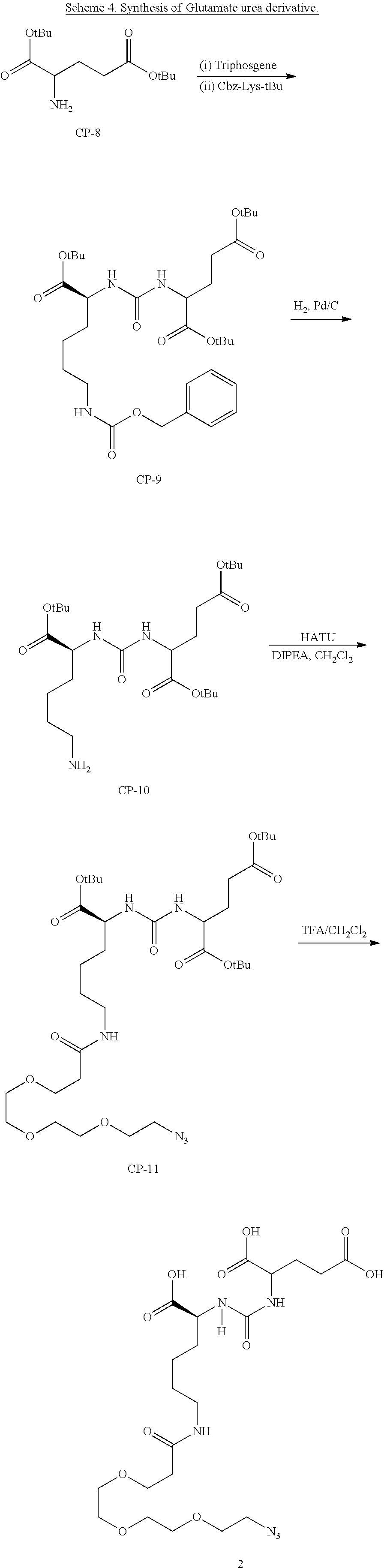

Synthesis of CP-9 (Scheme 4). Triphosgene (400 mg, 1.35 mmol) was added dropwise to a stirred solution of CP-8 (1.2 g, 4.05 mmol) in dry DCM under argon at 0° C. After addition of DIPEA (1.5 g, 11.62 mmol), the mixture was stirred for 4 h at 0° C. After 4 h, the reaction was brought to room temperature and lysine derivative, tert-butyl N⁶-((benzyloxy)carbonyl)-L-lysinate hydrochloride (1.0 g, 2.70 mmol) was added. The reaction mixture was further stirred for 1 h at room temperature, followed by evaporation of the solvent at reduced temperature and purification by column chromatography to yield CP-9 (580 mg, 69%) as a colourless oil. ¹H NMR (500 MHz, CDCl₃): δ 1.23-1.36 (m, 2H), 1.45 (s, 9H), 1.47 (s, 9H), 1.48 (s, 9H), 1.50-1.58 (m, 2H), 1.66-1.76 (m, 1H), 1.78-1.86 (m, 1H), 1.86-1.94 (m, 1H), 2.05 (dq, J=13.8, 6.7 Hz, 1H), 2.35-2.41 (m, 2H), 3.18 (t, J=6.7 Hz, 2H), 4.21-4.33 (m, 2H), 5.11 (s, 2H), 7.29-7.38 (m, 5H). ¹³C NMR (125 MHz, CDCl₃): δ 22.2, 27.8, 27.8, 27.8, 28.1, 29.1, 31.4, 32.4, 40.5, 52.6, 53.1, 66.2, 80.2, 81.3, 81.9, 127.7, 127.8, 128.2, 136.6, 156.5, 157.1, 172.1, 172.5, 172.8. ESI-MS (m/z): calcd. For [M+H]⁺ 622.37; found: 622.37.

Synthesis of CP-10 (Scheme 4). Compound CP-9 (460 mg, 0.74 mmol) was dissolved in 30 mL MeOH and purged with argon. Next, 10% Pd/C (40 mg, 0.037 mmol) was added and the reaction was stirred overnight under H₂ (836 Torr). The mixture was filtered over celite and the solvent was removed under reduced pressure to yield CP-10 as a yellow viscous oil (322 mg, 90%). ¹H NMR (400 MHz, MeOH-d₄): δ 1.19-1.35 (m, 2H), 1.45 (s, 9H), 1.47 (s, 9H), 1.48 (s, 9H), 1.57-1.69 (m, 2H), 1.75-1.86 (m, 2H), 2.02-2.10 (m, 1H), 2.27-2.38 (m, 2H), 2.85 (t, J=7.0 Hz, 1H), 3.35 (br. s, 2H), 4.14-4.22 (m, 2H). ¹³C NMR (100 MHz, MeOH-d₄): δ 23.8, 28.5, 28.5, 29.1, 32.6, 33.2, 54.2, 54.7, 61.6, 81.8, 82.7, 82.8, 160.0, 173.5, 173.8, 173.8. ESI-MS (m/z): calcd. For [M+H]⁺ 488.33; found: 488.34.

Synthesis of CP-11 (Scheme 4). 3-(2-(2-(2-azidoethoxy)ethoxy)ethoxy)propanoic acid (30 mg, 0.1232 mmol) was dissolved in dry DCM (5 mL) and stirred in a reaction vial at room temperature. Then, HATU (78 mg, 0.206 mmol) and DIPEA (27 mg, 0.206 mmol) were sequentially added. After stirring the reaction mixture for 10 minutes, CP-10 (50 mg, 0.103 mmol) dissolved in DCM was added dropwise to the reaction mixture via a syringe. The reaction was continued for 18 h (until completion), followed by evaporation of the solvent and purification of the resulting crude by RP-HPLC to yield pure compound CP-11 (56 mg, 76%). ¹H NMR (500 MHz, ACN-d₃): δ 1.31-1.37 (m, 2H), 1.42 (s, 9H), 1.43 (s, 9H), 1.43 (s, 9H), 1.44-1.50 (m, 2H), 1.53-1.63 (m, 1H), 1.66-1.80 (m, 2H), 1.96-2.02 (m, 1H), 2.18-2.32 (m, 2H), 2.37 (t, J=6.1 Hz, 2H), 3.12-3.18 (m, 2H), 3.37 (t, J=4.9 Hz, 2H), 3.56 (d, J=2.3 Hz, 4H), 3.59 (br. s., 4H), 3.61-3.64 (m, 2H), 3.66 (t, J=5.9 Hz, 2H), 4.05 (br. s, 1H), 4.14 (br. s, 1H), 5.44 (br. s, 1H), 5.50 (br. s, 1H), 6.77 (br. s, 1H). ¹³C NMR (125 MHz, ACN-d₃): δ 23.4, 28.3, 28.3, 28.4, 28.9, 29.8, 32.2, 32.6, 37.4, 39.6, 51.6, 54.0, 54.7, 67.9, 70.6, 71.0, 71.1, 71.2, 71.2, 81.0, 81.9, 82.3, 158.5, 172.6, 173.0, 173.1, 173.4. ESI-MS (m/z): calcd. For [M+H]⁺ 717.44; found: 717.44.

Synthesis of 2 (Scheme 4). TFA (1 mL) was added to a cooled solution (0° C.) of CP-11 (50 mg, 0.061 mmol) in DCM (3 mL). The reaction mixture was warmed up to room temperature and stirring was continued for another 3 h. After the reaction was complete, DCM and TFA were evaporated. The traces of TFA were removed by co-evaporation with DCM. The crude compound was dissolved in 3 mL ACN/H₂O (1:1), frozen with liquid nitrogen and lyophilized under high vacuum to afford a colourless viscous oil as product (30 mg, 90%). ¹H NMR (500 MHz, DMSO-d₆): δ 1.22-1.30 (m, 2H), 1.33-1.41 (m, 2H), 1.45-1.55 (m, 1H), 1.58-1.67 (m, 1H), 1.67-1.77 (m, 1H), 1.91 (td, J₁=14.1 Hz, J₂=6.5 Hz, 1H), 2.15-2.32 (m, 4H), 3.00 (dt, J₁=5.4 Hz, J₂=16.0 Hz, 2H), 3.39 (t, J=4.8 Hz, 2H), 3.45-3.62 (m, 12H), 4.00-4.06 (m, 1H), 4.06-4.12 (m, 1H), 6.28 (d, J=8.1 Hz, 1H), 6.32 (d, J=8.3 Hz, 1H), 7.80 (t, J=5.4 Hz, 1H). ¹³C NMR (125 MHz, DMSO-d₆): δ 22.6, 27.5, 28.8, 29.9, 31.8, 36.1, 38.3, 50.0, 51.6, 52.2, 66.9, 69.2, 69.5, 69.7, 69.7, 69.8, 157.3, 169.8, 173.7, 174.1, 174.5. ESI-MS (m/z): calcd. For [M+H]⁺ 549.25; found: 549.25.

### Synthesis of Folate Derivative

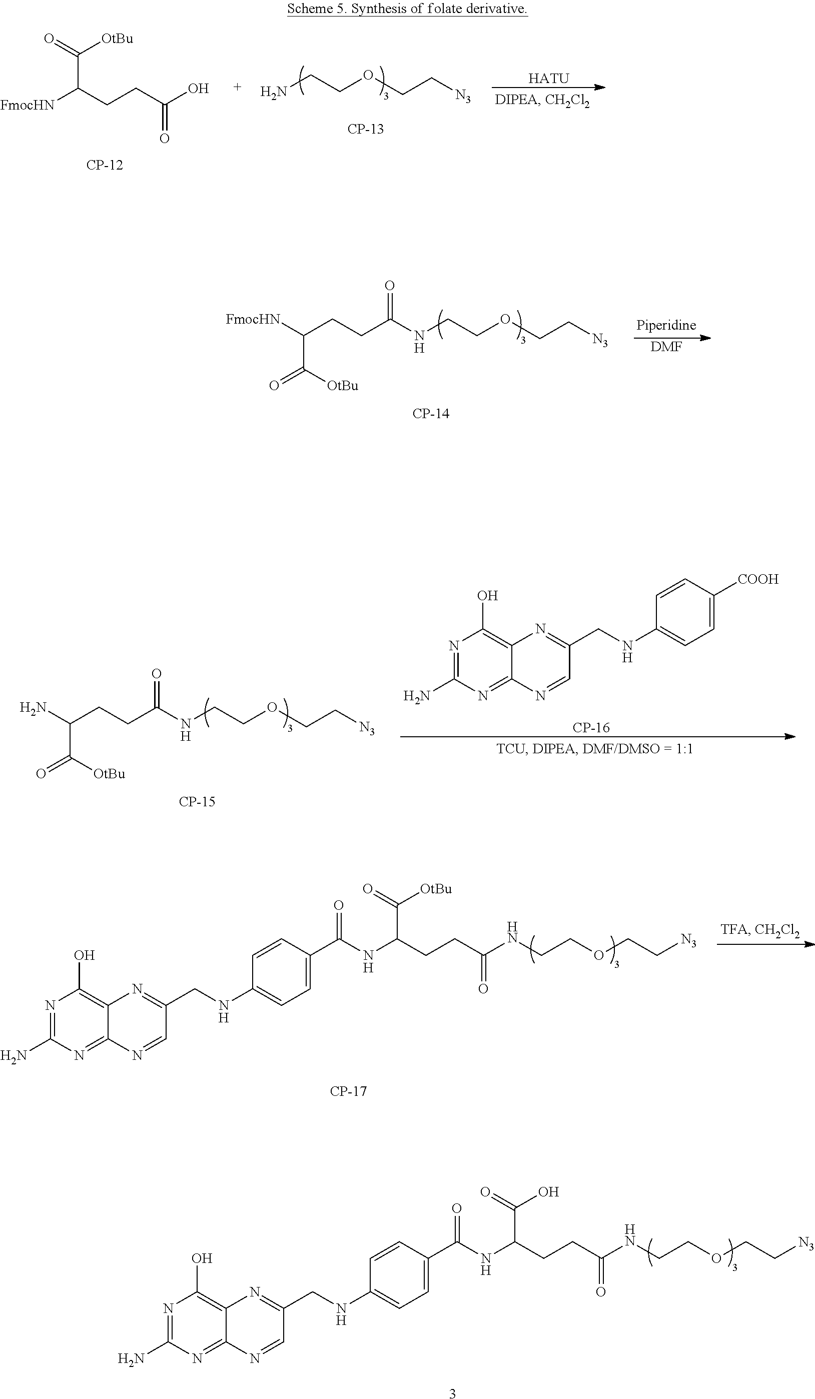

Synthesis of CP-14 (Scheme 5). To a solution of Fmoc-Glu-OtBu (CP-12) (212 mg, 0.5 mmol) in dry CH₂Cl₂ (10 mL), HATU (380.0 mg, 1.0 mmol) and DIPEA (130 mg, 1.0 mmol) were added. After 20 min stirring at room temperature, 11-Azido-3,6,9-trioxaundecan-1-amine (CP-13) (145.0 mg, 0.65 mmol) was added and the mixture was stirred at room temperature overnight. After completion of the reaction (monitored by TLC), the reaction mixture was concentrated under reduced pressure and purified by flash column chromatography (MeOH:CH₂Cl₂, 5:95). The product CP-14 was obtained as a yellowish viscous oil (280 mg, 90%). ¹H NMR (300 MHz, CDCl₃): δ 1.47 (s, 9H), 1.60 (m, 4H), 2.25 (m, 2H), 3.32-3.42 (m, 2H), 3.42-3.50 (m, 2H), 3.50-3.59 (m, 2H), 3.59-3.71 (m, 1OH), 4.18-4.28 (m, 2H), 4.33-4.46 (m, 2H), 5.68 (d, J=7.7 Hz, 1H), 7.29-7.36 (m, 2H), 7.36-7.46 (m, 2H), 7.62 (d, J=6.4 Hz, 2H), 7.78 (d, J=7.5 Hz, 2H).¹³C NMR (75 MHz, CDCl₃): δ 27.9, 28.6, 32.4, 39.2, 47.1, 50.6, 54.1, 66.9, 69.7, 69.9, 70.1, 70.4, 70.5, 70.6, 82.3, 119.9, 125.1, 127.0, 127.6, 141.2, 143.7, 143.9, 156.2, 171.1, 172.0. ESI-MS (m/z): calcd. For [M+H]⁺ 626.73, found: 626.67.

Synthesis of CP-15 (Scheme 5). Compound CP-14 (250 mg, 0.4 mmol) was dissolved in dry DMF (6.0 mL) and piperidine (6.9 mg, 0.08 mmol, 8 μL) was added. The reaction mixture stirred for 2 h at room temperature. After completion of the reaction, the reaction mixture was concentrated under reduced pressure. The crude mixture was purified by flash column chromatography (MeOH:CH₂Cl₂, 10:90). The amine product CP-15 was obtained as a colourless oil (120 mg, 75%). ¹H NMR (300 MHz, CDCl₃): δ 1.45 (s, 9H), 1.70-1.73 (m, 2H), 2.00-2.20 (m, 2H), 2.27-2.38 (m, 2H), 3.25-3.50 (m, 5H), 3.51-3.58 (m, 2H), 3.58-3.75 (m, 1OH), 5.30 (d, J=4.2 Hz, 2H), 6.53 (br. s, 1H). ESI-MS (m/z): calcd. For [M+H]⁺ 404.49, found: 404.52.

Synthesis of CP-17 (Scheme 5). To a suspension of pteroic acid (CP-16) (68 mg, 0.218 mmol) in a mixture of dry DMF/DMSO (1:1, 3.0 mL), TCTU (112 mg, 0.273 mmol) and DIPEA (35.4 mg, 0.273 mmol) were added. After 30 minutes stirring at room temperature, a solution of CP-15 (110 mg, 0.273 mmol) in dry DMF/DMSO (1:1, 4 mL) was added to the reaction vessel, and the mixture was stirred at room temperature for 12 h. After completion of the reaction (monitored by TLC), the reaction mixture was diluted with water (20 mL) and extracted with EtOAc (3×20 mL). The organic layers were combined and washed with saturated brine (2×20 mL). The crude product was purified by flash column chromatography (MeOH:CH₂Cl₂, 10:90). The pure product was obtained as a dark yellow solid (98 mg, 65%). ¹H NMR (300 MHz, DMSO-d₆): δ 1.23 (t, J=7.3 Hz, 2H), 1.38 (s, 9H) 1.75-2.06 (m, 2H) 2.06-2.27 (m, 2H) 3.17-3.67 (m, 16H), 4.10-4.27 (m, 1H) 4.48 (d, J=5.0 Hz, 2H) 6.63 (m, J=8.5 Hz, 2H) 6.97 (t, J=5.6 Hz, 1H) 7.64 (m, J=8.5 Hz, 2H) 7.93 (t, J=5.1 Hz, 1H) 8.20 (d, J=7.3 Hz, 1H) 8.64 (s, 1H). ESI-MS (m/z): calcd. For [M+H]⁺ 698.76, found 698.71.

Synthesis of 3 (Scheme 5). CP-17 (69.7 mg, 0.1 mmol) was dissolved in CHCl₃ (2.8 mL) and TFA (0.70 mL) and stirred at room temperature for 3 h. After completion of the deprotection, the solvents were evaporated under vacuum and the solid residue was washed with cold diethyl ether (3 mL×3). The crude compound was then dissolved in 3 mL ACN/H₂O (1:1), frozen with liquid nitrogen and lyophilized under high vacuum to afford compound 3 as a yellow fluffy powder (25 mg, 40%). ¹H NMR (500 MHz, DMSO-d₆): δ 1.85-1.95 (m, 1H) 1.99-2.08 (m, 1H), 2.13-2.26 (m, 2H), 3.17 (q, J=5.7 Hz, 2H), 3.33-3.40 (m, 3H), 3.43-3.63 (m, 11H), 4.22-4.31 (m, 1H), 4.50 (s, 2H), 6.64 (d, J=8.5 Hz, 2H), 7.15 (br. s., 2H), 7.65 (d, J=8.5 Hz, 2H), 7.88 (t, J=5.3 Hz, 1H), 8.19 (d, J=7.3 Hz, 1H), 8.66 (s, 1H). ¹³C NMR (125 MHz, DMSO-d₆): δ 26.5, 31.9, 38.5, 45.9, 50.0, 52.2, 69.1, 69.2, 69.5, 69.7, 69.7, 69.8, 111.0, 111.2, 121.4, 127.9, 128.9, 131.0, 148.4, 149.2, 150.7, 153.5, 159.8, 160.7, 166.2, 171.7, 173.8. ESI-MS (m/z): calcd. For [M+H]⁺: 642.65, found 642.64.

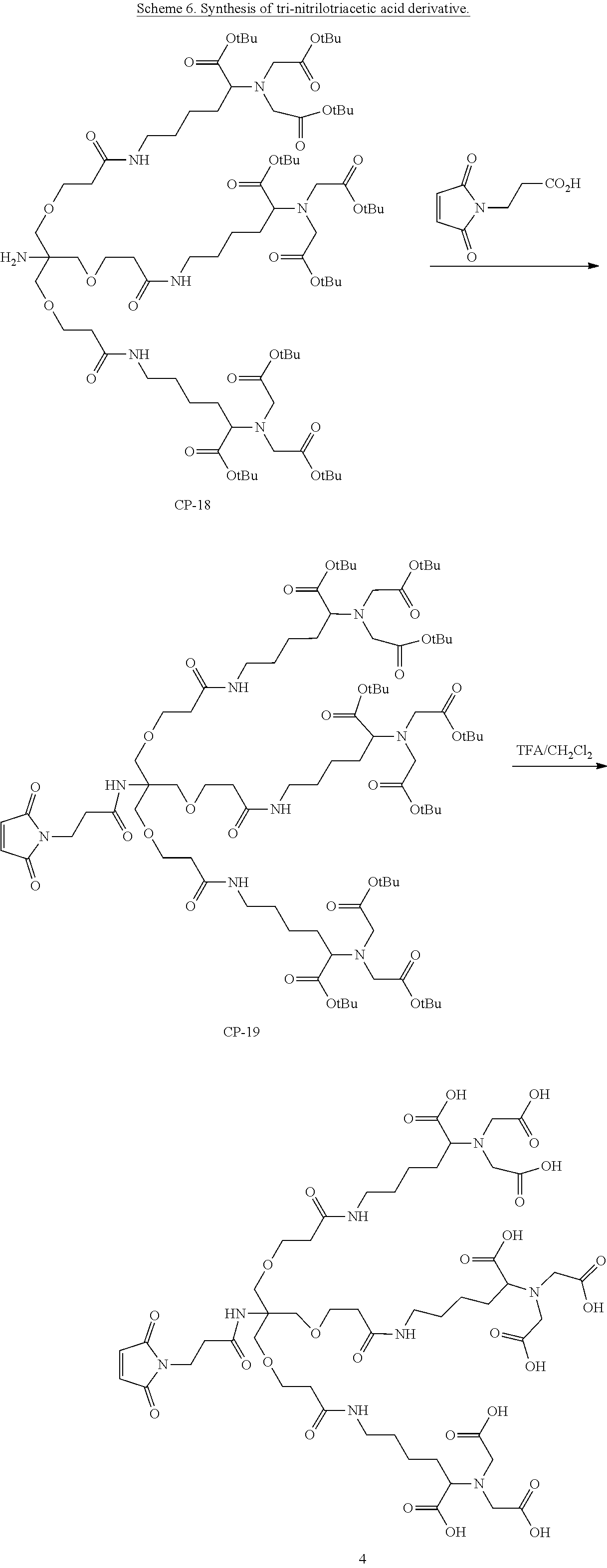

CP-18 was synthesized according to previously reported synthesis.

Synthesis of CP-19 (Scheme 6). EDC (22 mg, 0.118 mmol) and HOBt (7.8 mg, 0.058 mmol) were added to a stirred solution of 3-maleimidopropionic acid (20 mg, 0.118 mmol) in 10 mL dry THF under argon at 0° C. Then triethylamine (16 μL, 0.118 mmol) was added and pH of the resulting reaction mixture was adjusted to 7. After 15 min, CP-18 (140 mg, 0.09 mmol) was added and the reaction mixture was left to stir for 24 hours under argon at room temperature. After completion of the reaction, THF was removed under vacuum and the residue was purified by RP-HPLC to yield the product (95 mg, 61%). H NMR (400 MHz, DMSOd₆): δ 1.12-1.43 (m, 93H), 1.51 (m, 6H), 2.26 (t, J=6.35 Hz, 6H), 2.35 (t, J=7.48 Hz, 2H), 3.00 (m, 6H), 3.22 (t, J=7.41 Hz, 3H), 3.34 (d, J=17.13, 6H), 3.42 (d, J=17.17, 6H), 3.45-3.56 (m, 14H), 6.25 (br s, 1H), 6.99 (s, 2H), 7.24 (s, 1H), 7.78 (t, J=5.37 Hz, 3H); ¹³C NMR (100 MHz, DMSO-d6): δ 22.92, 27.77, 27.78, 28.90, 29.76, 33.95, 34.28, 35.95, 38.42, 53.28, 59.62, 64.59, 67.34, 68.20, 79.99, 80.31, 134.56, 169.85, 170.08, 170.70, 171.59, HRMS-ESI⁺(m/z): calcd. for [M+H]⁺ 1726.0468; found 1726.0532; calcd. for [M+Na]₊1748.0348; found 1748.0352.

Synthesis of Compound 4 (Scheme 6). The tert-butyl ester groups were deprotected by adding TFA (1.00 mL) to a solution of CP-19 (80.0 mg, 0.046 mmol) in DCM (2 mL) at 0° C. The reaction mixture was warmed up to the room temperature and stirring was continued for another 3.5 h. After the reaction was completed, DCM and TFA were evaporated. The traces of TFA were removed by co-evaporation with DCM. The crude compound was washed twice with cold diethyl ether, followed by dissolution in 3 mL ACN/H₂O (1:1), freezing with liquid nitrogen, and lyophilization under high vacuum to afford an off-white powder as product (48 mg, 85%). ¹H NMR (300 MHz, DMSOd₆)Q D Q 1.38 (br. s., 12H) 1.44-1.76 (m, 6H) 2.27 (t, J=5.7 Hz, 6H) 2.36 (t, J=7.3 Hz, 2H) 2.93-3.09 (m, 6H) 3.40 (t, J=7.0 Hz, 3H) 3.44-3.65 (m, 26H) 6.98 (s, 2H) 7.25 (s, 1H) 7.81 (br. s., 3H);¹³CNMR (100 MHz, D₂O): δ 23.01, 26.73, 28.02, 34.21, 34.90, 36.13, 38.79, 54.34, 60.12, 66.97, 67.48, 68.49, 134.44, 169.94, 171.37, 172.54, 172.99, 173.95; HRMS-ESI⁺(m/z): calcd. for [M+H]⁺ 1221.4902; found 1221.4898, calcd. for [M+Na]⁺1243.4735; found 1243.4718, calcd. for [M+K]⁺1259.4432; found 1259.4457.

### Example 18—Experimental Details—General Procedure for Synthesis of ODN-Ligand Conjugates

ODN-ligand conjugates (Scheme 2). 150 nmol alkyne modified oligodeoxynucleotide (Cy5-ODN₂ (Compound 220), FAM-ODN₂ (Compound 230), or TAMRA-ODN₂ (Compound 240), or ODN_{2b}) was dissolved in 160 μL MQ water, followed by the addition of ascorbic acid (20 μL, 0.9 μmol), TEAA buffer (40 μL, 2M, pH=7), and Cu-TBTA (80 μL, 0.9 μmol), the system was purged with argon and finally 1.5 μmol azide modified ligand (1, 2, or 3) in DMSO (200 μL) was added. The mixture was purged with argon again, sealed and placed on a shaker for 18 h. The progress of the reaction was monitored by analytical RP-HPLC. After completion of reaction, the crude was purified by RP-HPLC and characterized by ESI-MS. ESI (m/z): Cy5-ODN₂-An (Compound 209/210): calcd. [M+Na+]⁺9589.93; found 9589.66, TAMRA-ODN₂-Fol (Compound 213/214): calcd. [M−H⁺]⁻ 9839.0688; found 9839.0791, FAM-ODN₂-GLA (Compound 211/212): calcd. [M+K⁺]⁺9748.86; found 9748.45, ODN_{2b}-GLA (Compound 208): calcd. [M−H⁺]⁻ 12218.3678; found 12218.3818.

### Synthesis of tri-NTA-ODN₁(Compound 105)

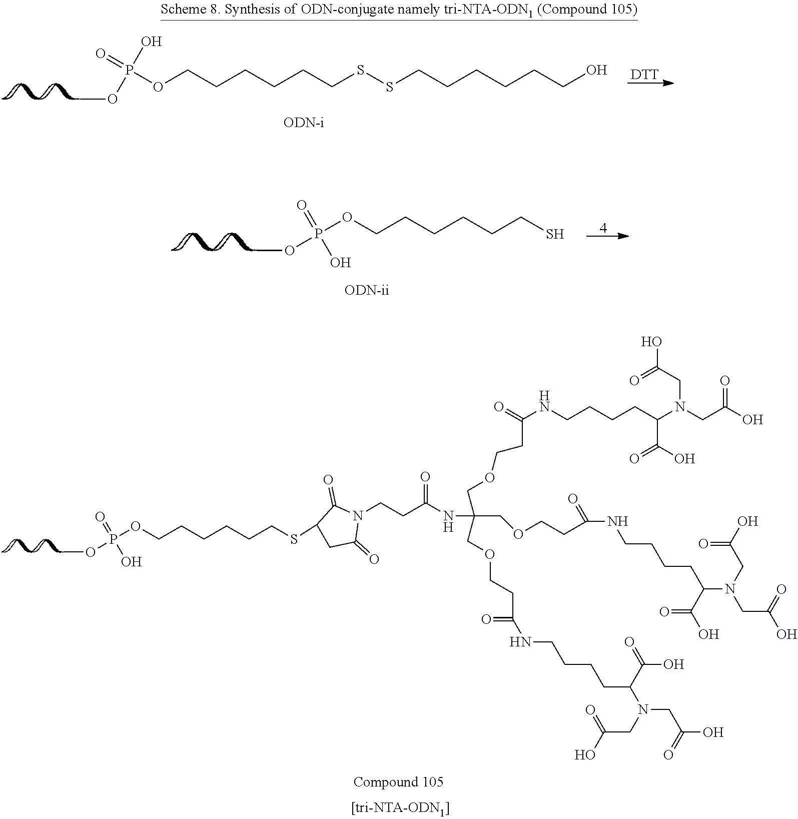

Tri-NTA-ODN₁ (Compound 105) (Scheme 8). DTT (10 μL, 1.0 M solution in water) was added to a solution of ODN-i (200 nmol) in 200 μL Tris buffer (50 mM, pH 8.3) and stirred for 1 hour. The reduced oligodeoxynucleotide (ODN-ii) was then desalted on a Sephadex™ G-25 column and dried under reduced pressure. ODN-ii was added to a solution of 4 (8 mg) in concentrated PBS x 10, pH 7. The reaction was stirred overnight, and then the product was purified using RP-HPLC. MALDI-TOF MS (m/z): ESI-MS (m/z): calcd. For [M+H⁺]⁺: 8877.14; found 8877.25.

### Example 19—Experimental Details—Biological Methods

### Bacterial Strain and Growth Conditions

E. coli K-12 strain KRX (Promega) was used for OmpC expression. The details of the expression of 3 copies of hexahistidine-tag at the 7^{th} loop of the OmpC have been described in our previously published paper.[2] Transformed bacteria with different OmpC constructs (OmpC or His-OmpC) were cultured to saturation in LB medium supplemented with ampicillin (100 μg/mL) at 30° C. Next, pre-cultured cells were diluted 1:100 in fresh LB medium supplemented with ampicillin, and incubated at 30° C. Once the cells reached the mid-exponential phase (OD₆₀₀ ≈0.6), protein expression was induced by the addition of 0.1% Rhamnose and 20 μM isopropyl-b-D-1-thiogalactopyranoside (IPTG) and incubated at 30° C. while shaking for ˜ 18 h. Then, the bacterial cells were harvested by centrifugation at 6,000 g for 4 min.

### Preparation of B-Probes and their Fluorescence Imaging

The bacterial cells were collected by centrifugation at 6,000 g for 4 min. Pellets were washed twice with PBS×1 buffer and re-suspended in the same buffer to an OD₆₀₀ of 0.3. To a 100 μL sample of the bacteria suspension, a pre-incubated sample of DNA (100 nM) and NiCl₂ (500 nM) was added, and the cells were incubated at room temperature for 1 h. Then the sample was washed twice with PBS, re-suspended in 200 μL PBS, and placed on a glass-bottom dish (P35G-1.5-14-C; MatTek) pre-coated with poly-L-lysine (Sigma Aldrich) and left to adhere for 1 h. Finally, the wells were vigorously washed with PBS three times and imaged using an Olympus IX51 fluorescent microscope. The samples were imaged using 100× objective lens.

### Fluorescence Emission Measurements of Various ODNs

Samples of ODN duplexes 3-6 or constructs 1-2 (20 μM) and NiCl₂.6H₂O (100 μM) were mixed in PBS buffer (pH=7.2) and allowed to stand at room temperature for 30 minutes. Then each duplex was diluted in PBS to a final concentration of 100 nM, 60 uL each was added to the black flat bottom coming 384 well plate and the emission spectra was immediately recorded. The fluorescence responses were measured using the excitation wavelength of 495 nm. These experiments were performed in triplicates.

### Quantification of the Amount of DNA Duplex Decorating the Bacterial Cell

To a 100 μl sample of the bacteria suspension in PBS buffer, a pre-incubated sample of duplex 1 (500 nM) and NiCl₂ (2.5 uM) was added, and the bacteria were incubated at room temperature for 1 h. Cells were collected by centrifugation at 6,000 g for 4 min and then the upper-layer supernatant was transferred to a new tube. The absorbance of the supernatant layer was measured and subtracted from the absorption spectra of duplex 1 (500 nM) in PBS buffer. The subtracted value obtained (˜50 nM) represents the amount of DNA actually bound to the bacterial surface. Concentrations of DNA were estimated based on the absorption band of TAMRA at 560 nm and using the molar extinction coefficient of the TAMRA at this wavelength (89000 M⁻¹ cm−1).

### Fluorescence Imaging of Bacteria-Cell Interaction

Cells (15,000 cells/well) were seeded onto glass bottom culture dishes (MatTek) and allowed to adhere overnight. MDA-MB-435 cells were seeded in MEM medium supplemented with 5% charcoal dextran-stripped calf serum, 1% non-essential amino acids, 1% L-glutamine, and 1% penicillin/streptomycin. KB cells were plated in folate-depleted RPMI supplemented with 10% FBS and 1% penicillin/streptomycin while LNCaP were maintained in RPMI, 10% FBS, 1% L-glutamine, and 1% penicillin/streptomycin. Following day, medium was removed, rinsed thrice with PBS and then the cells were incubated with B-probes 1-4 (prepared by incubating His-bacteria with duplexes 1-4 at 500 nM concentration for 1 h at 25° C. followed by washing with PBS buffer) at 37° C., 5% CO₂ in incubator for 20 min. Finally, the excess/unbound bacteria were gently washed off with PBS containing Ca⁺² and Mg⁺² (200 μL×3) and the cells were imaged using a fluorescence microscope with 60× objective lens. Negative control experiments were performed similarly using bacteria decorated with a duplexes generated from tri-NTA-ODN₁ (Compound 105) and TAMRA-ODN₂ (Compound 240), Cy5-ODN₂ (Compound 220) or FAM-ODN₂ (Compound 230).

### Displacement Experiment with Small Molecule that Disrupt the Bacteria-Cell Interaction

KB Cells maintained in folate-depleted RPMI supplemented with 10% FBS and 1% penicillin/streptomycin were seeded (15,000 cells/well) onto glass bottom culture dishes (MatTek) and allowed to attach overnight. The next day, medium was removed, and cells were rinsed with PBS (200 μL×3) followed by the incubation of cells with 1 μM of commercial folic acid in 200 μL PBS (taken from a stock of 5 mM in DMSO) for 15 min.³ at 37° C., 5% CO₂.Next, the folic acid solution was pipetted out and the resulting KB cells were incubated with B-probe 1 (prepared according to the procedure described above) for 20 min. Finally, the excess/unbound bacteria were gently washed off with PBS containing Ca⁺² and Mg⁺² (3×200 μL) and the cells were imaged using a fluorescence microscope with 60×objective lens.

### Fluorescence Imaging of Different Cancer Cells Interacting with B-Probe Mixtures

MDA-MB-435 cells, LNCaP and KB cells were maintained in their respective media as mentioned in the previous section. Cells (15,000 cells/well) were seeded onto glass bottom culture dishes (MatTek) and allowed to adhere overnight. The medium was removed from the cells and rinsed three times with PBS. On the other hand, B-probes 1-3 (prepared as described in the earlier section) were mixed (3×200 μL). A mixture of this bacterial sample (200 μL) was incubated separately with each cell line at 37° C. under 5% CO₂ condition. After 20 minutes, the unbound bacteria were gently washed off with PBS containing Ca⁺² and Mg⁺² (200 μL×3) and the cells were imaged using a fluorescence microscope and a 60×objective lens.

### Immunofluorescence Procedure

The cells LNCaP, KB and MDA-MB-435 were counted (30,000 cells/well) and seeded on glass-bottom dish (P35G-1.5-14-C; MatTek). LNCaP and KB cells were fixed with 4% PFA for 10 min. and blocked for 1 h at 37 C with blocking buffer (2% fetal bovine serum in PBS containing). Alexa Fluor 488 anti-human PSMA (BLG-342505, BioLegend) or PE anti-FOLR1 (BLG-908303, BioLegend) were incubated with the LNCaP and KB cells respectively following the standard procedures in 1:200 dilution in PBS at 0 C for 30 minutes. MDA-MB-435 cells, were fixed with 4% PFA for 10 min., permeabilized using 0.01% triton for 2 min., blocked for 1 h at 37 C with blocking buffer (2% fetal bovine serum in PBS). Followed by incubation with Alexa Fluor 647 anti-sigma receptor F-5 antibody (sc-166392 AF647, Santa Cruz Biotechnology Inc.), incubated at 1:200 dilution in PBS at 0 C for 30 minutes. Then, all the three antibody stained cell samples were washed twice with 200 μL of PBS and imaged using an Olympus IX51 fluorescent microscope using 60× magnification at ex/em of 470-495/510-550 nm, 530-550/590 or 620-660/700-775 for imaging LNCaP, KB and MDA-MB-435 cells, respectively.

### Fluorescence Imaging to Study Internalization of B-Probe into Cancer Cells

LNCaP cells were cultured as described above in RPMI medium containing 10% FBS, 1% L-glutamine, and 1% penicillin/streptomycin. The cells were plated at 20,000 cells/well in an eight-well chamber slide (Cellvis Cat #C8-1.5H—N, CA, USA) and transfected 24 h later with mCherry-CaaX Hras using Lipofectamine 2000 (Life Technologies, Carlsbad, CA) according to the manufacturer's guidelines. Next day, the transfected LNCaP cells were washed twice with PBS (300 μL), and B-probe 3 or 4 was added to the cells. Z-stacks (0.31 μm) of selected cells expressing mCherry-CaaX were acquired on the Carl Zeiss Ltd., cell discoverer 7 microscope (CD7). A Plan-Apochromat 50×/1.2NA objective with 2×tube lens (effective magnification of 100×) was used for image acquisition, and detection was done on a 14 bit Axiocam 702 CMOS camera (Carl Zeiss Ltd.). Imaging was performed using a combination of two LED modules simultaneously: 470 and 590 nm wavelengths with bandpass emission filters: 412-433; 501-547 and 617-758. The CD7 chamber was set to 37° C. with injected 5% CO₂. Images acquired from CD7 microscope were deconvolved using ZEN Blue 3.1 software using the “excellent slow constrained iterative algorithm” method. 3D-deconvolved images were then segmented and visualized in Imaris 9.8 (Bitplane) software. The bacteria and the membrane were segmented using the Imaris 3D surfaces module. After the segmentation, the membrane mask was shown once as transparent and once as opaque at the same image to show the bacterial entry into cell.

### Tissue Sample Preparation and Staining

All animal experiments were conducted in accordance with approved institutional animal care and use committee (IACUC) protocols. CD1-nude mice were purchased from Envigo RMS (Israel) Ltd. Mice were housed and handled in a specific-pathogen-free, temperature-controlled (22° C.±1° C.) mouse facility on a reverse 12/12 h light/dark cycle, Animals were fed a regular chow diet et libitum. MDA-MB-435 cells (5×10⁶ per mouse) were subcutaneously injected in the right flank of 6 week-old female CD1 nude mice. Once tumour reached a volume of approximately 750 mm³, mice were euthanized and tumours were extracted for analysis. After fixation with 4% PFA, the tissues were processed to paraffin embedded blocks, and 6 μm sections were taken for immunohistochemistry or bacteria/DNA staining. Before proceeding with immunohistochemistry or addition of bacteria, sections were de-paraffinized in xylene and treated with decreasing concentrations of ethanol. Post-fixation was done using ice-cold acetone for 7 min, and heat mediated antigen retrieval was performed in Tris/EDTA buffer (pH 9) for 10 min. All the sections were blocked using 20% normal horse serum (Vector Laboratories, CA, USA) and 0.2% Triton-X, for 90 min. After staining, the samples were imaged with Leica Mi8 microscope equipped with a motorized stage and a Leica DFC365 FX camera. Single×20 magnification images were tiled to receive a full scan of the tumor section.

### Fluorescence Immunohistochemistry

Sections were incubated with Alexa 647 conjugated sigma receptor (sc-166392 AF647, Santa Cruz Biotechnology, 1:50) or Alexa Fluor 647 conjugated normal mouse IgG2b (sc-24638, Santa Cruz Biotechnology, 1:50) at room temperature overnight under dark humidified chamber. Sections were washed 3 times with PBS for 5 min, covered using aqua-poly/mount coverslip (cat. no. 18606-20, Polysciences Inc.) and imaged with Leica Mi8 microscope equipped with a motorized stage and a Leica DFC365 FX camera. Single×20 magnification images were tiled to receive a full scan of the tumor section.

### Tissue Specimen Staining with Bacterial Probes

PAP pen (Cat. No. H-4000, Vector Laboratories, CA, USA) was used to create a hydrophobic boundary around tissue sections. The tissue sections were separately incubated with 200 μL of the following samples: B-probe 2 and control B-probe (prepared by decorating His-bacteria with duplex tri-NTA-ODN₁:Cy5-ODN₂)(Compound 105:Compound 220) for 1 h. After washing off the unbound E. coli with PBS buffer, the sections were covered using aqua-poly/mount coverslip (cat. no. 18606-20, Polysciences Inc.) and imaged.

### Tissue Specimen Staining with Duplex 2

Sections were incubated with duplex 2 (50 nM with 250 nM of Ni⁺²) at room temperature for 1 h in the dark. Samples were then washed 3 times with PBS for 5 min each, covered using aqua-poly/mount coverslip and imaged with Leica Mi8 microscope.

### Gel Electrophoresis for Analysing the Constructs 1 and 2

The 2% agarose gels were prepared by mixing 2% (w/v) of agarose in 50 mL Tris/Borate/EDTA (TBE). The mixture was heated in microwave until a clear solution was obtained. To the agarose gel, 2 drops of ethidium bromide was added and the solution was poured in the mould and allowed to solidify by cooling. Individual samples of ODNs to be tested were prepared in Tris buffer (12 mM, pH 7.5, 137.5 mM LiCl) at a final concentration of 12 μM each. For self-assembly, the ODN strands were mixed in equal molar ratios in tris buffer at a final concentration of 12 μM, heated at 95° C. for 4 minutes followed by gradual cooling to room temperature. The 12 μM samples in a volume of 10 μL was mixed with 2 μL loading dye (6×) such that final concentration of samples is 10 μM each for running the gel. Electrophoresis was carried out at 100 V for 90 minutes using a Mini-PROTEAN Tetracell system (Bio-Rad, CA). Gels were visualized with the ChemiDoc XRS imaging system (Bio-Rad, CA).

1. A DNA construct comprising:
a. a first compound comprising a first oligonucleotide (ODN-1) covalently bound to a His-tag specific binder, either directly or through a first linker;
b. a second compound comprising a second oligonucleotide (ODN-2) covalently bound to a synthetic agent, either directly or through a second linker, wherein said second oligonucleotide is complementary to said first oligonucleotide, and wherein said second oligonucleotide comprises a first hanging strand (a first toehold region), and
c. a third compound comprising a DNA duplex (dsDNA) appended with a second hanging strand complementary to said first hanging strand, and further appended with at least two fluorescent dyes.

2. The construct of claim 1,
wherein the first compound is bound to the second compound through hybridization of ODN-1 and ODN-2, and the third compound is bound to the second compound through hybridization of the first hanging strand and the second hanging strand;
wherein the His-tag specific binder is capable of binding to an affinity tag comprising a poly-histidine peptide;
wherein said ODN-1 is 5-100 bases long;
wherein said second oligonucleotide (ODN-2) is longer than said first oligonucleotide (ODN-1),
wherein the first and/or the second hanging strand comprises between 5-50 oligonucleotides;
wherein said synthetic agent of said second compound is bound to the 3′ end or to the 5′ end of said second oligonucleotide,
wherein said synthetic agent of said second compound is a chemical or a biological moiety;
wherein said synthetic agent of said second compound is naturally occurring or a synthetic compound;
wherein said synthetic agent of said second compound comprises a cancer cell binder, a CSP binder, a protein binder, a protein ligand, an anticancer agent, a growth factor, an angiogenic factor, a cytokine, a hormone, a DNA molecule, a siRNA molecule, an oligosaccharide, a protein receptor, an immune activator, an immune suppressor, an antibody, a small molecule, a drug, or a derivative thereof,
wherein said synthetic agent of said second compound can interact with a specific CSP on a cancer cell,
wherein said DNA duplex (dsDNA) comprises at least 4 fluorescent dyes;
wherein said fluorescent dyes of said DNA duplex (dsDNA) are located 4-6 bases apart from each other;
said fluorescent dyes of said DNA duplex (dsDNA) are located on the longer oligonucleotide strand that comprises the second hanging strand (ssDNA-long),
or any combination thereof.

3. The construct of claim 2,
wherein said ODN-1 is 5-25 bases long;
wherein said synthetic agent of said second compound comprises a CSP binder, a cancer cell binder, or a protein binder,
preferably wherein said CSP binder, a cancer cell binder, or a protein binder comprises a biotin, a folate, an anisamide, or a glutamate urea,
wherein said CSP of said CSP binder is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof,
wherein said cancer cell of said cancer cell binder, is KB cell (cervical cancer cell), MDA-MB-435 (melanoma cell), or LNCaP (prostate cancer cell);
wherein the first and/or the second hanging strand comprises between 10-20 oligonucleotides (e.g., 16);
wherein ssDNA-long comprises between 10-50 oligonucleotides including the second hanging strand (e.g., 45);
or any combination thereof.

4. The construct of claim 1,
wherein said His-tag specific binder comprises a moiety represented by the structure of formula E: 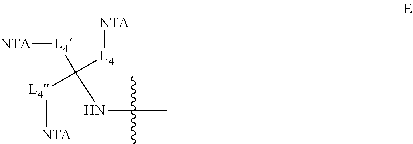
wherein
L₄, L₄′, and L₄″ are each independently a substituted or unsubstituted linear or branched alkyl chain of 1-20 carbon atoms, substituted or unsubstituted linear or branched alkyl ether chain of 1-20 carbon atoms, substituted or unsubstituted linear or branched alkyl phosphate chain of 1-20 carbon atoms, substituted or unsubstituted linear or branched alkyl amide chain of 1-20 carbon atoms, substituted or unsubstituted linear or branched alkyl diamide chain of 1-20 carbon atoms, substituted or unsubstituted linear or branched alkyl amine chain of 1-20 carbon atoms or any combination thereof,
formula E(a): 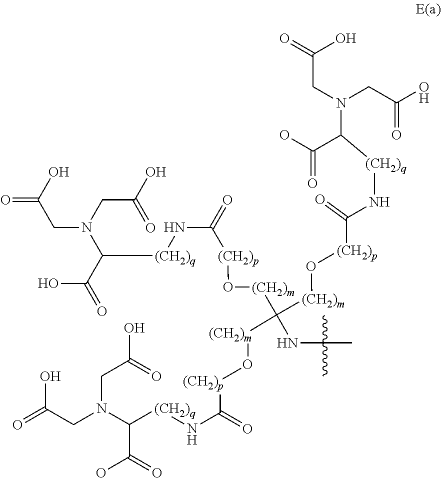
wherein
m, p and q are each independently an integer number between 1 and 8; or formula E(b):

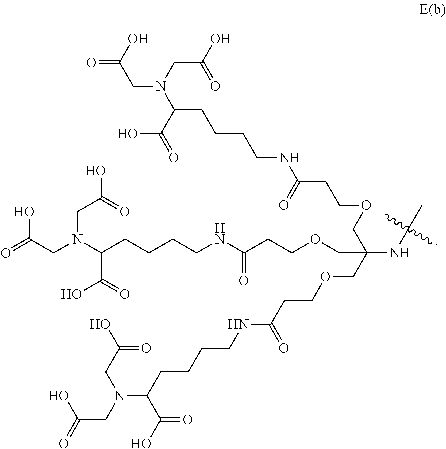 [SMILES: *NC(COCCC(=O)NCCCCC(C(=O)O)N(CC(=O)O)CC(=O)O)(COCCC(=O)NCCCCC(C(=O)O)N(CC(=O)O)CC(=O)O)COCCC(=O)NCCCCC(C(=O)O)N(CC(=O)O)CC(=O)O]

5. The construct of claim 1,
wherein said first linker comprises at least one oligoethyleneglycol (OEG) moiety, at least one phosphate moiety, at least one thioalkyl moiety or any combination thereof;
said fist linker comprises the following monomer:
—[(CH₂O)ₖ—PO₃H]ₗ—; or
said first linker is represented by the following formula:
—[(CH₂O)ₖ—PO₃H]ₗ—(CH₂)_{w}—S—
wherein
k and l are each independently an integer number between 0 and 10; and
w is an integer number between 1 and 10.

6. The construct of claim 1, wherein said fluorescent dyes are selected from a group comprising:
dansyl, fluorescein (6-FAM), FAM, cyanine dyes (e.g. Cy3, Cy5, Cy7, etc), sulfoindocyanine, nile red, Rhodamine dyes (e.g., Rhodamine 123, Rhodamine Red-X, etc.), perylene, fluorenyl, coumarin, 7-methoxycoumarin (Mca), dabcyl, NBD, Nile blue, TAMRA, BODIPY dyes, FITC (Fluorescein isothiocyanate), Thiazole orange, Quinoline blue, Thiazole red, phycoerythrin (PE), Acridine Orange, Alexa Fluor dyes (e.g., Alexa Fluor 488, Alexa Fluor 555, Alexa Fluor 647, etc.), Cascade Blue, DAPI (4′,6-diamidino-2-phenylindole), DiI (1,1′-Dioctadecyl-3,3,3′,3′-tetramethylindocarbocyanine perchlorate), Ethidium Bromide, GFP (Green Fluorescent Protein), Hoechst dyes (e.g., Hoechst 33342, Hoechst 33258, etc.), Indo-1, Lucifer Yellow, MitoTracker dyes (e.g., MitoTracker Green, MitoTracker Red, etc.), Oregon Green, Propidium Iodide, SYBR Green, Texas Red, YOYO-1, ZsGreen or derivative thereof.

7. The construct of claim 1, wherein said first compound is represented by the structure of the nickel complexes of the following compounds:

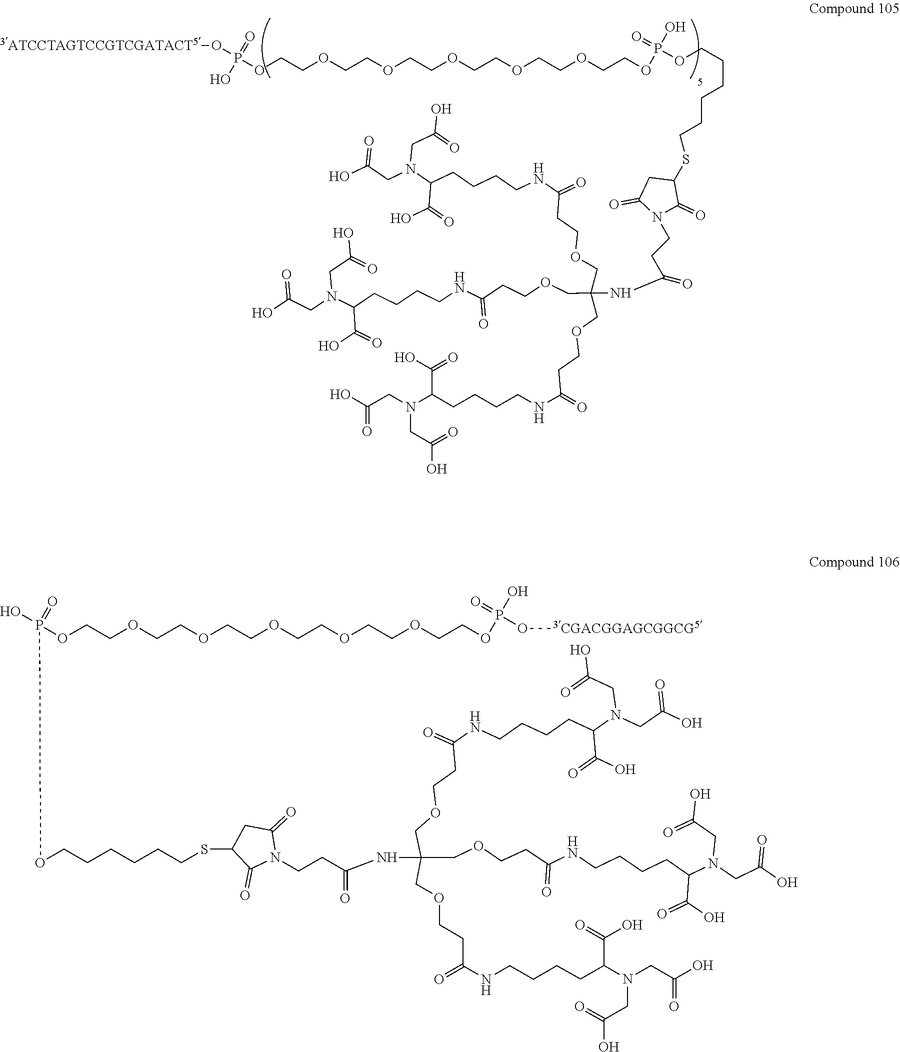

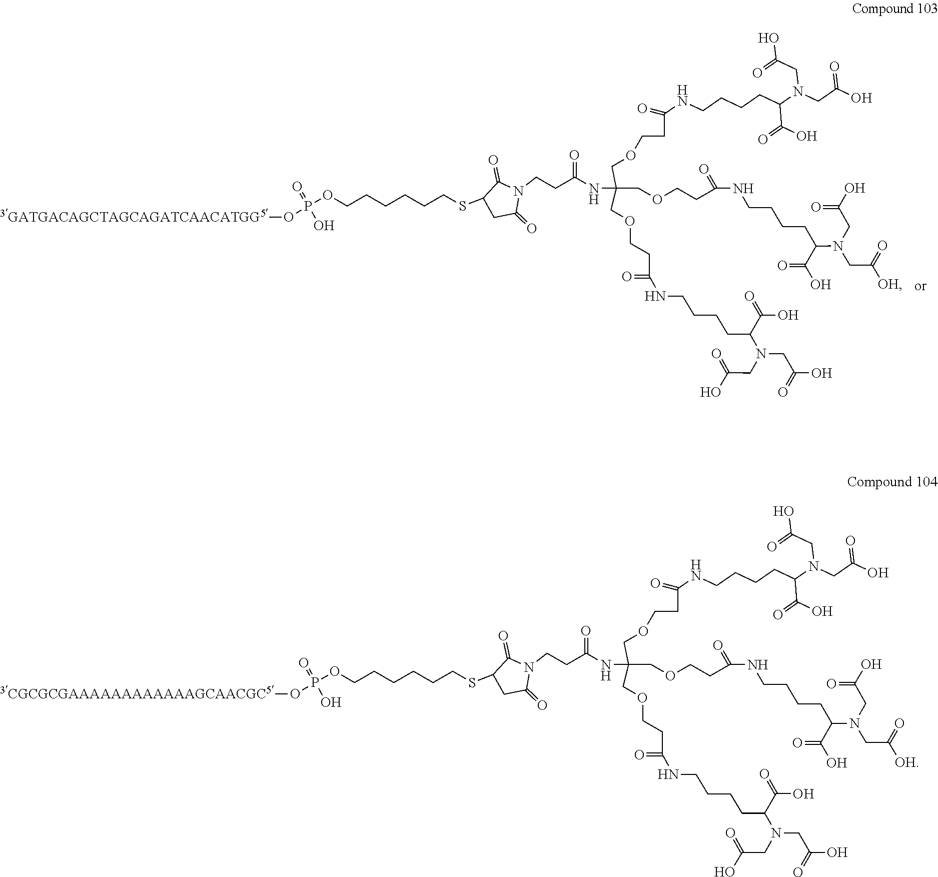

8. The construct of claim 1, wherein the second compound is represented by the structure of the following compounds:

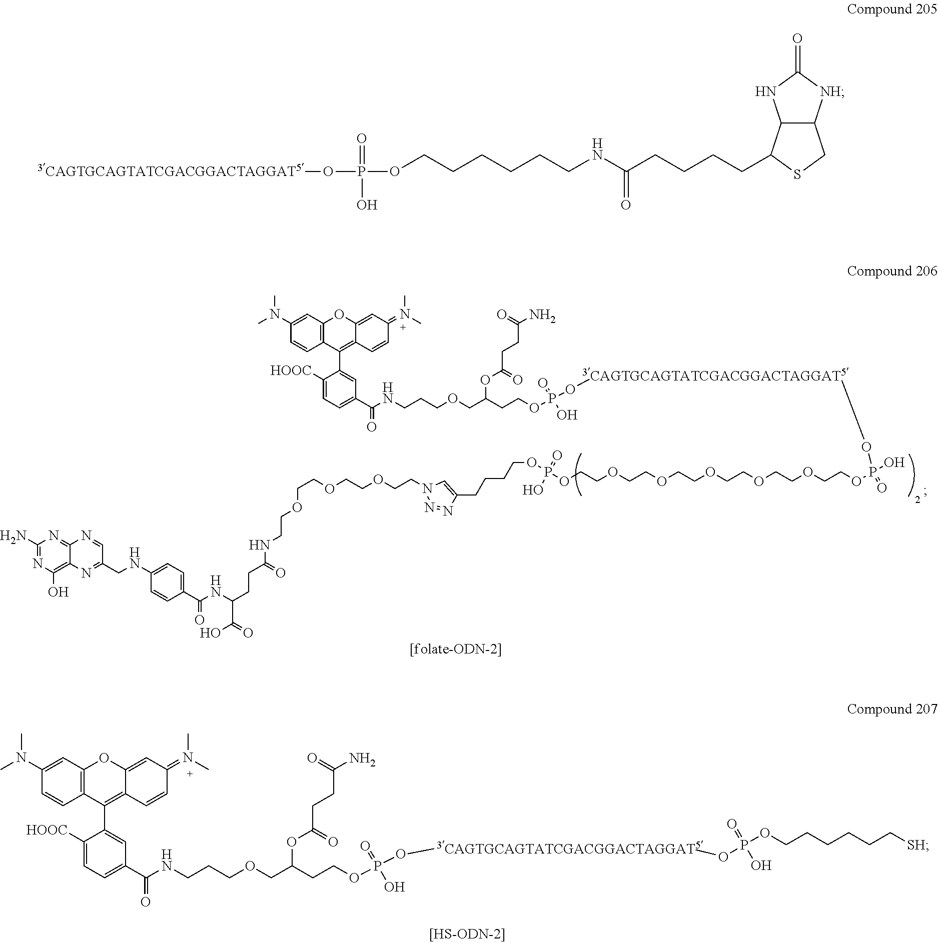

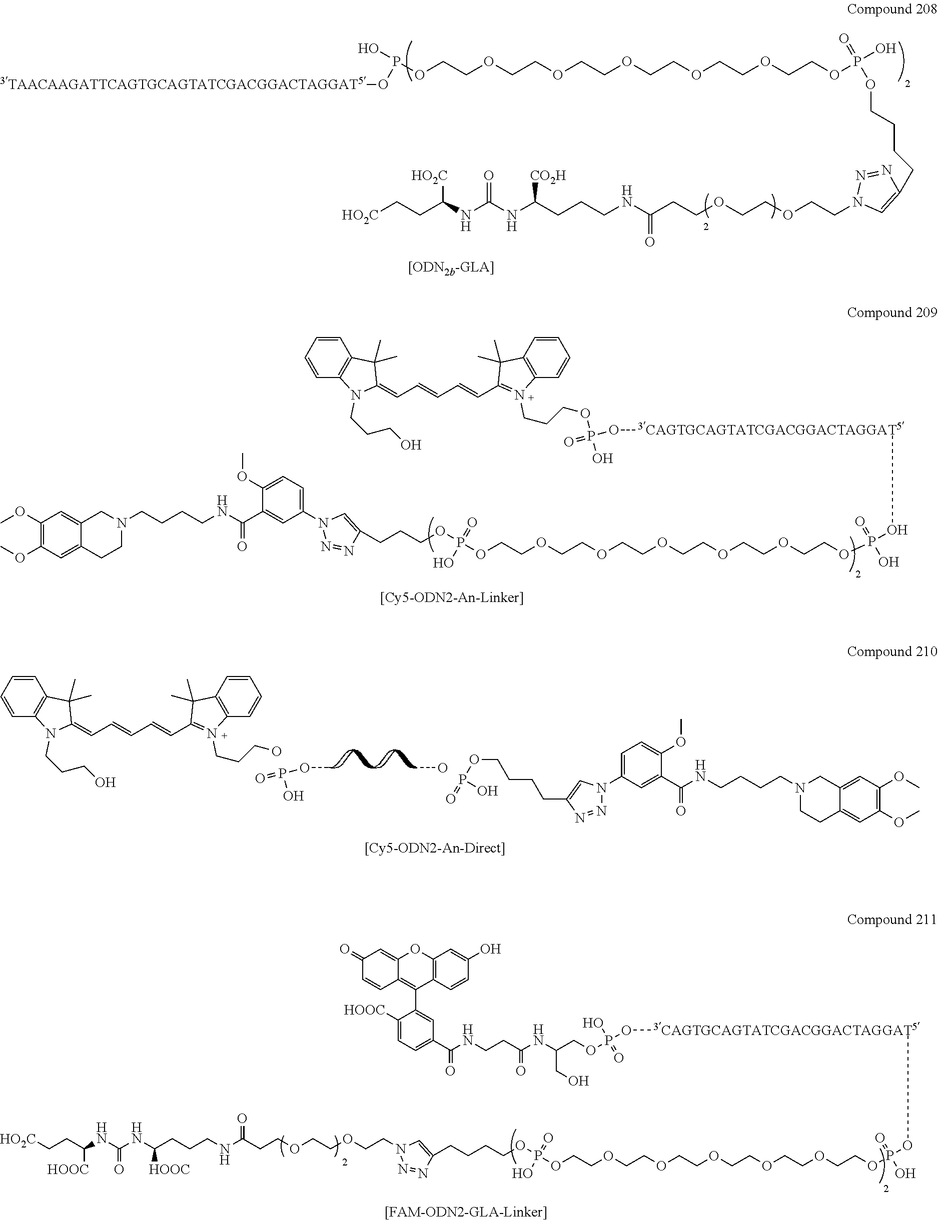

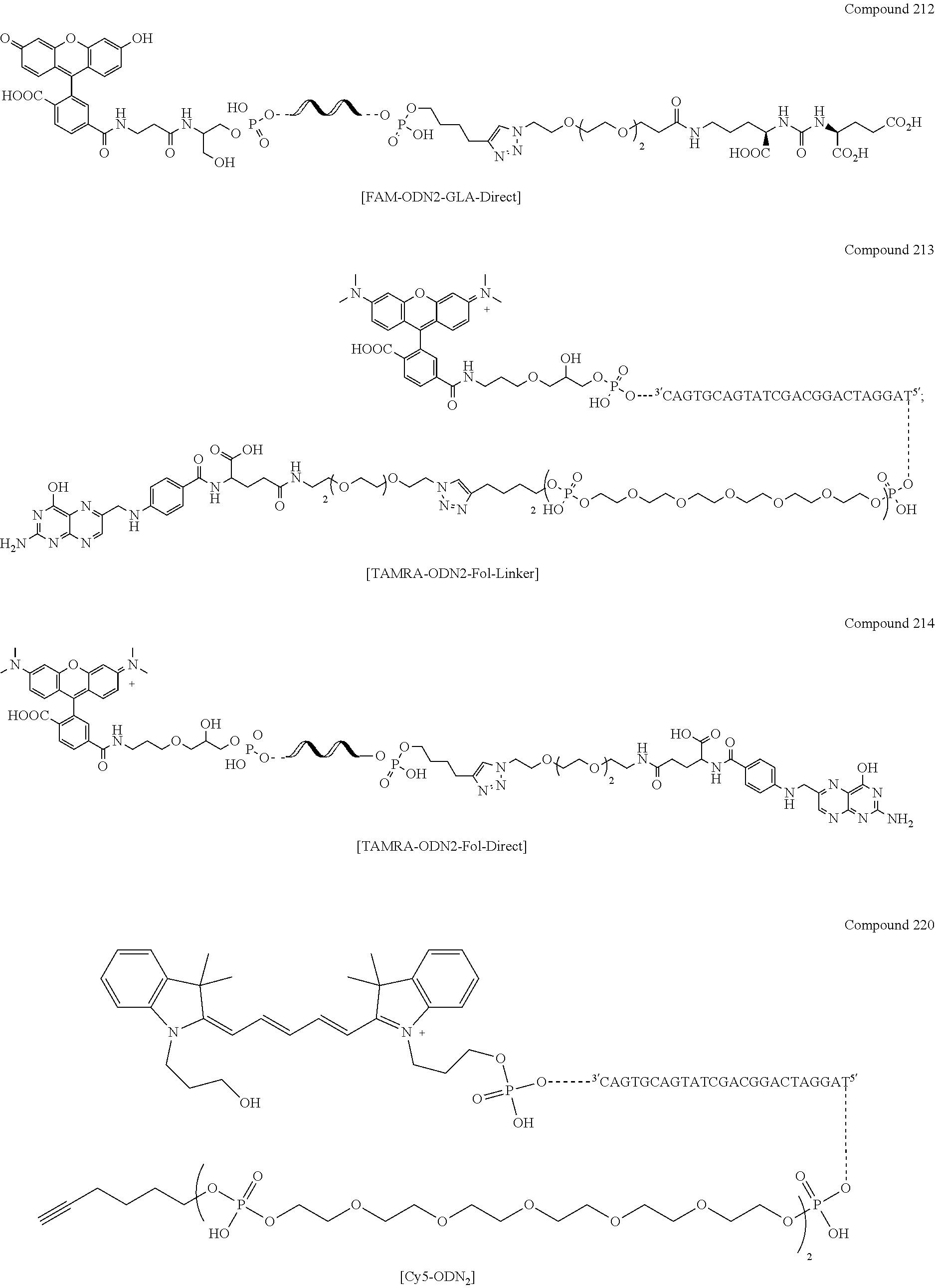

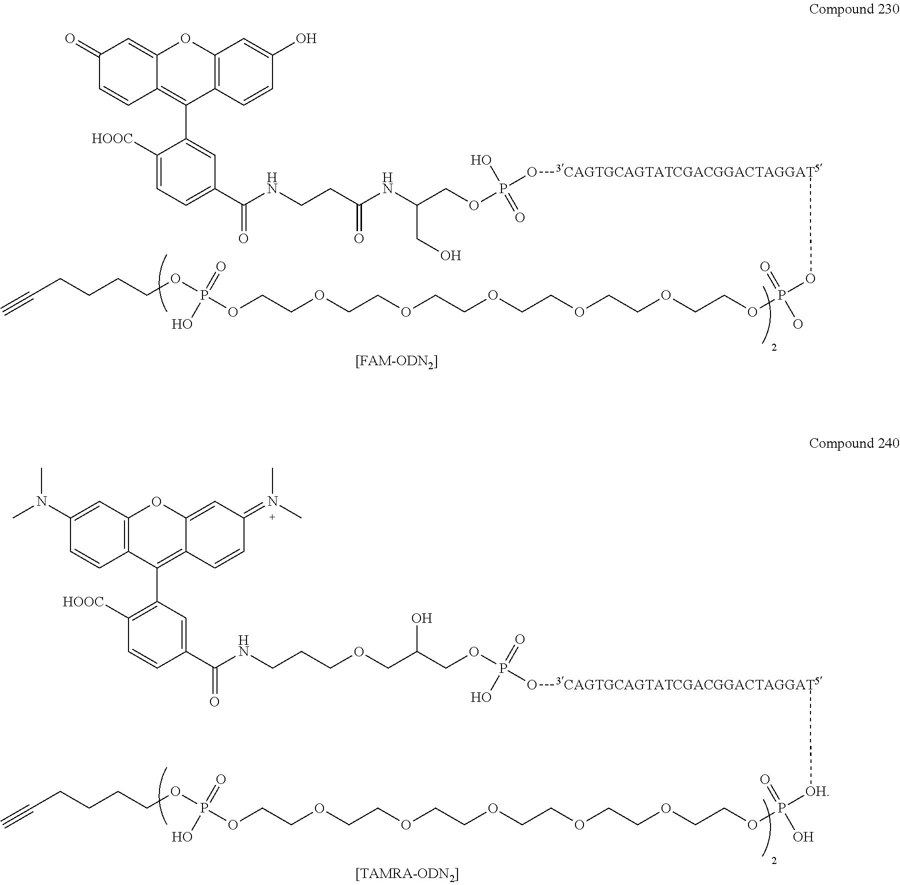

9. The construct of claim 1, wherein the DNA duplex (dsDNA) comprises a longer oligonucleotide strand (ssDNA-long) represented by a sequence comprising at least 80% homology to SEQ ID NO. 22 or 23;
the DNA duplex (dsDNA) comprises a shorter oligonucleotide strand (ssDNA-short) represented by a sequence comprising at least 80% homology to SEQ ID NO.: 24;
or combination thereof.

10. The construct of claim 1, comprising the following compounds:

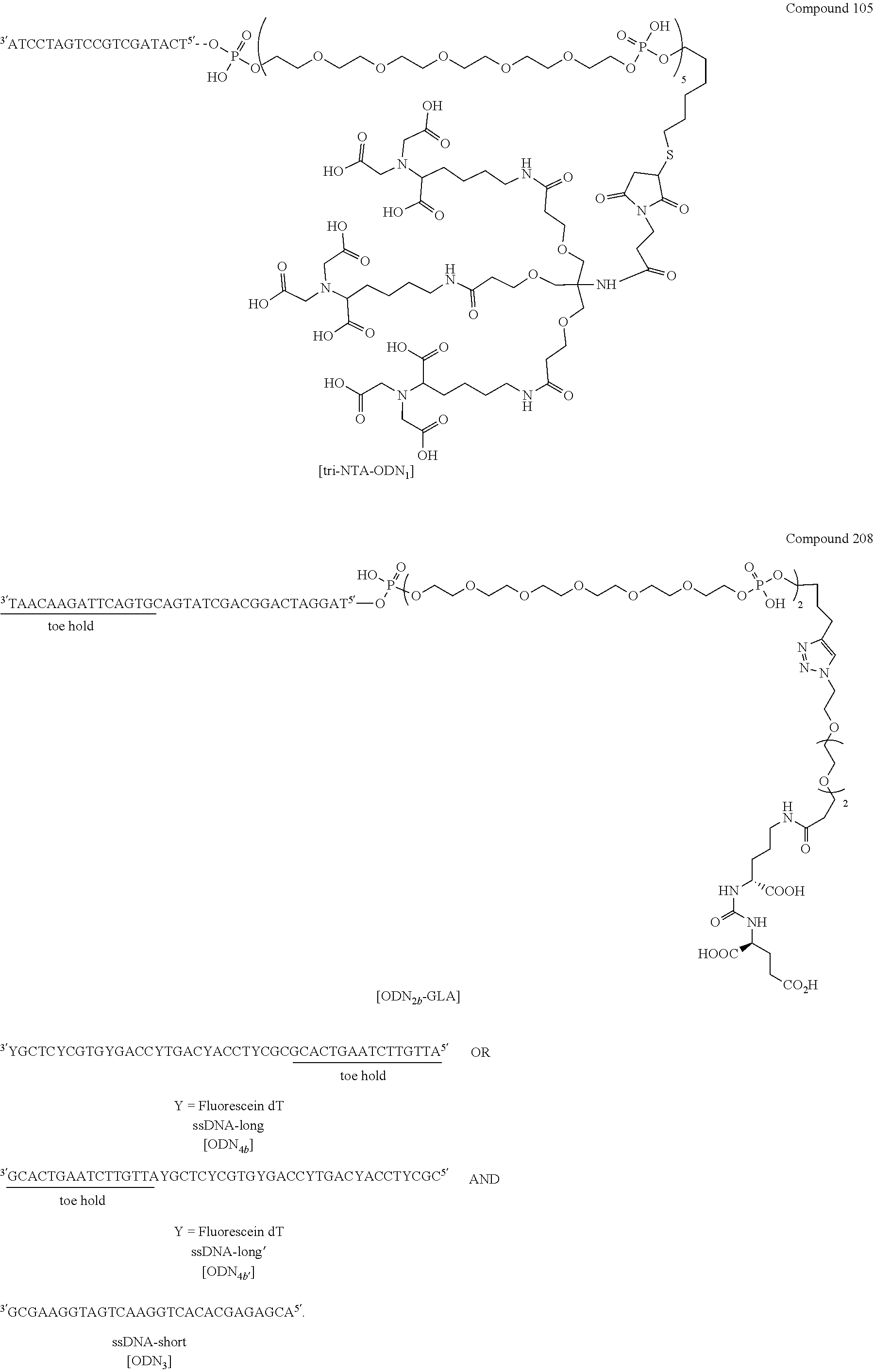

11. A system comprising:
a. a recombinant cell ectopically expressing a polypeptide, wherein said polypeptide comprises a membranal anchoring domain and an extracellular binding domain;
b. the DNA construct of claim 1;
wherein the first compound of said DNA construct is bound to the second compound through hybridization of ODN-1 and ODN-2, and
wherein the third compound of said DNA construct is bound to the second compound through hybridization of the first hanging strand and the second hanging strand;
wherein said His-tag specific binder of said DNA construct, comprises affinity to said extracellular binding domain of said polypeptide; and
wherein said DNA construct is bound to said recombinant cell in the presence of Ni²⁺ ions.

12. The system of claim 11,
wherein the system does not perturb said cell's function,
wherein said system can be reversibly modified,
wherein said recombinant cell is a bacteria,
wherein said polypeptide is a cell surface protein (CSP) comprising a histidine tag (e.g., His-OmpC),
wherein said extracellular binding domain of said polypeptide comprises a poly-histidine tag,
wherein said membranal anchoring domain of said polypeptide comprises a transmembranal protein or a part of it, an artificial polypeptide, or a combination thereof,
or any combination thereof.

13. The system of claim 12, wherein
said transmembranal protein comprises an outer membrane protein C (OmpC); receptor tyrosine kinases (RTKs); Ion channel linked receptors; Enzyme-linked receptors; G protein-coupled receptors or any combination thereof,
said bacteria is a His-OmpC expressing bacteria,
or combination thereof.

14. The system of claim 11, further comprising a fourth compound comprising a third oligonucleotide (ODN-3),
wherein said ODN-3 is complementary to said ODN-2, and/or
wherein said ODN-3 comprises higher affinity to said ODN-2 than the affinity of said ODN-2 to said ODN-1.

15. A recombinant cell bound to the DNA construct of claim 1,
said recombinant cell is ectopically expressing a polypeptide, which comprises a membranal anchoring domain and an extracellular binding domain, and
said extracellular binding domain comprises a poly-histidine affinity tag, which is bound to said DNA construct, via the binding of the His-tag specific binder to the poly histidine affinity tag of the polypeptide, in the presence of Ni²⁺ ions.

16. The recombinant cell of claim 15,
wherein the cell is a bacteria,
wherein said polypeptide is a cell surface protein (CSP),
wherein said polypeptide comprises an outer membrane protein C (OmpC); receptor tyrosine kinases (RTKs); Ion channel linked receptors; Enzyme-linked receptors; G protein-coupled receptors or any combination thereof,
wherein said membranal anchoring domain of said polypeptide comprises a transmembranal protein or a part of it, an artificial polypeptide, or a combination thereof,
or any combination thereof.

17. The recombinant cell of claim 16, wherein
said cell surface protein (CSP) is a histidine tagged outer membrane protein C (His-OmpC);
said bacteria is a His-OmpC expressing bacteria,
or combination thereof.

18. A method for labeling a cancer cell, said method comprises incubating the recombinant cell bound to the DNA construct of claim 15 with a cancer cell, wherein said cancer cell comprises a CSP, and the synthetic agent of said DNA construct of said recombinant cell, is a CSP binder, which comprises binding affinity to said CSP.

19. The method of claim 18,
wherein the labeling is carried out in a cellular environment;
wherein the CSP binder targets a small-molecule binding site in the cancer cell CSP;
wherein said recombinant cell is a native cell, a living cell or an engineered cell, preferably a bacteria;
wherein said CSP is overexpressed or selectively expressed in said cancer cell;
wherein the interaction between the recombinant cell and the cancer cell is multivalent;
or any combination thereof.

20. The method of claim 19,
wherein said CSP binder comprises a biotin, a folate, an anisamide, a glutamate urea, or derivative thereof;
wherein said cancer cell is KB cell (cervical cancer cell), MDA-MB-435 (melanoma cell), or LNCaP (prostate cancer cell);
or combination thereof.

21. A method for binding a first cell to a second cell, said method comprises incubating the cell of claim 15 (a first cell) with a second cell, wherein the second cell comprises a CSP, and said synthetic agent of said DNA construct of said first cell, comprises a CSP binder, which comprises a binding affinity to said CSP.

22. The method of claim 21,
wherein said first cell is a native cell, a living cell or an engineered cell, preferably a bacteria;
wherein said second cell is a cancer cell;
wherein said CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof;
wherein said CSP is selectively expressed or overexpressed in said second cell;
wherein said CSP binder comprises a biotin, a folate, an anisamide, a glutamate urea, an antibody or derivative thereof;
wherein the method is taking place in a cellular environment;
wherein the interaction between the first cell and the second cell is multivalent;
or any combination thereof.

23. The method of claim 22,
wherein said cancer cell is KB cell (cervical cancer cell), MDA-MB-435 (melanoma cell), or LNCaP (prostate cancer cell).

24. A method for binding a cell to a protein of interest (POI), said method comprises incubating a sample comprising a POI with the cell of claim 15, wherein said synthetic agent of said DNA construct is a protein binder, which comprises binding affinity to said POI.

25. The method of claim 24
wherein the method is taking place in a cellular environment;
wherein said POI is a cell surface protein (CSP);
wherein said protein binder is selective to said POI;
wherein said protein binder is a cell surface protein (CSP) binder, a small molecule ligand, an antibody, a peptide, a polypeptide, a protein or a part thereof,
or any combination thereof.

26. The method of claim 25
wherein said CSP is a polypeptide or a protein, which is overexpressed or selectively expressed on the surface of a cell, preferably a cancer cell;

27. A method for detecting and/or labeling a protein of interest (POI) in a cellular environment, said method comprises:
a. imaging a sample comprising a protein of interest (POI) in cellular environment;
b. incubating the sample of (a) with the cell of claim 15, wherein said synthetic agent of said DNA construct of said cell is a protein binder, which comprises affinity to said POI;
c. washing the sample of (b) from excess of said cell; and
d. imaging the fluorescence of said sample;
wherein increase in the fluorescence signal is indicative of the presence of said POI in said cellular environment, thereby detecting and/or labeling said protein of interest (POI) in said cellular environment.

28. The method of claim 27
wherein said cellular environment comprises living cells;
wherein said POI is a cell surface protein (CSP);
wherein said protein binder is selective to said POI;
wherein said fluorescence signal is measured by a fluorescence microscope or by recording the emission with a spectrophotometer at a particular wavelength;
or any combination thereof.

29. The method of claim 28
wherein said CSP is a polypeptide or a protein, which is overexpressed or selectively expressed on the surface of a cell, preferably a cancer cell; and/or
wherein said CSP is a G protein-coupled receptor (GPCR), Receptor tyrosine kinase (RTK), Programmed Cell Death protein 1 (PD-1), an Adhesion protein (e.g., Integrin), Antigenic protein (e.g., CD antigen) or derivative thereof.

30. A method for measuring the interaction between a protein of interest (POI) and a potential ligand for said POI, said method comprises
a. imaging a sample comprising a protein of interest (POI);
b. incubating the sample comprising a POI with the cell of claim 15 and with a potential ligand for said POI, wherein said synthetic agent of said DNA construct of said cell comprises affinity to said POI;
c. washing the sample from excess of said cell and ligand;
d. measuring the fluorescence imaging of said sample;
e. comparing the measured fluorescence of the sample of (d) with the fluorescence measured from incubating a control sample comprising a protein of interest (POI) with the cell of claim 15 followed by washing excess of said cell (i.e. a control);
wherein reduction in the fluorescence signal with respect to the control is indicative of the interaction between said POI and said potential ligand.

31. The method of claim 30,
wherein said POI is a cell surface protein (CSP);
wherein the potential ligand is a protein binder, a peptide, small molecule, modulator, agonist, antagonist, or any combination thereof,
wherein said synthetic agent is a protein binder,
wherein said potential ligand is added after, before or concurrently with said cell in step (b);
wherein said fluorescence signal is measured by a fluorescence microscope or by recording the emission with a spectrophotometer at a particular wavelength;
or any combination thereof.

32. The method of claim 31,
wherein said protein binder is selective to said POI.

33. A method for cell-based screening for potential ligands for a protein of interest (POI), said method comprises:
a. imaging a sample comprising a protein of interest (POI);
b. incubating the sample comprising a POI with the cell of claim 15 and with a potential ligand, wherein said synthetic agent of said DNA construct of said cell comprises affinity to said POI;
c. washing the sample from excess of said cell and ligand;
d. measuring the fluorescence imaging of said sample;
e. comparing the measured fluorescence of the sample of (d) with the fluorescence measured from incubating a sample comprising a protein of interest (POI) with the cell of claim 15 followed by washing excess of said cell (i.e. a control);
wherein reduction in the fluorescence signal with respect to the control is indicative of the interaction between said POI and said potential ligand thereby screening for potential ligands for said POI.

34. The method of claim 33,
wherein said cell-based screening is performed in living cells;
wherein said potential ligand is a protein binder, a peptide, small molecule, modulator, agonist or antagonist
wherein said synthetic agent is a protein binder, a drug, or a small molecule;
wherein said potential ligand is added after, before or concurrently with said cell in step (b);
wherein said fluorescence signal is measured by a fluorescence microscope or by recording the emission with a spectrophotometer at a particular wavelength;
or any combination thereof.

35. The method of claim 34,
wherein said protein binder, drug or small molecule is selective to said POI.


SEQUENCE LISTING (25 sequences)

SEQ ID NO: 1 - DNA, 13 bp, synthetic construct
  misc_feature 1..13: note = Description of sequence: ODNia/Compound 100/Compound 106
  source 1..13: mol_type = other DNA
gcggcgaggcagc

SEQ ID NO: 2 - DNA, 25 bp, synthetic construct
  misc_feature 1..25: note = Description of sequence: Compound 101
  source 1..25: mol_type = other DNA
gtcacgtcatagctgcctgatccta

SEQ ID NO: 3 - DNA, 19 bp, synthetic construct
  misc_feature 1..19: note = Description of sequence: ODN-1/ODN-i/Compound 102/Compound 105
  source 1..19: mol_type = other DNA
tcatagctgcctgatccta

SEQ ID NO: 4 - DNA, 25 bp, synthetic construct
  misc_feature 1..25: note = Description of sequence: Compound 103
  source 1..25: mol_type = other DNA
ggtacaactagacgatcgacagtag

SEQ ID NO: 5 - DNA, 25 bp, synthetic construct
  misc_feature 1..25: note = Description of sequence: Compound 104
  source 1..25: mol_type = other DNA
cgcaacgaaaaaaaaaaaagcgcgc

SEQ ID NO: 6 - DNA, 25 bp, synthetic construct
  misc_feature 1..25: note = Description of sequence: ODN2
  source 1..25: mol_type = other DNA
taggatcaggcagctatgacgtgac

SEQ ID NO: 7 - DNA, 35 bp, synthetic construct
  misc_feature 1..35: note = Description of sequence: Compound 208
  source 1..35: mol_type = other DNA
taggatcaggcagctatgacgtgacttagaacaat

SEQ ID NO: 8 - DNA, 25 bp, synthetic construct
  misc_feature 1..25: note = Description of sequence: Compound 203
  source 1..25: mol_type = other DNA
ctactgtcgatcgtctagttgtacc

SEQ ID NO: 9 - DNA, 25 bp, synthetic construct
  misc_feature 1..25: note = Description of sequence: Compound 204
  source 1..25: mol_type = other DNA
gcgcgcttttttttttttcgttgcg

SEQ ID NO: 10 - DNA, 25 bp, synthetic construct
  misc_feature 1..25: note = Description of sequence: ODN-3
  source 1..25: mol_type = other DNA
gtcacgtcatagctgcctgatccta

SEQ ID NO: 11 - DNA, 54 bp, synthetic construct
  misc_feature 1..54: note = Description of sequence: OmpC_FpET21
  source 1..54: mol_type = other DNA
tttgtttaactttaagaaggagatatacatatgaaagttaaagtactgtccctc

SEQ ID NO: 12 - DNA, 52 bp, synthetic construct
  misc_feature 1..52: note = Description of sequence: OmpC_RpET21
  source 1..52: mol_type = other DNA
ttcctttcgggctttgttagcagccggatcttagaactggtaaaccagaccc

SEQ ID NO: 13 - protein, 11 aa, synthetic construct
  REGION 1..11: note = Description of sequence: OmpC-(6His)1
SAGHHHHHHGT

SEQ ID NO: 14 - DNA, 48 bp, synthetic construct
  misc_feature 1..48: note = Description of sequence: OmpC_His1
  source 1..48: mol_type = other DNA
catcatcaccatggtacctctaaaggtaaaaacctgggtcgtggctac

SEQ ID NO: 15 - DNA, 60 bp, synthetic construct
  misc_feature 1..60: note = Description of sequence: OmpC_His1R
  source 1..60: mol_type = other DNA
atggtgatgatgatgatgacccgcggaggtaccatggtgatgatggtgatgacccgcgga

SEQ ID NO: 16 - protein, 22 aa, synthetic construct
  REGION 1..22: note = Description of sequence: OmpC-(6His)2
SAGHHHHHHGTSAGHHHHHHGT

SEQ ID NO: 17 - DNA, 40 bp, synthetic construct
  misc_feature 1..40: note = Description of sequence: OmpC_His2FInverse
  source 1..40: mol_type = other DNA
caccatcacggtacctctaaaggtaaaaacctgggtcgtg

SEQ ID NO: 18 - DNA, 42 bp, synthetic construct
  misc_feature 1..42: note = Description of sequence: OmpC_His2RInverse
  source 1..42: mol_type = other DNA
gtgatggtgacccgcggaggtaccatggtgatgatggtgatg

SEQ ID NO: 19 - DNA, 40 bp, synthetic construct
  misc_feature 1..40: note = Description of sequence: OmpC_His3FInverse
  source 1..40: mol_type = other DNA
catcatcatggtacctctaaaggtaaaaacctgggtcgtg

SEQ ID NO: 20 - DNA, 42 bp, synthetic construct
  misc_feature 1..42: note = Description of sequence: OmpC_His3RInverse
  source 1..42: mol_type = other DNA
atgatgatgacccgcggaggtaccgtgatggtggtgatggtg

SEQ ID NO: 21 - protein, 33 aa, synthetic construct
  REGION 1..33: note = Description of sequence: OmpC-(6His)3
SAGHHHHHHGTSAGHHHHHHGTSAGHHHHHHGT

SEQ ID NO: 22 - DNA, 45 bp, synthetic construct
  misc_feature 1..45: note = Description of sequence: ODN4/ssDNA-long/FAM6-ODN2b
  source 1..45: mol_type = other DNA
  modified_base 20: mod_base = OTHER; note = Fluorescein thymine
  modified_base 25: mod_base = OTHER; note = Fluorescein thymine
  modified_base 30: mod_base = OTHER; note = Fluorescein thymine
  modified_base 35: mod_base = OTHER; note = Fluorescein thymine
  modified_base 40: mod_base = OTHER; note = Fluorescein thymine
  modified_base 45: mod_base = OTHER; note = Fluorescein thymine
attgttctaagtcacgcgcttccatcagttccagtgtgctctcgt

SEQ ID NO: 23 - DNA, 45 bp, synthetic construct
  misc_feature 1..45: note = Description of sequence: ssDNA-long'
  source 1..45: mol_type = other DNA
  modified_base 4: mod_base = OTHER; note = Fluorescein thymine
  modified_base 9: mod_base = OTHER; note = Fluorescein thymine
  modified_base 14: mod_base = OTHER; note = Fluorescein thymine
  modified_base 19: mod_base = OTHER; note = Fluorescein thymine
  modified_base 24: mod_base = OTHER; note = Fluorescein thymine
  modified_base 29: mod_base = OTHER; note = Fluorescein thymine
cgcttccatcagttccagtgtgctctcgtattgttctaagtcacg

SEQ ID NO: 24 - DNA, 29 bp, synthetic construct
  misc_feature 1..29: note = Description of sequence: ODN3/ssDNA-short
  source 1..29: mol_type = other DNA
acgagagcacactggaactgatggaagcg

SEQ ID NO: 25 - DNA, 39 bp, synthetic construct
  misc_feature 1..39: note = Description of sequence: ODN-v/FAM6-ODN2/Compound 250
  source 1..39: mol_type = other DNA
  modified_base 14: mod_base = OTHER; note = Fluorescein thymine
  modified_base 19: mod_base = OTHER; note = Fluorescein thymine
  modified_base 24: mod_base = OTHER; note = Fluorescein thymine
  modified_base 29: mod_base = OTHER; note = Fluorescein thymine
  modified_base 34: mod_base = OTHER; note = Fluorescein thymine
  modified_base 39: mod_base = OTHER; note = Fluorescein thymine
gctgcctcgccgcttccatcagttccagtgtgctctcgt